(19) United States
(12) Patent Application Publication
(10) Pub. No.: US 2018/0023081 A1
(43) Pub. Date: Jan. 25, 2018

(54) LNA OLIGONUCLEOTIDES WITH ALTERNATING FLANKS

(71) Applicants: Bristol-Myers Squibb Company, Princeton, NJ (US); Roche Innovation Center Copenhagen A/S, Horsholm (DK)

(72) Inventors: Peter HAGEDORN, Horsholm (DK); Richard E. OLSON, Orange, CT (US); Anja Mølhart HØG, Hillerød (DK); Marianne Lerbech JENSEN, Køge (DK); Dong LI, East Lyme, CT (US); Niels Fisker NIELSEN, Kgs. Lyngby (DK)

(21) Appl. No.: 15/548,391

(22) PCT Filed: Feb. 4, 2016
(86) PCT No.: PCT/US2016/016657

§ 371 Date: Aug. 2, 2017

Related U.S. Application Data
(60) Provisional application No. 62/112,526, filed on Feb. 5, 2015.
(60) Provisional application No. 62/156,684, filed on May 4, 2015.
(60) Provisional application No. 62/237,922, filed on Oct. 6, 2015.
(60) Provisional application No. 62/238,941, filed on Oct. 8, 2015.
(60) Provisional application No. 62/279,614, filed on Jan. 15, 2016.

(51) Int. Cl. C12N 15/113

(52) U.S. Cl. CPC C12N 15/113; C12N 2310/3231; C12N 2310/343; C12N 2310/315; C12N 2310/351; C12N 2310/11; C12N 2310/346

(57) ABSTRACT

The present invention relates to LNA oligomers having two flanks, wherein one or both flanks comprise alternating LNA and DNA nucleosides.

### REFERENCE TO EARLIER FILED APPLICATIONS

This application is a non-provisional application claiming the benefit of U.S. Provisional Application No. 62/112,058, filed Feb. 4, 2015, U.S. Provisional Application No. 62/156,684, filed May 4, 2015, U.S. Provisional Application No. 62/237,922, filed Oct. 6, 2015, U.S. Provisional Application No. 62/238,941, filed Oct. 8, 2015, and U.S. Provisional Application No. 62/279,614, filed Jan. 15, 2016, all of which are incorporated herein by reference in their entireties.

### REFERENCE TO SEQUENCE LISTING SUBMITTED ELECTRONICALLY VIA EFS-WEB

The content of the electronically submitted sequence listing (Name: 3338_046PC05_SL.txt, Size: 245,908 bytes; and Date of Creation: Feb. 4, 2016) submitted in this application is incorporated herein by reference in its entirety.

### FIELD OF INVENTION

The present invention relates to LNA oligonucleotides (oligomers) having two flanks, wherein one or both flanks comprise both LNA and DNA nucleosides.

### BACKGROUND

Antisense oligonucleotides have been studied as potential drugs for diseases such as cancers (including lung cancer, colorectal carcinoma, pancreatic carcinoma, malignant glioma and malignant melanoma), diabetes, Amyotrophic lateral sclerosis (ALS), Duchenne muscular dystrophy and diseases such as asthma, arthritis and pouchitis with an inflammatory component. Antisense therapy can treat genetic disorders or infections when the genetic sequence of a particular gene is known to be associated with a particular disease. A strand of nucleic acid (DNA, RNA or a chemical analog) can bind to the corresponding sequence (e.g. mRNA, pre-mRNA, etc.) and modulate the gene expression (e.g. effectively turn the gene “off”).

While significant progress has been made, there remain challenges to antisense oligonucleotide development. One such challenge is off-target, non-specific binding of oligonucleotides, which can potentially lead to undesired toxicities in vivo. Therefore, there still is a need to develop improved oligomers with less toxicity and increased safety profiles.

### SUMMARY OF INVENTION

The present invention is based on the discovery that a particular design of an oligomer (e.g., DNA nucleosides within the wing regions of LNA gapmer oligonucleotides) can reduce a potential off-target binding (e.g., toxicity) of the oligomer while retaining the affinity (e.g., potency) to its intended target.

The invention provides for an oligomer of at least 10 contiguous nucleotides in length, comprising region A, region B, and region C (A-B-C), wherein region B comprises at least 5 consecutive nucleoside units and is flanked at 5′ by the A region of 1-8 contiguous nucleoside units and at 3′ by the C region of 1-8 contiguous nucleoside units, wherein the B region, when formed in a duplex with a complementary RNA, is capable of recruiting RNaseH, and wherein region A and region C are selected from the group consisting of:

(i) region A comprising a 5′ LNA nucleoside unit, a 3′ LNA nucleoside unit, and at least one DNA nucleoside unit between the 5′ LNA nucleoside unit and the 3′ LNA nucleoside unit, and region C comprising at least two 3′ LNA nucleoside units;
(ii) region A comprising at least one 5′ LNA nucleoside unit, and region C comprising a 5′ LNA nucleoside unit, at least two terminal 3′ LNA nucleoside units, and at least one DNA nucleoside unit between the 5′ LNA nucleoside unit and the 3′ LNA nucleoside units; and
(iii) region A comprising a 5′ LNA nucleoside unit, a 3′ LNA nucleoside unit, and at least one DNA nucleoside unit between the 5′ LNA nucleoside unit and the 3′ LNA nucleoside unit, and region C comprising a 5′ LNA nucleoside unit, at least two terminal 3′ LNA nucleoside units, and at least one DNA nucleoside unit between the 5′ LNA nucleoside unit and the 3′ LNA nucleoside units.

Also provided is a conjugate comprising the oligomer described herein covalently attached to at least one non-nucleotide or non-polynucleotide moiety.

Some embodiments of the application include a pharmaceutical composition comprising the oligomer or the conjugate and a pharmaceutically acceptable excipient, carrier, or diluent.

The invention provides for the use of the oligomer or the conjugate of the invention as a medicament for treatment of a disease or condition associated with the target of the oligomer or the oligomer or the conjugate for use in treating a disease or condition associated with the target of the oligomer.

In one embodiment, the invention provides for the oligomer or the conjugate of the invention as a medicament. In another embodiment, the invention provides for the oligomer or the conjugate of the invention for use in the treatment of a disease or condition.

In other embodiments, the invention provides for a method for inhibiting the expression of a RNA in a target cell which is expressing said target, said method comprising administering an oligomer, or a conjugate or the pharmaceutical composition of the invention in an effective amount to the cell. In some embodiments the method is in vivo. In some embodiments the method is in vitro.

In some embodiments, the oligomer or conjugate of the invention is administered via intracerebroventricular (ICV) administration. In some embodiments, the oligomer or conjugate of the invention is administered via intracerebral administration. In some embodiments, the oligomer or conjugate of the invention is administered via epidural administration. In some embodiments, the oligomer or conjugate of the invention is administered via intrathecal administration.

The invention provides for an in vivo or in vitro method for modulating the expression of a RNA target cell which is expressing said target, the method comprising administering an oligomer, or a conjugate or the pharmaceutical composition of the invention in an effective amount to said cell.

Certain embodiments of the application also include a method of reducing off-target binding of an oligomer comprising administering an oligomer disclosed herein, wherein the administering of the oligomer results in less hybridization to unintended targets compared to the administration of a corresponding oligomer with a different design (e.g., a design of a traditional gapmer (e.g. G-H-I described elsewhere herein or fully modified).

In some embodiments, the oligomer is from 10 to 50 nucleotides in length. In some embodiments, the oligomers of the invention comprise DNA and LNA nucleosides only, such as DNA and beta-D-oxy-nucleosides only. In some embodiments, the oligomers of the invention do not comprise a 2′ substituted nucleoside. In some embodiments, the oligomers of the invention do not comprise a 2′-O-MOE nucleoside.

### EMBODIMENTS

E1. An oligomer of at least 10 contiguous nucleotides in length, comprising region A, region B, and region C (A-B-C), wherein region B comprises at least 5 consecutive nucleoside units and is flanked at 5′ by region A of 1-8 contiguous nucleoside units and at 3′ by region C of 1-8 contiguous nucleoside units, wherein region B, when formed in a duplex with a complementary RNA, is capable of recruiting RNaseH, and wherein region A and region C are selected from the group consisting of:

(i) region A comprises a 5′ LNA nucleoside unit and a 3′ LNA nucleoside unit, and at least one DNA nucleoside unit between the 5′ LNA nucleoside unit and the 3′ LNA nucleoside unit, and, region C comprises at least two 3′ LNA nucleosides; or
(ii) region A comprises at least one 5′ LNA nucleoside and region C comprises a 5′ LNA nucleoside unit, at least two terminal 3′ LNA nucleoside units, and at least one DNA nucleoside unit between the 5′ LNA nucleoside unit and the 3′ LNA nucleoside units, and
(iii) region A comprises a 5′ LNA nucleoside unit and a 3′ LNA nucleoside unit, and at least one DNA nucleoside unit between the 5′ LNA nucleoside unit and the 3′ LNA nucleoside unit; and region C comprises a 5′ LNA nucleoside unit, at least two terminal 3′ LNA nucleoside units, and at least one DNA nucleoside unit between the 5′ LNA nucleoside unit and the 3′ LNA nucleoside units.

E2. The oligomer according to embodiment 1, wherein region A or region C comprises 1, 2, or 3 DNA nucleoside units.

E3. The oligomer according to embodiment 1, wherein region A and region C comprise 1, 2, or 3 DNA nucleoside units.

E4. The oligomer according to any one of embodiments 1-3, wherein region B comprises at least five consecutive DNA nucleoside units.

E5. The oligomer according to embodiment 4, wherein region B comprises 6, 7, 8, 9, 10, 11, 12, 13 or 14 consecutive DNA nucleoside units.

E6. The oligomer according to any one of embodiments 1-5, wherein region B is 8, 9 10, 11, or 12 nucleotides in length.

E7. The oligomer according to any one of embodiments 1-6, wherein region A comprises two 5′ terminal LNA nucleoside units.

E8. The oligomer according to any one of embodiments 1-7, wherein region A has formula 5′ [LNA]₁₋₃ [DNA]₁₋₃ [LNA]₁₋₃, or 5′[LNA]₁₋₂[DNA]₁₋₂[LNA]₁₋₂[DNA]₁₋₂[LNA]₁₋₂.

E9. The oligomer according to any one of embodiments 1-8 wherein region C has formula [LNA]₁₋₃[DNA]₁₋₃[LNA]₂₋₃3′, or [LNA]₁₋₂[DNA]₁₋₂[LNA]₁-[DNA]₁₋₂[LNA]₂₋₃3.

E10. The oligomer according to any one of embodiments 1-9, wherein region A has formula 5′ [LNA]₁₋₃ [DNA]₁₋₃ [LNA]₁₋₃, or 5′ [LNA]₁₋₂[DNA]₁₋₂[LNA]₁₋₂[DNA]₁₋₂[LNA]₁₋₂, and region C comprises 2, 3, 4 or 5 consecutive LNA nucleoside units.

E11. The oligomer according to any one of embodiments 1-9, wherein region C has formula [LNA]₁₋₃[DNA]₁₋₃[LNA]₂₋₃3′ or [LNA]₁₋₂[DNA]₁₋₂[LNA]₁₋₂[DNA]₁₋₂[LNA]₂₋₃3′, and region A comprises 1, 2, 3, 4 or 5 consecutive LNA nucleoside units.

E12. The oligomer according to any one of embodiments 1-11, wherein region A has a sequence of LNA and DNA nucleosides, 5′-3′ selected from the group consisting of L, LL, LDL, LLL, LLDL, LDLL, LDDL, LLLL, LLLLL, LLLDL, LLDLL, LDLLL, LLDDL, LDDLL, LLDLD, LDLLD, LDDDL, LLLLLL, LLLLDL, LLLDLL, LLDLLL, LDLLLL, LLLDDL, LLDLDL, LLDDLL, LDDLLL, LDLLDL, LDLDLL, LDDDLL, LLDDDL, and LDLDLD, wherein L represents a LNA nucleoside, and D represents a DNA nucleoside.

E13. The oligomer according to any one of embodiments 1-12, wherein region C has a sequence of LNA and DNA nucleosides, 5′-3′ selected from the group consisting of LL, LLL, LLLL, LDLL, LLLLL, LLDLL, LDLLL, LDDLL, LDDLLL, LLDDLL, LDLDLL, LDDDLL, LDLDDLL, LDDLDLL, LDDDLLL, and LLDLDLL.

E14. The oligomer according to any one of embodiments 1-13, wherein region A has a sequence of LNA and DNA nucleosides, 5′-3′ selected from the group consisting of LDL, LLDL, LDLL, LDDL, LLLDL, LLDLL, LDLLL, LLDDL, LDDLL, LLDLD, LDLLD, LDDDL, LLLLDL, LLLDLL, LLDLLL, LDLLLL, LLLDDL, LLDLDL, LLDDLL, LDDLLL, LDLLDL, LDLDLL, LDDDLL, LLDDDL, and LDLDLD, and region C has a sequence of LNA and DNA nucleosides, 5′-3′ selected from the group consisting of LDLL, LLLLL, LLDLL, LDLLL, LDDLL, LDDLLL, LLDDLL, LDLDLL, LDDDLL, LDLDDLL, LDDLDLL, LDDDLLL, and LLDLDLL.

E15. The oligomer according to any one of embodiments 1-11, wherein the contiguous nucleotides comprise a sequence of nucleosides, 5-3′, selected from the group consisting of LLDDDLLDDDDDDDDLL, LDLLDLDDDDDDDDDLL, LLLDDDDDDDDDDLDLL, LLLDDDDDDDDDLDDLL, LLLDDDDDDDDLDDDLL, LLLDDDDDDDDLDLDLL, LLLDLDDDDDDDDDLLL, LLLDLDDDDDDDDLDLL, LLLLDDDDDDDDDLDLL, LLLLDDDDDDDDLDDLL, LLLDDDLDDDDDDDDLL, LLLDDLDDDDDDDDDLL, LLLDDLLDDDDDDDDLL, LLLDDLLDDDDDDDLLL, LLLLLDDDDDDDLDDLL, LDLLLDDDDDDDDDDLL, LDLLLDDDDDDDLDDLL, LDLLLLDDDDDDDDDLL, LLDLLLDDDDDDDDDLL, LLLDLDDDDDDDDDDLL, LLLDLDDDDDDDLDDLL, LLLDLLDDDDDDDDDLL, LLLLDDDDDDDLDDDLL, LLLLLDDDDDDDDDLDLL, LLLLDDDDDDDDDDLDLL, LLLDDDDDDDDDDDLDLL, LLDLDDDDDDDDDDLDLL, LDLLLDDDDDDDDDLDLL, LLLDDDDDDDDDDLDDLL, LLLDDDDDDDDDLDDDLL, LLLDDDDDDDDLDLDDLL, LLLLDDDDDDDDDLDDLL, LLLLDDDDDDDDDLDLLL, LLLLDDDDDDDDLDDDLL, LLLLDDDDDDDDLDDLLL, LLLLDDDDDDDDLDLDLL, LLLLDDDDDDDLDDLDLL, LLLLDDDDDDDLDLDDLL, LLDLLDDDDDDDDDDDLL, LLDLLLDDDDDDDDLDLL, LLLDLDDDDDDDDDDDLL, LLLDLDDDDDDDDDLDLL, LLLDLDDDDDDDDLDDLL, LLLDLDDDDDDDLDLDLL, LLLLDDDDDDDDDLLDLL, LLLLLDDDDDDDDDLDLLL, LLLLLDDDDDDDDDLDDLL, LLLLDDDDDDDDDDLLDLL, LLLLDDDDDDDDDDLDLLL, LLLLDDDDDDDDDDLDDLL, LLLDDDDDDDDDDDLLDLL, LLLDDDDDDDDDDDLDLLL, LLLLLDDDDDDDDDLLDLL, LLLDDDDDDDDDDDLDDLL, LLDLLDDDDDDDDDLDDLL, LLLDLDDDDDDDDDDLDLL, LLLDLDDDDDDDDDLDDLL, LLLLDDDDDDDDDLDLDLL, LLLLDDDDDDDDLLDLDLL, LDLLLDDDDDDDDDDLLDLL, LLDLLDDDDDDDDDDLLDLL, LLDLDDDDDDDDDDDDLLLL, LLDDLDDDDDDDDDDDLLLL, LLLDLDDDDDDDDDDDLLLL, LLDLDDDDDDDDDDDDDLLL, LLDLLDDDDDDDDDDDLLLL, LLDDLDDDDDDDDDDDDLLL, LLLDDDDDDDDDDDLDDLLL, LLLDLDDDDDDDDDDDDLLL, LLDLLDDDDDDDDDDDDLLL, LLLLDDDDDDDDDDDLLDLL, LLLLDDDDDDDDDDLLDDLL, LLLDDLDDDDDDDDDLDLLL, LLDDLDLDDDDDDDDDLLLL, LLDDLLDDDDDDDDDLDLLL, LLLDLDDDDDDDDDLDLDLL, LLDLLDDDDDDDDDLDDLLL, LLLDLDDDDDDDDDDLDLLL, LLDLDDLDDDDDDDDDLLLL, LLLLDDDDDDDDDLDLDDLL, LLLDLDDDDDDDDDLDDLLL, LLDLDLDDDDDDDDDDLLLL, LLDLLDDDDDDDDDDLDLLL, LLDLDLDDDDDDDDDLLDLL, LLDDLLDDDDDDDDDLLDLL, LLLLDDDDDDDDDLDDLDLL, LLLDDLDDDDDDDDDLLDLL, LLDLLDDDDDDDDDLLDDLL, LLDLDLDDDDDDDDDLDLLL, LLLDLDDDDDDDDDLLDDLL, LLDDLLDDDDDDDDDDLLLL, LLDLLDDDDDDDDDLDLDLL, LLLLDDDDDDDDDDLDDLLL, LLLDDLDDDDDDDDDDLLLL, LLLDLDDDDDDDDDDLLDLL, LLLLDDDDDDDDDDLDLDLL, LLLLDDDDDDDDDDDLDLLL, and LLDDLLDDDDDDDDDDLDLL; wherein L represents a beta-D-oxy LNA nucleoside, and D represents a DNA nucleoside.

E16. The oligomer according to any one of embodiments 1-11, wherein the contiguous nucleotides comprise an alternating sequence of LNA and DNA nucleoside units, 5-3′, selected from the group consisting of: 2-3-2-8-2, 1-1-2-1-1-9-2, 3-10-1-1-2, 3-9-1-2-2, 3-8-1-3-2, 3-8-1-1-1-1-2, 3-1-1-9-3, 3-1-1-8-1-1-2, 4-9-1-1-2, 4-8-1-2-2, 3-3-1-8-2, 3-2-1-9-2, 3-2-2-8-2, 3-2-2-7-3, 5-7-1-2-2, 1-1-3-10-2, 1-1-3-7-1-2-2, 1-1-4-9-2, 2-1-3-9-2, 3-1-1-10-2, 3-1-1-7-1-2-2, 3-1-2-9-2, 4-7-1-3-2, 5-9-1-1-2, 4-10-1-1-2, 3-11-1-1-2, 2-1-1-10-1-1-2, 1-1-3-9-1-1-2, 3-10-1-2-2, 3-9-1-3-2, 3-8-1-1-1-2-2, 4-9-1-2-2, 4-9-1-1-3, 4-8-1-3-2, 4-8-1-2-3, 4-8-1-1-1-1-2, 4-7-1-2-1-1-2, 4-7-1-1-1-2-2, 2-1-2-11-2, 2-1-3-8-1-1-2, 3-1-1-11-2, 3-1-1-9-1-1-2, 3-1-1-8-1-2-2, 3-1-1-7-1-1-1-1-2, 4-9-2-1-2, 4-7-1-3-3, 5-9-1-1-3, 5-9-1-2-2, 4-10-2-1-2, 4-10-1-1-3, 4-10-1-2-2, 3-11-2-1-2, 3-11-1-1-3, 5-9-2-1-2, 3-11-1-2-2, 2-1-2-9-1-2-2, 3-1-1-10-1-1-2, 3-1-1-9-1-2-2, 4-9-1-1-1-1-2, 4-8-2-1-1-1-2, 1-1-3-10-2-1-2, 2-1-2-10-2-1-2, 2-1-1-12-4, 2-2-1-11-4, 3-1-1-11-4, 2-1-1-13-3, 2-1-2-11-4, 2-2-1-12-3, 3-11-1-2-3, 3-1-1-12-3, 2-1-2-12-3, 4-11-2-1-2, 4-10-2-2-2, 3-2-1-9-1-1-3, 2-2-1-1-1-9-4, 2-2-2-9-1-1-3, 3-1-1-9-1-1-1-1-2, 2-1-2-9-1-2-3, 3-1-1-10-1-1-3, 2-1-1-2-1-9-4, 4-9-1-1-1-2-2, 3-1-1-9-1-2-3, 2-1-1-1-1-10-4, 2-1-2-10-1-1-3, 2-1-1-1-1-9-2-1-2, 2-2-2-9-2-1-2, 4-9-1-2-1-1-2, 3-2-1-9-2-1-2, 2-1-2-9-2-2-2, 2-1-1-1-1-9-1-1-3, 3-1-1-9-2-2-2, 2-2-2-10-4, 2-1-2-9-1-1-1-1-2, 4-10-1-2-3, 3-2-1-10-4, 3-1-1-10-2-1-2, 4-10-1-1-1-1-2, 4-11-1-1-3, and 2-2-2-10-1-1-2; wherein the first numeral represents an number of LNA units, the next a number of DNA units, and alternating LNA and DNA regions thereafter.

E17. The oligomer according to any one of embodiments 1-16, wherein the maximum number of contiguous LNA nucleosides in regions A and C is 2 or 3.

E18. The oligomer according to any one of embodiments 1-17, wherein the maximum number of contiguous DNA nucleosides in regions A and C is 1 or 2.

E19. The oligomer according to any one of embodiments 1-18, wherein the LNA nucleoside units present in regions A and C are beta-D LNA units, such as beta-D-oxy LNA nucleoside units or (S)cEt nucleoside LNA units.

E20. The oligomer according to any one of embodiments 1-18, wherein the LNA nucleoside units present in regions A and C are beta-D-oxy LNA nucleoside units.

E21. The oligomer according to any one of embodiments 1-20, wherein the oligomer comprises at least one phosphorothioate internucleoside linkage.

E22. The oligomer according to any one of embodiments 1-20, wherein the internucleoside linkages within region B are phosphorothioate internucleoside linkages.

E23. The oligomer according to any one of embodiments 1-20, wherein all the internucleoside linkages within regions A, B and C are phosphorothioate internucleoside linkages.

E24. A conjugate comprising the oligomer according to any one 1-23 covalently attached to at least one non-nucleotide or non-polynucleotide moiety.

E25. The conjugate according to embodiment 24, wherein the at least one non-nucleotide or non-polynucleotide moiety is selected from the group consisting of proteins, peptides, glycoproteins, antibodies, antibody binding domains, fatty acids, sterols, sugar residues, a GalNAc conjugate such as a trivalent GalNAc cluster, polymers, PEG, and two or more combinations thereof.

E26. The conjugate according to embodiment 24 or 25, wherein the at least one non-nucleotide or non-polynucleotide moiety is covalently attached to the oligomer via a biocleavable linker, such as a region of 1, 2, 3, 4 or 5 phosphodiester linked DNA nucleotides.

E27. A pharmaceutical composition comprising the oligomer of any one of embodiments 1-22, or the conjugate of any one of embodiments 24-26, and a pharmaceutically acceptable excipient, carrier, or diluent.

E28. Use of the oligomer of any one of embodiments 1-23, or the conjugate of any one of embodiments 24-26 for the manufacture of a medicament in treating a disease or condition.

E29. An in vivo or in vitro method for modulating the expression of an RNA in a cell which expresses said RNA, said method comprising contacting an effective amount of the oligomer of any one of embodiments 1-23, the conjugate of any one of embodiments 24-26 or the pharmaceutical composition of embodiment 27 with said cell.

E30. A method of producing an oligomer having a reduced non-specific binding comprising synthesizing the oligomer of any one of embodiments 1 to 23.

E31. A method of treating a disease or condition in a subject in need thereof while reducing the toxicity of the treatment comprising administering the oligomer of any one of embodiments 1-23, the conjugate of any one of embodiments 24-26, or the pharmaceutical composition of embodiment 27 to the subject, wherein the administering treats the disease or condition while reducing the toxicity of the treatment compared to a treatment using a corresponding reference oligomer, which reference oligomer comprises formula G-H-I, wherein region H is identical to region B, and regions G and I comprise all LNA nucleoside units.

E32. The oligomer of any one of embodiments 1 to 23 for use in treating a neurological disease or condition in a subject in need thereof, wherein calcium oscillations in neuronal cells that are in contact with the oligomer are about 70% or higher, about 75% or higher, about 80% or higher, about 85% or higher, about 90% or higher, about 95% or higher, about 96% or higher, about 97% or higher, about 98% or higher, about 99% or higher, about 100% or higher, about 120% or higher, about 140% or higher, about 160% or higher, about 180% or higher, about 200% or higher, about 220% or higher, about 240% or higher, or about 250% or higher compared to the calcium oscillations in vehicle control cells.

E33. The oligomer of any one of embodiments 1 to 23 for use in treating a neurological disease or condition having the sequence score greater than or equal to 0.2, greater than or equal to 0.25, greater than or equal to 0.3, greater than or equal to 0.35, greater than or equal to 0.4, greater than or equal to 0.45, greater than or equal to 0.5, greater than or equal to 0.55, greater than or equal to 0.6, greater than or equal to 0.65, greater than or equal to 0.7, greater than or equal to 0.75, greater than or equal to 0.8, greater than or equal to 0.85, greater than or equal to 0.9, greater than or equal to 0.95, greater than or equal to 1.0, greater than or equal to 1.5, greater than or equal to 2.0, greater than or equal to 3.0, or greater than or equal to 4.0.

### BRIEF DESCRIPTION OF FIGURES

FIG. 1 shows exemplary oligomers, designs (ASO Sequence), and chemical structure of the oligomers. FIG. 1 lists the oligomer name, antisense oligomer (ASO) identification number, ASO sequence, SEQ ID Number, target start and end positions on the MAPT pre-mRNA sequence and chemical structure.

FIG. 2 shows exemplary oligomers targeting nucleotides 134,947 to 138,940 of SEQ ID NO: 1. FIG. 2 lists the SEQ ID number, oligomer name, ASO identification number, ASO sequence, target start and end positions on the MAPT pre-mRNA sequence, target start on the mature mRNA sequence and normalized Tau/Tuj-1 and Tuj-1 immunocytochemistry values (as discussed in Example 2 below).

FIG. 3 shows the impact of Tau antisense oligonucleotides on spontaneous calcium oscillations in primary neurons and IC₅₀ values of Tau neurons. FIG. 3 lists the ASO identification number, ASO sequence, SEQ ID Number, target start and end positions on the MAPT pre-mRNA sequence, calcium oscillation data as a percent of control (as discussed in Example 3 below) and IC₅₀ values of Tau neurons (as also discussed in Example 3 below).

FIG. 4 shows the in vivo tolerability of exemplary oligomers and brain tau mRNA reduction. FIG. 4 lists the ASO identification number, ASO sequence, SEQ ID Number, target start and end positions on the MAPT pre-mRNA sequence, in vivo acute tolerability score (as discussed in Example 5 below) and the percent of brain MAPT mRNA remaining after administration (as also discussed in Example 5 below).

FIG. 5 shows the composition of the sequence of the selected oligomer and the sequence of Rho A which aligns with a portion of the MAPT genomic sequence (SEQ ID NO: 1). The RhoA sequence is listed as actttatttccaaatacacttcttt (SEQ ID NO: 267). The mismatches between the selected oligomers and the Rho A sequence were highlighted. The sequence of ASO-000757 has one mismatch compared to the corresponding RhoA sequence; The sequences of ASO-0001967, ASO-000755, and ASO-001941 have two mismatches compared to the corresponding RhoA sequence; and The sequeces of ASO-000753, ASO-002038, ASO-001933, and ASO-001940 have four mismatches compared to the corresponding RhoA sequence. FIG. 5 shows that the traditional gapmers (i.e., ASO-000757, ASO-000755, and ASO-000753) are not tolerated beyond 4 weeks following a single 100 μg ICV bolus dose while the alternating flank gapmers exhibit tolerability beyond 4 weeks. Tubulin inhibition was highly correlated, in this data set, to long term tolerability. Rho A reduction greater than 25% (i.e., ASO-000757, ASO-000755, and ASO-000753) was also correlated with lack of long term tolerability (greater than 4 weeks following a single ICV bolus injection of 100 μg of each ASO shown).

FIGS. 6A and 6B show exemplary oligomers, designs, and their chemical structures. FIG. 6A lists the antisense oligomer (ASO) identification number, SEQ ID Number, ASO sequence, target start and end positions on the Tau pre-mRNA sequence, IC₅₀ values of Tau neurons (as discussed in Example 8 below) and percent Tau inhibition (as also discussed in Example 8 below). FIG. 6B shows the specific chemical structure of the oligomers shown in FIG. 6A and lists the antisense oligomer (ASO) identification number, ASO sequence, target start and end positions on the Tau pre-mRNA sequence and chemical structure.

FIG. 7 shows that an exemplary alternating flank oligomer, e.g., ASO-001933, produces dose responsive brain hTau protein reduction after a single ICV injection in hTau mouse brain. Saline or 50, 100, 150 or 200 μg of Tau ASO was injected ICV in hTau mice (n=10 per group). X-axis shows the doses of ASO-001933, and the Y-axis shows the hTau protein expression after the ASO injection compared to the hTau protein expression after the saline injection (% of saline).

FIG. 8A is an image of brain regions showing pathologic Tau accumulation in PSP.

FIG. 8B shows regional Tau mRNA changes in a control monkey (left) or in a monkey that had received two single 16 mg intrathecal bolus doses of ASO-001933, one week apart (right). The Tau mRNA changes were assessed two weeks post-dosing by fluorescence in situ hybridization (FISH) using Tau mRNA specific probes in substantia nigra, pontine nucleus and central cerebellar dentate nucleus. Tau mRNA accumulation is shown as lighter shades.

FIG. 9A shows Tau protein reduction in brain following intrathecal dosing of ASO-001933 in nonhuman primates (NHPs). Regional Tau mRNA changes in a control monkey or a monkey that had received two single 8 mg intrathecal bolus doses of ASO-001933, two weeks apart, were assessed 4, 8, or 12 weeks post-dosing by Tau ELISAs (BT2/HT7 or Tau12/BT2). The regional Tau mRNA changes were measured in pons, cerebellum (CBL), parietal cortex (ParC), frontal cortex (FrC), occipital cortex (OccC), temporal cortex (TemC), and hippocampus (Hipp).

FIG. 9B shows Tau protein reduction in cerebrospinal fluid (CSF) following intrathecal dosing of ASO-001933 in nonhuman primates (NHPs). Y-axis shows percent baseline of Tau protein reduction in CSF, and X-axis shows weeks post last dose.

FIG. 10A shows that ASO-002038 (Tau ASO) produces durable, dose responsive brain hTau mRNA reduction after a single intracerebroventricular (ICV) injection in hTau mouse brain. Saline or 25, 50, 100, and 150 μg of Tau ASO was injected ICV in hTau mice (n=10 per group). The frontal cortical region was dissected 1 week post dose to determine total Tau mRNA levels by qRT-PCR. 1-way ANOVA analysis was used ***p<0.001. Error bars represent mean+/−SEM.

FIG. 10B shows that ASO-002038 (Tau ASO) produces durable, dose responsive brain hTau mRNA reduction after a single intrathecal (IT) injection in surgical lumbar catheterized rats. Saline or 400, 900, and 1500 μg of Tau ASO was injected IT in rats (n=10 per group). The frontal cortical region was dissected 1 week post dose to determine total Tau mRNA levels by qRT-PCR. 1-way ANOVA analysis was used ***p<0.001. Error bars represent mean+/−SEM.

FIGS. 11A and 11B show exemplary oligomers, designs, and chemical structures tested by QUANTIGENE® analysis. FIG. 11A lists the antisense oligomer (ASO) identification number, SEQ ID Number, ASO sequence, target start and end positions on the Tau pre-mRNA sequence, start position on the mature mRNA sequence, and QUANTIGENE® expression of mRNA (as discussed in Example 10 below). FIG. 11B shows the specific chemical structure of the oligomers shown in FIG. 11A and lists the antisense oligomer (ASO) identification number, ASO sequence, target start and end positions on the Tau pre-mRNA sequence and chemical structure.

### DETAILED DESCRIPTION OF INVENTION

### I. Definitions

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. Unless otherwise indicated, nucleotide sequences are written left to right in 5′ to 3′ orientation. Amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower). For example, if it is stated that “the oligomer reduces expression of the target protein in a cell following administration of the oligomer by at least about 60%,” it is implied that the target protein levels are reduced by a range of 50% to 70%.

The term “nucleic acids” or “nucleotides” is intended to encompass plural nucleic acids. In some embodiments, the term “nucleic acids” or “nucleotides” refers to a target sequence, e.g., pre-mRNAs, mRNAs, or DNAs in vivo or in vitro. When the term refers to the nucleic acids or nucleotides in a target sequence, the nucleic acids or nucleotides can be naturally occurring sequences within a cell. In other embodiments, “nucleic acids” or “nucleotides” refers to a sequence in the oligomers of the invention. When the term refers to a sequence in the oligomers, the nucleic acids or nucleotides are not naturally occurring. In one embodiment, the nucleic acids or nucleotides in the oligomers are produced synthetically or recombinantly, but are not a naturally occurring sequence or a fragment thereof. In another embodiment, the nucleic acids or nucleotides in the oligomers contain at least one nucleotide analog that is not naturally occurring in nature. The term “nucleic acid” or “nucleoside” refers to a single nucleic acid segment, e.g., a DNA, an RNA, or an analog thereof, present in a polynucleotide. “Nucleic acid” or “nucleoside” includes naturally occurring nucleic acids or non-naturally occurring nucleic acids. In some embodiments, the terms “nucleotide”, “unit” and “monomer” are used interchangeably. It will be recognized that when referring to a sequence of nucleotides or monomers, what is referred to is the sequence of bases, such as A, T, G, C or U, and analogs thereof.

The term “nucleotide” as used herein, refers to a glycoside comprising a sugar moiety, a base moiety and a covalently linked group (linkage group), such as a phosphate or phosphorothioate internucleotide linkage group, and covers both naturally occurring nucleotides, such as DNA or RNA, and non-naturally occurring nucleotides comprising modified sugar and/or base moieties, which are also referred to as “nucleotide analogs” herein. Herein, a single nucleotide (unit) can also be referred to as a monomer or nucleic acid unit. In certain embodiments, the term “nucleotide analogs” refers to nucleotides having modified sugar moieties. Non-limiting examples of the nucleotides having modified sugar moieties (e.g., LNA) are disclosed elsewhere herein, e.g., 2′-O-methyl, 2′-fluoro (2′-F), 2′-O-methoxyethyl (2′-MOE), and 2′,4′-constrained 2′-O-ethyl (cEt). In other embodiments, the term “nucleotide analogs” refers to nucleotides having modified base moieties. The nucleotides having modified base moieties include, but are not limited to, 5-methylcytosine, isocytosine, pseudoisocytosine, 5-bromouracil, 5-propynyluracil, 6-aminopurine, 2-aminopurine, inosine, diaminopurine, and 2-chloro-6-aminopurine.

The term “nucleoside” as used herein is used to refer to a glycoside comprising a sugar moiety and a base moiety, and can therefore be used when referring to the nucleotide units, which are covalently linked by the internucleotide linkages between the nucleotides of the oligomer. In the field of biotechnology, the term “nucleotide” is often used to refer to a nucleic acid monomer or unit, and as such in the context of an oligonucleotide can refer to the base—such as the “nucleotide sequence”, typically refer to the nucleobase sequence (i.e. the presence of the sugar backbone and internucleoside linkages are implicit). Likewise, particularly in the case of oligonucleotides where one or more of the internucleoside linkage groups are modified, the term “nucleotide” can refer to a “nucleoside” for example the term “nucleotide” can be used, even when specifying the presence or nature of the linkages between the nucleosides.

The term “nucleotide length” as used herein means the total number of the nucleotides (monomers) in a given sequence. For example, the sequence of AtTTCcaaattcactTTtAC (SEQ ID NO: 91) has 20 nucleotides; thus the nucleotide length of the sequence is 20. The term “nucleotide length” is therefore used herein interchangeably with “nucleotide number.”

As one of ordinary skill in the art would recognize, the 5′ terminal nucleotide of an oligonucleotide does not comprise a 5′ internucleotide linkage group, although it may or may not comprise a 5′ terminal group.

As used herein, a “coding region” or “coding sequence” is a portion of polynucleotide which consists of codons translatable into amino acids. Although a “stop codon” (TAG, TGA, or TAA) is typically not translated into an amino acid, it can be considered to be part of a coding region, but any flanking sequences, for example promoters, ribosome binding sites, transcriptional terminators, introns, untranslated regions (“UTRs”), and the like, are not part of a coding region. The boundaries of a coding region are typically determined by a start codon at the 5′ terminus, encoding the amino terminus of the resultant polypeptide, and a translation stop codon at the 3′ terminus, encoding the carboxyl terminus of the resulting polypeptide.

The term “non-coding region” as used herein means a nucleotide sequence that is not a coding region. Examples of non-coding regions include, but are not limited to, promoters, ribosome binding sites, transcriptional terminators, introns, untranslated regions (“UTRs”), non-coding exons and the like. Some of the exons can be wholly or part of the 5′ untranslated region (5′ UTR) or the 3′ untranslated region (3′ UTR) of each transcript. The untranslated regions are important for efficient translation of the transcript and for controlling the rate of translation and half-life of the transcript.

The term “region” when used in the context of a nucleotide sequence refers to a section of that sequence. For example, the phrase “region within a nucleotide sequence” or “region within the complement of a nucleotide sequence” refers to a sequence shorter than the nucleotide sequence, but longer than at least 10 nucleotides located within the particular nucleotide sequence or the complement of the nucleotides sequence, respectively. The term “sub-sequence” or “subsequence” can also refer to a region of a nucleotide sequence.

The term “downstream,” when referring to a nucleotide sequence, means that a nucleic acid or a nucleotide sequence is located 3′ to a reference nucleotide sequence. In certain embodiments, downstream nucleotide sequences relate to sequences that follow the starting point of transcription. For example, the translation initiation codon of a gene is located downstream of the start site of transcription.

The term “upstream” refers to a nucleotide sequence that is located 5′ to a reference nucleotide sequence.

As used herein, the term “regulatory region” refers to nucleotide sequences located upstream (5′ non-coding sequences), within, or downstream (3′ non-coding sequences) of a coding region, and which influence the transcription, RNA processing, stability, or translation of the associated coding region. Regulatory regions can include promoters, translation leader sequences, introns, polyadenylation recognition sequences, RNA processing sites, effector binding sites, UTRs, and stem-loop structures. If a coding region is intended for expression in a eukaryotic cell, a polyadenylation signal and transcription termination sequence will usually be located 3′ to the coding sequence.

The term “transcript” as used herein can refer to a primary transcript that is synthesized by transcription of DNA and becomes a messenger RNA (mRNA) after processing, i.e., a precursor messenger RNA (pre-mRNA), and the processed mRNA itself. The term “transcript” can be interchangeably used with “pre-mRNA” and “mRNA.” After DNA strands are transcribed to primary transcripts, the newly synthesized primary transcripts are modified in several ways to be converted to their mature, functional forms to produce different proteins and RNAs such as mRNA, tRNA, rRNA, lncRNA, miRNA and others. Thus, the term “transcript” can include exons, introns, 5′ UTRs, and 3′ UTRs.

The term “expression” as used herein refers to a process by which a polynucleotide produces a gene product, for example, a RNA or a polypeptide. It includes, without limitation, transcription of the polynucleotide into messenger RNA (mRNA) and the translation of an mRNA into a polypeptide. Expression produces a “gene product.” As used herein, a gene product can be either a nucleic acid, e.g., a messenger RNA produced by transcription of a gene, or a polypeptide which is translated from a transcript. Gene products described herein further include nucleic acids with post transcriptional modifications, e.g., polyadenylation or splicing, or polypeptides with post translational modifications, e.g., methylation, glycosylation, the addition of lipids, association with other protein subunits, or proteolytic cleavage.

The terms “identical” or percent “identity” in the context of two or more nucleic acids refer to two or more sequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid or nucleotide sequences.

One such non-limiting example of a sequence alignment algorithm is the algorithm described in Karlin et al., 1990, Proc. Natl. Acad. Sci., 87:2264-2268, as modified in Karlin et al., 1993, Proc. Natl. Acad. Sci., 90:5873-5877, and incorporated into the NBLAST and XBLAST programs (Altschul et al., 1991, Nucleic Acids Res., 25:3389-3402). In certain embodiments, Gapped BLAST can be used as described in Altschul et al., 1997, Nucleic Acids Res. 25:3389-3402. BLAST-2, WU-BLAST-2 (Altschul et al., 1996, Methods in Enzymology, 266:460-480), ALIGN, ALIGN-2 (Genentech, South San Francisco, Calif.) or Megalign (DNASTAR) are additional publicly available software programs that can be used to align sequences. In certain embodiments, the percent identity between two nucleotide sequences is determined using the GAP program in the GCG software package (e.g., using a NWSgapdna.CMP matrix and a gap weight of 40, 50, 60, 70, or 90 and a length weight of 1, 2, 3, 4, 5, or 6). In certain alternative embodiments, the GAP program in the GCG software package, which incorporates the algorithm of Needleman and Wunsch (J. Mol. Biol. (48):444-453 (1970)) can be used to determine the percent identity between two amino acid sequences (e.g., using either a BLOSUM 62 matrix or a PAM250 matrix, and a gap weight of 16, 14, 12, 10, 8, 6, or 4 and a length weight of 1, 2, 3, 4, 5). Alternatively, in certain embodiments, the percent identity between nucleotide or amino acid sequences is determined using the algorithm of Myers and Miller (CABIOS, 4:11-17 (1989)). For example, the percent identity can be determined using the ALIGN program (version 2.0) and using a PAM120 with residue table, a gap length penalty of 12 and a gap penalty of 4. One skilled in the art can determine appropriate parameters for maximal alignment by particular alignment software. In certain embodiments, the default parameters of the alignment software are used.

In certain embodiments, the percentage identity “X” of a first nucleotide sequence to a second nucleotide sequence is calculated as 100×(Y/Z), where Y is the number of amino acid residues scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

Different regions within a single polynucleotide target sequence that align with a polynucleotide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

As used herein, the terms “homologous” and “homology” are interchangeable with the terms “identity” and “identical.”

In determining the degree of “complementarity” between oligomers of the invention (or regions thereof) and the target region of the target nucleic acid, the degree of “complementarity” (also, “homology” or “identity”) is expressed as the percentage identity (or percentage homology) between the sequence of the oligomer (or region thereof) and the sequence of the target region (or the reverse complement of the target region) that best aligns therewith. The percentage is calculated by counting the number of aligned bases that are identical between the two sequences, dividing by the total number of contiguous monomers in the oligomer, and multiplying by 100. In such a comparison, if gaps exist, it is preferable that such gaps are merely mismatches rather than areas where the number of monomers within the gap differs between the oligomer of the invention and the target region.

The term “complement” as used herein indicates a sequence that is complementary to a reference sequence. It is well known that complementarity is the base principle of DNA replication and transcription as it is a property shared between two DNA or RNA sequences, such that when they are aligned antiparallel to each other, the nucleotide bases at each position in the sequences will be complementary, much like looking in the mirror and seeing the reverse of things. Therefore, for example, the complement of a sequence of 5′ “ATGC” 3′ can be written as 3′ “TACG” 5′ or 5′ “GCAT” 3′. The terms “reverse complement”, “reverse complementary” and “reverse complementarity” as used herein are interchangeable with the terms “complement”, “complementary” and “complementarity.”

The term “design” or “oligomer design” or “ASO Sequence” as used herein refers to a pattern of nucleotides (e.g., DNA) and nucleotide analogs (e.g., LNA) in a given sequence. As used herein, the design of an oligomer is shown by a combination of upper case letters and lower case letters. For example, an oligomer sequence of tatttccaaattcactttta (SEQ ID NO: 337) can have oligomer designs of ASO-002350 (TAtTTccaaattcactTTTA) (SEQ ID NO: 141), ASO-002374 (TAtTTccaaattcacTtTTA) (SEQ ID NO: 142), ASO-002386 (TATTtccaaattcaCTttTA) (SEQ ID NO: 143), ASO-002227 (TATtTccaaattcactTTTA) (SEQ ID NO: 144), ASO-002245 (TAttTCcaaattcactTTTA) (SEQ ID NO: 145), ASO-002261 (TATtTccaaattcacTTtTA) (SEQ ID NO: 146), ASO-002276 (ATttCcaaattcactTTTA) (SEQ ID NO: 147), ASO-002228 (TATTtccaaattcaCtTtTA) (SEQ ID NO: 148), ASO-002255 (TATTtccaaattcactTTTA) (SEQ ID NO: 338), ASO-002285 (TATTtccaaattcacTTtTA) (SEQ ID NO: 149), ASO-002230 (TATTtccaaattcacTtTTA) (SEQ ID NO: 150), ASO-002256 (TATTtccaaattcAcTttTA) (SEQ ID NO: 151), or ASO-002279 (TATTtccaaattcActTtTA) (SEQ ID NO: 152), wherein the upper case letter indicates a nucleotide analog (e.g., LNA) and the lower case letter indicates a nucleotide (e.g., DNA)

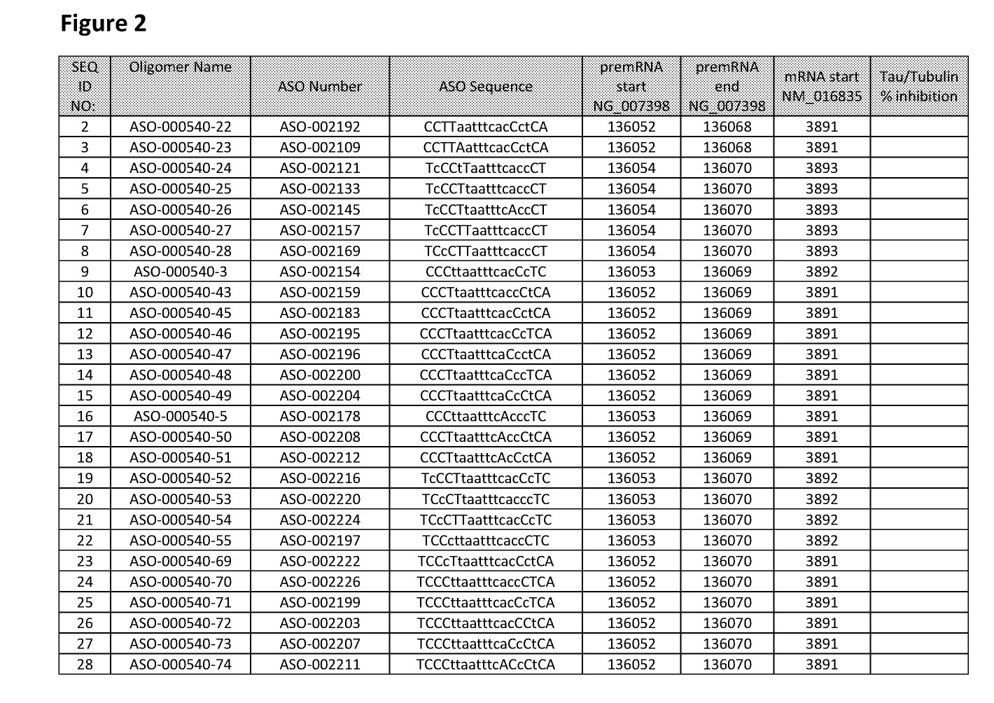

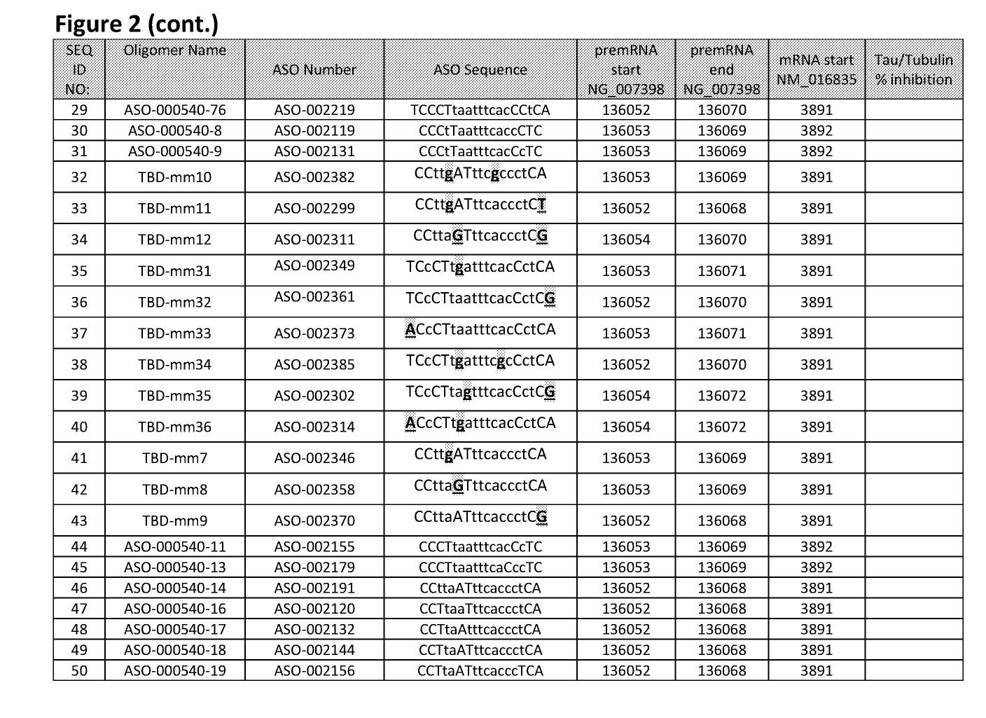

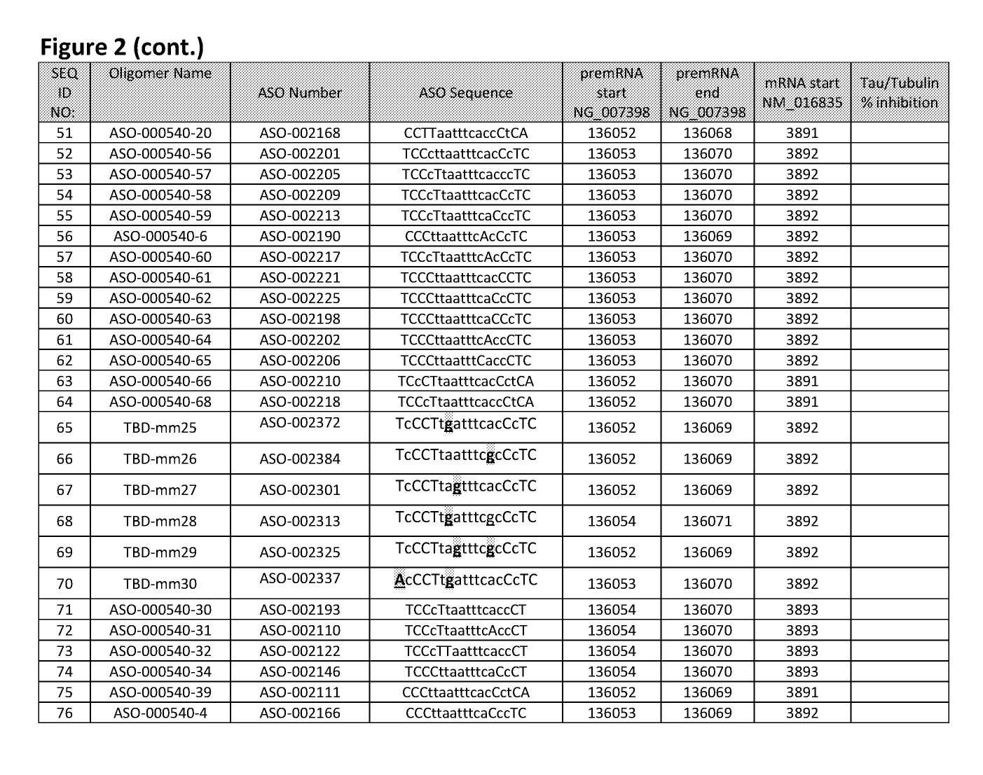

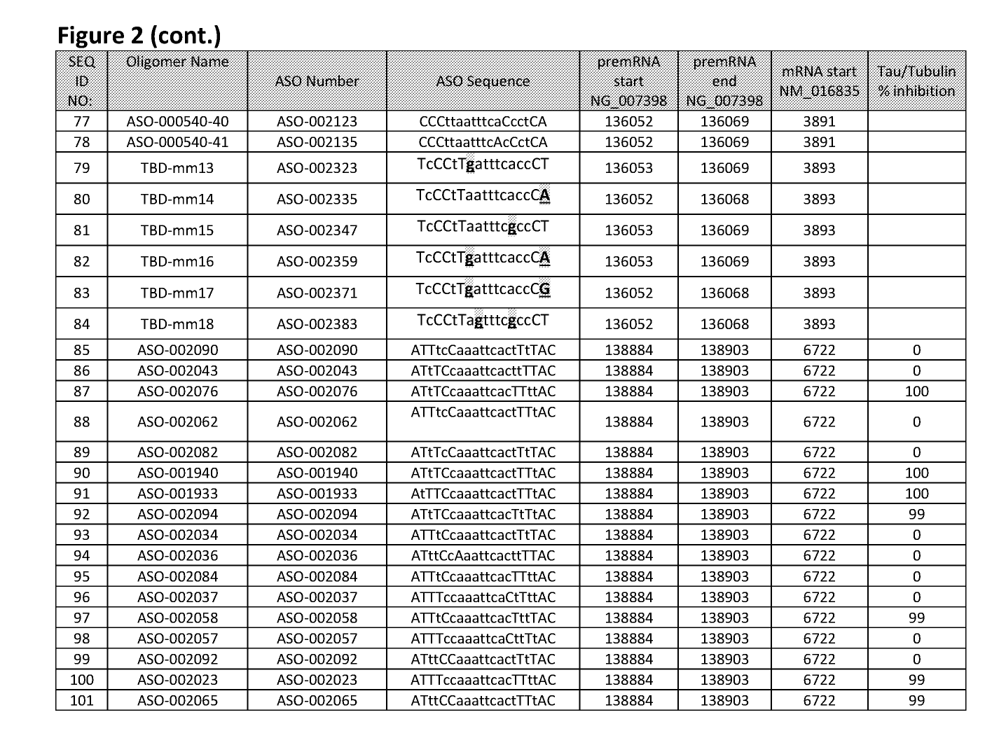

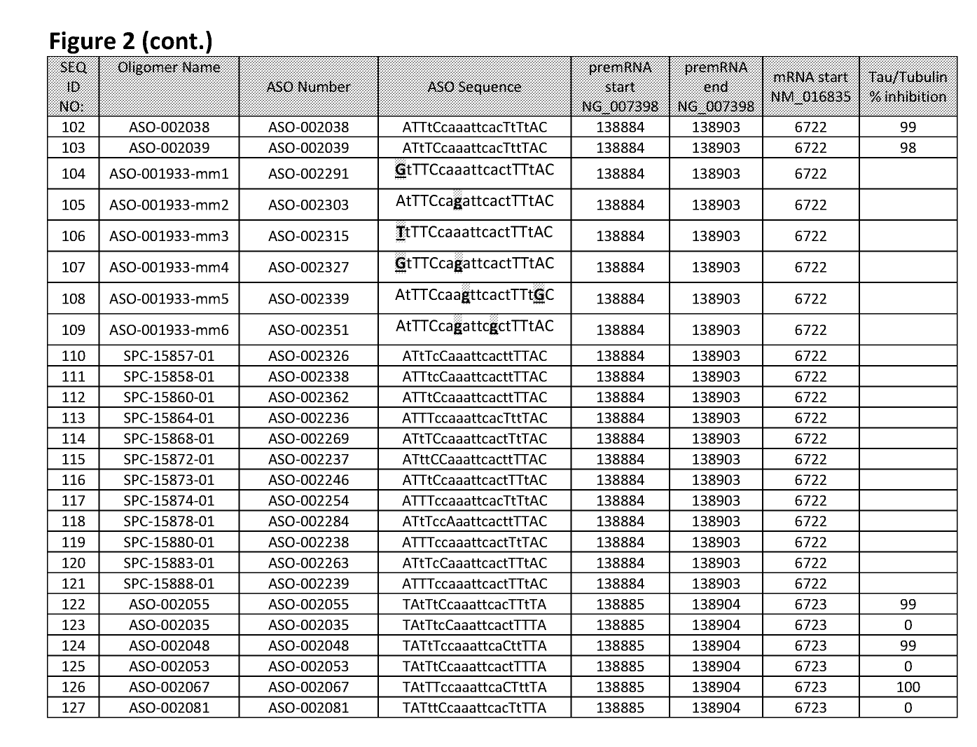

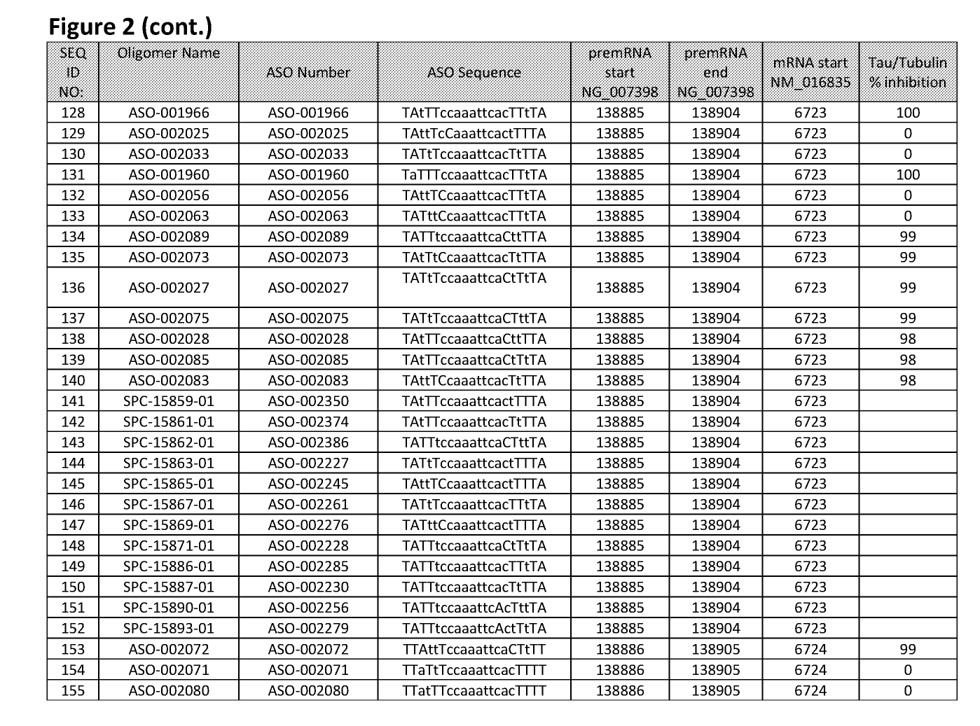

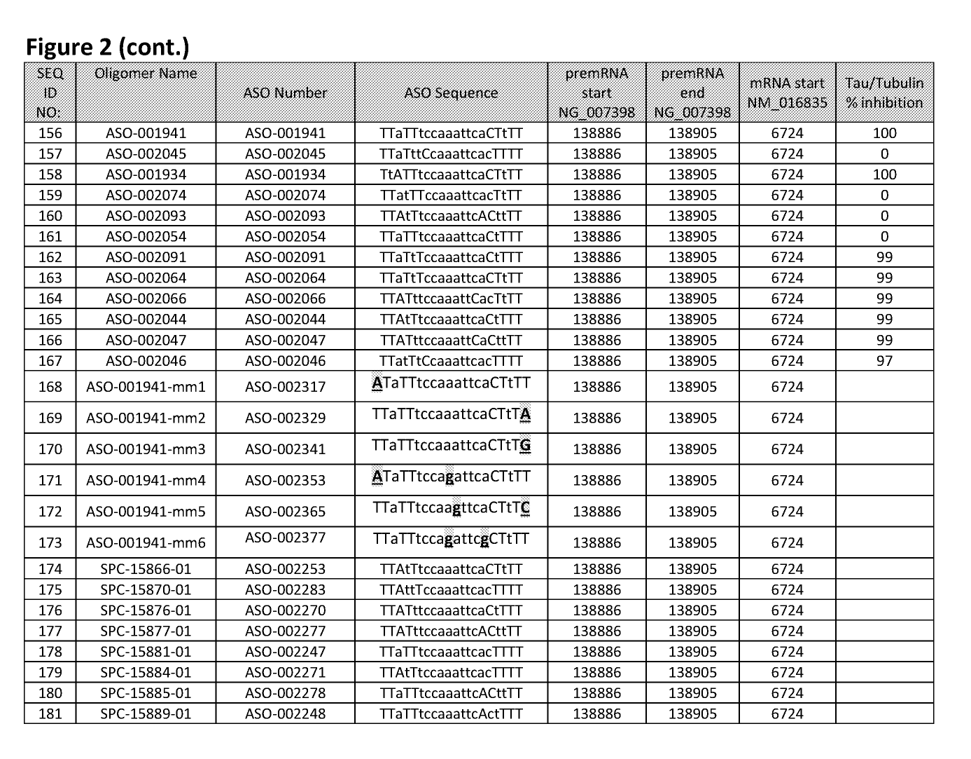

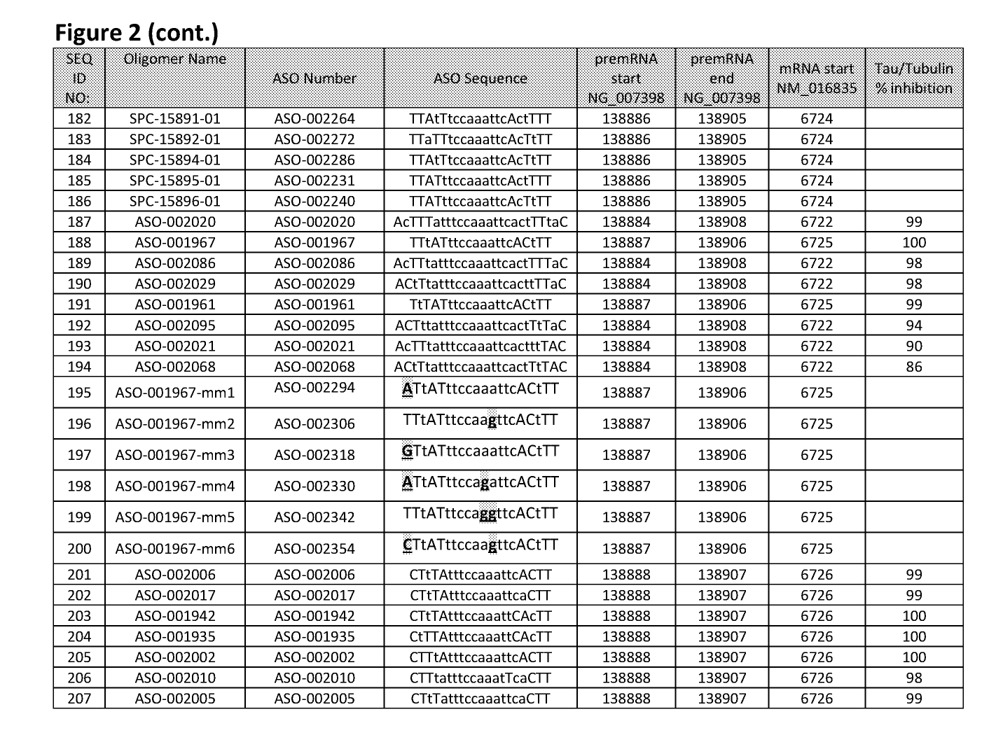

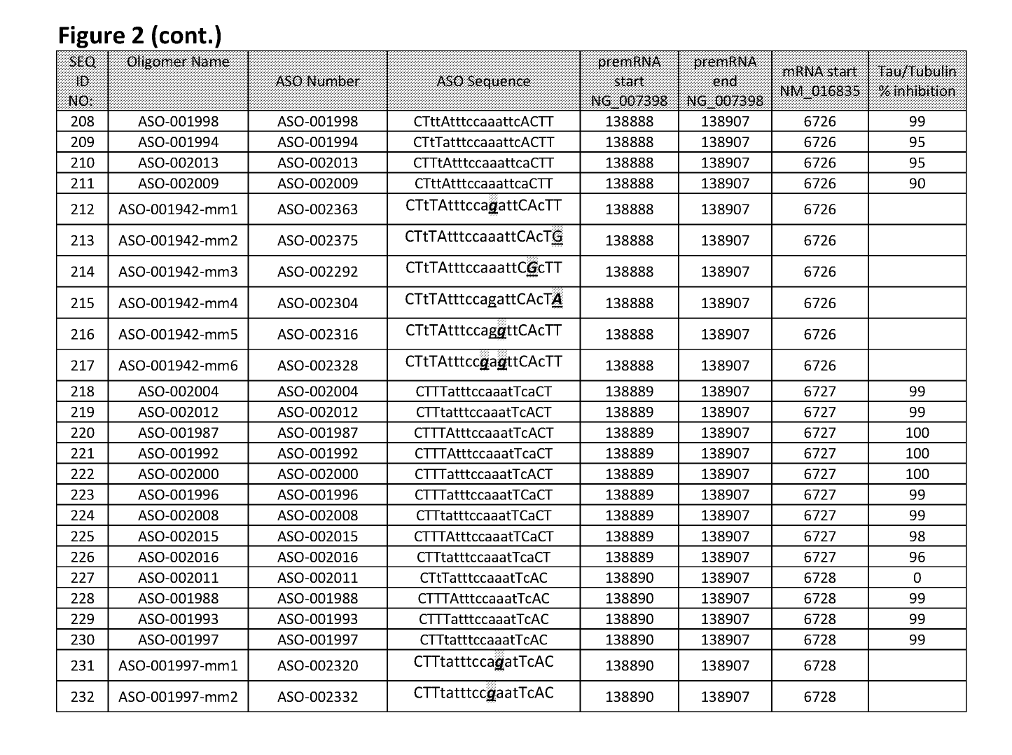

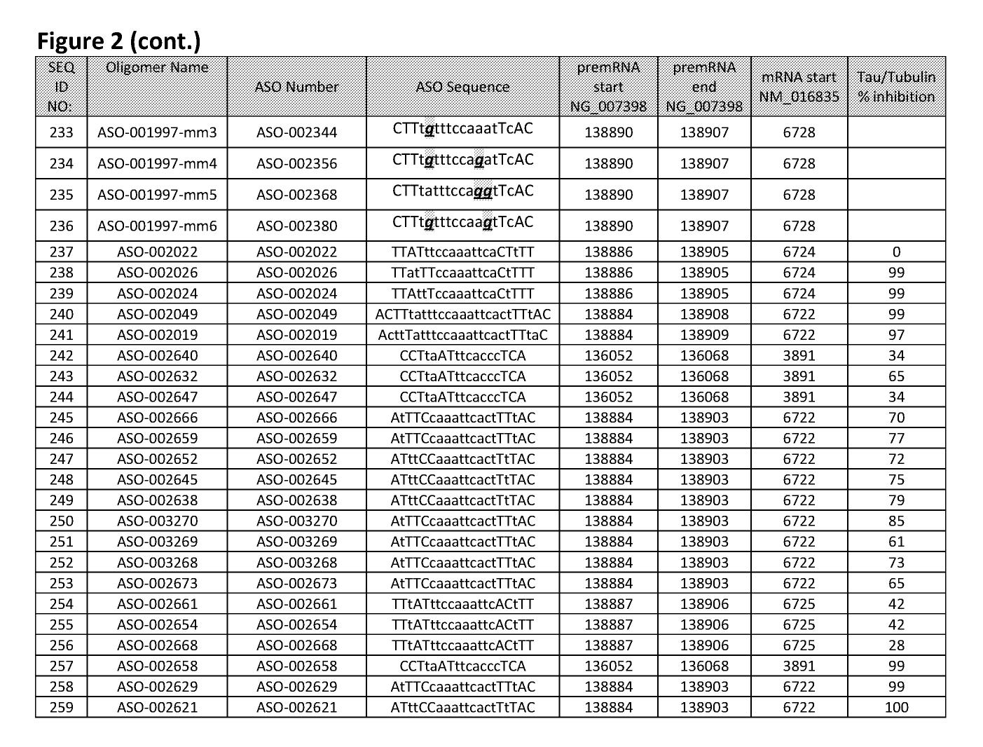

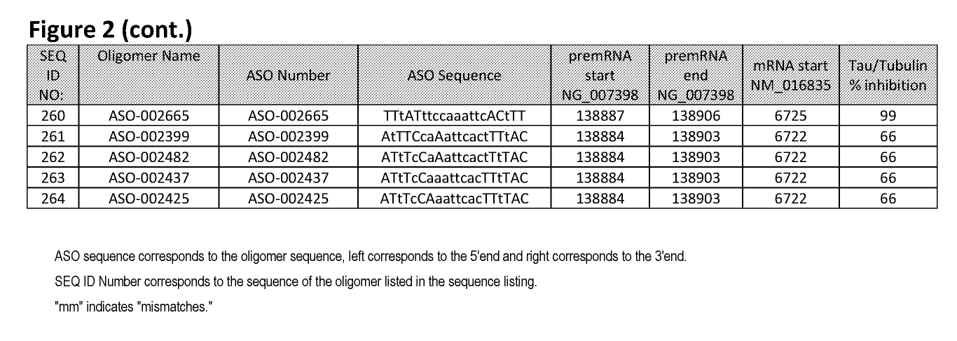

The term “chemical structure” of an oligomer as used herein refers to a detailed description of the components of the oligomers, e.g., nucleotides (e.g., DNA), nucleotide analogs (e.g., beta-D-oxy-LNA), nucleotide base (e.g., A, T, G, C, U, or MC), and backbone structure (e.g., phosphorothioate or phosphorodiester). For example, a chemical structure of ASO-002350 can be OxyTs OxyAs DNAts OxyTs OxyTs DNAcs DNAcs DNAas DNAas DNAas DNAts DNAts DNAcs DNAas DNAcs DNAts OxyTs OxyTs OxyTs OxyAs. FIGS. 2, 16B, and 20B lists non-limiting examples of chemical structures that can be applied to any one of the oligomers disclosed herein.

“Potency” is normally expressed as an IC₅₀ or EC₅₀ value, in μM, nM or pM unless otherwise stated. Potency can also be expressed in terms of percent inhibition. IC50 is the median inhibitory concentration of a therapeutic molecule. EC50 is the median effective concentration of a therapeutic molecule relative to a vehicle or saline control. In functional assays, IC50 is the concentration that reduces a biological response, e.g., transcription of mRNA or protein expression, by 50% of the biological response that is achieved without the therapeutic molecule. In functional assays, EC50 is the concentration of a therapeutic molecule that produces 50% of the biological response, e.g., transcription of mRNA or protein expression. IC50 or EC50 can be calculated by any number of means known in the art.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, sports animals, and zoo animals including, e.g., humans, non-human primates, dogs, cats, guinea pigs, rabbits, rats, mice, horses, cattle, bears, and so on.

The term “pharmaceutical composition” refers to a preparation which is in such form as to permit the biological activity of the active ingredient to be effective, and which contains no additional components which are unacceptably toxic to a subject to which the composition would be administered. Such composition can be sterile.

An “effective amount” of an oligomer as disclosed herein is an amount sufficient to carry out a specifically stated purpose. An “effective amount” can be determined empirically and in a routine manner, in relation to the stated purpose.

Terms such as “treating” or “treatment” or “to treat” or “alleviating” or “to alleviate” refer to both (1) therapeutic measures that cure, slow down, lessen symptoms of, and/or halt progression of a diagnosed pathologic condition or disorder and (2) prophylactic or preventative measures that prevent and/or slow the development of a targeted pathologic condition or disorder. Thus, those in need of treatment include those already with the disorder; those prone to have the disorder; and those in whom the disorder is to be prevented. In certain embodiments, a subject is successfully “treated” for a disease or condition disclosed elsewhere herein according to the methods provided herein if the patient shows, e.g., total, partial, or transient alleviation or elimination of symptoms associated with the disease or disorder.

### II. The Oligomer

The present invention employs oligomeric compounds (referred to herein as oligomers) having an improved oligomer design. The oligomer design is improved such that the design provides at least one enhanced property. For example, while not bound by any theory, the oligomers of the present disclosure have a reduced non-specific binding, but retain the affinity to the target nucleic acid. The reduced non-specific binding of the oligomer can result in less toxicity of the oligomers, especially when administered in vivo. Therefore, the oligomers can be used in modulating the function of nucleic acid molecules in vivo. The term “oligomer” in the context of the present invention, refers to a molecule formed by covalent linkage of two or more nucleotides (i.e., an oligonucleotide).

The oligomer comprises a contiguous nucleotide sequence of from about 10 to about 50, such as 10-20, 16-20, 15-25, 10-30, 10-35, 10-40, or 10-45 nucleotides in length. The terms “antisense oligomer,” “antisense oligonucleotide,” and “ASO” as used herein are interchangeable with the term “oligomer.”

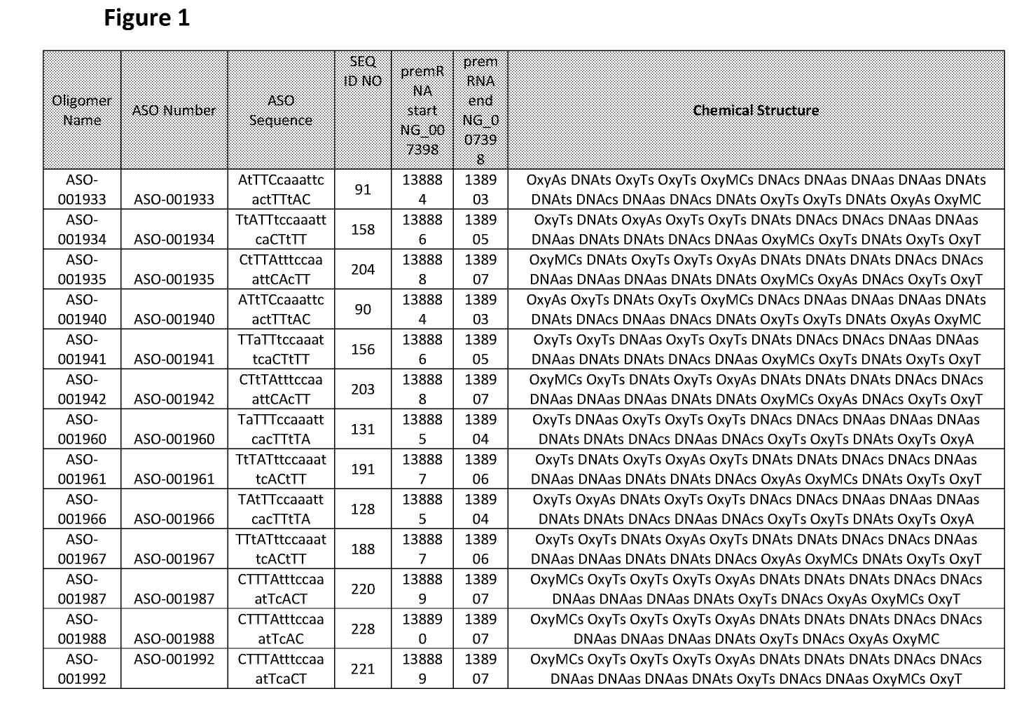

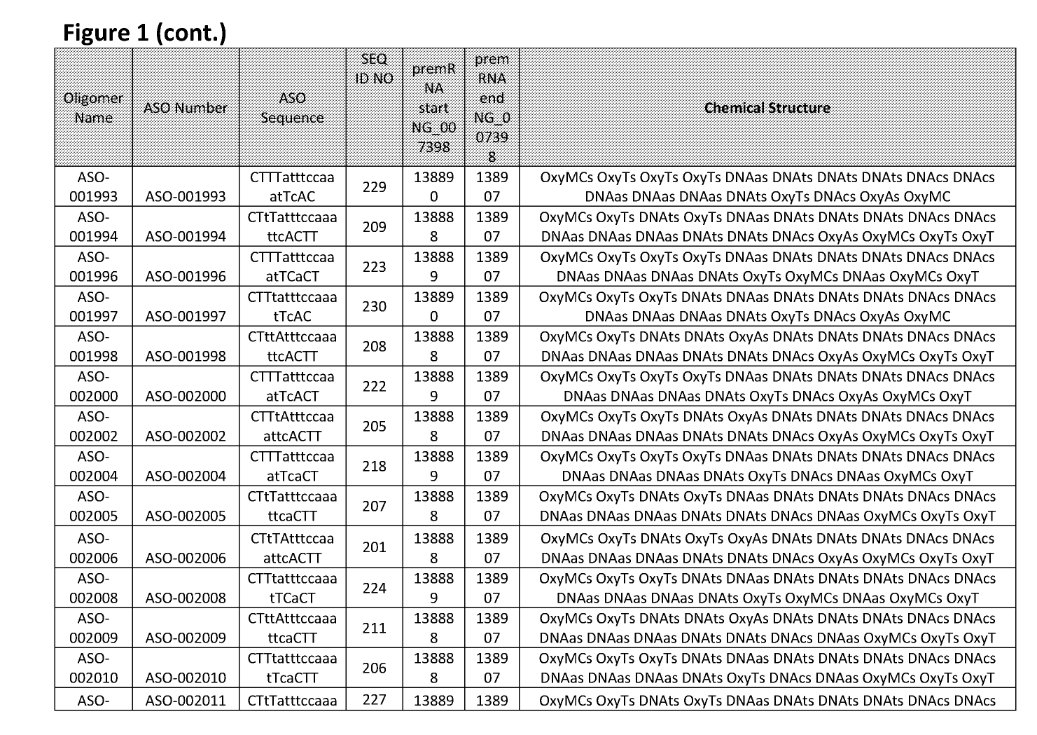

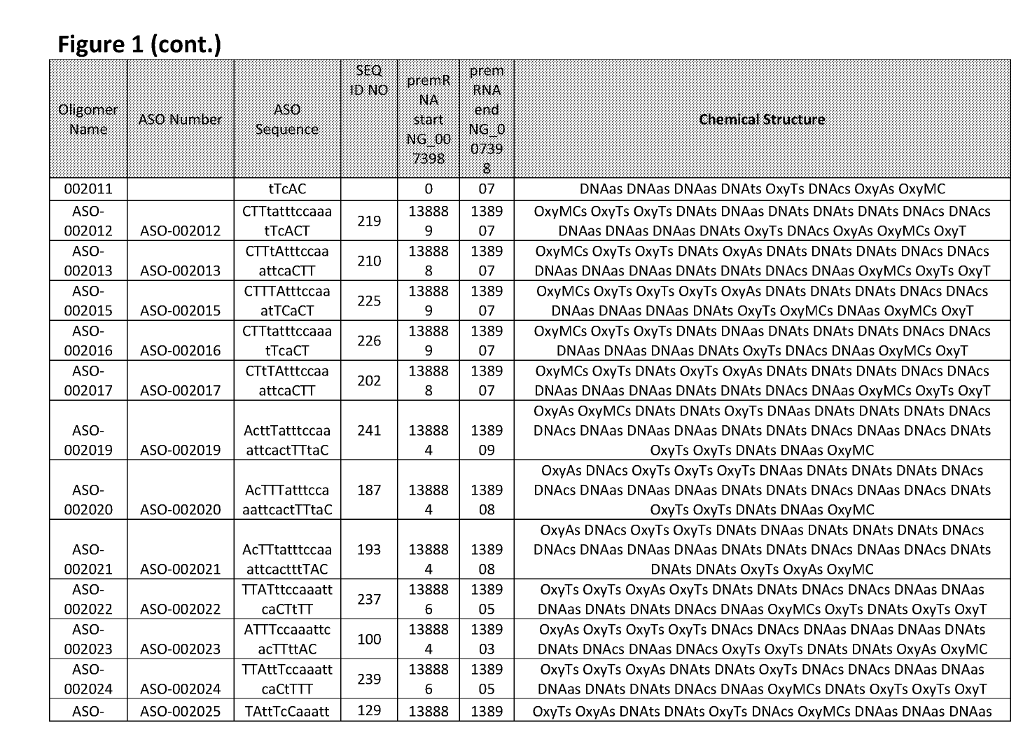

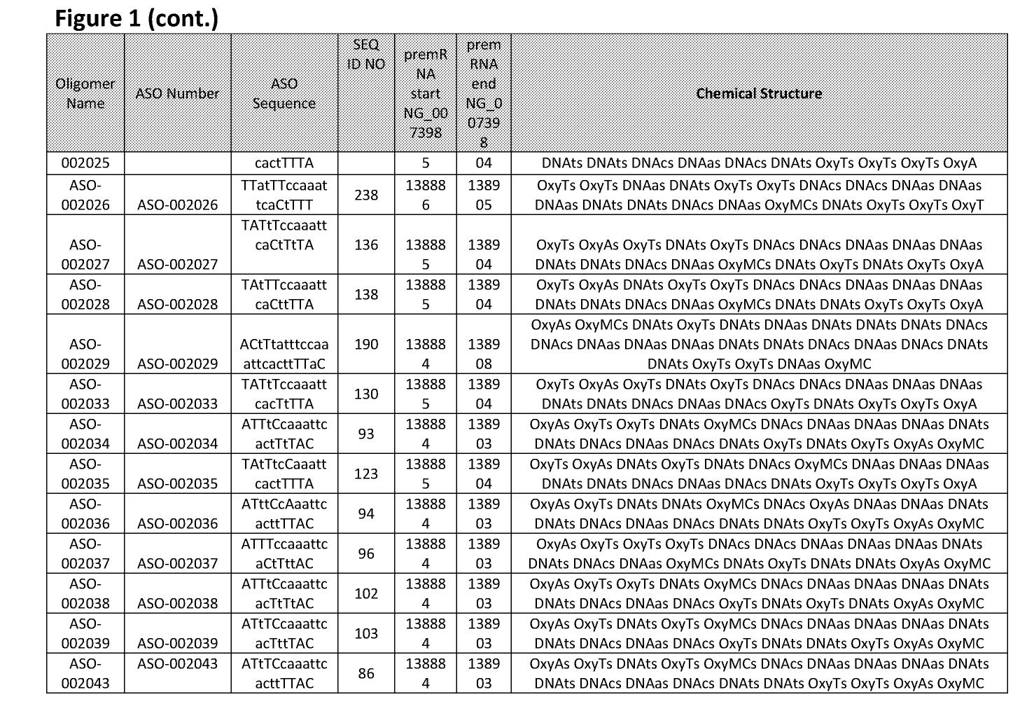

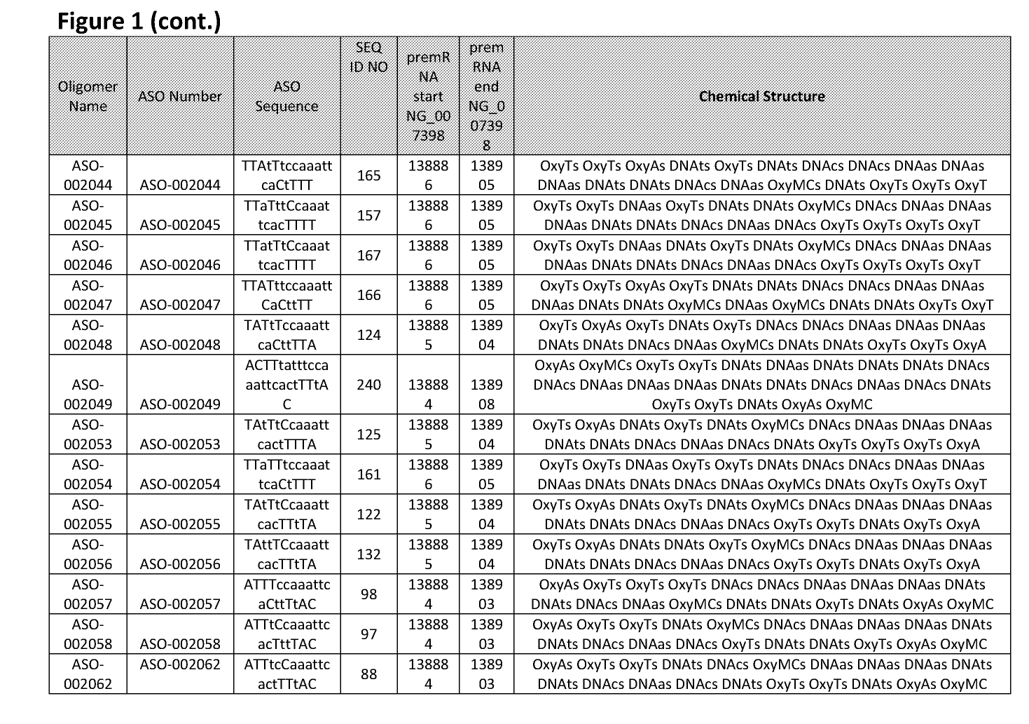

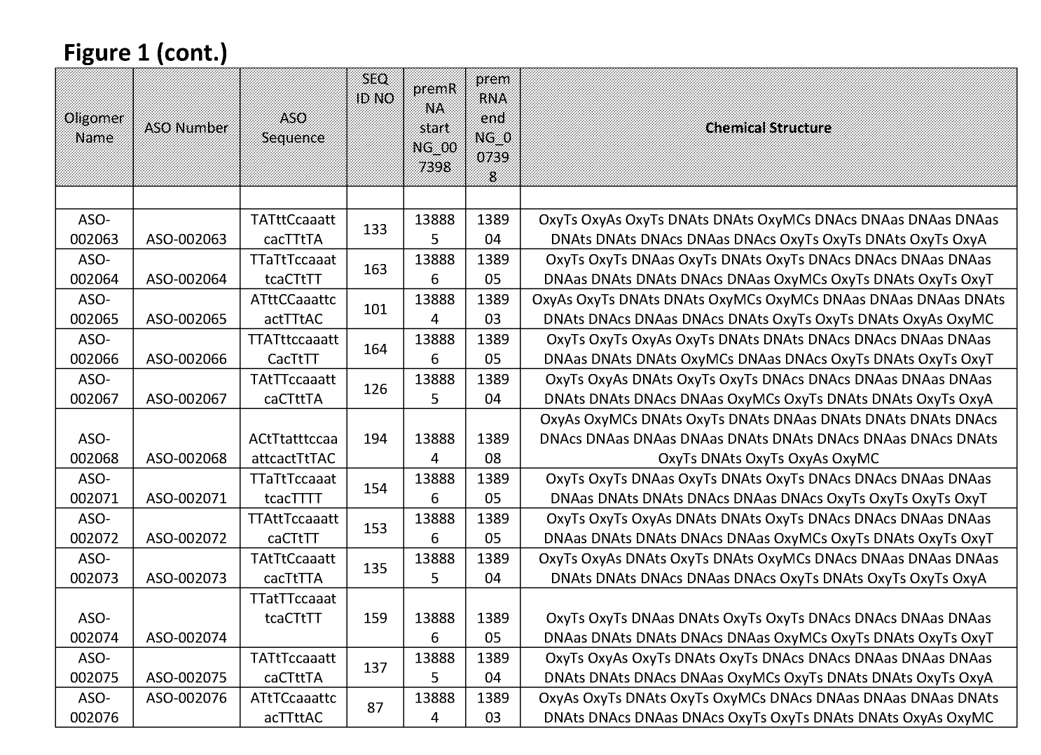

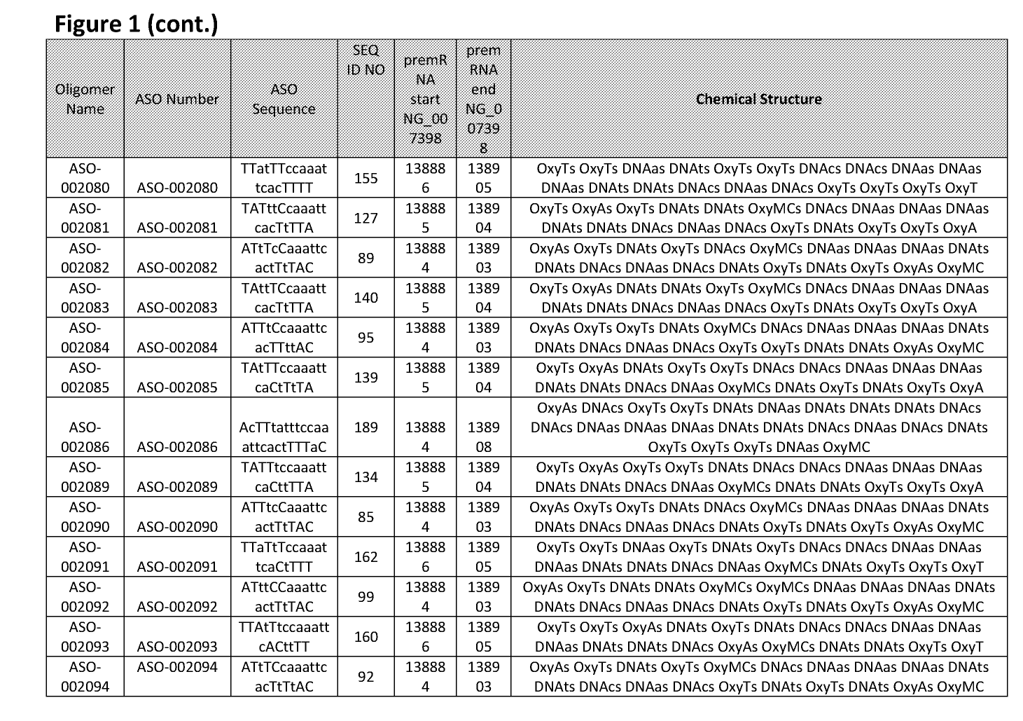

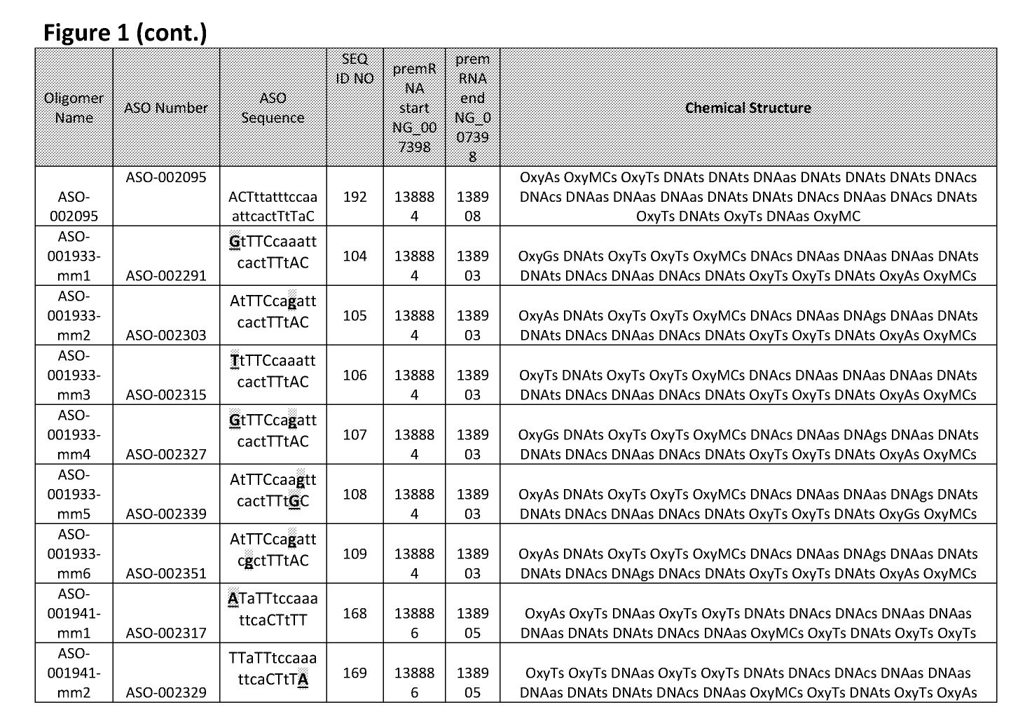

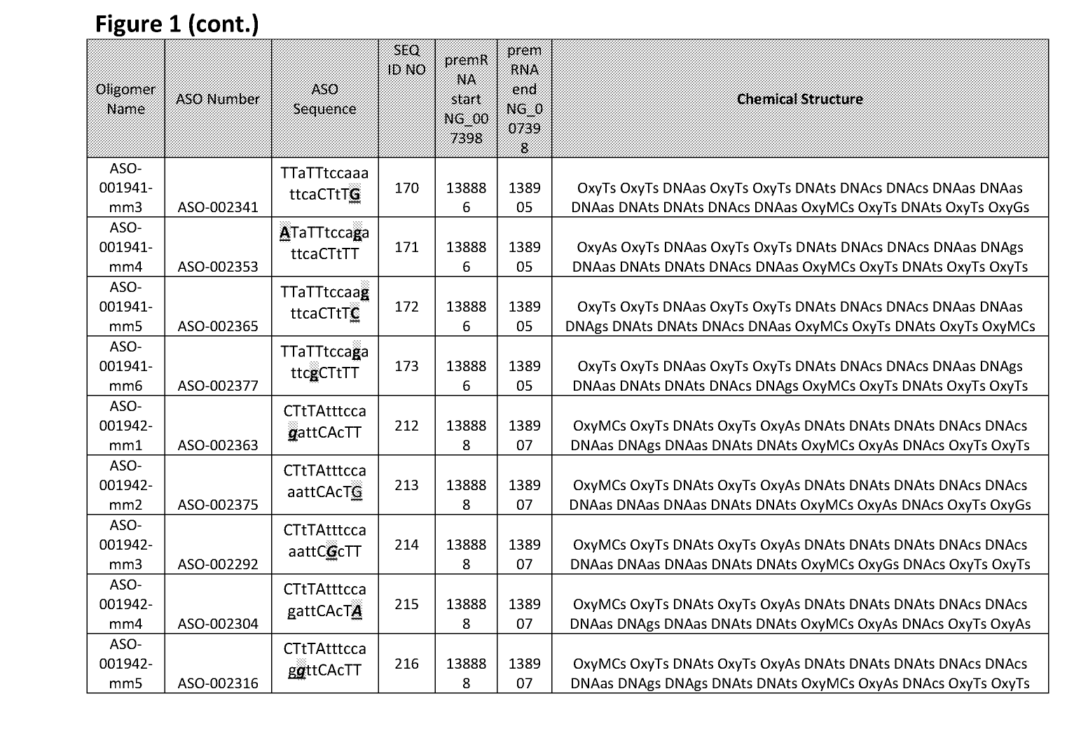

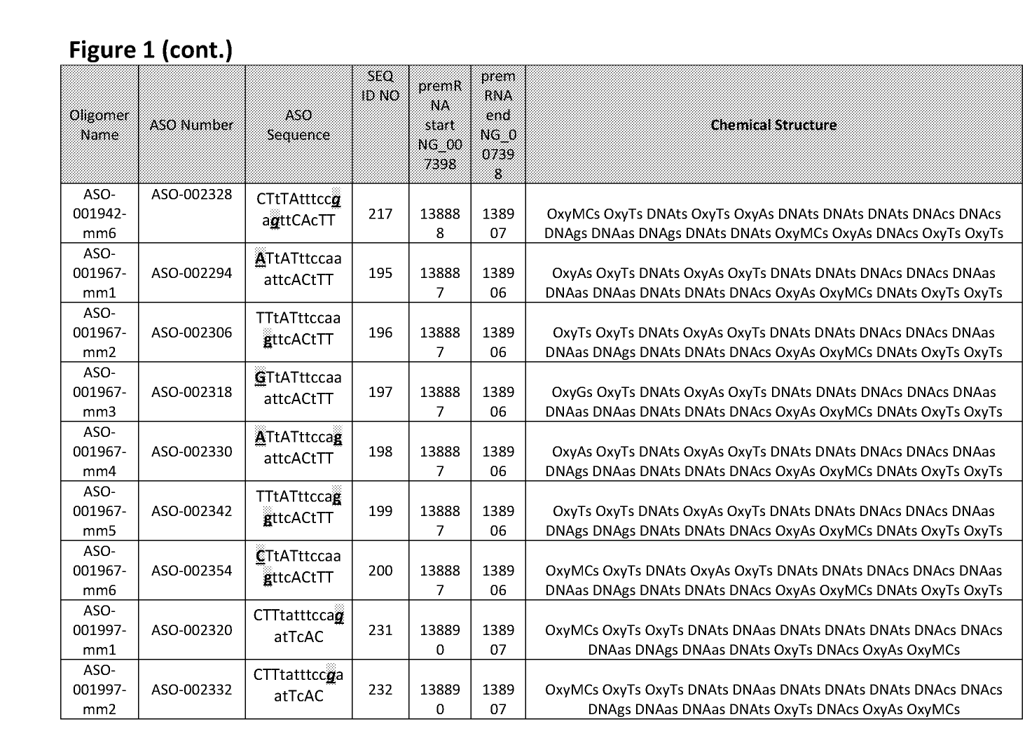

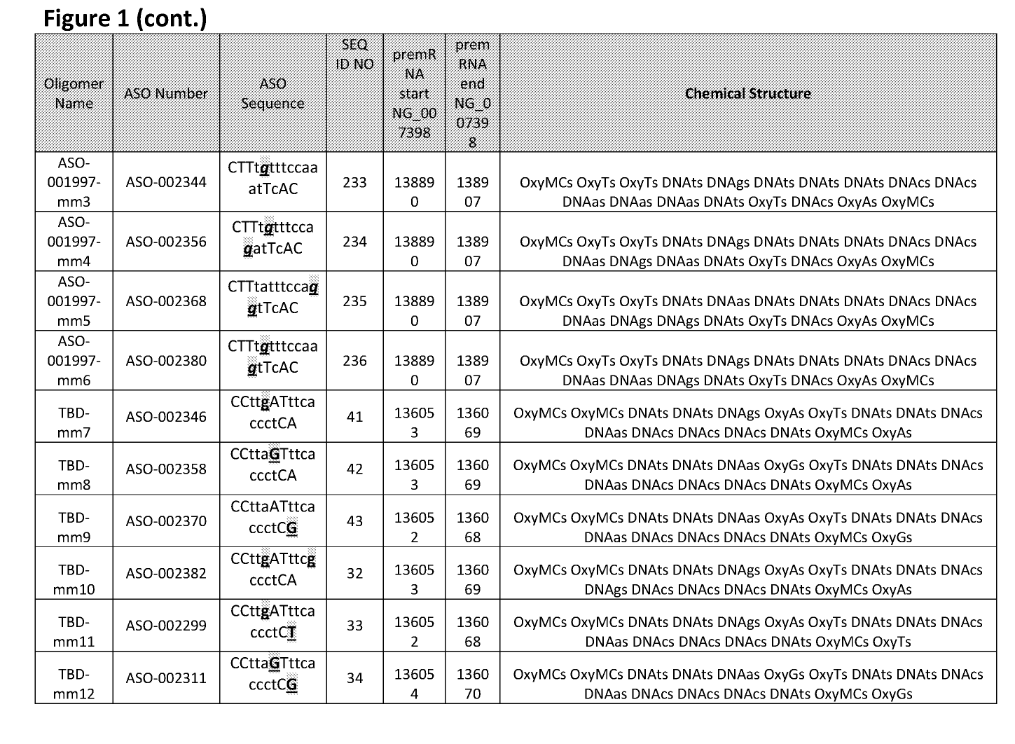

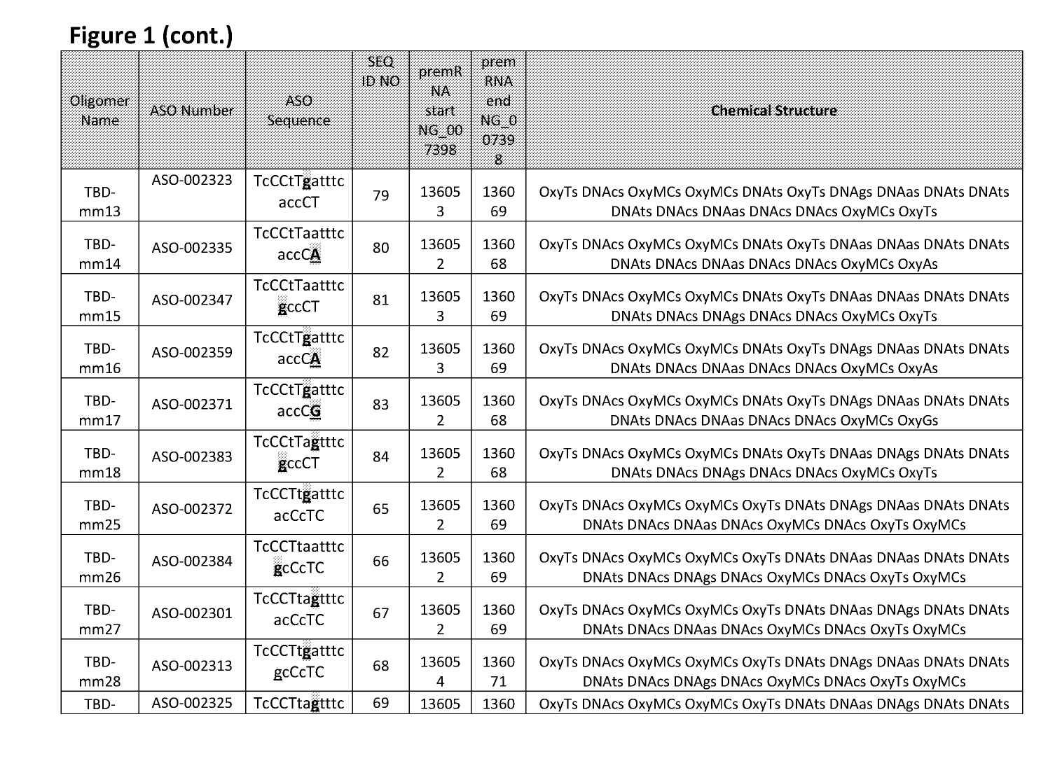

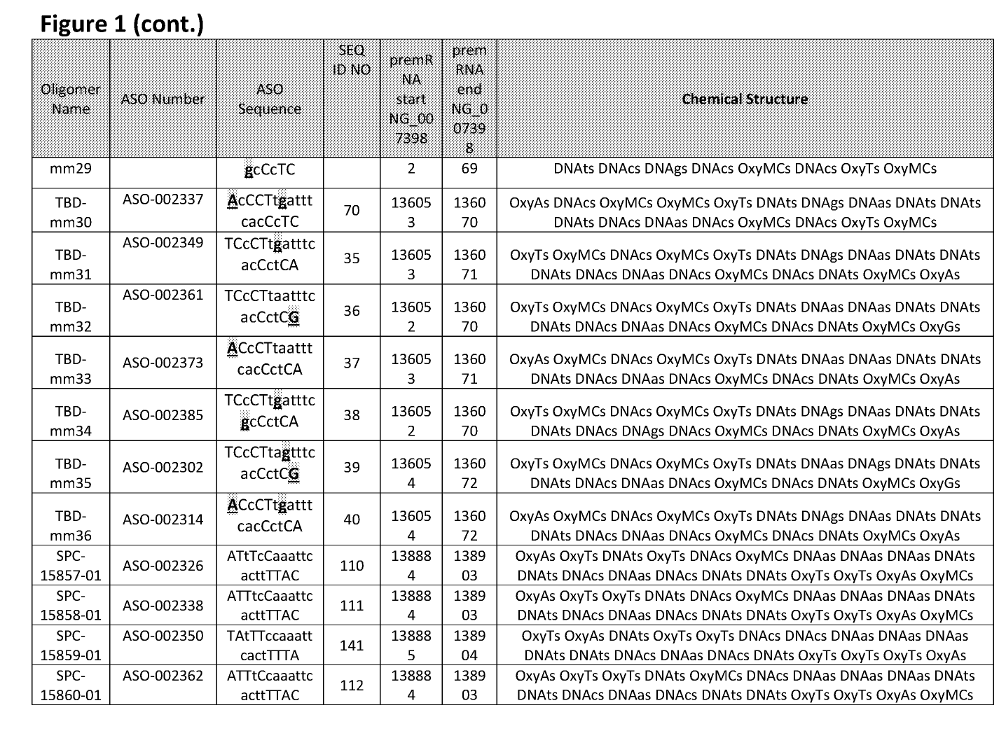

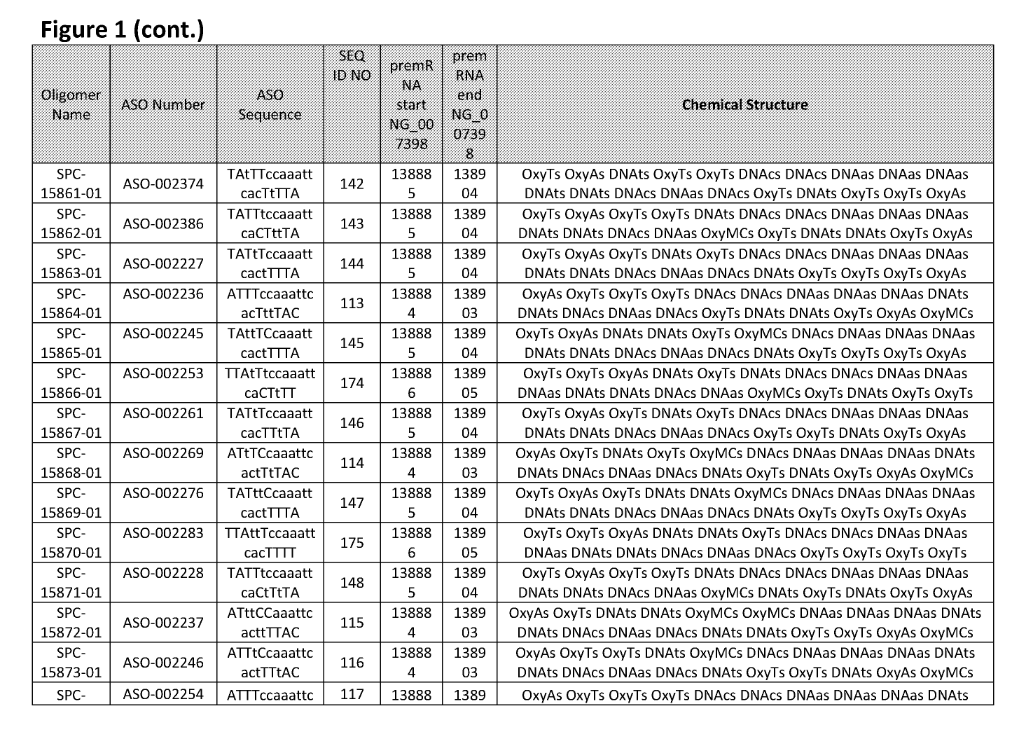

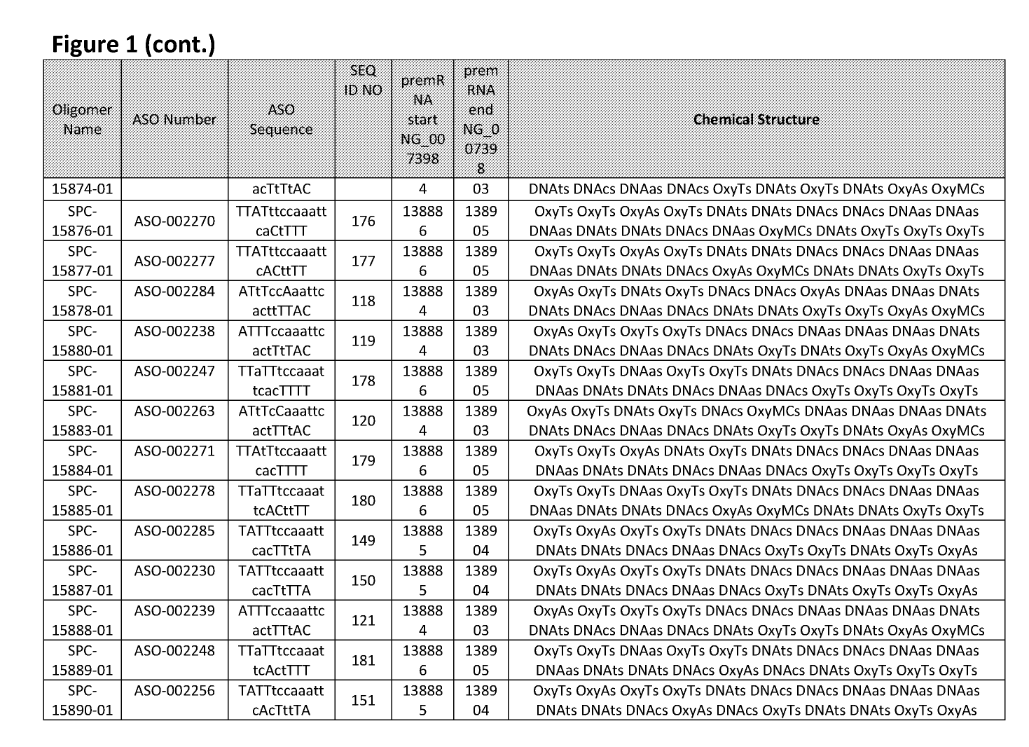

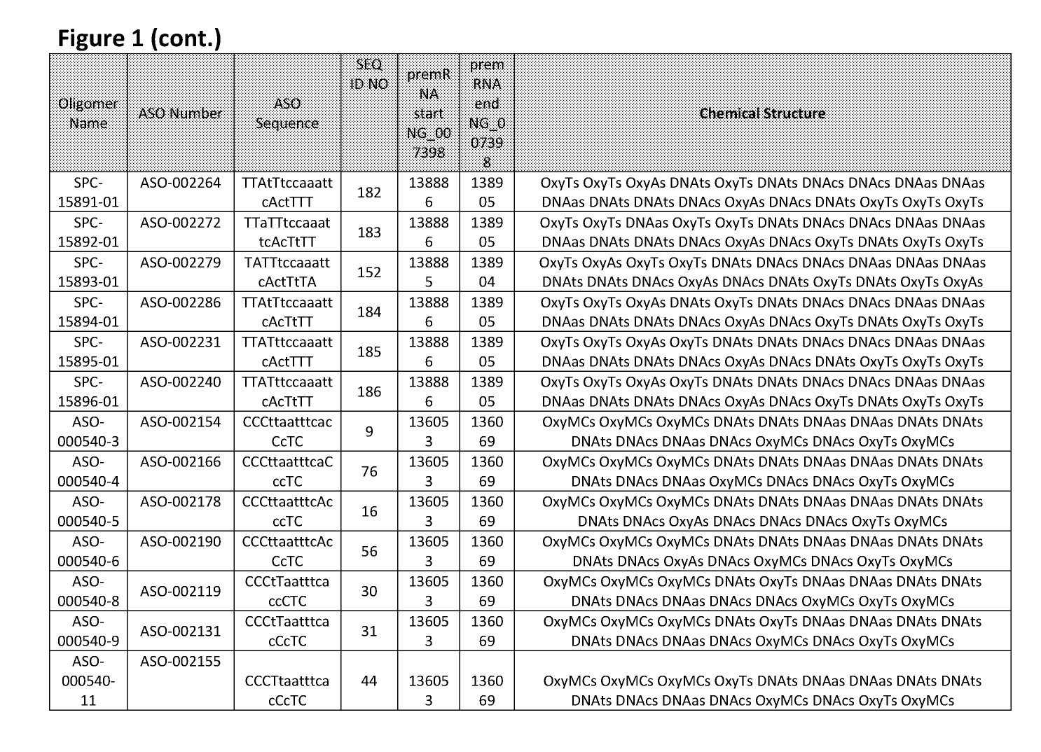

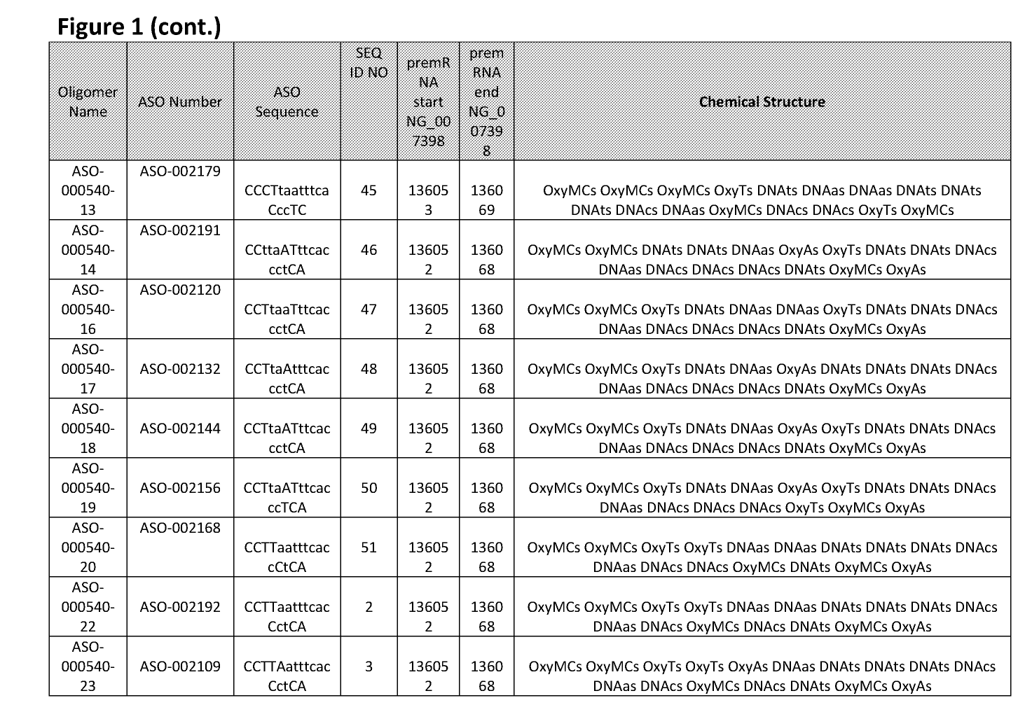

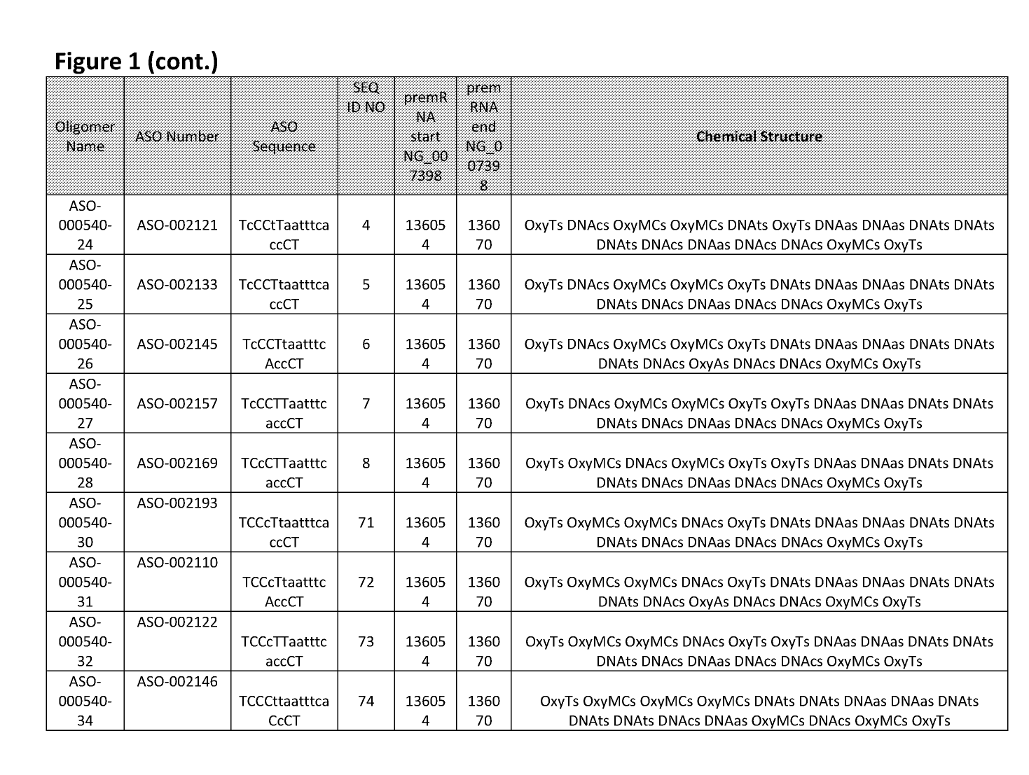

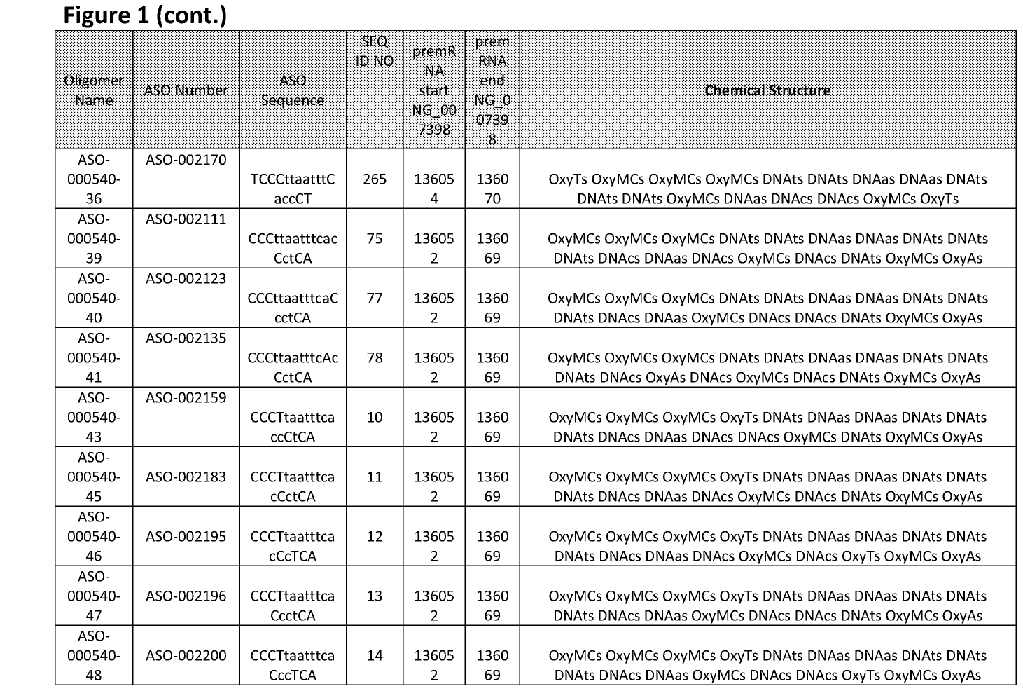

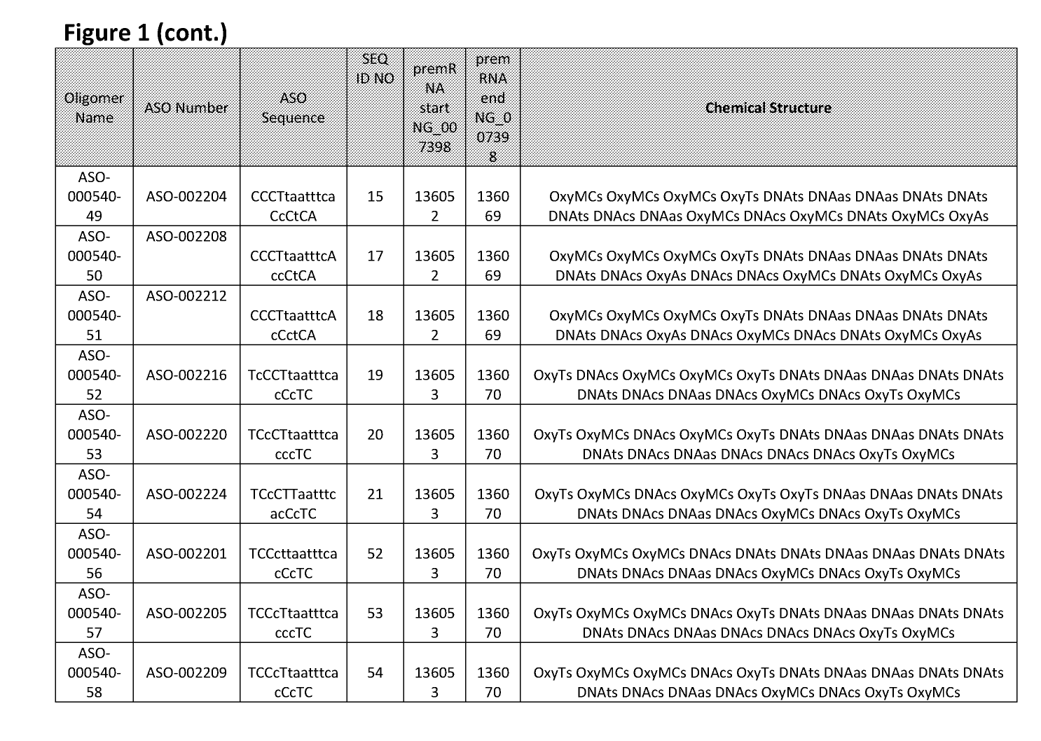

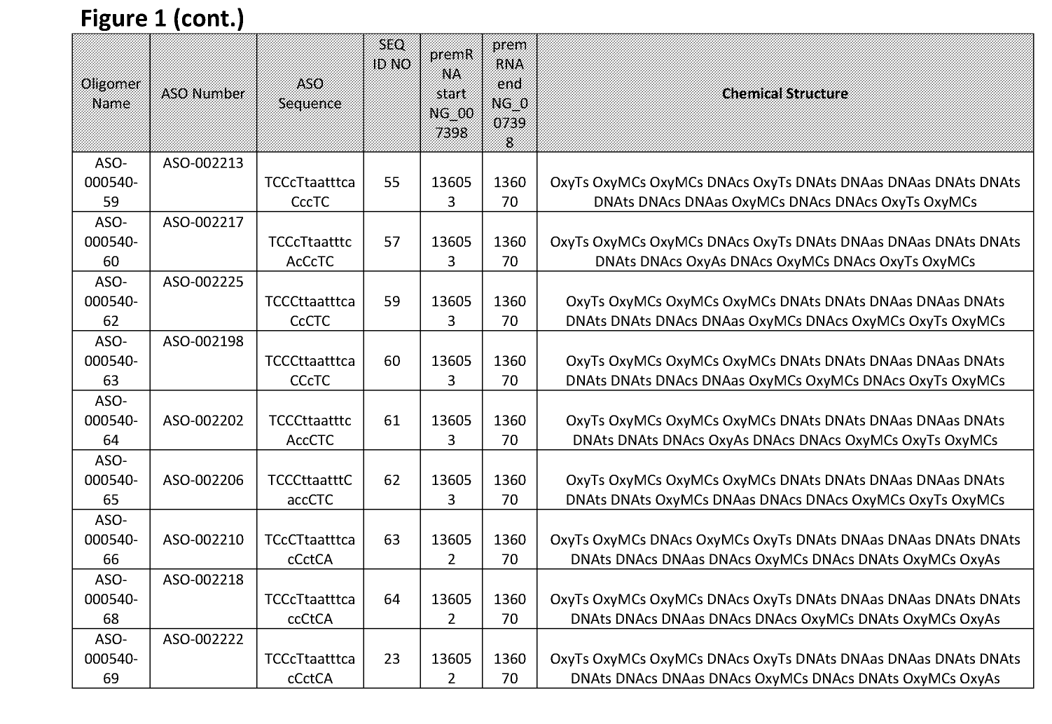

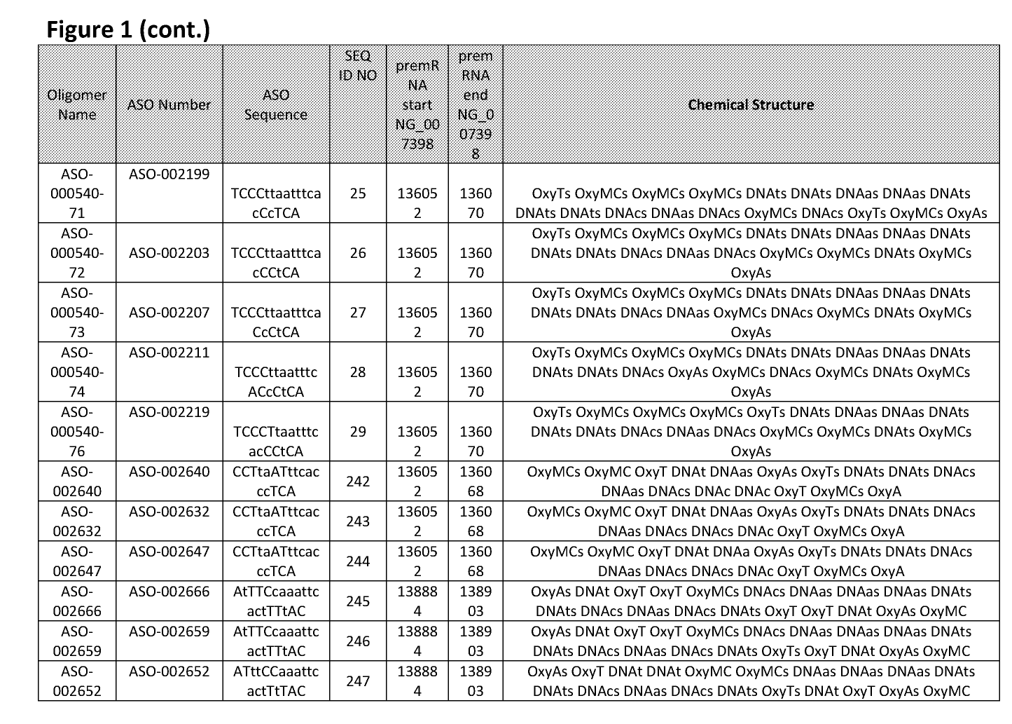

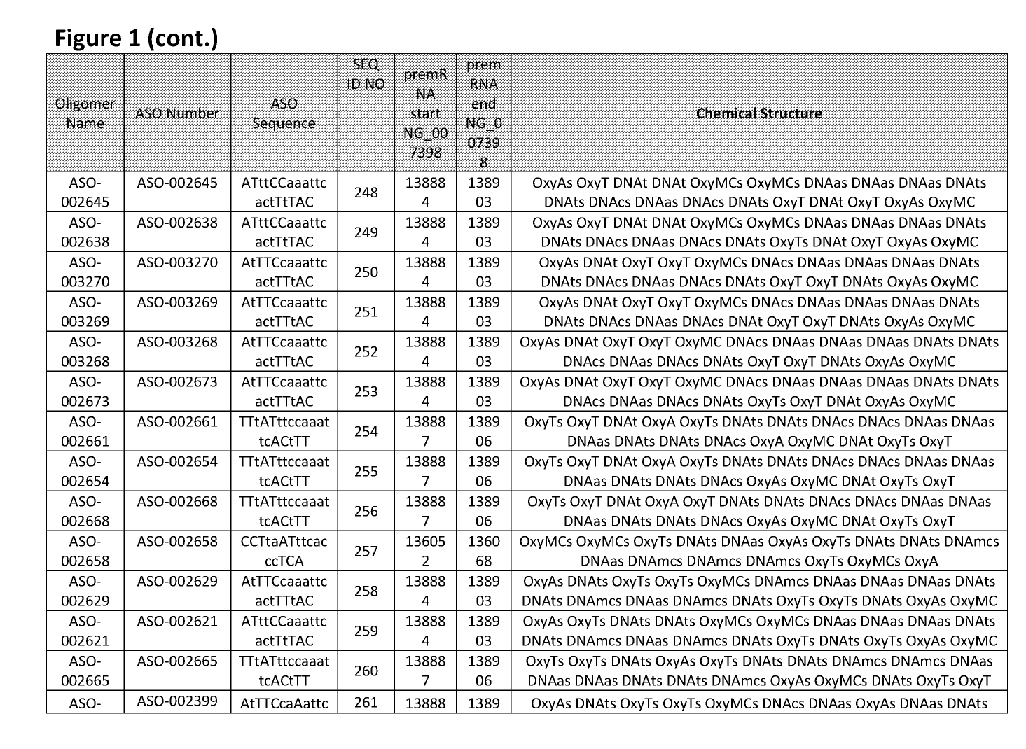

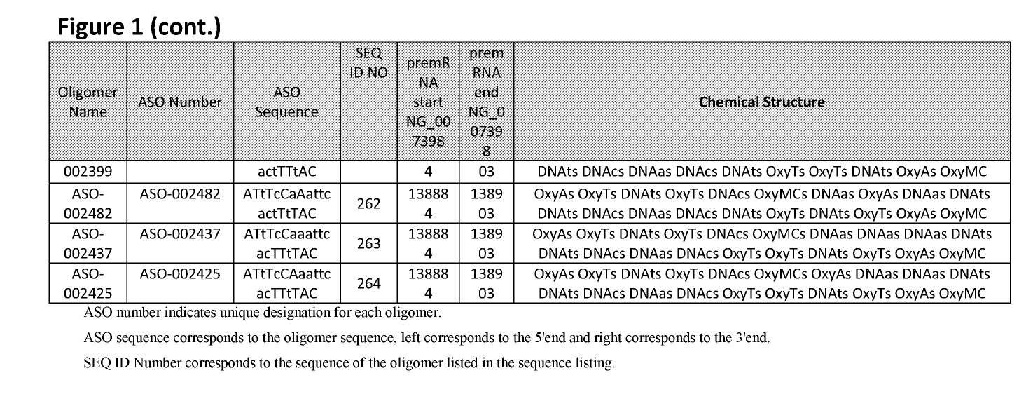

A reference to a SEQ ID number includes a particular sequence, but does not include an oligomer design as shown in FIGS. 1, 6B, and 11B. Furthermore, the oligomers disclosed in the figures herein show a representative design, but are not limited to the specific design shown in the tables. Herein, a single nucleotide (unit) can also be referred to as a monomer or unit. When this specification refers to a specific ASO number (or oligomer name), the reference includes the specific oligomer design. For example, when a claim (or this specification) recites SEQ ID NO: 241, it includes the nucleotide sequence of actttatttccaaattcacttttac. When a claim (or the specification) recites ASO-002019, it includes the nucleotide sequence of actttatttccaaattcacttttac with the oligomer design shown in the figures (i.e., ActtTatttccaaattcactTTtaC). Alternatively, ASO-002019 can be written as ActtTatttccaaattcactTTtaC, wherein the upper case letter is a modified nucleotide (e.g., LNA) and the lower case letter is a non-modified nucleotide (e.g., DNA). ASO-002019 can also be written as SEQ ID NO: 241, wherein each of the first nucleotide, the fifth nucleotide, the 21^{st} nucleotide, the 22^{nd} nucleotide, and the 25^{th} nucleotide from the 5′ end is a modified nucleotide, e.g., LNA, and each of the other nucleotides is a non-modified nucleotide (e.g., DNA). The oligomers of the invention can also be written as SEQ ID NO: 241 with the chemical structure shown in FIG. 1, i.e., OxyAs OxyMCs DNAts DNAts OxyTs DNAas DNAts DNAts DNAts DNAcs DNAcs DNAas DNAas DNAas DNAts DNAts DNAcs DNAas DNAcs DNAts OxyTs OxyTs DNAts DNAas OxyMC.

In various embodiments, the oligomer of the invention does not comprise RNA (units). In some embodiments, the oligomer comprises one or more DNA units. In one embodiment, the oligomer according to the invention is a linear molecule or is synthesized as a linear molecule. In some embodiments, the oligomer is a single stranded molecule, and does not comprise short regions of, for example, at least 3, 4 or 5 contiguous nucleotides, which are complementary to equivalent regions within the same oligomer (i.e. duplexes)—in this regard, the oligomer is not (essentially) double stranded. In some embodiments, the oligomer is essentially not double stranded. In some embodiments, the oligomer is not a siRNA. In various embodiments, the oligomer of the invention can consist entirely of the contiguous nucleotide region. Thus, in some embodiments the oligomer is not substantially self-complementary

In certain embodiments, the oligomers of the invention can be used in treating a neurological disease or condition in a subject in need thereof, wherein calcium oscillations in neuronal cells that are in contact with the oligomer are about 70% or higher, about 75% or higher, about 80% or higher, about 85% or higher, about 90% or higher, about 95% or higher, about 96% or higher, about 97% or higher, about 98% or higher, about 99% or higher, about 100% or higher, about 120% or higher, about 140% or higher, about 160% or higher, about 180% or higher, about 200% or higher, about 220% or higher, about 240% or higher, or about 250% or higher compared to the calcium oscillations in vehicle control cells.

In other embodiments, the oligomers of the invention can be used in treating a neurological disease or condition, wherein the oligomer has a sequence score greater than or equal to 0.2, greater than or equal to 0.25, greater than or equal to 0.3, greater than or equal to 0.35, greater than or equal to 0.4, greater than or equal to 0.45, greater than or equal to 0.5, greater than or equal to 0.55, greater than or equal to 0.6, greater than or equal to 0.65, greater than or equal to 0.7, greater than or equal to 0.75, greater than or equal to 0.8, greater than or equal to 0.85, greater than or equal to 0.9, greater than or equal to 0.95, greater than or equal to 1.0, greater than or equal to 1.5, greater than or equal to 2.0, greater than or equal to 3.0, or greater than or equal to 4.0.

### II.A. The Target

A target nucleic acid is an intended target of the oligomer of the invention, and may for example, be a gene, a RNA, a mRNA, and pre-mRNA, a mature mRNA or a cDNA sequence. For in vivo or in vitro application, the oligonucleotide of the invention is typically capable of modulating the expression of the intended target nucleic acid in a cell which is expressing the target nucleic acid. The contiguous sequence of nucleobases of the oligomers is typically complementary to the target nucleic acid, as measured across the length of the oligonucleotide, optionally with the exception of one or two mismatches, and optionally excluding nucleotide based linker regions which may link the oligonucleotide to an optional functional group such as a conjugate. The target nucleic acid may, in some embodiments, be a RNA or DNA that is associated with any disease or condition. In some embodiments, the target nucleic acid may be RNA, such as a messenger RNA (e.g., a mature mRNA or a pre-mRNA).

In some embodiments, the oligomer of the invention is capable of hybridizing to the target nucleic acid under physiological condition, i.e., in vivo condition. In some embodiments, the oligomer of the invention is capable of hybridizing to the target nucleic acid in vitro. In some embodiments, the oligomer of the invention is capable of hybridizing to the target nucleic acid in vitro under stringent conditions. Stringency conditions for hybridization in vitro are dependent on, inter alia, productive cell uptake, RNA accessibility, temperature, free energy of association, salt concentration, and time (see, e.g., Stanley T Crooks, Antisense Drug Technology: Principles, Strategies and Applications, 2^{nd} Edition, CRC Press (2007))). Generally, conditions of high to moderate stringency are used for in vitro hybridization to enable hybridization between substantially similar nucleic acids, but not between dissimilar nucleic acids. An example of stringent hybridization conditions include hybridization in 5× saline-sodium citrate (SSC) buffer (0.75 M sodium chloride/0.075 M sodium citrate) for 1 hour at 40° C., followed by washing the sample 10 times in 1×SSC at 40° C. and 5 times in 1×SSC buffer at room temperature. In vivo hybridization conditions consist of intracellular conditions (e.g., physiological pH and intracellular ionic conditions) that govern the hybridization of antisense oligonucleotides with target sequences. In vivo conditions can be mimicked in vitro by relatively low stringency conditions. For example, hybridization can be carried out in vitro in 2×SSC (0.3 M sodium chloride/0.03 M sodium citrate), 0.1% SDS at 37° C. A wash solution containing 4×SSC, 0.1% SDS can be used at 37° C., with a final wash in 1×SSC at 45° C.

In some embodiments, the oligomers of the invention bind to the target nucleic acid sequence and inhibit or reduce expression of the target nucleic acid transcript by at least 10% or 20% compared to the normal (i.e., control) expression level in the cell, e.g., at least 30%, 40%, 50%, 60%, 70%, 80%, 90% or 95% compared to the normal expression level (such as the expression level in the absence of the oligomer(s) or conjugate(s)) in the cell.

In certain embodiments, the oligomers of the invention bind to the target nucleic acid transcript and inhibit or reduce expression of the target nucleic acid mRNA by at least about 10% or about 20% compared to the normal (i.e. control) expression level in the cell, e.g., at least about 30%, about 40%, about 50%, about 60%, about 70%, about 80%, about 90% or about 95% compared to the normal expression level (such as the expression level in the absence of the oligomer(s) or conjugate(s)) in the cell. In certain embodiments, the oligomer reduces expression of target protein in a cell following administration of the oligomer by at least 60%, at least 70%, at least 80%, or at least 90% compared to a cell not exposed to the oligomer (i.e., control). In some embodiments, the oligomer reduces expression of target protein in a cell following administration of the oligomer by at least about 60%, at least about 70%, at least about 80%, or at least about 90% compared to a cell not exposed to the oligomer (i.e., control).

In certain embodiments, the oligomer of the invention has at least one property selected from: (1) reduces expression of target mRNA in a cell, compared to a control cell that has not been exposed to the oligomer; (2) does not significantly reduce calcium oscillations in a cell; (3) does not significantly reduce tubulin intensity in a cell; (4) reduces expression of Tau target protein in a cell; and (5) any combinations thereof compared to a control cell that has not been exposed to the oligomer.

Calcium oscillations are important for the proper functions of neuronal cells. Networks of cortical neurons have been shown to undergo spontaneous calcium oscillations resulting in the release of the neurotransmitter glutamate. Calcium oscillations can also regulate interactions of neurons with associate glia, in addition to other associated neurons in the network, to release other neurotransmitters in addition to glutamate. Regulated calcium oscillations are required for homeostasis of neuronal networks for normal brain function. (See, Shashank et al., Brain Research, 1006(1): 8-17 (2004); Rose et al., Nature Neurosci., 4:773-774 (2001); Zonta et al., JPhysiol Paris., 96(3-4):193-8 (2002); Pasti et al., J Neurosci., 21(2): 477-484 (2001).) Glutamate also activates two distinct ion channels, α-amino-3-hydroxy-5-methyl-4-isoxazolepropionic acid (AMPA) receptors and N-methyl-D-aspartate (NMDA) receptors.

In some embodiments, the calcium oscillations measured in the present methods are AMPA-dependent calcium oscillations. In some embodiments, the calcium oscillations are NMDA-dependent calcium oscillations. In some embodiments, the calcium oscillations are gamma-aminobutyric acid (GABA)-dependent calcium oscillations. In some embodiments, the calcium oscillations can be a combination of two or more of AMPA-dependent, NMDA-dependent or GABA-dependent calcium oscillations.

In certain embodiments, the calcium oscillations measured in the present methods are AMPA-dependent calcium oscillations. In order to measure AMPA-dependent calcium oscillations, the calcium oscillations can be measured in the presence of Mg²⁺ ions (e.g., MgCl₂). In certain embodiments, the method further comprises adding Mg²⁺ ions (e.g., MgCl₂) at an amount that allows for detection of AMPA-dependent calcium oscillations. In some embodiments, the effective ion concentration amount allowing for detection of AMPA-dependent calcium oscillations is at least about 0.5 mM. In other embodiments, the effective ion concentration amount to induce AMPA-dependent calcium oscillations is at least about 0.6 mM, at least about 0.7 mM, at least about 0.8 mM, at least about 0.9 mM, at least about 1 mM, at least about 1.5 mM, at least about 2.0 mM, at least about 2.5 mM, at least about 3.0 mM, at least about 4 mM, at least about 5 mM, at least about 6 mM, at least about 7 mM, at least about 8 mM, at least about 9 mM, or at least about 10 mM. In a particular embodiment, the concentration of Mg²⁺ ions (e.g., MgCl₂) useful for the methods is 1 mM. In certain embodiments, the concentration of Mg²⁺ ions (e.g., MgCl₂) useful for the present methods is about 1 mM to about 10 mM, about 1 mM to about 15 mM, about 1 mM to about 20 mM, or about 1 mM to about 25 mM. Mg²⁺ ions can be added by the addition of magnesium salts, such as magnesium carbonate, magnesium chloride, magnesium citrate, magnesium hydroxide, magnesium oxide, magnesium sulfate, and magnesium sulfate heptahydrate.

In some embodiments, calcium oscillations are measured in the present method through the use of fluorescent probes which detect the fluctuations of intracellular calcium levels. For example, detection of intracellular calcium flux can be achieved by staining the cells with fluorescent dyes which bind to calcium ions (known as fluorescent calcium indicators) with a resultant, detectable change in fluorescence (e.g., Fluo-4 AM and Fura Red AM dyes available from Molecular Probes. Eugene, Oreg., United States of America).

In other embodiments, the oligomers of the invention do not significantly reduce the tubulin intensity in a cell. In some embodiments, tubulin intensity is greater than or equal to 95%, greater than or equal to 90%, greater than or equal to 85%, greater than or equal to 80%, greater than or equal to 75%, or greater than or equal to 70% of tubulin intensity in a cell not exposed to the oligomer (or exposed to saline).

In some embodiments, such property is observed when using from 0.04 nm to 400 μM, concentration of the oligomer of the invention. In the same or a different embodiment, the inhibition or reduction of expression of target mRNA and/or target protein in the cell results in less than 100%, such as less than 98%, less than 95%, less than 90%, less than 80%, such as less than 70%, mRNA or protein levels compared to cells not exposed to the oligomer. Modulation of expression level can be determined by measuring target protein levels, e.g., by methods such as SDS-PAGE followed by western blotting using suitable antibodies raised against the target protein. Alternatively, modulation of expression levels can be determined by measuring levels of mRNA, e.g., by northern blot or quantitative RT-PCR. When measuring inhibition via mRNA levels, the level of down-regulation when using an appropriate dosage, such as from about 0.04 nm to about 400 μM concentration, is, in some embodiments, typically to a level of from about 10-20% the normal levels in the cell in the absence of the oligomer of the invention.

In certain embodiments, the oligomer of the invention has an in vivo tolerability less than or equal to a total score of 4, wherein the total score is the sum of a unit score of five categories, which are 1) hyperactivity; 2) decreased activity and arousal; 3) motor dysfunction and/or ataxia; 4) abnormal posture and breathing; and 5) tremor and/or convulsions, and wherein the unit score for each category is measured on a scale of 0-4. In certain embodiments, the in vivo tolerability is less than or equal to the total score of 3, the total score of 2, the total score of 1, or the total score of 0. In some embodiment, the assessment for in vivo tolerability is determined as described in Example 1 below.

In some embodiments, the oligomer can tolerate 1, 2, 3, or 4 (or more) mismatches, when hybridizing to the target sequence and still sufficiently bind to the target to show the desired effect, i.e., down-regulation of the target mRNA and/or protein. Mismatches may, for example, be compensated by increased length of the oligomer nucleotide sequence and/or an increased number of nucleotide analogs, which are disclosed elsewhere herein.

In some embodiments, the oligomer of the invention comprises no more than 3 mismatches when hybridizing to the target sequence. In other embodiments, the contiguous nucleotide sequence comprises no more than 2 mismatches when hybridizing to the target sequence. In other embodiments, the contiguous nucleotide sequence comprises no more than 1 mismatch when hybridizing to the target sequence. In some embodiments the region within the complement or the region can consist of 11, 12, 13, 14, 15, 16, 17, 18, 19, 20, 21, 22, 23, 24, 25, 26, 27, 28, or 29 contiguous nucleotides, such as from 12-22, such as from 14-21 nucleotides. Suitably, in some embodiments, the region is of the same length as the contiguous nucleotide sequence of the oligomer of the invention.

However, it is recognized that, in some embodiments, the nucleotide sequence of the oligomer can comprise additional 5′ or 3′ nucleotides, such as, independently, 1, 2, 3, 4 or 5 additional nucleotides 5′ and/or 3′, which are non-complementary to the target sequence. In this respect the oligomer of the invention, can, in some embodiments, comprise a contiguous nucleotide sequence which is flanked 5′ and/or 3′ by additional nucleotides.

In some embodiments, the oligomer of the invention has a sequence score greater than or equal to 0.2, wherein the sequence score is calculated by formula I:

$$\frac{\begin{matrix}\# of C nucleotides and analogs thereof - \\ \# of G nucleotides and analogs thereof\end{matrix}}{Total nucleotide length ( number )} . \qquad ( I )$$

In some embodiments, the method comprises measuring a sequence calculated by formula (IA):

$$\frac{\begin{matrix}\# of C nucleotides and 5 \text{-} methylcytosine nucleotides - \\ \# of G nucleotides\end{matrix}}{Total nucleotide length} . \qquad ( IA )$$

In other embodiments, the oligomer of the invention has a sequence score greater than or equal to 0.2.

In these embodiments, a sequence score of greater than or equal to a cut off value corresponds to a reduced neurotoxicity of the oligomer. A sequence score of greater than or equal to a cut off value corresponds to a reduced neurotoxicity of the oligomer.

In certain embodiment, the oligomer of the invention has a sequence score greater than or equal to about 0.1, 0.2, 0.25, 0.3, 0.35, 0.4, 0.45, 0.5, 0.55, 0.6, 0.65, 0.7, 0.75, 0.8, 0.85, 0.9, 0.95, or 1.0. In all of these embodiments, when the sequence score is greater than or equal to the cut off value, the oligomer is considered to have reduced neurotoxicity.

### II.B. Oligomer Length

In some embodiments, the oligomers can comprise a contiguous nucleotide sequence of a total of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 contiguous nucleotides in length.

In some embodiments, the oligomers comprise a contiguous nucleotide sequence of a total of about 10-22, such as 10-21 or 12-18, such as 13-17 or 12-16, such as 13, 14, 15, 16, 17, 18, 19, 20, or 21 contiguous nucleotides in length.

In some embodiments, the oligomers comprise a contiguous nucleotide sequence of a total of 10, 11, 12, 13, or 14 contiguous nucleotides in length.

In some embodiments, the oligomer according to the invention consists of no more than 22 nucleotides, such as no more than 21 or 20 nucleotides, such as no more than 18 nucleotides, such as 15, 16 or 17 nucleotides. In some embodiments the oligomer of the invention comprises less than 22 nucleotides. It should be understood that when a range is given for an oligomer, or contiguous nucleotide sequence length, the range includes the lower and upper lengths provided in the range, for example from (or between) 10-50, includes both 10 and 50.

### II.C. Nucleosides and Nucleoside Analogs

In one aspect of the invention, the oligomers comprise one or more non-naturally occurring nucleotide analogs. “Nucleotide analogs” as used herein are variants of natural nucleotides, such as DNA or RNA nucleotides, by virtue of modifications in the sugar and/or base moieties. Analogs could in principle be merely “silent” or “equivalent” to the natural nucleotides in the context of the oligonucleotide, i.e. have no functional effect on the way the oligonucleotide works to inhibit target gene expression. Such “equivalent” analogs can nevertheless be useful if, for example, they are easier or cheaper to manufacture, or are more stable to storage or manufacturing conditions, or represent a tag or label. In some embodiments, however, the analogs will have a functional effect on the way in which the oligomer works to inhibit expression; for example by producing increased binding affinity to the target and/or increased resistance to intracellular nucleases and/or increased ease of transport into the cell. Specific examples of nucleoside analogs are described by e.g. Freier & Altmann; Nucl. Acid Res., 1997, 25, 4429-4443 and Uhlmann; Curr. Opinion in Drug Development, 2000, 3(2), 293-213.

The oligomers of the invention comprise at least two LNA nucleosides such as at least three, at least four, at least five, at least six, at least seven, at least eight, at least nine, or at least ten LNA nucleosides. In some embodiments, the oligomer includes four, six, eight, or ten LNA nucleosides.

In some embodiments, the oligomer of the invention comprises at least one “cEt” or “constrained ethyl” means a bicyclic nucleoside having a sugar moiety comprising a bridge connecting the 4′-carbon and the 2′-carbon, wherein the bridge has the formula: 4′-CH(CH3)-0-2′.

“2′-O-methoxyethyl” (also 2′-MOE and 2′-O(CH2)2-OCH3 and MOE) refers to an O-methoxy-ethyl modification at the 2′ position of a furanosyl ring. A 2′-O-methoxyethyl modified sugar is a modified sugar.

As used herein, “2′-F” refers to modification of the 2′ position of the furanosyl sugar ring to comprise a fluoro group.

As used herein, “2′-OMe” or “2′-OCH3” or “2′-O-methyl” each refers to modification at the 2′ position of the furanosyl sugar ring to comprise a —OCH3 group.

The oligomer can thus comprise a simple sequence of natural occurring nucleotides—for example, 2′-deoxynucleotides (referred to herein generally as “DNA”), but also possibly ribonucleotides (referred to herein generally as “RNA”), or a combination of such naturally occurring nucleotides and one or more non-naturally occurring nucleotides, i.e. nucleotide analogs. Such nucleotide analogs can suitably enhance the affinity of the oligomer for the target sequence.

Examples of suitable nucleotide analogs are provided by WO2007/031091, which is incorporated by reference in its entirety, or are referenced therein.

Incorporation of affinity-enhancing nucleotide analogs in the oligomer, such as LNA or 2′-substituted sugars, can allow the size of the specifically binding oligomer to be reduced, and can also reduce the upper limit to the size of the oligomer before non-specific or aberrant binding takes place.

In some embodiments, the oligomer comprises at least one LNA. Additional details of the LNA compound are disclosed elsewhere herein. In some embodiments the oligomer comprises at least 2 LNAs. In some embodiments, the oligomer comprises from 3-10 LNAs, e.g., 6 or 7 LNAs, e.g., at least 3 or at least 4, or at least 5, or at least 6, or at least 7, or 8 LNAs. In some embodiments all the nucleotides analogs can be LNA.

In a specific embodiment, the oligomer of the invention includes a bicyclic sugar. Non-limiting examples of the bicyclic sugar includes cEt, 2′,4′-constrained 2′-O-methoxyethyl (cMOE), LNA, α-LNA, β-LNA, 2′-0,4′-C-ethylene-bridged nucleic acids (ENA), amino-LNA, oxy-LNA, or thio-LNA.

The term “thio-LNA” comprises a locked nucleotide in which Y in general Formula III below is selected from S or —CH₂—S—. Thio-LNA can be in both beta-D and alpha-L-configuration.

The term “amino-LNA” comprises a locked nucleotide in which Y in general Formula III below is selected from —N(H)—, N(R)—, CH₂—N(H)—, and —CH₂—N(R)— where R is selected from hydrogen and C₁₋₄-alkyl. Amino-LNA can be in both beta-D and alpha-L-configuration.

The term “oxy-LNA” comprises a locked nucleotide in which Y in general Formula III below represents —O—. Oxy-LNA can be in both beta-D and alpha-L-configuration.

The term “ENA” comprises a locked nucleotide in which Y in general Formula III below is —CH₂—O— (where the oxygen atom of —CH₂—O— is attached to the 2′-position relative to the base B). R^{e} is hydrogen or methyl.

In some exemplary embodiments LNA is selected from beta-D-oxy-LNA, alpha-L-oxy-LNA, beta-D-amino-LNA and beta-D-thio-LNA, in particular beta-D-oxy-LNA.

It will be recognized that when referring to a nucleotide sequence motif or nucleotide sequence, which consists of only nucleotides, the oligomers of the invention which are defined by that sequence, can comprise a corresponding nucleotide analog in place of one or more of the nucleotides present in the sequence, such as LNA units or other nucleotide analogs, including cEt, cMOE, α-LNA, β-LNA, ENA, amino-LNA, oxy-LNA, thio-LNA, which raise the duplex stability/Tₘ of the oligomer/target duplex (i.e. affinity enhancing nucleotide analogs).

In some embodiments, any mismatches between the nucleotide sequence of the oligomer and the target sequence are found in regions outside the affinity enhancing nucleotide analogs, such as region B as referred to herein, and/or region D as referred to herein, and/or at the site of non-modified such as DNA nucleotides in the oligonucleotide, and/or in regions which are 5′ or 3′ to the contiguous nucleotide sequence.

Examples of such modification of the nucleotide include modifying the sugar moiety to provide a 2′-substituent group or to produce a bridged (locked nucleic acid) structure which enhances binding affinity and can also provide increased nuclease resistance.

In one embodiment, a nucleotide analog is oxy-LNA (such as beta-D-oxy-LNA, and alpha-L-oxy-LNA), and/or amino-LNA (such as beta-D-amino-LNA and alpha-L-amino-LNA) and/or thio-LNA (such as beta-D-thio-LNA and alpha-L-thio-LNA) and/or ENA (such as beta-D-ENA and alpha-L-ENA). In a particular embodiment, a nucleotide analog is beta-D-oxy-LNA.

In some embodiments, the oligomer according to the invention comprises at least one Locked Nucleic Acid (LNA) unit, such as 1, 2, 3, 4, 5, 6, 7, 8, 9, or 10 LNA units, such as from 3-7 or 4-8 LNA units, or 3, 4, 5, 6, 7, or 8 LNA units. In some embodiments, all the nucleotide analogs are LNA. In some embodiments, the oligomer can comprise both beta-D-oxy-LNA, and one or more of the following LNA units: thio-LNA, amino-LNA, oxy-LNA, and/or ENA in either the beta-D or alpha-L configurations or combinations thereof. In some embodiments all LNA cytosine units are 5′-methylcytosine. In some embodiments of the invention, the oligomer can comprise both LNA and DNA units. In certain embodiments, the combined total of LNA and DNA units is 10-50, 10-30, such as 10-25, e.g., 10-22, such as 10-21. In some embodiments of the invention, the nucleotide sequence of the oligomer, such as the contiguous nucleotide sequence consists of at least one LNA and the remaining nucleotide units are DNA units. In some embodiments the oligomer comprises only LNA nucleotide analogs and naturally occurring nucleotides (such as RNA or DNA, e.g., DNA nucleotides), optionally with modified internucleotide linkages such as phosphorothioate.

The term “nucleobase” refers to the base moiety of a nucleotide and covers both naturally occurring as well as non-naturally occurring variants. Thus, “nucleobase” covers not only the known purine and pyrimidine heterocycles but also heterocyclic analogs and tautomeres thereof.

Examples of nucleobases include, but are not limited to adenine, guanine, cytosine, thymidine, uracil, xanthine, hypoxanthine, 5-methylcytosine, isocytosine, pseudoisocytosine, 5-bromouracil, 5-propynyluracil, 6-aminopurine, 2-aminopurine, inosine, diaminopurine, and 2-chloro-6-aminopurine.

In some embodiments, at least one of the nucleobases present in the oligomer is a modified nucleobase selected from the group consisting of 5-methylcytosine, isocytosine, pseudoisocytosine, 5-bromouracil, 5-propynyluracil, 6-aminopurine, 2-aminopurine, inosine, diaminopurine, and 2-chloro-6-aminopurine.

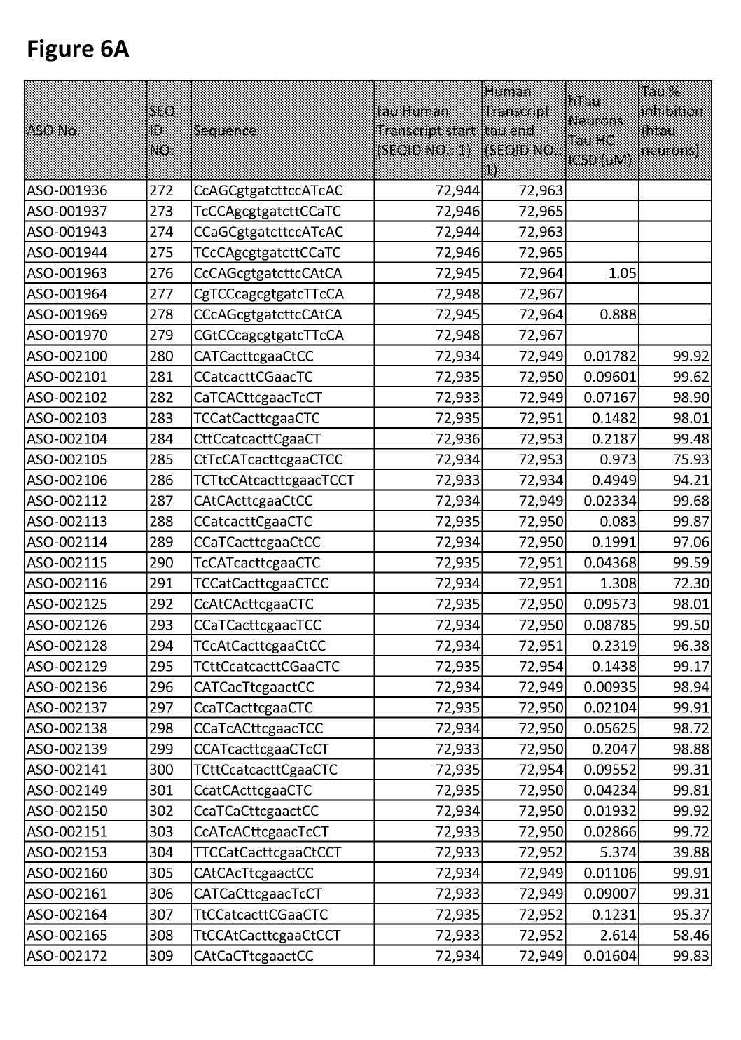

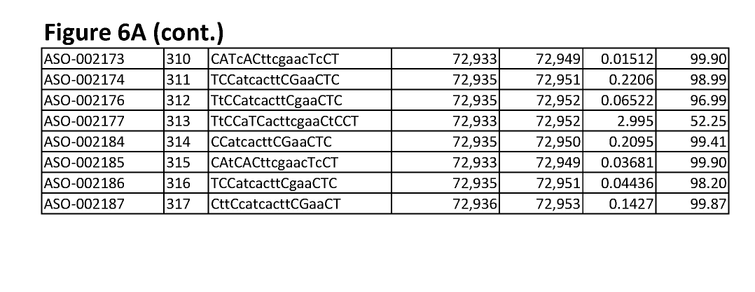

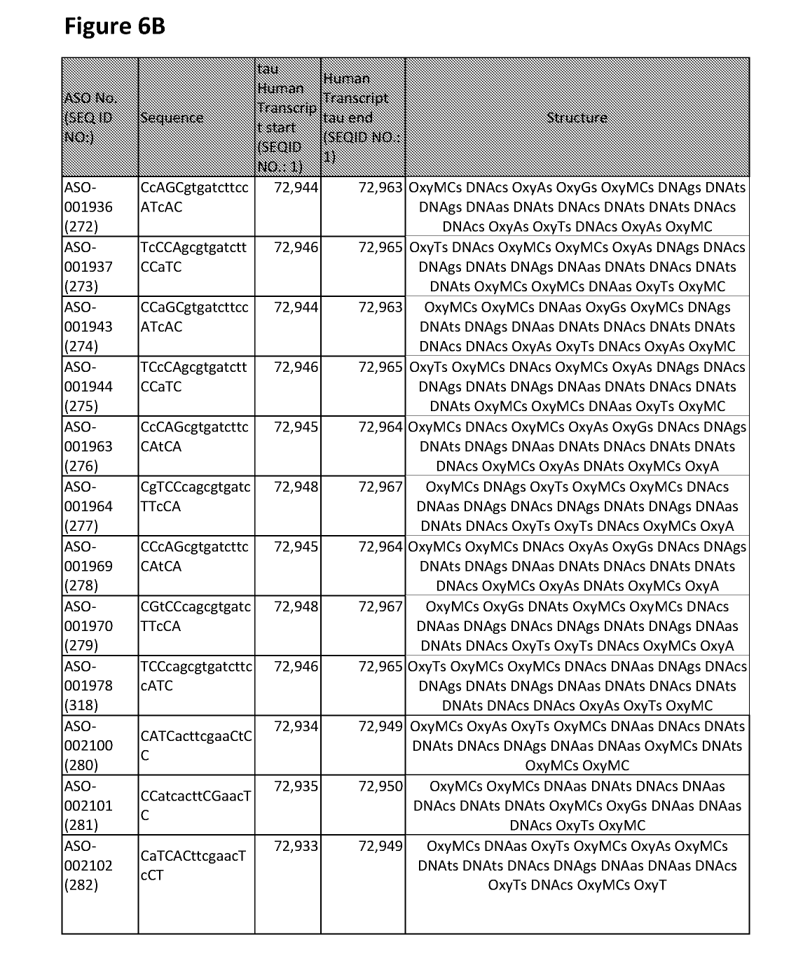

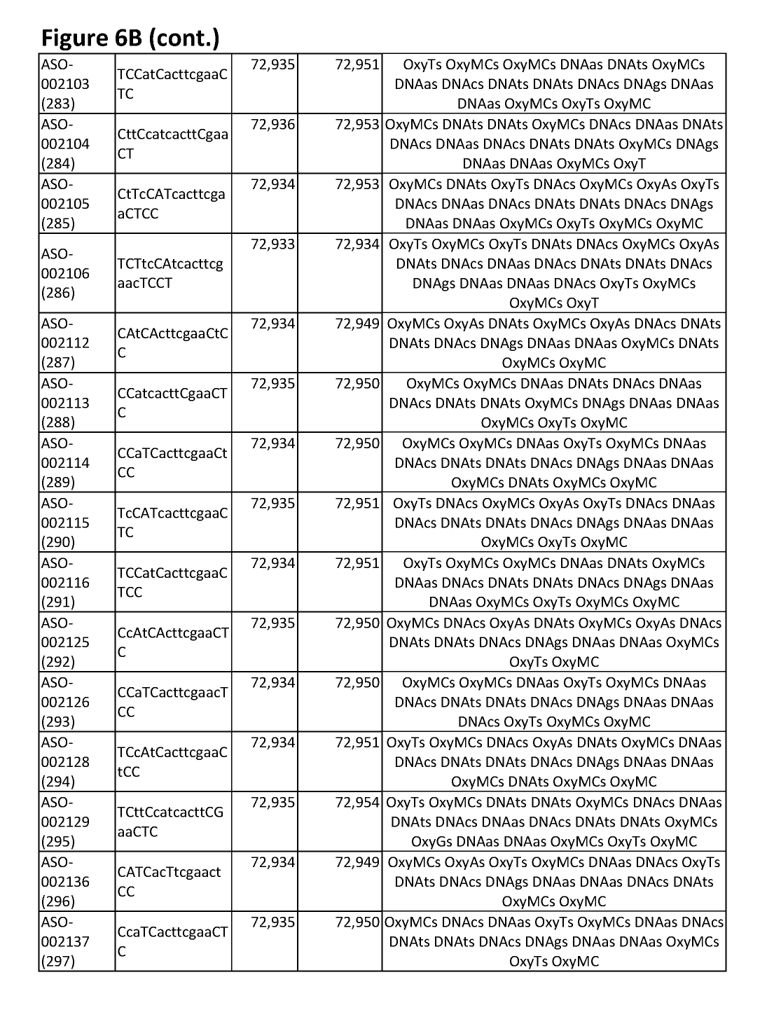

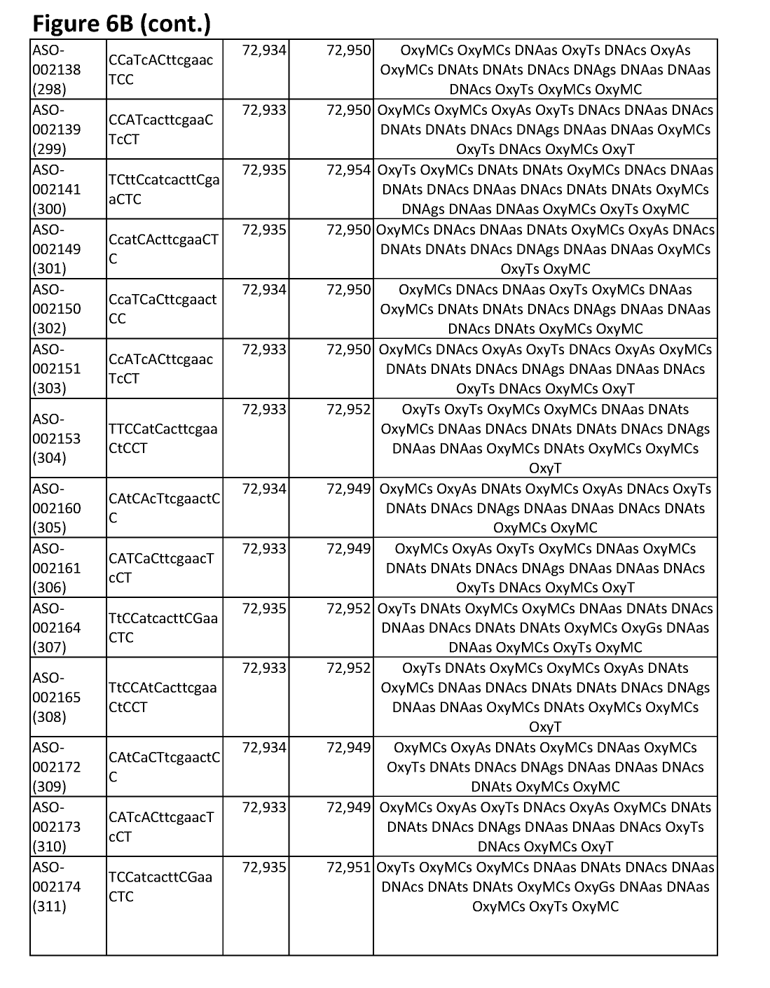

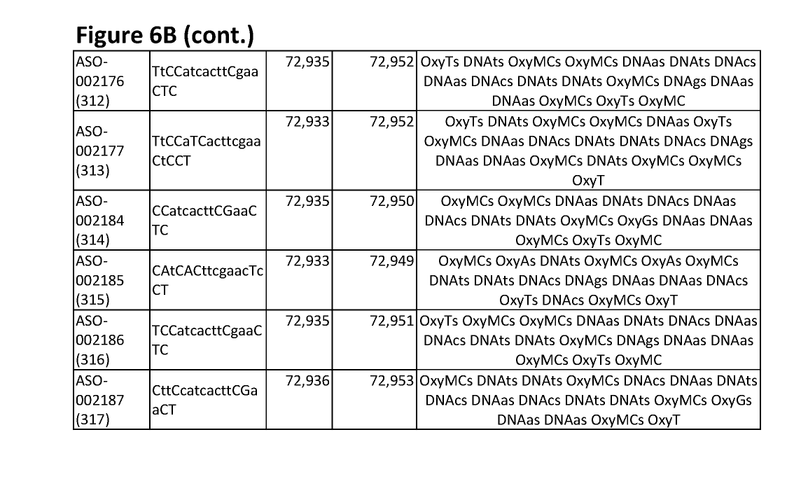

In certain embodiments, the present invention includes oligomers comprising nucleotide analogs. In some embodiments, the nucleotide analog comprises a modified nucleobase such as 5-methylcytosine. In other embodiments, the nucleotide analog comprise a modified nucleobases such as 5-methylcytosine, isocytosine, pseudoisocytosine, 5-bromouracil, 5-propynyluracil, 6-aminopurine, 2-aminopurine, inosine, diaminopurine, and 2-chloro-6-aminopurine. In certain embodiments, the oligomers have a chemical structure as disclosed in FIG. 1 or FIG. 6B.

### II.D. LNA

The term “LNA” refers to a bicyclic nucleoside analog, known as “Locked Nucleic Acid”. It can refer to an LNA monomer, or, when used in the context of an “LNA oligonucleotide,” LNA refers to an oligonucleotide containing one or more such bicyclic nucleotide analogs. LNA nucleotides are characterized by the presence of a linker group (such as a bridge) between C2′ and C4′ of the ribose sugar ring—for example as shown as the biradical R⁴* -R²* as described below.

LNAs may have the structure of the general formula V:

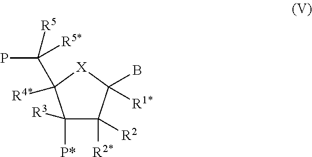

wherein for all chiral centers, asymmetric groups can be found in either R or S orientation;
wherein X is selected from —O—, —S—, —N(RN*)—, —C(R6R6*)—, such as, in some embodiments —O—;

B is selected from hydrogen, optionally substituted C1-4-alkoxy, optionally substituted C1-4-alkyl, optionally substituted C1-4-acyloxy, nucleobases including naturally occurring and nucleobase analogs, DNA intercalators, photochemically active groups, thermochemically active groups, chelating groups, reporter groups, and ligands; in some embodiments, B is a nucleobase or nucleobase analog;

P designates an internucleotide linkage to an adjacent monomer, or a 5′-terminal group, such internucleotide linkage or 5′-terminal group optionally including the sub stituent R5 or equally applicable the substituent R5*;

P* designates an internucleotide linkage to an adjacent monomer, or a 3′-terminal group;

R4* and R2* together designate a bivalent linker group consisting of 1-4 groups/atoms selected from —C(RaRb)—, —C(Ra)═C(Rb)—, —C(Ra)═N—, —O—, —Si(Ra)2-, —S—, —SO2-, —N(Ra)—, and >C═Z, wherein Z is selected from —O—, —S—, and —N(Ra)—, and Ra and Rb each is independently selected from hydrogen, optionally substituted C1-12-alkyl, optionally substituted C2-12-alkenyl, optionally substituted C2-12-alkynyl, hydroxy, optionally substituted C1-12-alkoxy, C2-12-alkoxyalkyl, C2-12-alkenyloxy, carboxy, C1-12-alkoxycarbonyl, C1-12-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C1-6-alkyl)amino, carbamoyl, mono- and di(C1-6-alkyl)-amino-carbonyl, amino-C1-6-alkyl-aminocarbonyl, mono- and di(C1-6-alkyl)amino-C1-6-alkyl-aminocarbonyl, C1-6-alkyl-carbonylamino, carbamido, C1-6-alkanoyloxy, sulphono, C1-6-alkylsulphonyloxy, nitro, azido, sulphanyl, C1-6-alkylthio, halogen, DNA intercalators, photochemically active groups, thermochemically active groups, chelating groups, reporter groups, and ligands, where aryl and heteroaryl can be optionally substituted and where two geminal substituents Ra and Rb together can designate optionally substituted methylene (═CH2), wherein for all chiral centers, asymmetric groups can be found in either R or S orientation, and; each of the substituents R1*, R2, R3, R5, R5*, R6 and R6*, which are present is independently selected from hydrogen, optionally substituted C1-12-alkyl, optionally substituted C2-12-alkenyl, optionally substituted C2-12-alkynyl, hydroxy, C1-12-alkoxy, C2-12-alkoxyalkyl, C2-12-alkenyloxy, carboxy, C1-12-alkoxycarbonyl, C1-12-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C1-6-alkyl)amino, carbamoyl, mono- and di(C1-6-alkyl)-amino-carbonyl, amino-C1-6-alkyl-aminocarbonyl, mono- and di(C1-6-alkyl)amino-C1-6-alkyl-aminocarbonyl, C1-6-alkyl-carbonylamino, carbamido, C1-6-alkanoyloxy, sulphono, C1-6-alkylsulphonyloxy, nitro, azido, sulphanyl, C1-6-alkylthio, halogen, DNA intercalators, photochemically active groups, thermochemically active groups, chelating groups, reporter groups, and ligands, where aryl and heteroaryl can be optionally substituted, and where two geminal substituents together can designate oxo, thioxo, imino, or optionally substituted methylene; wherein RN is selected from hydrogen and C1-4-alkyl, and where two adjacent (non-geminal) substituents can designate an additional bond resulting in a double bond; and RN*, when present and not involved in a biradical, is selected from hydrogen and C1-4-alkyl; and basic salts and acid addition salts thereof. For all chiral centers, asymmetric groups can be found in either R or S orientation.

In some embodiments, R4* and R2* together designate a biradical consisting of a groups selected from the group consisting of C(RaRb)—C(RaRb)—, C(RaRb)—O—, C(RaRb)—NRa—, C(RaRb)—S—, and C(RaRb)—C(RaRb)—O—, wherein each Ra and Rb can optionally be independently selected. In some embodiments, Ra and Rb can be, optionally independently selected from the group consisting of hydrogen and C1-6alkyl, such as methyl, such as hydrogen.

In some embodiments, R⁴* and R²* together designate the biradical —O—CH(CH₂OCH₃)— (2′O-methoxyethyl bicyclic nucleic acid—Seth at al., 2010, J. Org. Chem)—in either the R- or S-configuration.

In some embodiments, R⁴* and R²* together designate the biradical —O—CH(CH₂CH₃)— (2′O-ethyl bicyclic nucleic acid—Seth at al., 2010, J. Org. Chem).—in either the R- or S-configuration.

In some embodiments, R⁴* and R²* together designate the biradical —O—CH(CH₃)—.—in either the R- or S-configuration. In some embodiments, R⁴* and R²* together designate the biradical —O—CH₂—O—CH₂— (Seth at al., 2010, J. Org. Chem).

In some embodiments, R⁴* and R²* together designate the biradical —O—NR—CH₃—(Seth at al., 2010, J. Org. Chem).

In some embodiments, the LNA units have a structure selected from the following group:

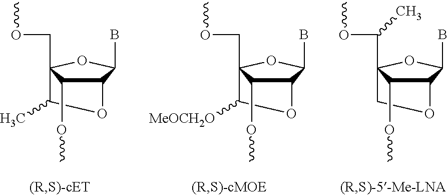

In some embodiments, the oligomer of the invention does not comprise (R, S)cET, cMOE or 5′Me-LNA units. In some embodiments, the oligomer of the invention does not comprise (S)cET LNA units.

In some embodiments, R¹*, R², R³, R⁵, R⁵* are independently selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆ alkoxyl, substituted C₁₋₆ alkoxyl, acyl, substituted acyl, C₁₋₆ aminoalkyl or substituted C₁₋₆ aminoalkyl. For all chiral centers, asymmetric groups can be found in either R or S orientation.

In some embodiments, R¹*, R², R³, R⁵, R⁵* are hydrogen.

In some embodiments, R¹*, R², R³ are independently selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆ alkoxyl, substituted C₁₋₆ alkoxyl, acyl, substituted acyl, C₁₋₆ aminoalkyl or substituted C₁₋₆ aminoalkyl. For all chiral centers, asymmetric groups can be found in either R or S orientation.

In some embodiments, R¹*, R², R³ are hydrogen.

In some embodiments, R⁵ and R⁵* are each independently selected from the group consisting of H, —CH₃, —CH₂—CH₃, —CH₂—O—CH₃, and —CH═CH₂. Suitably in some embodiments, either R⁵ or R⁵* are hydrogen, whereas the other group (R⁵ or R⁵* respectively) is selected from the group consisting of C₁₋₅ alkyl, C₂₋₆ alkenyl, C₂₋₆ alkynyl, substituted C₁₋₆ alkyl, substituted C₂₋₆ alkenyl, substituted C₂₋₆ alkynyl or substituted acyl (—C(═O)—); wherein each substituted group is mono or poly substituted with substituent groups independently selected from halogen, C₁₋₆ alkyl, substituted C₁₋₆ alkyl, C₂₋₆ alkenyl, substituted C₂₋₆ alkenyl, C₂₋₆ alkynyl, substituted C₂₋₆ alkynyl, OJ₁, SJ₁, NJ₁J₂, N₃, COOJ₁, CN, O—C(═O)NJ₁J₂, N(H)C(═NH)NJ,J₂ or N(H)C(═X)N(H)J₂ wherein X is O or S; and each J₁ and J₂ is, independently, H, C₁₋₆ alkyl, substituted C₁₋₆ alkyl, C₂₋₆ alkenyl, substituted C₂₋₆ alkenyl, C₂₋₆ alkynyl, substituted C₂₋₆ alkynyl, C₁₋₆ aminoalkyl, substituted C₁₋₆ aminoalkyl or a protecting group. In some embodiments either R⁵ or R⁵* is substituted C₁₋₆ alkyl. In some embodiments either R⁵ or R⁵* is substituted methylene wherein preferred substituent groups include one or more groups independently selected from F, NJ₁J₂, N₃, CN, OJ₁, SJ₁, O—C(═O)NJ₁J₂, N(H)C(═NH)NJ, J₂ or N(H)C(O)N(H)J₂. In some embodiments each J₁ and J₂ is, independently H or C₁₋₆ alkyl. In some embodiments either R⁵ or R⁵* is methyl, ethyl or methoxymethyl. In some embodiments either R⁵ or R⁵* is methyl. In a further embodiment either R⁵ or R⁵* is ethylenyl. In some embodiments either R⁵ or R⁵* is substituted acyl. In some embodiments either R⁵ or R⁵* is C(═O)NJ₁J₂. For all chiral centers, asymmetric groups can be found in either R or S orientation. Such 5′ modified bicyclic nucleotides are disclosed in WO 2007/134181, which is hereby incorporated by reference in its entirety.

In some embodiments B is a nucleobase, including nucleobase analogs and naturally occurring nucleobases, such as a purine or pyrimidine, or a substituted purine or substituted pyrimidine, such as a nucleobase referred to herein, such as a nucleobase selected from the group consisting of adenine, cytosine, thymine, adenine, uracil, and/or a modified or substituted nucleobase, such as 5-thiazolo-uracil, 2-thio-uracil, 5-propynyluracil, 2′thio-thymine, 5-methyl cytosine, 5-thiozolo-cytosine, 5-propynyl-cytosine, and 2,6-diaminopurine.

In some embodiments, R⁴* and R²* together designate a biradical selected from —C(R^{a}R^{b})—O—, —C(R^{a}R^{b})—C(R^{c}R^{d})—O—, —C(R^{a}R^{b})—C(R^{c}R^{d})—C(R^{e}R^{f})—O—, —C(R^{a}R^{b})—O—C(R^{c}R^{d})—, —C(R^{a}R^{b})—O—C(R^{c}R^{d})—O—, —C(R^{a}R^{b})—C(R^{c}R^{d})—, —C(R^{a}R^{b})—C(R^{c}R^{d})—C(R^{e}R^{f})—, —C(R^{a})═C(R^{b})—C(R^{c}R^{d})—, —C(R^{a}R^{b})—N(R^{c})—, —C(R^{a}R^{b})—C(R^{c}R^{d})— N(R^{e})—, —C(R^{a}R^{b})—N(R^{c})—O—, and —C(R^{a}R^{b})—S—, —C(R^{a}R^{b})—C(R^{c}R^{d})—S—, wherein R^{a}, R^{b}, le, R^{d}, R^{e}, and R^{f} each is independently selected from hydrogen, optionally substituted C₁₋₁₂-alkyl, optionally substituted C₂₋₁₂-alkenyl, optionally substituted C₂₋₁₂-alkynyl, hydroxy, C₁₋₁₂-alkoxy, C₂-12-alkoxyalkyl, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl can be optionally substituted and where two geminal substituents R^{a} and R^{b} together can designate optionally substituted methylene (═CH₂). For all chiral centers, asymmetric groups can be found in either R or S orientation.

In a further embodiment R⁴* and R²* together designate a biradical (bivalent group) selected from —CH₂—O—, —CH₂—S—, —CH₂—NH—, —CH₂—N(CH₃)—, —CH₂—CH(CH₃)—, —CH₂—CH₂—NH—, —CH₂—CH₂—CH₂—, —CH₂—CH₂—CH₂—O—, —CH₂—CH₂—CH(CH₃)—, —CH═CH—CH₂—, —CH₂—N(CH₃)—O—, —CH₂—CH(CH₃)—O—, and —CH(CH₂—O—CH₃)—O—, and/or, —CH₂—CH₂—, and —CH═CH— For all chiral centers, asymmetric groups can be found in either R or S orientation.

In some embodiments, R⁴* and R²* together designate the biradical C(R^{a}R^{b})—N(R^{c})—O—, wherein R^{a} and R^{b} are independently selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆ alkoxyl, substituted C₁₋₆ alkoxyl, acyl, substituted acyl, C₁₋₆ aminoalkyl or substituted C₁₋₆ aminoalkyl, such as hydrogen, and; wherein R^{e} is selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆ alkoxyl, substituted C₁₋₆ alkoxyl, acyl, substituted acyl, C₁₋₆ aminoalkyl or substituted C₁₋₆ aminoalkyl, such as hydrogen.

In some embodiments, R⁴* and R²* together designate the biradical C(R^{a}R^{b})—O—C(R^{c}R^{d})—O—, wherein R^{a}, R^{b}, le, and R^{d} are independently selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆ alkoxyl, substituted C₁₋₆ alkoxyl, acyl, substituted acyl, C₁₋₆ aminoalkyl or substituted C₁₋₆ aminoalkyl, such as hydrogen.

In some embodiments, R⁴* and R²* form the biradical —CH(Z)—O—, wherein Z is selected from the group consisting of C₁₋₆ alkyl, C₂₋₆ alkenyl, C₂₋₆ alkynyl, substituted C₁₋₆ alkyl, substituted C₂₋₆ alkenyl, substituted C₂₋₆ alkynyl, acyl, substituted acyl, substituted amide, thiol or substituted thio; and wherein each of the substituted groups, is, independently, mono or poly substituted with optionally protected substituent groups independently selected from halogen, oxo, hydroxyl, OJ₁, NJ₁J₂, SJ₁, N₃, OC(═X)J₁, OC(═X)NJ₁J₂, NJ³C(═X)NJ₁J₂ and CN, wherein each J₁, J₂ and J₃ is, independently, H or C₁₋₆ alkyl, and X is O, S or NJ₁. In some embodiments Z is C₁₋₆ alkyl or substituted C₁₋₆ alkyl. In some embodiments Z is methyl. In some embodiments Z is substituted C₁₋₆ alkyl. In some embodiments the substituent group is C₁₋₆ alkoxy. In some embodiments Z is CH₃OCH₂—. For all chiral centers, asymmetric groups can be found in either R or S orientation. Such bicyclic nucleotides are disclosed in U.S. Pat. No. 7,399,845 which is hereby incorporated by reference in its entirety. In some embodiments, R¹*, R², R³, R⁵, R⁵* are hydrogen. In some embodiments, R¹*, R², R³* are hydrogen, and one or both of R⁵, R⁵* can be other than hydrogen as referred to above and in WO 2007/134181, which is incorporated by reference herein in its entirety.

In some embodiments, R⁴* and R²* together designate a biradical which comprise a substituted amino group in the bridge such as consist of or comprise the biradical —CH₂—N(R^{c})—, wherein R^{c} is C₁₋₁₂ alkyloxy. In some embodiments R⁴* and R²* together designate a biradical -Cq₃q₄-NOR—, wherein q₃ and q₄ are independently selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆ alkoxyl, substituted C₁₋₆ alkoxyl, acyl, substituted acyl, C₁₋₆ aminoalkyl or substituted C₁₋₆ aminoalkyl; wherein each substituted group is, independently, mono or poly substituted with substituent groups independently selected from halogen, OJ₁, SJ₁, NJ₁J₂, COOJ₁, CN, 0-C(═O)NJ₁J₂, N(H)C(═NH)N J₁J₂ or N(H)C(═X═N(H)J₂ wherein X is O or S; and each of J₁ and J₂ is, independently, H, C₁₋₆ alkyl, C₂₋₆ alkenyl, C₂₋₆ alkynyl, C₁₋₆ aminoalkyl or a protecting group. For all chiral centers, asymmetric groups can be found in either R or S orientation. Such bicyclic nucleotides are disclosed in WO2008/150729 which is hereby incorporated by reference in its entirety. In some embodiments, R¹*, R², R³, R⁵, R⁵* are independently selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆ alkoxyl, substituted C₁₋₆ alkoxyl, acyl, substituted acyl, C₁₋₆ aminoalkyl or substituted C₁₋₆ aminoalkyl. In some embodiments, R¹*, R², R³, R⁵, R⁵* are hydrogen. In some embodiments, R¹*, R², R³ are hydrogen and one or both of R⁵, R⁵* can be other than hydrogen as referred to above and in WO 2007/134181. In some embodiments R⁴* and R²* together designate a biradical (bivalent group) C(R^{a}R^{b})—O—, wherein R^{a} and R^{b} are each independently halogen, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₁-C₁₂ alkoxy, substituted C₁-C₁₂ alkoxy, OJ₁SJ₁, SOJ₁, SO₂J₁, NJ₁J₂, N₃, CN, C(═O)OJ₁, C(═O)NJ₁J₂, C(═O)J₁, O—C(═O)NJ₁J₂, N(H)C(═NH)NJ₁J₂, N(H)C(═O)NJ₁J₂ or N(H)C(═S)NJ₁J₂; or R^{a} and R^{b} together are ═C(q3)(q4); q₃ and q₄ are each, independently, H, halogen, C₁-C₁₂alkyl or substituted C₁-C₁₂ alkyl; each substituted group is, independently, mono or poly substituted with substituent groups independently selected from halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl, OJ₁, SJ₁, NJ₁J₂, N₃, CN, C(═O)OJ₁, C(═O)NJ₁J₂, C(═O)J₁, O—C(═O)NJ₁J₂, N(H)C(═O)NJ₁J₂ or N(H)C(═S)NJ₁J₂. and; each J₁ and J₂ is, independently, H, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl, C₁-C₆ aminoalkyl, substituted C₁-C₆ aminoalkyl or a protecting group. Such compounds are disclosed in WO2009006478A, hereby incorporated in its entirety by reference.

In some embodiments, R⁴* and R²* form the biradical -Q-, wherein Q is C(q₁)(q₂)C(q₃)(q₄), C(q₁)═C(q₃), C[═C(q₁)(q₂)]-C(q₃)(q₄) or C(q₁)(q₂)-C[═C(q₃)(q₄)]; q₁, q2, q3, q₄ are each independently. H, halogen, C₁₋₁₂ alkyl, substituted C₁₋₁₂ alkyl, C₂₋₁₂ alkenyl, substituted C₁₋₁₂ alkoxy, OJ₁, SJ₁, 50J₁, 50₂J₁, NJ₁J₂, N₃, CN, C(═O)OJ₁, C(═O)-NJ₁J₂, C(═O) J₁, —C(═O)NJ₁J₂, N(H)C(═NH)NJ₁J₂, N(H)C(═O)NJ₁J₂ or N(H)C(═S)NJ₁J₂; each J₁ and J₂ is, independently, H, C₁₋₆ alkyl, C₂₋₆ alkenyl, C₂₋₆ alkynyl, C₁₋₆ aminoalkyl or a protecting group; and, optionally wherein when Q is C(q₁)(q₂)(q₃)(q₄) and one of q₃ or q₄ is CH₃ then at least one of the other of q₃ or q₄ or one of q₁ and q₂ is other than H. In some embodiments, R¹*, R², R³, R⁵, R⁵* are hydrogen. For all chiral centers, asymmetric groups can be found in either R or S orientation. Such bicyclic nucleotides are disclosed in WO2008/154401 which is hereby incorporated by reference in its entirety. In some embodiments, R¹*, R², R³, R⁵, R⁵* are independently selected from the group consisting of hydrogen, halogen, C₁₋₆ alkyl, substituted C₁₋₆ alkyl, C₂₋₆ alkenyl, substituted C₂₋₆ alkenyl, C₂₋₆ alkynyl or substituted C₂₋₆ alkynyl, C₁₋₆ alkoxyl, substituted C₁₋₆ alkoxyl, acyl, substituted acyl, C₁₋₆ aminoalkyl or substituted C₁₋₆ aminoalkyl. In some embodiments, R¹*, R², R³, R⁵, R⁵* are hydrogen. In some embodiments, R¹*, R², R³ are hydrogen and one or both of R⁵, R⁵* can be other than hydrogen as referred to above and in WO 2007/134181 or WO2009/067647 (alpha-L-bicyclic nucleic acids analogs).

Further bicyclic nucleoside analogs and their use in antisense oligonucleotides are disclosed in WO2011/115818, WO2011/085102, WO2011/017521, WO09/100320, WO10/036698, WO09/124295 & WO09/006478, each of which are incorporated by reference herein in their entireties. Such nucleoside analogs can in some aspects be useful in the compounds of present invention.

In some embodiments the LNA used in the oligonucleotide compounds of the invention has the structure of the general formula VI:

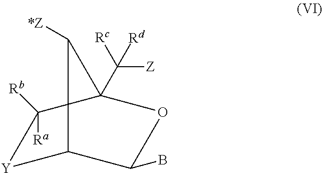

wherein Y is selected from the group consisting of —O—, —CH₂O—, —S—, —NH—, N(R^{e}) and/or —CH₂—; Z and Z* are independently selected among an internucleotide linkage, R^{H}, a terminal group or a protecting group; B constitutes a natural or non-natural nucleotide base moiety (nucleobase), and R^{H} is selected from hydrogen and C₁₋₄-alkyl; R^{a}, R^{b} R^{c}, R^{d} and R^{e} are, optionally independently, selected from the group consisting of hydrogen, optionally substituted C₁₋₁₂-alkyl, optionally substituted C₂₋₁₂-alkenyl, optionally substituted C₂₋₁₂-alkynyl, hydroxy, C₁₋₁₂-alkoxy, C₂₋₁₂-alkoxyalkyl, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl can be optionally substituted and where two geminal substituents R^{a} and R^{b} together can designate optionally substituted methylene (═CH₂); and R^{H} is selected from hydrogen and C₁₋₄-alkyl. In some embodiments R^{a}, R^{b} R^{c}, R^{d} and R^{e} are, optionally independently, selected from the group consisting of hydrogen and C₁₋₆ alkyl, such as methyl. For all chiral centers, asymmetric groups can be found in either R or S orientation, for example, two exemplary stereochemical isomers include the beta-D and alpha-L isoforms, which can be illustrated as follows:

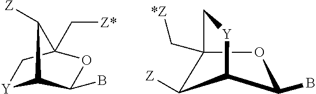

Specific exemplary LNA units are shown below:

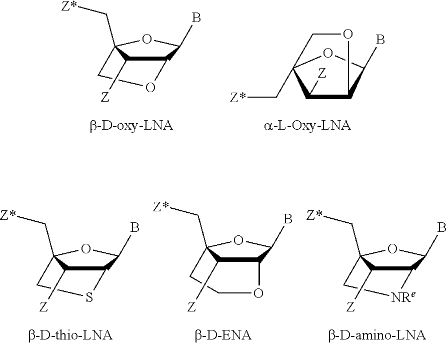

In some embodiments, the oligomer of the invention comprises beta-D-oxy-LNA units, and if present, other LNA units selected from the group consisting of alpha-L-LNA, beta-D-thio-LNA, beta-D-amino LNA and beta-D-ENA-LNA. In some embodiments, all LNA units present in the oligomer of the invention are beta-D-oxy-LNA units

In other embodiments, the oligomers of the invention comprise nucleotides with modified sugar moieties as described in FIGS. 1, 2, 3, 4, 6A, 6B, 11A and 11B.

### II.E. RNase Recruitment

It is recognized that an oligomeric compound can function via non RNase mediated degradation of target mRNA, such as by steric hindrance of translation, or other methods, however, in one aspect, the oligomers of the invention are capable of recruiting an endoribonuclease (RNase), such as RNaseH.

In one aspect, the oligomer, or contiguous nucleotide sequence, comprises a region of at least 7 consecutive nucleotide units, such as at least 8 or at least 9 consecutive nucleotide units (residues), in certain embodiments including 7, 8, 9, 10, 11, 12, 13, 14, 15, 16, 17, 18, 19, 20, 21, 22, or 23 consecutive nucleotides, which, when formed in a duplex with the complementary target RNA is capable of recruiting RNase. The contiguous sequence which is capable of recruiting RNase can be region B as referred to in the context of a gapmer as described herein. In some embodiments the size of the contiguous sequence which is capable of recruiting RNase, such as region B, can be higher, such as 10, 11, 12, 13, 14, 15, 16, 17, 18, 19, 20, 21, 22, or 23 nucleotide units.

U.S. Pat. No. 6,617,442, which is incorporated by reference herein in its entirety, provides in vitro methods for determining RNaseH activity, which can be used to determine the ability to recruit RNaseH. Therefore, in one embodiment, an oligomer of the invention is capable of recruiting RNaseH. In another embodiment, the invention includes a method of identifying an oligomer which is capable of utilizing RNaseH mechanism, e.g., recruiting RNaseH.

Oligomers can be screened to identify those which are effective in recruiting RNaseH. The ability of oligomers to recruit RNaseH can be determined by measuring the binding of the oligomers to RNaseH. The methods of determining binding of the oligomers to RNaseH are well known in the art. For example, the oligomers can be radiolabeled and binding of the oligomers to RNaseH can be detected by autoradiography. In some embodiments, fusion proteins of RNaseH with glutathione-S-transferase or small peptide tags can be prepared and immobilized to a solid phase such as beads. Labeled or unlabeled oligomers to be screened for binding to this enzyme can then be incubated with the solid phase. Oligomers which bind to the enzyme immobilized to the solid phase can then be identified either by detection of bound label or by eluting specifically the bound oligomers from the solid phase. Another method involves screening of oligomer libraries for binding partners. Recombinant tagged or labeled RNaseH is used to select oligomers from the library which interact with the enzyme. Sequencing of the oligomers leads to identification of those oligomers which will be more effective as antisense molecules.

An oligomer is deemed capable of recruiting RNaseH if, when provided with the complementary RNA target, it has an initial rate, as measured in pmol/l/min, of at least 1%, such as at least 5%, such as at least 10% or, more than 20% of the of the initial rate determined using DNA only oligonucleotide, having the same base sequence but containing only DNA monomers, with no 2′ substitutions, with phosphorothioate linkage groups between all monomers in the oligonucleotide—using the methodology provided by Example 91-95 of U.S. Pat. No. 6,617,442.

In some embodiments, an oligomer is deemed essentially incapable of recruiting RNaseH if, when provided with the complementary RNA target, and RNaseH, the RNaseH initial rate, as measured in pmol/l/min, is less than 1%, such as less than 5%, such as less than 10% or less than 20% of the initial rate determined using the equivalent DNA only oligonucleotide, with no 2′ substitutions, with phosphorothioate linkage groups between all nucleotides in the oligonucleotide, using the methodology provided by Example 91-95 of U.S. Pat. No. 6,617,442.

In other embodiments, an oligomer is deemed capable of recruiting RNaseH if, when provided with the complementary RNA target, and RNaseH, the RNaseH initial rate, as measured in pmol/l/min, is at least 20%, such as at least 40%, such as at least 60%, such as at least 80% of the initial rate determined using the equivalent DNA only oligonucleotide, with no 2′ substitutions, with phosphorothioate linkage groups between all nucleotides in the oligonucleotide, using the methodology provided by Example 91-95 of U.S. Pat. No. 6,617,442.

Typically the region of the oligomer which forms the consecutive nucleotide units which, when formed in a duplex with the complementary target RNA is capable of recruiting RNase consists of nucleotide units which form a DNA/RNA like duplex with the RNA target—and include both DNA units and LNA units which are in the alpha-L configuration, particularly preferred being alpha-L-oxy LNA.

In some embodiments, the monomers which are capable of recruiting RNase are selected from the group consisting of DNA monomers, alpha-L-LNA monomers, C4′ alkylayted DNA monomers (see PCT/EP2009/050349 and Vester et al., Bioorg. Med. Chem. Lett. 18 (2008) 2296-2300, hereby incorporated by reference in its entirety), and UNA (unlinked nucleic acid) nucleotides (see Fluiter et al., Mol. Biosyst., 2009, 10, 1039, hereby incorporated by reference). UNA is unlocked nucleic acid, typically where the C2-C3 C—C bond of the ribose has been removed, forming an unlocked “sugar” residue.

### II.F. Oligomer Design

The oligomer of the invention can comprise a nucleotide sequence which comprises both nucleotides and nucleotide analogs, and can be in the form of a gapmer, blockmer, mixmer, headmer, tailmer, or totalmer. Examples of configurations of a gapmer, blockmer, mixmer, headmer, tailmer, or totalmer that can be used with the oligomer of the invention are described in U.S. Patent Appl. Publ. No. 2012/0322851, which is incorporated by reference herein in its entirety.

Non-limiting examples of the oligomers are disclosed in the Figures herein to show representative designs and sequences, but the oligomer designs are not limited with the particular sequences disclosed herein.

A gapmer oligomer is an oligomer which comprises a contiguous stretch of nucleotides which is capable of recruiting an RNase, such as RNaseH, such as a region of at least 5 DNA nucleotides, at least 6 DNA nucleotides, or at least 7 DNA nucleotides, which is flanked both 5′ and 3′ by regions of affinity enhancing (e.g., 1-6) nucleotide analogs 5′ and 3′ to the contiguous stretch of nucleotides which is capable of recruiting RNase.

The term “gapmer” as used herein can include a traditional gapmer (e.g., oligomer with a design of G-H-I, wherein region H comprises DNAs only, and regions G and I comprise LNAs only) or a gapmer with an alternating flank at either wing position. The term “alternating flank” as referred to herein means a contiguous sequence of at least three nucleosides comprising LD₁₋₆L in any portion of the sequence wherein L is LNA and D is DNA (e.g., LDL, LDDL, LDDDL, LDDDDL, LDDDDDL, or LDDDDDDL).

In some embodiments, in addition to enhancing affinity of the oligomer for the target region, some nucleoside analogs also mediate RNase (e.g., RNaseH) binding and cleavage. Since α-L-LNA monomers recruit RNaseH activity to a certain extent, in some embodiments, gap regions (e.g., region B as referred to herein) of oligomers containing α-L-LNA monomers consist of fewer monomers recognizable and cleavable by the RNaseH, and more flexibility in the mixmer construction is introduced.

The oligomer of the invention comprises region A, region B, and region C (A-B-C), wherein region B comprises at least 5, at least 6, at least 7, at least 8, at least 9, or at least 10 consecutive nucleoside units and is flanked at 5′ by region A of 1-8 contiguous nucleoside units and at 3′ by region C of 1-8 contiguous nucleoside units, wherein region B, when formed in a duplex with a complementary RNA, is capable of recruiting RNaseH, and wherein at least one of region A and region C comprises at least one terminal LNA nucleotide, a DNA nucleoside, and a further LNA nucleoside adjacent to region B.

In some embodiments, region A comprises a 5′ LNA nucleoside unit and a 3′ LNA nucleoside unit, and at least one DNA nucleoside unit between the 5′ LNA nucleoside unit and the 3′ LNA nucleoside unit, and region C comprises at least two 3′ LNA nucleoside units.

In some embodiments, region C comprises a 5′ LNA nucleoside unit, at least two terminal 3′ LNA nucleoside units, and at least one DNA nucleoside unit between the 5′ LNA nucleoside unit and the 3′ LNA nucleoside units, and region A comprises at least one 5′ LNA nucleoside unit.

In some embodiments, region A comprises a 5′ LNA nucleoside unit and a 3′ LNA nucleoside unit, and at least one DNA nucleoside unit between the 5′ LNA nucleoside unit and the 3′ LNA nucleoside unit; and region C comprises a 5′ LNA nucleoside unit, at least two terminal 3′ LNA nucleoside units, and at least one DNA nucleoside unit between the 5′ LNA nucleoside unit and the 3′ LNA nucleoside units.

In certain embodiments, an oligomer design can provide an enhanced property, e.g., reducing non-specific binding while retaining affinity to the target nucleic acid. For example, while not being bound by any theory, the present disclosure provides an oligomer that retains affinity for a target nucleic acid that is comparable to a corresponding reference gapmer, but exhibits reduced non-specific binding to non-target nucleic acids compared to the corresponding reference gapmer. In other embodiments, the oligomers of the invention further comprise one or more enhanced properties, e.g., stability, less toxicity, etc.

In some embodiments, the oligomers of the invention have reduced toxicity in vitro and/or in vivo compared to a corresponding reference oligomer that does not have the design described herein (e.g., a gapmer with one or two alternating flanks). In certain embodiments, the alternating flank gapmer exhibits less off target binding compared to a corresponding reference oligomer that does not have the design described herein. In some embodiments, the corresponding reference oligomer has the same sequence as the oligomer of the present invention, but has a gapmer design of G-H-I, wherein region H is identical to region B, region G has the same sequence as region A except that the nucleosides in region G are all LNAs, and region I has the same sequence as region C except that the nucleosides in region I are all LNAs.

In some embodiments, either region A or region C comprises 1, 2, or 3 DNA nucleoside units. In some embodiments, region A and region C each comprise 1, 2, or 3 DNA nucleoside units.

In some embodiments, region B comprises at least five consecutive DNA nucleoside units. In some embodiments, region B comprises 6, 7, 8, 9, 10, 11, 12, 13 or 14 consecutive DNA nucleoside units. In some embodiments, region B is 8, 9 10, 11, or 12 nucleotides in length.

In some embodiments, region A comprises two 5′ terminal LNA nucleoside units.

In some embodiments, region A has formula 5′ [LNA]₁₋₃ [DNA]₁₋₃ [LNA]₁₋₃, or 5′ [LNA]₁₋₂[DNA]₁₋₂ [LNA]₁₋₂ [DNA]₁₋₂ [LNA]₁₋₂.

The term [LNA]x-y as used herein means the number of LNA units in a contiguous sequence. The minimum number of LNA units that can be present in the sequence is x and the maximum number of LNA units that can be present in the sequence is y.

When region A of 1-8 contiguous nucleoside units has formula 5′ [LNA]₁₋₃[DNA]₁₋₃[LNA]₁₋₃, the total number of [LNA][DNA][LNA] may not be higher than 8 contiguous nucleoside units (e.g., 3-3-2, 3-2-3, or 2-3-3) while one or two of the first [LNA], [DNA] and the second [LNA] can be as high as 3.

In some embodiments, region C has formula [LNA]₁₋₃ [DNA]₁₋₃ [LNA]₂₋₃3′, or [LNA]₁₋₂ [DNA]₁₋₂ [LNA]₁₋₂ [DNA]₁₋₂ [LNA]₂₋₃ 3′.

In some embodiments, region A has formula 5′ [LNA]₁₋₃ [DNA]₁₋₃ [LNA]₁₋₃ or 5′ [LNA]₁₋₂ [DNA]₁₋₂ [LNA]₁₋₂ [DNA]₁₋₂ [LNA]₁₋₂, and region C comprises 2, 3, 4 or 5 consecutive LNA nucleoside units.

In some embodiments, region C has formula [LNA]₁₋₃ [DNA]₁₋₃ [LNA]₂₋₃3 ‘ or [LNA]₁₋₂ [DNA]₁₋₂ [LNA]₁₋₂ [DNA]₁₋₂ [LNA]₂₋₃3’, and region A comprises 1, 2, 3, 4 or 5 consecutive LNA nucleoside units.

In some embodiments, region A has a sequence of LNA and DNA nucleosides, 5′-3′ selected from the group consisting of L, LL, LDL, LLL, LLDL, LDLL, LDDL, LLLL, LLLLL, LLLDL, LLDLL, LDLLL, LLDDL, LDDLL, LLDLD, LDLLD, LDDDL, LLLLLL, LLLLDL, LLLDLL, LLDLLL, LDLLLL, LLLDDL, LLDLDL, LLDDLL, LDDLLL, LDLLDL, LDLDLL, LDDDLL, LLDDDL, and LDLDLD, wherein L represents a LNA nucleoside, and D represents a DNA nucleoside.

In some embodiments, region C has a sequence of LNA and DNA nucleosides, 5′-3′ selected from the group consisting of LL, LLL, LLLL, LDLL, LLLLL, LLDLL, LDLLL, LDDLL, LDDLLL, LLDDLL, LDLDLL, LDDDLL, LDLDDLL, LDDLDLL, LDDDLLL, and LLDLDLL.

In some embodiments, region A has a sequence of LNA and DNA nucleosides, 5′-3′ selected from the group consisting of LDL, LLDL, LDLL, LDDL, LLLDL, LLDLL, LDLLL, LLDDL, LDDLL, LLDLD, LDLLD, LDDDL, LLLLDL, LLLDLL, LLDLLL, LDLLLL, LLLDDL, LLDLDL, LLDDLL, LDDLLL, LDLLDL, LDLDLL, LDDDLL, LLDDDL, and LDLDLD, and region C has a sequence of LNA and DNA nucleosides, 5′-3′ selected from the group consisting of LDLL, LLLLL, LLDLL, LDLLL, LDDLL, LDDLLL, LLDDLL, LDLDLL, LDDDLL, LDLDDLL, LDDLDLL, LDDDLLL, and LLDLDLL.

In some embodiments, the contiguous nucleotides comprises an alternating sequence of LNA and DNA nucleoside units, 5-3′, selected from the group consisting of: 2-3-2-8-2, 1-1-2-1-1-9-2, 3-10-1-1-2, 3-9-1-2-2, 3-8-1-3-2, 3-8-1-1- 1-1-2, 3-1-1-9-3, 3-1-1-8-1-1-2, 4-9-1-1-2, 4-8-1-2-2, 3-3-1-8-2, 3-2-1-9-2, 3-2-2-8-2, 3-2-2-7-3, 5-7-1-2-2, 1-1-3-10-2, 1-1-3-7-1-2-2, 1-1-4-9-2, 2-1-3-9-2, 3-1-1-10-2, 3-1-1-7-1-2-2, 3-1-2-9-2, 4-7-1-3-2, 5-9-1-1-2, 4-10-1-1-2, 3-11-1-1-2, 2-1-1-10-1-1-2, 1-1-3-9-1-1-2, 3-10-1-2-2, 3-9-1-3-2, 3-8-1-1-1-2-2, 4-9-1-2-2, 4-9-1-1-3, 4-8-1-3-2, 4-8-1-2-3, 4-8-1-1-1-1-2, 4-7-1-2-1-1-2, 4-7-1-1-1-2-2, 2-1-2-11-2, 2-1-3-8-1-1-2, 3-1-1-11-2, 3-1-1-9-1-1-2, 3-1-1-8-1-2-2, 3-1-1-7-1-1-1-1-2, 4-9-2-1-2, 4-7-1-3-3, 5-9-1-1-3, 5-9-1-2-2, 4-10-2-1-2, 4-10-1-1-3, 4-10-1-2-2, 3-11-2-1-2, 3-11-1-1-3, 5-9-2-1-2, 3-11-1-2-2, 2-1-2-9-1-2-2, 3-1-1-10-1-1-2, 3-1-1-9-1-2-2, 4-9-1-1-1-1-2, 4-8-2-1-1-1-2, 1-1-3-10-2-1-2, 2-1- 2-10-2-1-2, 2-1-1-12-4, 2-2-1-11-4, 3-1-1-11-4, 2-1-1-13-3, 2-1-2-11-4, 2-2-1-12-3, 3-11-1-2-3, 3-1-1-12-3, 2-1-2-12-3, 4-11-2-1-2, 4-10-2-2-2, 3-2-1-9-1-1-3, 2-2-1-1-1-9-4, 2-2-2-9-1-1-3, 3-1-1-9-1-1-1-1-2, 2-1-2-9-1-2-3, 3-1-1-10-1-1-3, 2-1-1-2-1-9-4, 4-9-1-1-1-2-2, 3-1-1-9-1-2-3, 2-1-1-1-1-10-4, 2-1-2-10-1-1-3, 2-1-1-1-1-9-2-1-2, 2-2-2-9-2-1-2, 4-9-1-2-1-1-2, 3-2-1-9-2-1-2, 2-1-2-9-2-2-2, 2-1-1-1-1-9-1-1-3, 3-1-1-9-2-2-2, 2-2-2-10-4, 2-1- 2-9-1-1-1-1-2, 4-10-1-2-3, 3-2-1-10-4, 3-1-1-10-2-1-2, 4-10-1-1-1-1-2, 4-11-1-1-3, and 2-2-2-10-1-1-2; wherein the first numeral represents the number of LNA units, the next the number of DNA units, and alternating LNA and DNA regions thereafter.

In some embodiments, the maximum number of contiguous LNA nucleosides in regions A and C is 2 or 3. In some embodiments, the maximum number of contiguous DNA nucleosides in regions A and C is 1 or 2.

In some embodiments, the LNA nucleoside units present in regions A and C are beta-D LNA units, such as beta-D-oxy LNA nucleoside units or (S)cEt nucleoside LNA units.

In some embodiments, the oligomer comprises at least one phosphorothioate internucleoside linkage.

In some embodiments, the internucleoside linkages within region B are phosphorothioate internucleoside linkages.

In some embodiments, all the internucleoside linkages within regions A, B and C are phosphorothioate internucleoside linkages.

In one embodiment, the oligomer of the invention is a gapmer. A gapmer oligomer is an oligomer which comprises a contiguous stretch of nucleotides which is capable of recruiting an RNase, such as RNaseH, such as a region of at least 7 DNA nucleotides, referred to herein in as region B (B), wherein region B is flanked both 5′ and 3′ by regions of affinity enhancing nucleotide analogs, such as from 1-10 nucleotide analogs 5′ and 3′ to the contiguous stretch of nucleotides which is capable of recruiting RNase—these regions are referred to as regions A (A) and C (C) respectively.

In the gapmers of the invention, at least one of region A or C, or both regions A and C, comprise at least one DNA nucleoside.

In certain embodiments, the gapmer comprises a (poly)nucleotide sequence of formula (5′ to 3′), A-B-C, wherein: region A (A) (5′ region or a first wing sequence) comprises at least one nucleotide analog, such as at least one LNA unit, such as from 1-10 nucleotide analogs, such as LNA units, and; region B (B) comprises at least seven consecutive nucleotides which are capable of recruiting RNase (when formed in a duplex with a complementary RNA molecule, such as the pre-mRNA or mRNA target), such as DNA nucleotides, and; region C (C) (3′ region or a second wing sequence) comprises at least one nucleotide analog, such as at least one LNA unit, such as from 1-10 nucleotide analogs, such as LNA units; wherein regions A and C may include at any position in A and C 1-2 insertions of DNA nucleotide regions (e.g., DNA gapmers), in which these DNA insertions may each be 1-3 DNA units long.

In certain other embodiments, the gapmer comprises a (poly)nucleotide sequence of formula (5′ to 3′), A-B-C, or optionally A-B-C-D or D-A-B-C, wherein: region A (A) (5′ region) comprises at least one nucleotide analog, such as at least one LNA unit, such as from 1-10 nucleotide analogs, such as LNA units, and; region B (B) comprises at least seven consecutive nucleotides which are capable of recruiting RNase (when formed in a duplex with a complementary RNA molecule, such as the mRNA target), such as DNA nucleotides, and; region C (C) (3′ region) comprises at least one nucleotide analog, such as at least one LNA unit, such as from 1-10 nucleotide analogs, such as LNA units, and; region D (D), when present comprises 1, 2 or 3 nucleotide units, such as DNA nucleotides.

In some embodiments, region A comprises 1, 2, 3, 4, 5, 6, 7, 8, 9, or 10 nucleotide analogs, such as LNA units, such as from 2-5 nucleotide analogs, such as 2-5 LNA units, such as 2-5 nucleotide analogs, such as 3-5 LNA units; and/or region C consists of 1, 2, 3, 4, 5, 6, 7, 8, 9, or 10 nucleotide analogs, such as LNA units, such as from 2-5 nucleotide analogs, such as 2-5 LNA units, such as 2-5 nucleotide analogs, such as 3-5 LNA units.

In some embodiments B comprises 5, 6, 7, 8, 9, 10, 11, 12, 13, 14, 15, 16, 17, 18, 19, 20, 21, 22, or 23 consecutive nucleotides which are capable of recruiting RNase, or from 8-14, or from 7-10, or from 7-9, such as 8, such as 9, such as 10, or such as 14 consecutive nucleotides which are capable of recruiting RNase. In some embodiments region B comprises at least seven DNA nucleotide unit, such as 7-23 DNA units, such as from 7-20 DNA units, such as from 7-14 DNA units, such as from 8-14 DNA units, such as 7, 8, 9, 10, 11, 12, 13, or 14 DNA units.

In some embodiments region A comprises 3, 4, or 5 nucleotide analogs, such as LNA, region B consists of 7, 8, 9, 10, 11, 12, 13, or 14 DNA units, and region C consists of 3, 4, or 5 nucleotide analogs, such as LNA. Such designs include (A-B-C) 5-10-5, 3-14-3, 3-10-3, 3-10-4, 4-10-3, 3-9-3, 3-9-4, 4-9-3, 3-8-3, 3-8-4, 4-8-3, 3-7-3, 3-7-4, 4- 7-3, and can further include region D, which can have one to 3 nucleotide units, such as DNA units.

In some embodiments, the oligomer of the invention has the formula of 5′-A-B-C-3′, wherein
(i) B is a contiguous sequence of 7 to 23 DNA units;
(ii) A is a first wing sequence of 1 to 10 nucleotides, wherein the first wing sequence comprises one or more nucleotide analogs and optionally one or more DNA units (e.g., DNA gapmer) and wherein at least one of the nucleotide analogs is located at the 5′ end of A; and
(iii) C is a second wing sequence of 1 to 10 nucleotides, wherein the second wing sequence comprises one or more nucleotide analogs and optionally one or more DNA units (e.g., DNA gapmer) and wherein at least one of the nucleotide analogs is located at the 3′ end of C.

In other embodiments, the oligomer has the formula of 5′-A-B-C-3′, wherein B is a contiguous sequence of 7 to 23 DNA units, A is LmDnLoDpLq and C is Lm′Dn′Lo′Dp′Lq′ and wherein L is a nucleotide analog; D is a DNA unit; m and q′ are 1 to 6 units; n, p, n′, and p′ are 0 to 2 units; and o, q, m′, and o′ are 0 to 5.

In some embodiments, the first wing sequence (A in the formula) comprises a combination of nucleotide analogs and DNA unit selected from (i) 1-9 nucleotide analogs and 1 DNA unit; (ii) 1-8 nucleotide analogs and 1-2 DNA units; (iii) 1-7 nucleotide analogs and 1-3 DNA units; (iv) 1-6 nucleotide analogs and 1-4 DNA units; (v) 1-5 nucleotide analogs and 1-5 DNA units; (vi) 1-4 nucleotide analogs and 1-6 DNA units; (vii) 1-3 nucleotide analogs and 1-7 DNA units; (viii) 1-2 nucleotide analogs and 1-8 DNA units; and (ix) 1 nucleotide analog and 1-9 DNA units.

In certain embodiments, the second wing sequence (C in the formula) comprises a combination of nucleotide analogs and DNA unit selected from (i) 1-9 nucleotide analogs and 1 DNA unit; (ii) 1-8 nucleotide analogs and 1-2 DNA units; (iii) 1-7 nucleotide analogs and 1-3 DNA units; (iv) 1-6 nucleotide analogs and 1-4 DNA units; (v) 1-5 nucleotide analogs and 1-5 DNA units; (vi) 1-4 nucleotide analogs and 1-6 DNA units; (vii) 1-3 nucleotide analogs and 1-7 DNA units; (viii) 1-2 nucleotide analogs and 1-8 DNA units; and (ix) 1 nucleotide analog and 1-9 DNA units.

In some embodiments, region A in the oligomer formula has a sub-formula selected from L, LL, LDL, LLL, LLDL, LDLL, LDDL, LLLL, LLLLL, LLLDL, LLDLL, LDLLL, LLDDL, LDDLL, LLDLD, LDLLD, LDDDL, LLLLLL, LLLLDL, LLLDLL, LLDLLL, LDLLLL, LLLDDL, LLDLDL, LLDDLL, LDDLLL, LDLLDL, LDLDLL, LDDDLL, LLDDDL, and LDLDLD, and region C in the oligomer formula has a sub-formula selected from L, LL, LDL, LLL, LLDL, LLLL, LDLL, LDDL, LLDD, LLLLL, LLLLD, LLLDL, LLDLL, LDLLL, LLDDL, LDDLL, LLDLD, LDLLD, LDDDL, LLLLLL, LLLLDL, LLLDLL, LLDLLL, LDLLLL, LLLDDL, LLDLDL, LLDDLL, LDDLLL, LDLLDL, LDLDLL, LDDDLL, LLDDDL, and LDLDLD, wherein at least one of regions A and C comprises at least one DNA nucleoside D.

In some embodiments, region A in the oligomer formula has a sub-formula selected from the group consisting of LDL, LLDL, LDLL, LDDL, LLLDL, LLDLL, LDLLL, LLDDL, LDDLL, LLDLD, LDLLD, LDDDL, LLLLDL, LLLDLL, LLDLLL, LDLLLL, LLLDDL, LLDLDL, LLDDLL, LDDLLL, LDLLDL, LDLDLL, LDDDLL, LLDDDL, and LDLDLD, and region C in the oligomer formula has a sub-formula selected from the group consisting of LDL, LLDL, LDLL, LDDL, LLDD, LLLLD, LLLDL, LLDLL, LDLLL, LLDDL, LDDLL, LLDLD, LDLLD, LDDDL, LLLLDL, LLLDLL, LLDLLL, LDLLLL, LLLDDL, LLDLDL, LLDDLL, LDDLLL, LDLLDL, LDLDLL, LDDDLL, LLDDDL, and LDLDLD.

In some embodiments, region A in the oligomer has a sub-formula selected from the group consisting of LDL, LLDL, LDLL, LDDL, LLLDL, LLDLL, LDLLL, LLDDL, LDDLL, LLDLD, LDLLD, LDDDL, LLLLDL, LLLDLL, LLDLLL, LDLLLL, LLLDDL, LLDLDL, LLDDLL, LDDLLL, LDLLDL, LDLDLL, LDDDLL, LLDDDL, and LDLDLD.

In some embodiments, region C in the oligomer formula has a sub-formula selected from the group consisting of LDL, LLDL, LDLL, LDDL, LLDD, LLLLD, LLLDL, LLDLL, LDLLL, LLDDL, LDDLL, LLDLD, LDLLD, LDDDL, LLLLDL, LLLDLL, LLDLLL, LDLLLL, LLLDDL, LLDLDL, LLDDLL, LDDLLL, LDLLDL, LDLDLL, LDDDLL, LLDDDL, and LDLDLD.

In some embodiments, region A in the oligomer has a sub-formula selected from the group consisting of LDL, LLDL, LDLL, LDDL, LLLDL, LLDLL, LDLLL, LLDDL, LDDLL, LLDLD, LDLLD, LDDDL, LLLLDL, LLLDLL, LLDLLL, LDLLLL, LLLDDL, LLDLDL, LLDDLL, LDDLLL, LDLLDL, LDLDLL, LDDDLL, LLDDDL, and LDLDLD, and region B is a length of 8, 9, 10, 11 or 12 (or 13 or 14) contiguous DNA nucleosides, and region C in the oligomer has a sub-formula selected from the group consisting of LL, LDL, LLL, LLDL, LLLL, LDLL, LDDL, LLDD, LLLLL, LLLLD, LLLDL, LLDLL, LDLLL, LLDDL, LDDLL, LLDLD, LDLLD, LDDDL, LLLLLL, LLLLDL, LLLDLL, LLDLLL, LDLLLL, LLLDDL, LLDLDL, LLDDLL, LDDLLL, LDLLDL, LDLDLL, LDDDLL, LLDDDL, and LDLDLD.

In some embodiments, region A in the oligomer formula has a sub-formula selected from the group consisting of L, LL, LDL, LLL, LLDL, LDLL, LDDL, LLLL, LLLLL, LLLDL, LLDLL, LDLLL, LLDDL, LDDLL, LLDLD, LDLLD, LDDDL, LLLLLL, LLLLDL, LLLDLL, LLDLLL, LDLLLL, LLLDDL, LLDLDL, LLDDLL, LDDLLL, LDLLDL, LDLDLL, LDDDLL, LLDDDL, and LDLDLD, and region B is a length of 8, 9, 10, 11 or 12 contiguous DNA nucleosides, and region C in the oligomer has a sub-formula selected from the group consisting of LDL, LLDL, LDLL, LDDL, LLDD, LLLLD, LLLDL, LLDLL, LDLLL, LLDDL, LDDLL, LLDLD, LDLLD, LDDDL, LLLLDL, LLLDLL, LLDLLL, LDLLLL, LLLDDL, LLDLDL, LLDDLL, LDDLLL, LDLLDL, LDLDLL, LDDDLL, LLDDDL, and LDLDLD.

In some embodiments, region C in the oligomer formula has a sub-formula selected from the group consisting of LDL, LLDL, LDLL, LDDL, LLDD, LLLLD, LLLDL, LLDLL, LDLLL, LLDDL, LDDLL, LLDLD, LDLLD, LDDDL, LLLLDL, LLLDLL, LLDLLL, LDLLLL, LLLDDL, LLDLDL, LLDDLL, LDDLLL, LDLLDL, LDLDLL, LDDDLL, LLDDDL, and LDLDLD.

In certain embodiments, the oligomer has the formula of 5′ A-B-C 3′, wherein region B is a contiguous sequence of 7 to 23 DNA units, such as 8, 9, 10, 11, 12, 13 or 14 DNA units, region A has a formula of LLDLL or LDLLL, and region C has the formula of LLDLL, and wherein L is an LNA unit and D is a DNA unit.

In some embodiments, the oligomer has a formula (5′-3′) selected from the group consisting of LLDDDLLDDDDDDDDLL, LDLLDLDDDDDDDDDLL, LLLDDDDDDDDDDLDLL, LLLDDDDDDDDDLDDLL, LLLDDDDDDDDLDDDLL, LLLDDDDDDDDLDLDLL, LLLDLDDDDDDDDDLLL, LLLDLDDDDDDDDLDLL, LLLLDDDDDDDDDLDLL, LLLLDDDDDDDDLDDLL, LLLDDDLDDDDDDDDLL, LLLDDLDDDDDDDDDLL, LLLDDLLDDDDDDDDLL, LLLDDLLDDDDDDDLLL, LLLLLDDDDDDDLDDLL, LDLLLDDDDDDDDDDLL, LDLLLDDDDDDDLDDLL, LDLLLLDDDDDDDDDLL, LLDLLLDDDDDDDDDLL, LLLDLDDDDDDDDDDLL, LLLDLDDDDDDDLDDLL, LLLDLLDDDDDDDDDLL, LLLLDDDDDDDLDDDLL, LLLLLDDDDDDDDDLDLL, LLLLDDDDDDDDDDLDLL, LLLDDDDDDDDDDDLDLL, LLDLDDDDDDDDDDLDLL, LDLLLDDDDDDDDDLDLL, LLLDDDDDDDDDDLDDLL, LLLDDDDDDDDDLDDDLL, LLLDDDDDDDDLDLDDLL, LLLLDDDDDDDDDLDDLL, LLLLDDDDDDDDDLDLLL, LLLLDDDDDDDDLDDDLL, LLLLDDDDDDDDLDDLLL, LLLLDDDDDDDDLDLDLL, LLLLDDDDDDDLDDLDLL, LLLLDDDDDDDLDLDDLL, LLDLLDDDDDDDDDDDLL, LLDLLLDDDDDDDDLDLL, LLLDLDDDDDDDDDDDLL, LLLDLDDDDDDDDDLDLL, LLLDLDDDDDDDDLDDLL, LLLDLDDDDDDDLDLDLL, LLLLDDDDDDDDDLLDLL, LLLLLDDDDDDDDDLDLLL, LLLLLDDDDDDDDDLDDLL, LLLLDDDDDDDDDDLLDLL, LLLLDDDDDDDDDDLDLLL, LLLLDDDDDDDDDDLDDLL, LLLDDDDDDDDDDDLLDLL, LLLDDDDDDDDDDDLDLLL, LLLLLDDDDDDDDDLLDLL, LLLDDDDDDDDDDDLDDLL, LLDLLDDDDDDDDDLDDLL, LLLDLDDDDDDDDDDLDLL, LLLDLDDDDDDDDDLDDLL, LLLLDDDDDDDDDLDLDLL, LLLLDDDDDDDDLLDLDLL, LDLLLDDDDDDDDDDLLDLL, LLDLLDDDDDDDDDDLLDLL, LLDLDDDDDDDDDDDDLLLL, LLDDLDDDDDDDDDDDLLLL, LLLDLDDDDDDDDDDDLLLL, LLDLDDDDDDDDDDDDDLLL, LLDLLDDDDDDDDDDDLLLL, LLDDLDDDDDDDDDDDDLLL, LLLDDDDDDDDDDDLDDLLL, LLLDLDDDDDDDDDDDDLLL, LLDLLDDDDDDDDDDDDLLL, LLLLDDDDDDDDDDDLLDLL, LLLLDDDDDDDDDDLLDDLL, LLLDDLDDDDDDDDDLDLLL, LLDDLDLDDDDDDDDDLLLL, LLDDLLDDDDDDDDDLDLLL, LLLDLDDDDDDDDDLDLDLL, LLDLLDDDDDDDDDLDDLLL, LLLDLDDDDDDDDDDLDLLL, LLDLDDLDDDDDDDDDLLLL, LLLLDDDDDDDDDLDLDDLL, LLLDLDDDDDDDDDLDDLLL, LLDLDLDDDDDDDDDDLLLL, LLDLLDDDDDDDDDDLDLLL, LLDLDLDDDDDDDDDLLDLL, LLDDLLDDDDDDDDDLLDLL, LLLLDDDDDDDDDLDDLDLL, LLLDDLDDDDDDDDDLLDLL, LLDLLDDDDDDDDDLLDDLL, LLDLDLDDDDDDDDDLDLLL, LLLDLDDDDDDDDDLLDDLL, LLDDLLDDDDDDDDDDLLLL, LLDLLDDDDDDDDDLDLDLL, LLLLDDDDDDDDDDLDDLLL, LLLDDLDDDDDDDDDDLLLL, LLLDLDDDDDDDDDDLLDLL, LLLLDDDDDDDDDDLDLDLL, LLLLDDDDDDDDDDDLDLLL, and LLDDLLDDDDDDDDDDLDLL; wherein L represents a LNA nucleoside, and D represents a DNA nucleoside. In some embodiments, L represents a beta-D-oxy LND nucleoside. It can be recognized that in some embodiments, region B of consecutive DNA nucleosides can be varied, e.g. 7, 8, 9, 10, 11, 12, 13 or 14 DNA nucleotides in length. The internucleoside linkages of the oligomers of the invention can, for example be phosphorothioate internucleoside linkages.

In some embodiments, the oligomer of the invention has an alternating flank design of LNA and DNA nucleoside units, selected from the group consisting of (5′-3′) 2-3-2-8-2, 1-1-2-1-1-9-2, 3-10-1-1-2, 3-9-1-2-2, 3-8-1-3-2, 3-8-1-1-1-1-2, 3-1-1-9-3, 3-1-1-8-1-1-2, 4-9-1-1-2, 4-8-1-2-2, 3-3-1-8-2, 3-2-1-9-2, 3-2-2-8-2, 3-2-2-7-3, 5-7-1-2-2, 1-1-3-10-2, 1-1-3-7-1-2-2, 1-1-4-9-2, 2-1-3-9-2, 3-1-1-10-2, 3-1-1-7-1-2-2, 3-1-2-9-2, 4-7-1-3-2, 5-9-1-1-2, 4-10-1-1-2, 3-11-1-1-2, 2-1-1-10-1-1-2, 1-1-3-9-1-1-2, 3-10-1-2-2, 3-9-1-3-2, 3-8-1-1-1-2-2, 4-9-1-2-2, 4-9-1-1-3, 4-8-1-3-2, 4-8-1-2-3, 4-8-1-1-1-1- 2, 4-7-1-2-1-1-2, 4-7-1-1-1-2-2, 2-1-2-11-2, 2-1-3-8-1-1-2, 3-1-1-11-2, 3-1-1-9-1-1-2, 3-1-1-8-1-2-2, 3-1-1-7-1-1-1-1-2, 4-9-2-1-2, 4-7-1-3-3, 5-9-1-1-3, 5-9-1-2-2, 4-10-2-1-2, 4-10-1-1-3, 4-10-1-2-2, 3-11-2-1-2, 3-11-1-1-3, 5-9-2-1-2, 3-11-1-2-2, 2-1-2-9-1-2-2, 3-1-1-10-1-1-2, 3-1-1-9-1-2-2, 4-9-1-1-1-1-2, 4-8-2-1-1-1-2, 1-1-3-10-2-1-2, 2-1-2-10-2-1-2, 2-1-1-12-4, 2-2-1-11-4, 3-1-1-11-4, 2-1-1-13-3, 2-1-2-11-4, 2-2-1-12-3, 3-11-1-2-3, 3-1-1-12-3, 2-1-2-12-3, 4-11-2-1-2, 4-10-2-2-2, 3-2-1-9-1-1-3, 2-2-1-1-1-9-4, 2-2-2-9-1-1-3, 3-1-1-9-1-1-1-1-2, 2-1-2-9-1-2-3, 3-1-1-10-1-1-3, 2-1-1-2-1-9-4, 4-9-1-1-1-2-2, 3-1-1-9-1-2-3, 2-1-1-1-1-10-4, 2-1-2-10-1-1-3, 2-1-1-1-1-9-2-1-2, 2-2-2-9-2-1-2, 4-9-1-2-1-1-2, 3- 2-1-9-2-1-2, 2-1-2-9-2-2-2, 2-1-1-1-1-9-1-1-3, 3-1-1-9-2-2-2, 2-2-2-10-4, 2-1-2-9-1-1-1-1-2, 4-10-1-2-3, 3-2-1-10-4, 3-1-1-10-2-1-2, 4-10-1-1-1-1-2, 4-11-1-1-3, and 2-2-2-10-1-1-2; wherein the first numeral represents the number of LNA units, the next the number of DNA units, and alternating LNA and DNA regions thereafter. The LNA units can for example all be beta-D-oxy LNA units.

In other embodiments, the oligomers of the invention are gapmers having the formula of 5′ A-B-C 3′, wherein the oligomer has 12 to 25 nucleotides in length, A is a first wing sequence having the formula of LₘdₙLₒdₚL_{q}, C is a second wing sequence having the formula of L_{q′}d_{p′}L_{o′}d_{n′}L_{m′}, wherein each wing independently has 1-17 nucleotides in length and is optionally interrupted by DNA spacers dₙ, dₚ, d_{n′} and d_{p′}, each of which independently has 0 to 3 DNA units, with each wing flanking an all DNA gap of 7 to 23 nucleotides;
wherein m and m′ are at least 1;
and n, n′, p and p′ are independently 0-3 units;
such that m+n+o+p+q=1-17; and independently m′+n′+o′+p′+q′=1-17;
or (m+n+o+p+q) and (m′+n′+o′+p′+q′) are independently 1, 2, 3, 4, 5, 6, 7, 8, 9, 10, 11, 12, 13, 14, 15, 16 or 17;
or B comprises a DNA gap of 7, 8, 9, 10, 11, 12, 13, 14, 15, 16, 17, 18, 19, 20, 21, 22 or 23;

Further gapmer designs are disclosed in WO2004/046160, which is hereby incorporated by reference in its entirety. WO2008/113832 hereby incorporated by reference in its entirety, refers to ‘shortmer’ gapmer oligomers. In some embodiments, oligomers presented herein can be such shortmer gapmers.

In some embodiments the oligomer comprises a contiguous nucleotide sequence of a total of 10, 11, 12, 13, 14, 15, 16, 17, 18, 19, or 20 nucleotide units, wherein the contiguous nucleotide sequence is of formula (5′-3′), A-B-C, or optionally A-B-C-D or D-A-B-C, wherein; A consists of 1, 2, 3, 4, or 5 nucleotide analog units, such as LNA units; B consists of 7, 8, 9, 10, 11, 12, 13, or 14 contiguous nucleotide units which are capable of recruiting RNase when formed in a duplex with a complementary RNA molecule (such as a mRNA target); and C consists of 1, 2, 3, 4, or 5 nucleotide analog units, such as LNA units. When present, D consists of a single DNA unit.

In some embodiments A comprises 1 LNA unit. In some embodiments A comprises 2 LNA units. In some embodiments A comprises 3 LNA units. In some embodiments A comprises 4 LNA units. In some embodiments A comprises 5 LNA units. In some embodiments C comprises 2 LNA units. In some embodiments C comprises 3 LNA units. In some embodiments C comprises 4 LNA units. In some embodiments C comprises 5 LNA units. In some embodiments B comprises 7 nucleotide units. In some embodiments B comprises 8 nucleotide units. In some embodiments B comprises 9 nucleotide units. In certain embodiments, region B comprises 10 nucleoside units. In certain embodiments, region B comprises 11 nucleoside units. In certain embodiments, region B comprises 12 nucleoside units. In certain embodiments, region B comprises 13 nucleoside units. In certain embodiments, region B comprises 14 nucleoside units. In certain embodiments, region B comprises 7-23 DNA monomers. In some embodiments B comprises from 7-23 DNA units, such as 7, 8, 9, 10, 11, 12, 13, 14, 15, 16, 17, 18, 19, 20, 21, 22, or 23 DNA units. In some embodiments B consists of DNA units. In some embodiments B comprises at least one LNA unit which is in the alpha-L configuration, such as 2, 3, 4, 5, 6, 7, 8, 9, 10, 11, 12, 13, 14, 15, 16, 17, 18, 19, 20, 21, 22, or 23 LNA units in the alpha-L-configuration. In some embodiments B comprises at least one alpha-L-oxy LNA unit or wherein all the LNA units in the alpha-L-configuration are alpha-L-oxy LNA units. In some embodiments, the oligomer contains 10 DNA units in B, LDLLL in A (first wing) and LLDLL in C (second wing). In yet other embodiments, the oligomer contains 9 DNA units in B, LDDLL in A, LDLDLL in C. In still other embodiments, the oligomer contains 10 DNA units in B, LLDLL in A, and LLDLL in C. In further embodiments, the oligomer contains 9 DNA units in B, LLLLL in A, and LDDLL in C. In certain embodiments, each of regions A and C comprises three LNA monomers, and region B consists of 7 or 8 or 9 or 10 or 11 or 12 or 13 or 14 nucleoside monomers, for example, DNA monomers.

### II.H. Internucleotide Linkages

The monomers of the oligomers described herein are coupled together via linkage groups. Suitably, each monomer is linked to the 3′ adjacent monomer via a linkage group.

The person having ordinary skill in the art would understand that, in the context of the present invention, the 5′ monomer at the end of an oligomer does not comprise a 5′ linkage group, although it may or may not comprise a 5′ terminal group.

The terms “linkage group” or “internucleotide linkage” are intended to mean a group capable of covalently coupling together two nucleotides. Specific and preferred examples include phosphate groups and phosphorothioate groups.

The nucleotides of the oligomer of the invention or contiguous nucleotides sequence thereof are coupled together via linkage groups. Suitably each nucleotide is linked to the 3′ adjacent nucleotide via a linkage group.

Suitable internucleotide linkages include those listed within WO2007/031091, for example the internucleotide linkages listed on the first paragraph of page 34 of WO2007/031091 (hereby incorporated by reference in its entirety).

Examples of suitable internucleotide linkages that can be used with the invention include phosphodiester linkage, a phosphotriester linkage, a methylphosphonate linkage, a phosphoramidate linkage, a phosphorothioate linkage, and combinations thereof.

It is, in some embodiments, preferred to modify the internucleotide linkage from its normal phosphodiester to one that is more resistant to nuclease attack, such as phosphorothioate or boranophosphate—these two, being cleavable by RNaseH, also allow that route of antisense inhibition in reducing the expression of the target gene.

Suitable sulphur (S) containing internucleotide linkages as provided herein may be preferred. Phosphorothioate internucleotide linkages are also preferred, particularly for the gap region (B) of gapmers. Phosphorothioate linkages can also be used for the flanking regions (A and C, and for linking A or C to D, and within region D, as appropriate).

Regions A, B and C, can, however, comprise internucleotide linkages other than phosphorothioate, such as phosphodiester linkages, particularly, for instance when the use of nucleotide analogs protects the internucleotide linkages within regions A and C from endo-nuclease degradation—such as when regions A and C comprise LNA nucleotides.

The internucleotide linkages in the oligomer can be phosphodiester, phosphorothioate or boranophosphate so as to allow RNaseH cleavage of targeted RNA. Phosphorothioate is preferred, for improved nuclease resistance and other reasons, such as ease of manufacture.

In one aspect of the oligomer of the invention, the nucleotides and/or nucleotide analogs are linked to each other by means of phosphorothioate groups.

It is recognized that the inclusion of phosphodiester linkages, such as one or two linkages, into an otherwise phosphorothioate oligomer, particularly between or adjacent to nucleotide analog units (typically in region A and or C) can modify the bioavailability and/or bio-distribution of an oligomer—see WO2008/113832, hereby incorporated by reference.

In some embodiments, such as the embodiments referred to above, where suitable and not specifically indicated, all remaining linkage groups are either phosphodiester or phosphorothioate, or a mixture thereof.

In some embodiments all the internucleotide linkage groups are phosphorothioate.

When referring to specific gapmer oligonucleotide sequences, such as those provided herein it will be understood that, in various embodiments, when the linkages are phosphorothioate linkages, alternative linkages, such as those disclosed herein can be used, for example phosphate (phosphodiester) linkages can be used, particularly for linkages between nucleotide analogs, such as LNA, units. Likewise, when referring to specific gapmer oligonucleotide sequences, such as those provided herein, when the C residues are annotated as 5′methyl modified cytosine, in various embodiments, one or more of the Cs present in the oligomer can be unmodified C residues.

US Publication No. 2011/0130441, which was published Jun. 2, 2011 and is incorporated by reference herein in its entirety, refers to oligomeric compounds having at least one bicyclic nucleoside attached to the 3′ or 5′ termini by a neutral internucleoside linkage. The oligomers of the invention can therefore have at least one bicyclic nucleoside attached to the 3′ or 5′ termini by a neutral internucleoside linkage, such as one or more phosphotriester, methylphosphonate, MMI, amide-3, formacetal or thioformacetal. The remaining linkages can be phosphorothioate.

In some embodiments, the oligomers of the invention have internucleotide linkages described in FIG. 2, 6B or 11B. As used herein, e.g., FIG. 2, 6B or 11B, phosphorothioate linkages are indicated as “s”, and phosphorodiester linkages are indicated by the absence of “s.”

### III. Conjugates

In the context the term “conjugate” is intended to indicate a heterogeneous molecule formed by the covalent or non-covalent attachment (“conjugation”) of the oligomer as described herein to one or more non-nucleotide, or non-polynucleotide moieties. Examples of non-nucleotide or non-polynucleotide moieties include macromolecular agents such as proteins, fatty acid chains, sugar residues, glycoproteins, polymers, or combinations thereof. Typically proteins can be antibodies for a target protein. In some embodiments, typical polymers are polyethylene glycol.

Therefore, in various embodiments, the oligomer of the invention comprises both a polynucleotide region which typically consists of a contiguous sequence of nucleotides, and a further non-nucleotide region. When referring to the oligomer of the invention comprising a contiguous nucleotide sequence, the compound can comprise non-nucleotide components, such as a conjugate component.

The invention also provides for a conjugate comprising the oligomer according to the invention as herein described, and at least one non-nucleotide or non-polynucleotide moiety covalently attached to the oligomer. Therefore, in various embodiments where the oligomer of the invention comprises a specified nucleic acid or nucleotide sequence, as herein disclosed, the compound can also comprise at least one non-nucleotide or non-polynucleotide moiety (e.g., not comprising one or more nucleotides or nucleotide analogs) covalently attached to the oligomer.

Conjugation (to a conjugate moiety) can enhance the activity, cellular distribution or cellular uptake of the oligomer of the invention. Such moieties include, but are not limited to, antibodies, polypeptides, lipid moieties such as a cholesterol moiety, cholic acid, a thioether.

The oligomers of the invention can also be conjugated to active drug substances, for example, aspirin, ibuprofen, a sulfa drug, an antidiabetic, an antibacterial or an antibiotic.

In certain embodiments the conjugated moiety is a sterol, such as cholesterol.

### III.A. Activated Oligomers

The term “activated oligomer,” as used herein, refers to an oligomer of the invention that is covalently linked (i.e., functionalized) to at least one functional moiety that permits covalent linkage of the oligomer to one or more conjugated moieties, i.e., moieties that are not themselves nucleic acids or monomers, to form the conjugates herein described. Typically, a functional moiety will comprise a chemical group that is capable of covalently bonding to the oligomer via, e.g., a 3′-hydroxyl group or the exocyclic NH₂ group of the adenine base, a spacer that can be hydrophilic and a terminal group that is capable of binding to a conjugated moiety (e.g., an amino, sulfhydryl or hydroxyl group). In some embodiments, this terminal group is not protected, e.g., is an NH₂ group. In other embodiments, the terminal group is protected, for example, by any suitable protecting group such as those described in “Protective Groups in Organic Synthesis” by Theodora W Greene and Peter G M Wuts, 3rd edition (John Wiley & Sons, 1999).

In some embodiments, oligomers of the invention are functionalized at the 5′ end in order to allow covalent attachment of the conjugated moiety to the 5′ end of the oligomer. In other embodiments, oligomers of the invention can be functionalized at the 3′ end. In still other embodiments, oligomers of the invention can be functionalized along the backbone or on the heterocyclic base moiety. In yet other embodiments, oligomers of the invention can be functionalized at more than one position independently selected from the 5′ end, the 3′ end, the backbone and the base.

In some embodiments, activated oligomers of the invention are synthesized by incorporating during the synthesis one or more monomers that is covalently attached to a functional moiety. In other embodiments, activated oligomers of the invention are synthesized with monomers that have not been functionalized, and the oligomer is functionalized upon completion of synthesis.

### IV. Pharmaceutical Compositions and Administration Routes

The oligomer of the invention can be used in pharmaceutical formulations and compositions. Suitably, such compositions comprise a pharmaceutically acceptable diluent, carrier, salt or adjuvant.

The oligomer of the invention can be included in a unit formulation such as in a pharmaceutically acceptable carrier or diluent in an amount sufficient to deliver to a patient a therapeutically effective amount without causing serious side effects in the treated patient. However, in some forms of therapy, serious side effects may be acceptable in terms of ensuring a positive outcome to the therapeutic treatment.

The formulated drug may comprise pharmaceutically acceptable binding agents and adjuvants. Capsules, tablets, or pills can contain for example the following compounds: microcrystalline cellulose, gum or gelatin as binders; starch or lactose as excipients; stearates as lubricants; various sweetening or flavoring agents. For capsules the dosage unit may contain a liquid carrier like fatty oils. Likewise coatings of sugar or enteric agents may be part of the dosage unit. The oligonucleotide formulations can also be emulsions of the active pharmaceutical ingredients and a lipid forming a micellular emulsion.

The pharmaceutical compositions of the present invention can be administered in a number of ways depending upon whether local or systemic treatment is desired and upon the area to be treated. Administration can be (a) oral (b) pulmonary, e.g., by inhalation or insufflation of powders or aerosols, including by nebulizer; intratracheal, intranasal, (c) topical including epidermal, transdermal, ophthalmic and to mucous membranes including vaginal and rectal delivery; or (d) parenteral including intravenous, intraarterial, subcutaneous, intraperitoneal or intramuscular injection or infusion; or intracranial, e.g., intrathecal, intra-cerebroventricular, or intraventricular, administration. In one embodiment the oligomer is administered IV, IP, orally, topically or as a bolus injection or administered directly in to the target organ. In another embodiment, the oligomer is administered intrathecal or intra-cerebroventricular as a bolus injection.

Pharmaceutical compositions and formulations for topical administration can include transdermal patches, ointments, lotions, creams, gels, drops, sprays, suppositories, liquids and powders. Conventional pharmaceutical carriers, aqueous, powder or oily bases, thickeners and the like may be necessary or desirable. Examples of topical formulations include those in which the oligomer of the invention are in admixture with a topical delivery agent such as lipids, liposomes, fatty acids, fatty acid esters, steroids, chelating agents and surfactants. Compositions and formulations for oral administration include but are not limited to powders or granules, microparticulates, nanoparticulates, suspensions or solutions in water or non-aqueous media, capsules, gel capsules, sachets, tablets or minitablets. Compositions and formulations for parenteral, intrathecal, intra-cerebroventricular, or intraventricular administration can include sterile aqueous solutions which can also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids. Delivery of drug to the target tissue can be enhanced by carrier-mediated delivery including, but not limited to, cationic liposomes, cyclodextrins, porphyrin derivatives, branched chain dendrimers, polyethylenimine polymers, nanoparticles and microspheres (Dass C R. J Pharm Pharmacol 2002; 54(0:3-27).

The pharmaceutical formulations of the present invention, which can conveniently be presented in unit dosage form, can be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

For parenteral, subcutaneous, intradermal or topical administration the formulation can include a sterile diluent, buffers, regulators of tonicity and antibacterials. The active oligomers can be prepared with carriers that protect against degradation or immediate elimination from the body, including implants or microcapsules with controlled release properties. For intravenous administration the carriers can be physiological saline or phosphate buffered saline. International Publication No. WO2007/031091 (A2), published Mar. 22, 2007, further provides suitable pharmaceutically acceptable diluent, carrier and adjuvants—which are hereby incorporated by reference.

### VII. Methods of Using

The oligomers of the invention can be utilized as research reagents for, for example, diagnostics, therapeutics and prophylaxis.

In research, such oligomers can be used to specifically inhibit the synthesis of a target protein (typically by degrading or inhibiting the mRNA and thereby prevent protein formation) in cells and experimental animals thereby facilitating functional analysis of the target or an appraisal of its usefulness as a target for therapeutic intervention. Further provided are methods of down-regulating the expression of a target mRNA and/or a target protein in cells or tissues comprising contacting the cells or tissues, in vitro or in vivo, with an effective amount of one or more of the oligomers, conjugates or compositions of the invention.

In diagnostics the oligomers can be used to detect and quantitate target transcript expression in cell and tissues by northern blotting, in-situ hybridization or similar techniques.

For therapeutics, an animal or a human, suspected of having a disease or disorder, which can be treated by modulating the expression of a target transcript and/or target protein is treated by administering oligomeric compounds in accordance with this invention while reducing the toxicity of oligomeric compounds. Further provided are methods of treating a mammal, such as treating a human, suspected of having or being prone to a disease or condition, associated with expression of a target transcript and/or target protein by administering a therapeutically or prophylactically effective amount of one or more of the oligomers or compositions of the invention. The oligomer, a conjugate or a pharmaceutical composition according to the invention is typically administered in an effective amount. In some embodiments, the oligomer or conjugate of the invention is used in therapy.

In certain embodiments, the disease, disorder, or condition is associated with overexpression of the target gene transcript and/or target protein.

The invention also provides for methods of inhibiting (e.g., by reducing) the expression of target gene transcript and/or target protein in a cell or a tissue, the method comprising contacting the cell or tissue, in vitro or in vivo, with an effective amount of one or more oligomers, conjugates, or pharmaceutical compositions thereof, of the invention to affect degradation of expression of target gene transcript thereby reducing target protein.

The invention also provides for the use of the oligomer or conjugate of the invention as described for the manufacture of a medicament for the treatment of a disorder as referred to herein, or for a method of the treatment of as a disorder as referred to herein

The invention further provides for a method for inhibiting target protein in a cell which is expressing the target comprising administering an oligomer or a conjugate according to the invention to the cell so as to affect the inhibition of the target protein in the cell.

The invention also provides for a method for treating a disorder as referred to herein the method comprising administering an oligomer or a conjugate according to the invention as herein described and/or a pharmaceutical composition according to the invention to a patient in need thereof.

Also provided is a method of treating a disease or condition in a subject in need thereof while reducing the toxicity of the treatment comprising administering the oligomer, the conjugate, or the pharmaceutical composition to the subject, wherein the administering treats the disease or condition while reducing the toxicity of the treatment compared to a treatment using a corresponding reference oligomer, which reference oligomer comprises formula G-H-I, wherein region H is identical to region B, and regions G and I comprise the same sequence as regions A and C, but comprises all LNA nucleoside units.

The oligomers and other compositions according to the invention can be used for the treatment of conditions associated with over expression or expression of mutated version of target protein.

An interesting aspect of the invention is directed to the use of an oligomer (compound) as defined herein or a conjugate as defined herein for the preparation of a medicament for the treatment of a disease, disorder or condition as referred to herein.

The invention also relates to an oligomer, a composition or a conjugate as defined herein for use as a medicament.

A patient who is in need of treatment is a patient suffering from or likely to suffer from the disease or disorder.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Sambrook et al., ed. (1989) Molecular Cloning A Laboratory Manual (2nd ed.; Cold Spring Harbor Laboratory Press); Sambrook et al., ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (IRL Press) (1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al., eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986);); Crooks, Antisense drug Technology: Principles, strategies and applications, 2^{nd} Ed. CRC Press (2007) and in Ausubel et al. (1989) Current Protocols in Molecular Biology (John Wiley and Sons, Baltimore, Md.).

All of the references cited above, as well as all references cited herein, are incorporated herein by reference in their entireties.

The following description of the specific embodiments is offered by way of illustration and not by way of limitation. The following examples will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

Other embodiments of the invention will be apparent to those skilled in the art from consideration of the specification and practice of the invention disclosed herein. It is intended that the specification and examples be considered as exemplary only, with a true scope and spirit of the invention being indicated by the claims.

### EXAMPLES

### Example 1: Construction of Oligomers

A number of oligomers were designed to target the 3′ UTR of MAPT pre-mRNA. For example, the oligomers were constructed to target nucleotides 134,821-138,940 of SEQ ID NO: 1. The exemplary sequences of the oligomers are described in FIGS. 1, 2, 3, and 4. FIG. 1 shows non-limiting examples of the oligomer design for selected sequences. The same methods can be applied to any other sequences disclosed herein. The gapmers were constructed to contain locked nucleic acids —LNAs (upper case letters). For example, a gapmer can have Beta-deoxy LNA at the 5′ end and the 3′ end and have a phosphorothioate backbone. But the LNAs can also be substituted with any other nucleotide analog and the backbone can be other type of backbone (e.g., a phosphodiester linkage, a phosphotriester linkage, a methylphosphonate linkage, a phosphoramidate linkage, or combinations thereof).

The oligomers were synthesized using methods well known in the art. Exemplary methods of preparing such oligomers are described in Barciszewski et al., Chapter 10—“Locked Nucleic Acid Aptamers” in Nucleic Acid and Peptide Aptamers: Methods and Protocols, vol. 535, Gunter Mayer (ed.) (2009), the entire contents of which is hereby expressly incorporated by reference herein.

In FIG. 1, in the Sequence designation, upper case designates a modified nucleotide such as an LNA nucleotide (either Beta-D-Oxy, Alpha-L-Oxy, Beta-D-Amino or Beta-D-Thio LNA or other modified nucleotide such as cEt, cMOE, UNA or ENA) and lower case designates a DNA nucleotide. Thus a sequence represented by TCCCTtaatttcacCCtCA (SEQ ID NO. 29) represents a 5-9-2-1-2 19mer, e.g., LLLLLDDDDDDDDDLLDLL. Selected examples of alternating flank gapmers having a 7 nucleotide gap are ASO-002399, ASO-002482, ASO-002437, and ASO-002425. Any one of the oligomer sequences disclosed herein can have the chemical structure (design) of the alternating flank gapmer design shown in the figures.

In FIG. 1, the following designate the components of the oligonucleotides of the present invention, with oligonucleotides always depicted in the 5′ to 3′ direction. Therefore, the 5′ end of an oligomer hybridizes to the pre-mRNA end number in the figure and the 3′ end of the oligomer hybridizes to the pre-mRNA start number in the figure. A reference to a SEQ ID number includes a particular sequence, but does not include an oligomer design.

Beta-D-oxy LNA nucleotides are designated by OxyB where B designates a nucleotide base such as thymine (T), uridine (U), cytosine (C), methylcytosine (MC), adenine (A) or guanine (G), and thus include OxyA, OxyT, OxyMC, OxyC and OxyG.

Alpha-L-oxy LNA nucleotides are designated by AlfaOxyB where B designates a nucleotide base such as thymine (T), uridine (U), cytosine (C), methylcytosine (MC), adenine (A) or guanine (G), and thus include AlfaOxyA, AlfaOxyT, AlfaOxyMC, AlfaOxyC and AlfaOxyG. The letter M or m before C or c indicates 5-methylcytosine.

Beta-D-Amino LNA nucleotides are designated by AminoB where B designates a nucleotide base such as thymine (T), uridine (U), cytosine (C), methylcytosine (MC), adenine (A) or guanine (G), and thus include AminoA, AminoT, AminoMC, AminoC and AminoG. The letter M or m before C or c indicates 5-methylcytosine.

Beta-D-Thio-LNA nucleotides are designated by ThioB where B designates a nucleotide base such as thymine (T), uridine (U), cytosine (C), methylcytosine (MC), adenine (A) or guanine (G), and thus include ThioA, ThioT, ThioMC, ThioC and ThioG. The letter M or m before C or c indicates 5-methylcytosine.

DNA nucleotides are designated by DNAb, where the lower case b designates a nucleotide base such as thymine (T), uridine (U), cytosine (C), 5-methylcytosine (MC), adenine (A) or guanine (G), and thus include DNAa, DNAt, DNAc, DNAmc and DNAg. The letter M or m before C or c indicates 5-methylcytosine. Some examples of the oligomers containing 5 methyl cytosine include, but are not limited to, ASO-002658, ASO-002629, ASO-002621, and ASO-002665.

The letter “s” after the nucleotide designation indicates phosphorothioate linkage whereas absence of “s” indicates phosphodiester linkage.

Thus a 3-10-3 beta-D-oxy LNA-DNA-beta-D-oxy LNA gapmer with sequence ATTtccaaattcaCTT, with full phosphorothioate internucleotide linkages would be designated OxyAs OxyTs OxyTs DNAts DNAcs DNAcs DNAas DNAas DNAas DNAts DNAts DNAcs DNAas OxyMCs OxyTs OxyT. In some embodiments, the oligomers include a mix of phosphorothioate or phosphodiester internucleotide linkages. Non limiting examples of the oligomers having a mix of phosphorothioate or phosphodiester internucleotide linkages include ASO-002640, ASO-002632, ASO-002647, ASO-002666, ASO-002659, ASO-002652, ASO-002645, ASO-002638, ASO-003270, ASO-003269, ASO-003268, ASO-002673, ASO-002661, ASO-002654, and ASO-002668.

Preparation of Oligos with Mismatches

Oligos having mismatched bases at different locations were also prepared using standard methods well known in the art. Examples of oligomers with mismatched bases are provided in FIG. 1 or 2 as “mm.” The specific mismatched basepair are bolded, underlined, italicized, and highlighted.

### Example 2: In Vitro Reduction in Tau Protein

Each of the oligomers targeting the 3′ UTR of an MAPT transcript was tested for its ability to decrease Tau protein in mouse primary neurons expressing the entire human MAPT gene as a bacmid containing transgene (C57-b16 BAC-Tg hTau; Polydoro et. al., J. Neurosci. (2009) 29 (34): 10747-9). Primary hTau mouse embryonic forebrain neuronal cultures do not express endogenous mouse tau as mouse tau was knocked out. Primary neurons were generated by papain digestion according to manufacturer's protocol (Worthington Biochemical Corporation, LK0031050). Briefly, forebrains were dissected from hTau mouse E18 BAC-Tg embryos expressing the entire human microtubule-associated protein Tau (MAPT) gene on a murine MAPT-null background and were incubated at 37° C. for 30-45 minutes in papain/DNase/Earle's balanced salt solution (EBSS) solution. After trituration and centrifugation of cell pellet, the reaction was stopped by incubation with EBSS containing protease inhibitors, bovine serum albumin (BSA) and DNase. The cells were triturated and washed with Neurobasal (NB, Invitrogen) supplemented with 2% B-27, 100 μg/ml penicillin, 85 μg/ml streptomycin, 0.5 mM glutamine. The cells were plated in supplemented NB media onto poly-D-lysine-coated 96-well optical imaging plates (BD Biosciences) at 15,000 cells/well.

After obtaining the primary hTau mouse embryonic forebrain neuronal cultures expressing a human MAPT gene, the cultures were treated with oligomers to inhibit the Tau mRNA and protein expression. The cultures were then subject to immunocytochemistry and imaging to measure the inhibition. One day post plating (DIV 1), half of the supplemented neurobasal (NB) media on the primary hTau mouse embryonic forebrain neuronal cultures was removed and replaced with supplemented NB media containing various concentrations of LNA oligomers. Primary hTau neuronal cultures were cultured with LNA oligomers until 13 days post plating (DIV 13). On DIV 13, the cultures were rinsed with Dulbecco's phosphate-buffered saline lacking calcium and magnesium (DPBS, Invitrogen) and fixed in 4% paraformaldehyde/4% sucrose/DPBS for 15 min. Cultures were rinsed and then blocked and permeabilized in DPBS plus 0.1% Triton X-100 (TX-100) and 3% BSA for one hour at room temperature. Cultures were rinsed and then incubated for two hours at room temperature with primary antibody 1:500, Tau5 antibody to measure Tau protein, Invitrogen AHB0042; and 1:500, β-III tubulin (TuJ-1) antibody to measure neurite area, Abcam ab41489) in DPBS plus 3% BSA and 0.1% TX-100. Cultures were rinsed and incubated with Hoeschst 33342 nuclear dye (1:800, Invitrogen) and AlexaFluor fluorescence-conjugated secondary antibodies (Invitrogen, 1:500) in DPBS plus 3% BSA and 0.1% TX-100 for one hour at room temperature. Cultures were rinsed abundantly and stored in DPBS until imaging. Imaging was conducted using the Cellomics VTi automated immunofluorescence imaging system. In brief, using untreated wells, saturation levels for each fluorophore channel were set to 70%. Then 12 sequential images were acquired from each well, and total fluorescence intensity and total fluorescence area were calculated for both Tau and TuJ-1 proteins using the Cellomics VTi SpotDetector (version 4) image analysis software. To evaluate Tau protein reduction resulting from oligomer treatment, a Tau5 total fluorescence intensity-to-Tuj-1 total fluorescence area ratio (Tau/TuJ-1) was created for each well and then all data were normalized to the average Tau/Tuj-1 ratio of the untreated wells. To evaluate neurite/neuronal toxicity from oligomer treatment, the Tuj-1 total fluorescence area from each well was normalized to the average Tuj-1 total fluorescence area of the untreated wells. TuJ-1 intensity acts as an internal standard for each sample. Nuclei counts from each well were also acquired as an alternative measure of toxicity associated with LNA oligomer treatment. Data are expressed as mean±S.D. For immunocytochemistry, data points represent the mean±S.D. from wells treated in triplicate. Potency values were generated using wells treated with a broad concentration range of LNA oligomer, from which the resulting normalized Tau/Tuj-1 and Tuj-1 values were analyzed compared to normalized values from saline control samples. Analysis was done using non-linear regression with top and bottom values set at fixed values of 100% and 0%, respectively, where 100% inhibition represents a complete reduction of signal compared to the control sample (FIG. 2). For qPCR, data were analyzed using a one-way ANOVA with a Dunnett's multiple comparison test to compare saline- and LNA oligomer-treated groups. Statistical significance was set at a value of p<0.05.

The reduction of Tau protein by each oligomer was compared with saline. The results of the Tau protein reduction compared to Saline are shown in FIG. 2. If the Tau protein level in antisense oligonucleotide treated neurons was equal to or higher than in control cells, percent inhibition is expressed as zero inhibition. If present, ‘N.D.’ indicates ‘not determined’ and ‘TBD’ indicates ‘to be determined’. The target regions to which antisense oligomers are inhibitory are considered ‘hot-spots’ on the Tau transcript.

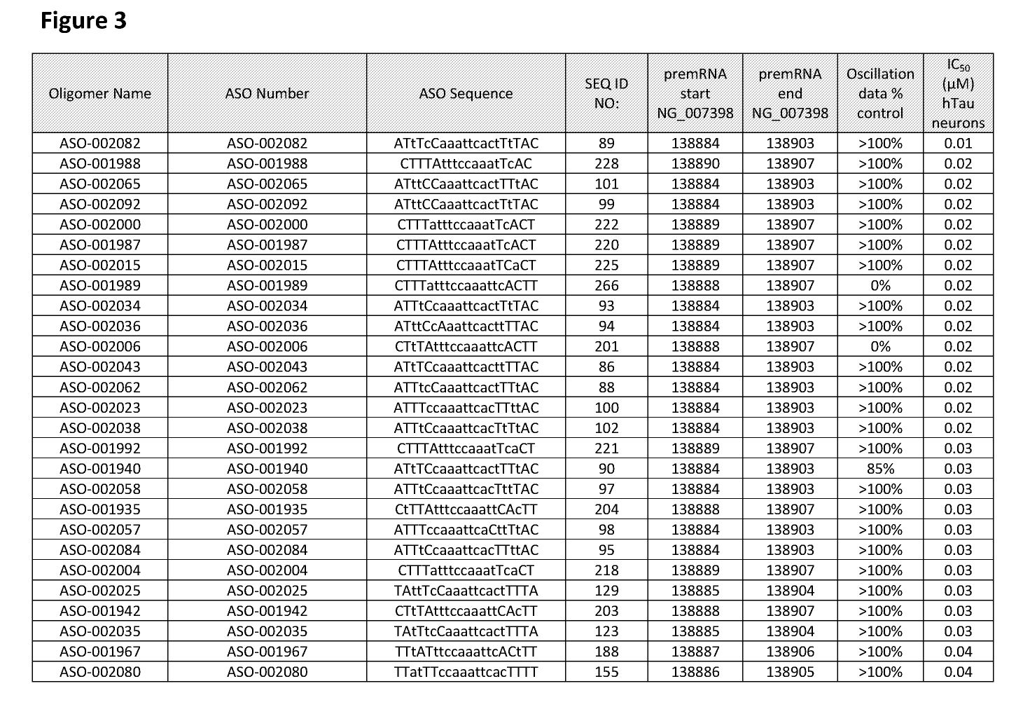

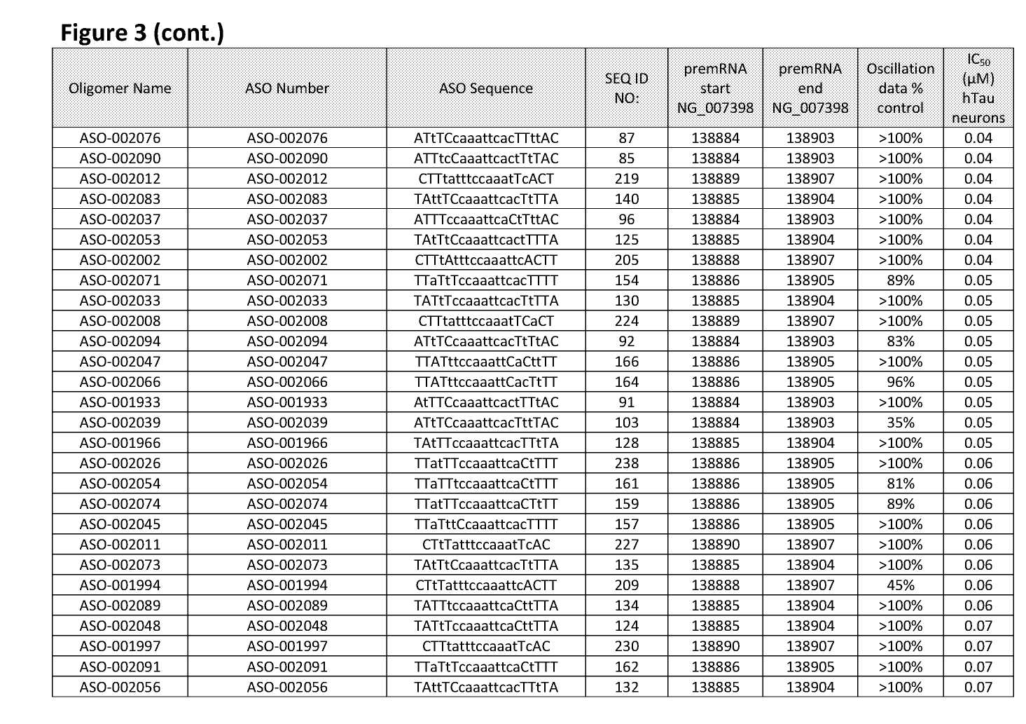

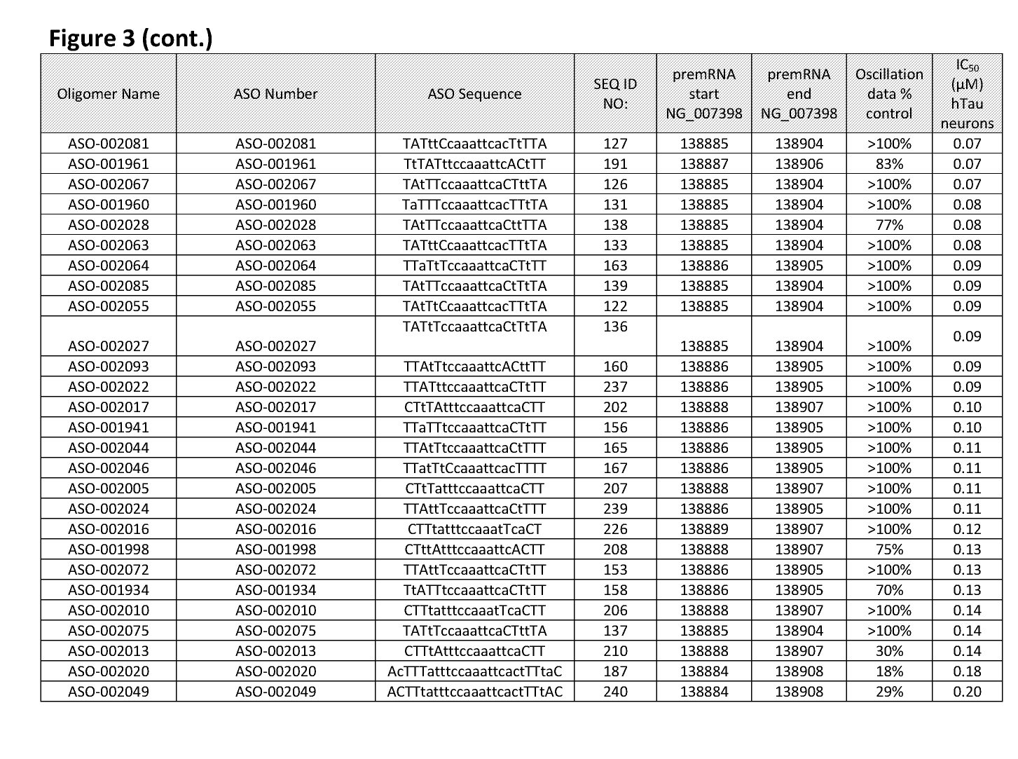

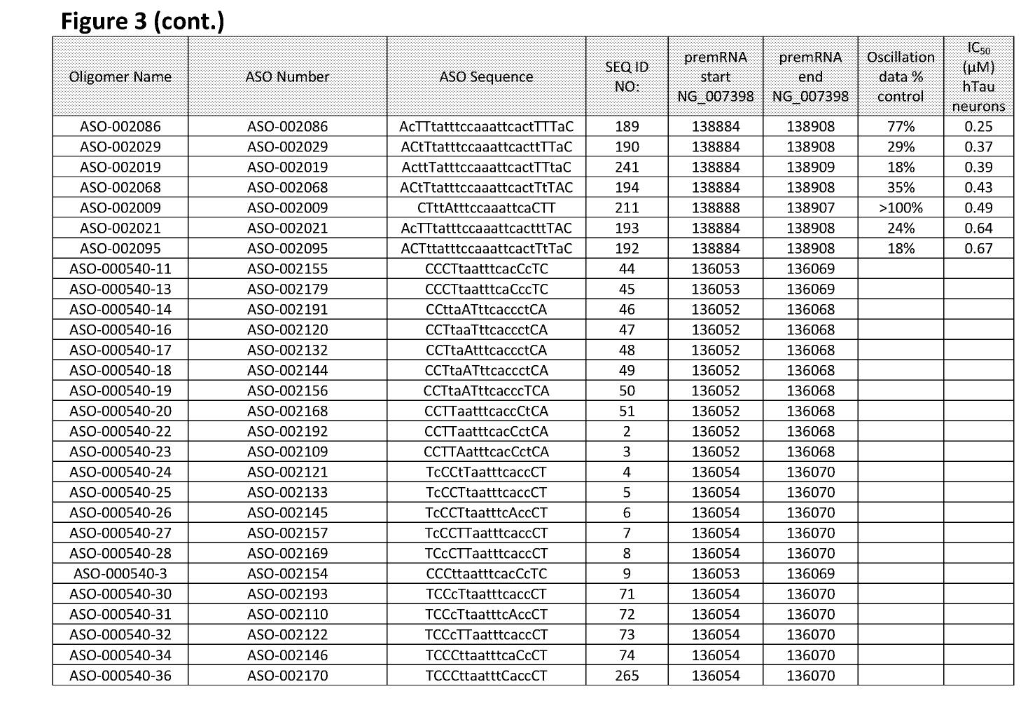

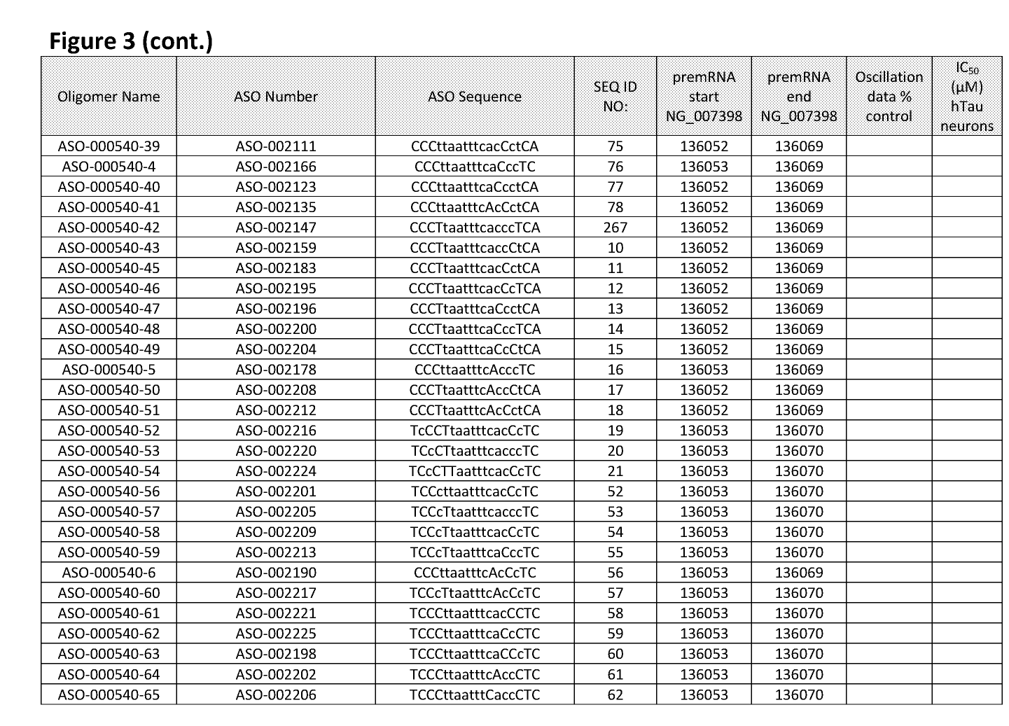

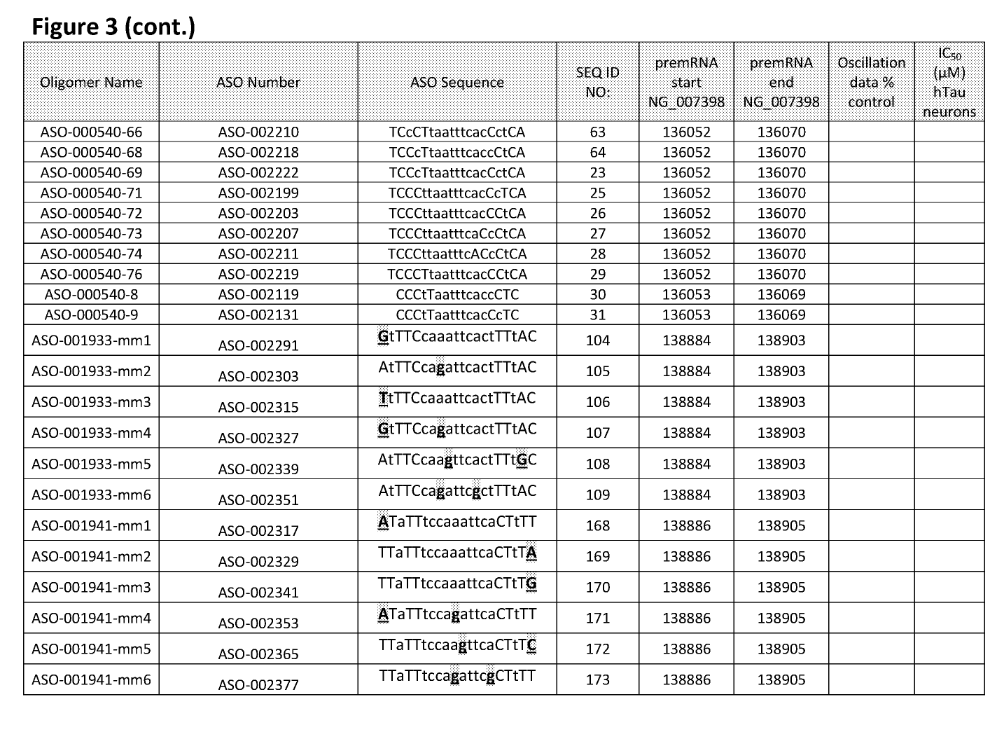

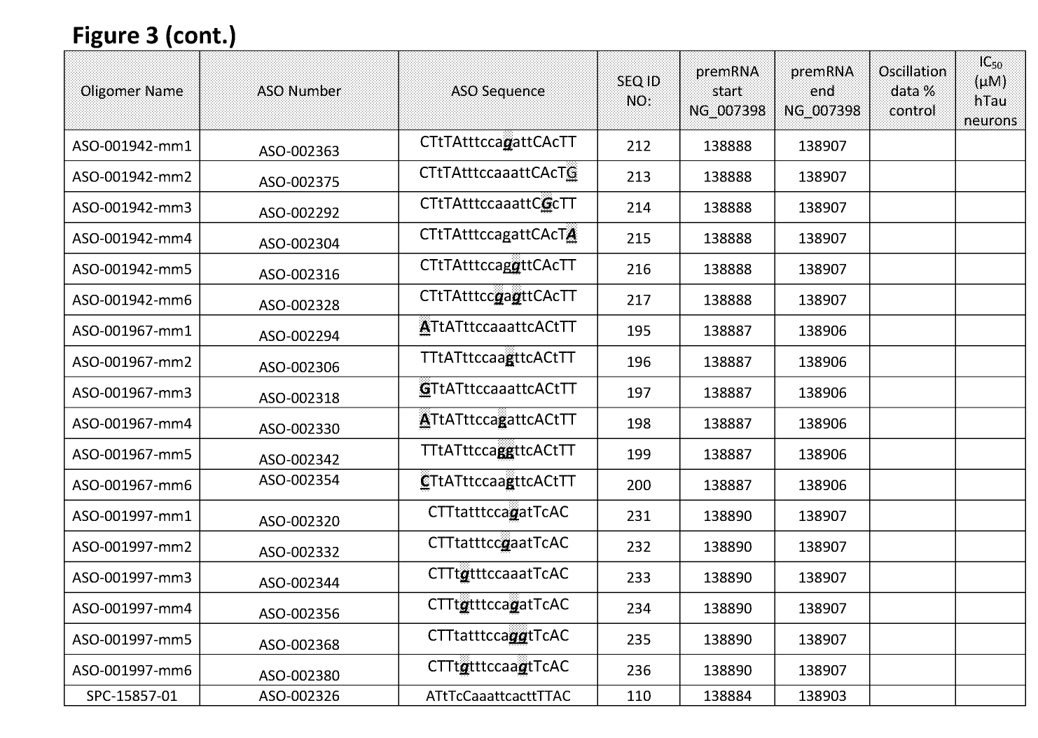

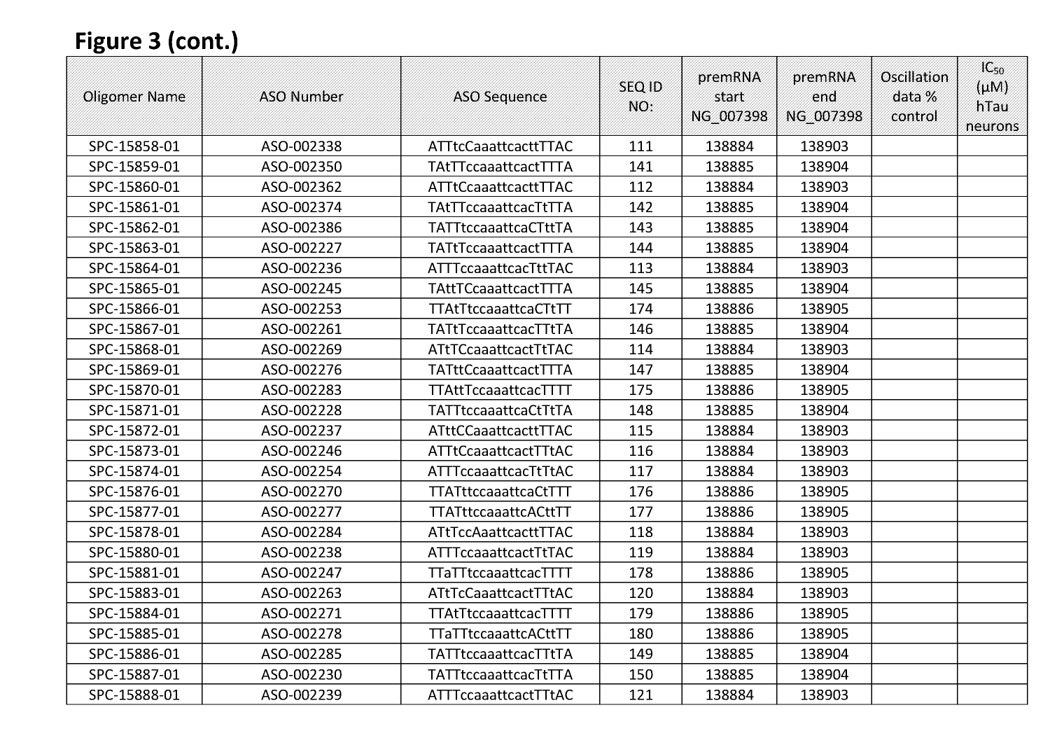

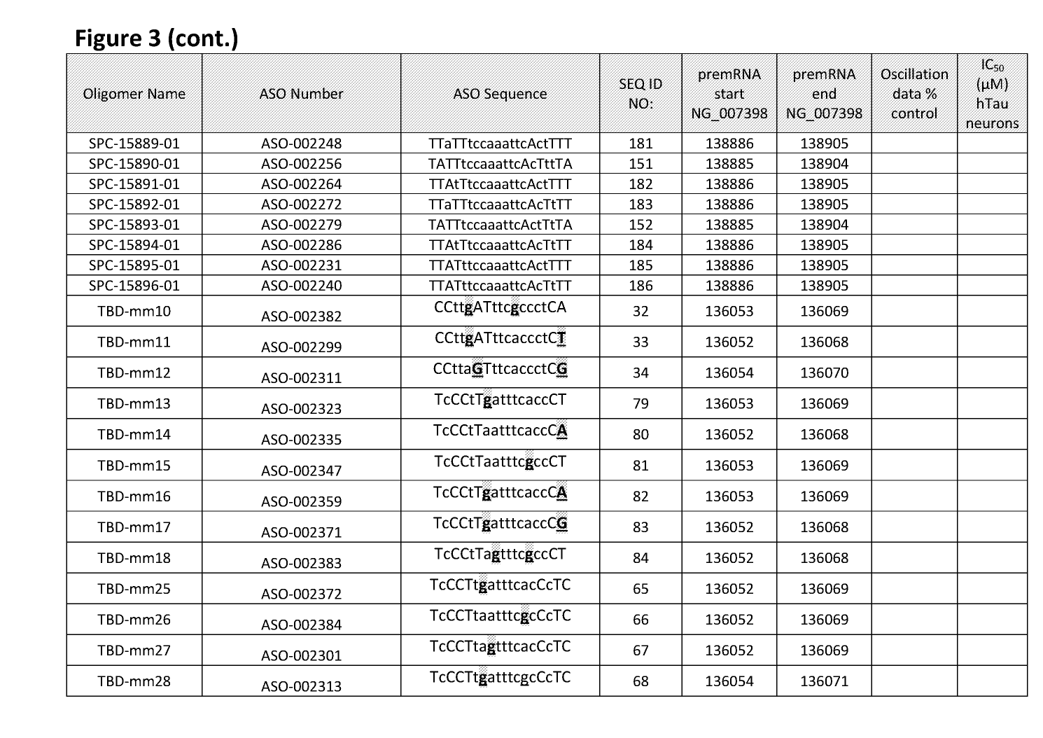

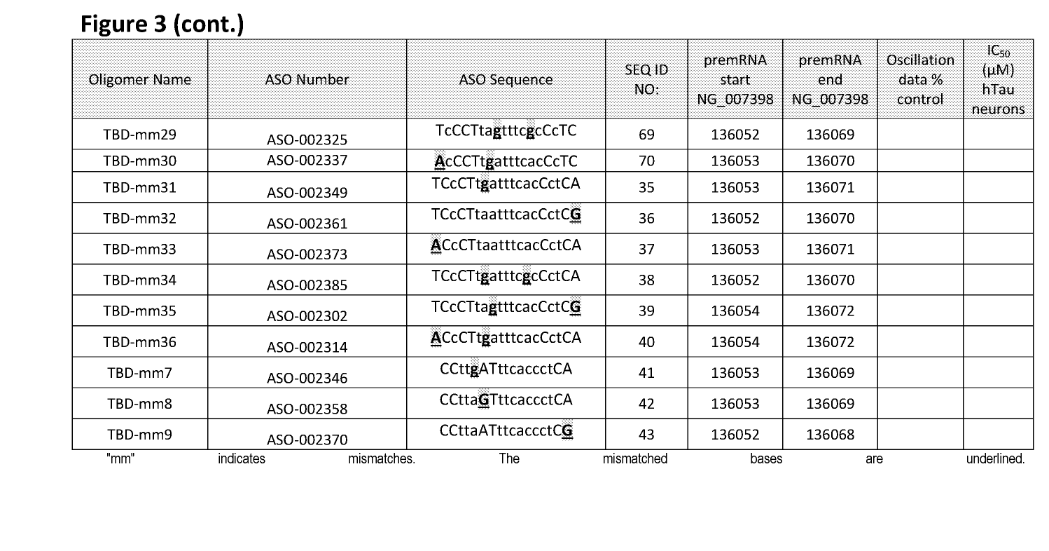

Oligomers were diluted in water and added to cells at 1 day post plating (DIV01) to a final concentration of 5 μM. For IC₅₀ determinations, neurons were treated with a top concentration of 5 μM and a concentration response dilution of 1:3 was used to define the IC₅₀ value. The calculated IC₅₀ value for certain oligomers is shown in FIG. 3.

### Example 3: Spontaneous Calcium Oscillation Measurement

The present application shows that a reduction of oscillations in intracellular free calcium concentration (calcium oscillation) corresponds to increased neurotoxicity of an oligomer to a cell. The amount of reduction and how it corresponds to an increase in neurotoxicity can be determined as described herein. To measure primary cortical neuron spontaneous calcium oscillation, rat primary cortical neurons were prepared from Sprague-Dawley rat embryos (E19). Cells were plated 25,000 cells/well onto 384 well poly-D-lysine coated fluorescent imaging plate reader (FLIPR) plates (Greiner Bio-One) in 25 μl/well Neurobasal media containing B27 supplement and 2 mM glutamine. Cells were grown for 11 days at 37° C. in 5% CO₂ and fed with 25 μl of additional media on DIVO4 and DIVO8 for use on DIV11. On the day of the experiment, media was removed from the plate and the cells were washed once with 50 μl/well of 37° C. assay buffer (Hank's Balanced Salt Solution with 2 mM CaCl₂ and 10 mM Hopes pH 7.4). Oscillations were tested in the presence and absence of 1 mM MgCl₂. Cells were loaded with a cell permanent fluorescent calcium dye, fluo-4 AM (Life Technologies). Fluo-4 AM was prepared at 2.5 mm in DMSO containing 20% plutonic F-127 then diluted 1:1000 in assay buffer. Cells were incubated 1 hr with 20 μl of 2.5 μM fluo-4 AM at 37° C. in 5% CO₂. After 1 hr 20 μl of room temperature assay buffer was added and the cells were allowed to equilibrate to room temperature for 10 additional minutes and placed in the FLIPR. Baseline signal (measurement of intracellular calcium) was read for 100 seconds (1 reading/second) before the addition of anti-sense oligomers. Oligomers were added with a 384 well head in the FLIPR in 20 μl of assay buffer at 75 μM for a final concentration of 25 μM. FLIPR signal was read for an additional 200 seconds (1 reading/second) after the addition of oligomer. A second 5 minute post addition plate read (300 one second points) on the FLIPR was conducted to allow for additional data capture. Raw data from 5 minute read was exported and, using Excel, spike amplitude and frequency was calculated. Calculations were performed by measuring the average FLIPR signal over the 300 second read for control (non-treated) wells. For treated wells, a scoring system was developed where a score of 1 was given for each 1 second read where signal increase greater than 50% of the average control value (calculated above). A score of 0 was given for each 1 second read that increase less than 50% of average control value. For each treatment a total score was calculated and converted to percent control for graphical purposes. If the antisense oligomer produced a calcium oscillation response greater than that of AMPA alone, percent of control is expressed as greater than 100% (FIG. 3).

Effect of oligomers on primary neuronal spontaneous calcium oscillations was measured under two conditions, in the presence and absence of 1 mM MgCl₂ as a source of Mg²⁺ ions, as described previously (Murphy et. al., J. Neurosci. 12, 4834-4845 (1992)). This was done to isolated N-methyl-D-aspartate (NMDA)- and α-amino-3-hydroxy-5-methyl-4-isoxazolepropionic acid (AMPA)-receptor mediated calcium oscillations.

Antisense oligomer inhibition of spontaneous calcium oscillations mediated by either NMDA or AMPA was assessed in the presence or absence of 1 mM MgCl₂ (representing 100% control in each case). Addition of 25 μM antisense oligomers (ASO) inhibited AMPA receptor but not NMDA receptor mediated oscillations. ASO, and other oligos that behaved similarly, were shown to negatively impact central nervous system (CNS) network activity in vivo and electrophysiologic spontaneous neuronal activity in vitro (data not shown). Tau antisense oligonucleotide impact on spontaneous calcium oscillations in primary neurons is summarized in FIG. 3. See Murphy, et al., J. Neurosci. 12, 4834-4845 (1992).

Calcium oscillation reduction was measured for the oligomers of the invention and summarized in FIG. 3. The oligomers showing greater than 25% of control in the calcium oscillation assay were selected for further analysis.

### Example 4: Sequence Score Calculation

The present application also shows that the sequence score of an oligomer, as calculated herein, corresponds to the neurotoxicity of the oligomer. In certain aspects of the invention, the higher the sequence score the less neurotoxic the oligomer. Different cut off values, over which the sequence score indicates that the oligomer has reduced neurotoxicity, can be determined as described herein.

The sequence score of each oligomer was calculated to predict the suitability and neurotoxicity of the oligomers. Sequence score is a mathematical calculation determined for all oligomers and is based on the percent of G and C nucleotides, or analogs thereof, within a given oligomer sequence. The following formula was applied to all oligomers in order to calculate sequence score:

$$\frac{number of C nucleotides - number of G nucleotides}{nucleotide length} \qquad ( I )$$

An example calculation is given for oligomer ASO-001967 (SEQ ID NO: 188): TTtATttccaaattcACtTT: 4-0/20=sequence score of 0.20

The sequence score of the selected oligomers were calculated for further studies. To determine the cut off value for the sequence score, an In vivo tolerability study was performed as shown in Example 5.

### Example 5: In Vivo Tolerability

The in vivo tolerability of the oligomers was tested to see how the oligomer was tolerated when injected into an animal. The in vivo tolerability of the oligomers allows for a prediction of how the oligomer will be tolerated when administered therapeutically to a subject such as a human.

### Subjects

In vivo tolerability of the oligomers were tested in mice and rats. Animals for Tau qPCR and behavioral studies were adult, C57Bl/₆J female mice (20-30 g; Jackson Laboratories, Bar Harbor, Me.) housed 3-4 per cage. Animals were held in colony rooms maintained at constant temperature (21±2° C.) and humidity (50±10%) and illuminated for 12 hours per day (lights on at 0600 hours). In some cases, male and female transgenic mice (30-40 g) expressing a tau transgene derived from a human PAC, H1 haplotype driven by the tau promoter (Polydoro et. al., J. Neurosci. (2009) 29(34): 10741-9), and in which the native mouse Tau gene was deleted, were used to assess pharmacodynamic endpoints and tissue drug concentrations. For intrathecal infusion studies, female Sprague-Dawley rats (180-225 g at testing; Harlan) were singly housed in colony rooms maintained at a constant temperature (21±2° C.) and humidity (50±10%) and illuminated for 12 hours per day (lights on at 0600 h). All animals had ad libitum access to food and water throughout the studies. Behavioral studies were conducted between 0700 and 1500 hours. Animals were maintained in accordance with the guidelines of the Animal Care and Use Committee of the Bristol-Myers Squibb Company, and the “Guide for Care and Use of Laboratory Animals” published by the National Institutes of Health. Research protocols were approved by the Bristol-Myers Squibb Company Animal Care and Use Committee.

### Administration Routes-Intra-Cerebroventricular or Intrathecal Injections.

The oligomers were administered to mice by either intracerebroventricular (ICV) injection or intrathecal injection. Intracerebroventricular injections were performed using a Hamilton micro syringe fitted with a 27 or 30-gauge needle, according to the method of Haley and McCormick. The needle was equipped with a polyethylene guard at 2.5 mm from the tip in order to limit its penetration into the brain. Mice were anesthetized using isoflurane anesthetic (1.5-4%). The mouse to be injected, weighing 20-30 g, was held by the loose skin at the back of the neck with the thumb and first fingers of one hand. Applying gentle but firm pressure, the head of the animal was then immobilized by pressing against a firm flat level surface. The needle tip was then inserted through the scalp and the skull, about 1 mm lateral and 1 mm caudal to bregma. Once the needle was positioned, antisense oligonucleotide was given in a volume of 5 microliters in saline vehicle and injected into the right (or left) lateral ventricle over 20-30 seconds. The needle was left in place for 10 seconds before removal. This procedure required no surgery or incision. Animals were warmed on heating pads until they recovered from the procedure. Brain tissue (right, frontal cortical region) was collected on dry ice or RNAlater for drug concentration analysis and Tau qPCR respectively at multiple time points following dosing, e.g., 1 week through 16 weeks post-dosing.

For intrathecal (IT) injections of mice, animals were maintained under light isoflurane anesthesia (1.5-5%). The mouse was held securely in one hand by the pelvic girdle and inserting a 30G ½ inch needle connected to a Hamilton syringe into the tissue between the dorsal aspects of L5 and L6, perpendicular to the vertebral column. When the needle enters the subarachnoid space, a sudden lateral movement of the tail was observed. This reflex was used as an indicator of successful placement of the needle for IT administration. A 5-10 μL volume of antisense oligonucleotide was injected slowly (over approximately 60 seconds) into the subarachnoid space.

For intrathecal injections in rat, intrathecal catheters were surgically implanted using methods described by Yaksh and Rudy, Physiol. Behay. (1976) 17(6): 1031-6. The rat was mounted to a stereotaxic frame with isoflurane anesthesia maintained through a nose cone. A skin incision was made beginning approximately at the line joining the ears and extending caudally about 3 cm along the midline. The muscle where it attached to the occipital crest of the skull was cut about 3 mm lateral on both sides of the muscle midline. Using retractors or forceps, the muscle was peeled caudally to expose the cisternal membrane at the base of the skull. The fascia and tissue were carefully removed from the membrane. The bent beveled end of a 16-22 gauge needle was used to make a 1-2 mm lateral incision in the cisternal membrane. A sterilized IT catheter, made of polyethylene tubing (PE10 tubing stretched to approximately 1.3 mm outer diameter), was inserted through the incision and carefully advanced caudally through the subarachnoid space while it was rotated between thumb and forefinger and while the base of the tail was gently pulled to align the spinal cord using the other hand. If any resistance was encountered, the catheter was retracted slightly, and slowly advanced again. Once the catheter had been advanced to the desired area, it was flushed with 20 μL sterile saline and the cranial end was passed through the skin using a 19 gauge needle about 1 cm from the incision. The catheter was plugged with a pin. Rats were given oral antibiotics for 5 days following the surgery. At least five days after surgery, a single antisense oligonucleotide injection was diluted in water and delivered via a programmable infusion pump (Knopf) at a rate of 10 μl/minute in a volume of 10 to 50 μl. A brief saline flush of 5 ul was given just prior to the antisense oligonucleotide delivery and a 10 μl saline flush was given just following the oligonucleotide delivery at a rate of 10 μl/minute to cover the dead volume of the catheter (6-7 μl). A saline flush of 20 ul was also given to animals 1-2×/week until used for an experiment.

Data was analyzed using the delta delta Ct method where each sample is first normalized to GAPDH and then expressed as percent of untreated control animals.

### Acute Tolerability Behavioral Assessments

For one hour following the single injection of antisense oligonucleotide ICV (intra-cerebroventricular) or IT (intrathecal), animals were observed for behavioral side effects and scored for the severity of side effects on a scale of zero (no side effects) to 20 (convulsions resulting in euthanasia). The tolerability scale was divided into 5 neurobehavioral categories: 1) hyperactivity 2) decreased activity and arousal 3) motor dysfunction/ataxia 4) abnormal posture and breathing and 5) tremor/convulsions. Each category was scored on a scale of 0-4, with the worst possible total score of 20. Animals were observed for changes in behavior in the home cage, and then they were removed from the home cage for more detailed observations which included measurement of grip strength and righting reflex.

### Novel Object Recognition

Short term recognition memory was measured using the novel object recognition (NOR) task. NOR testing was based on the spontaneous behavior of rodents to explore a novel object more than a familiar one (Dodart et. al., Neuroreport (1997) 8(5): 1173-8; Ennaceur and Delacour, Behay. Brain Res. (1988) 31 (1):47-59). After a one hour retention interval between training (T1) and testing (T2) sessions, mice remembering the objects from the training session will show a preference for the novel object on the test session. For these experiments, animals were handled for 3 days and habituated to the chamber (48 cm×38 cm×20 cm) on the day prior to the test session. The chamber was made of polyethylene and lined with vinyl flooring. On the test day, animals were placed in the rectangular test chamber and allowed to explore two identical objects (7.6 cm high×5.1 cm wide) for a 15 minute training period. One hour later, mice were placed back into the test chamber for a 10 minute test session, this time with one object they had observed during training and one novel object. Objects were cleaned thoroughly with 25% ethanol between training and testing sessions and between subjects, and were cleaned again at the end of the day with mild detergent. Object exploration was only considered when the animal's nose was pointed at the object. Exploration was recorded using ObjectScan tracking software (Cleversys, Reston, Va.). Data are reported as percent of time spent exploring objects (i.e., novel time/novel+familiar time*100).

### Morris Water Maze

Spatial learning and memory was assessed based on Morris Water Maze assay (Morris J. Neurosci. (1984) 11(1):47-60). Water maze represents a pool with the diameter of 120 cm. Water was made opaque using white, non-toxic tempura paint (20° C.±1). The pool was surrounded with distinct extra-maze cues.

Prior to hidden platform training, all mice were exposed to the water maze pool by allowing them to swim down the rectangular channel during 2 pre-training trials. The escape platform was placed in the middle of the channel. If a mouse was not able to find and mount the platform during 60 sec trial, it was guided to it and allowed to sit for up to 10 sec. After pre-training, mice underwent hidden platform training, during which a 10×10 cm platform was submerged 1.5 cm below the surface. The platform location remained the same throughout training whereas the drop location varied randomly between the four daily trials as well as across the 4 days of training. Mice received 2 sessions per day for 4 consecutive days. Each session consisted of 2 trials with a 10-min inter-trial interval. The maximum time allowed per trial was 60 sec. If a mouse did not find or mount the platform, it was guided to the platform by the experimenter. All mice were allowed to sit on the platform for 10 sec after each training trial.

For probe trials, the platform was removed and each mouse was allowed to swim for 60 sec. The drop location for the probe trials was 180° from the platform location used during hidden platform training. After 60 sec, mice were guided to the platform location before retrieval from the pool. For early memory retrieval mice were probed 2 h after the last hidden platform training; long term memory recall was assessed 16 h following the last hidden platform training. 2 h following the 16 h probe trial, all mice underwent the visible platform training, where a local cue (pole built using legos) was placed above the hidden platform. Mice were given 2 training trials. All behavior was recorded with a video tracking system (Cleversys Inc). Escape latencies, distance traveled, swim paths, swim speeds, and platform crossings were recorded automatically for subsequent analysis.

### Catwalk

The Catwalk (Noldus, The Netherlands) is an automated and computerized gait-analysis technique that allows objective quantification of multiple static and dynamic gait parameters. Mice were placed on one end of the catwalk and allowed free exploration for 3 min or until they have 5 compliant trials, whichever comes first. Data were exported and classified using the Catwalk software. An average of classified trials was used for data analysis. Measures of interest include but are not limited to: print position or the distance between the position of the hind paw and previous placement of the ipsilateral front paw, initial and terminal dual stances, paw swing speed, and paw stand or the duration of paw contact with the glass plate in a step cycle.

### Behavioral Statistics

Statistical analyses for all behavioral tests were conducted using GraphPad Prism (GraphPad Software, Inc., La Jolla, Calif.). For NOR, data were analyzed using either a paired t-test for within-group analyses or by an ANOVA followed by a Dunnett's post-hoc test for between group analyses. For MWM, a repeated MWM ANOVA was used to analyze the acquisition phase and a one-way ANOVA followed by Dunnett's post-hoc for probe trial analyses.

Brain Tau mRNA Analysis

### Brain Homogenization

Mouse brain tissue was homogenized in a 10× volume of a high salt/sucrose buffer (10 mM Tris-HCl, pH 7.4, 800 mM NaCl, 10% sucrose (w/v), 1 mM EGTA) supplemented with phosphatase inhibitor cocktail sets 2 and 3, 1 mM PMSF (Sigma, Saint Louis, Mo.), and complete protease inhibitor cocktail EDTA-free (Roche, Indianapolis, Ind.) using a Quiagen TissueLyzer II. The homogenate was centrifuged at 20,000×g for 20 minutes at 4° C. The supernatant was centrifuged at 100,000×g for 1 hour at 4° C. and the supernatant was analyzed.

### RT-PCR Assays

For cDNA synthesis and subsequent PCR, 300 ng of RNA was added to 1 well of a 96 well plate (Axygen, PCR-96-C-S). To each well 7.5 μl of master mix (54, of 2.5 mM NTP mix and 2.54, random primers per reaction) was added and the plate was centrifuged at 1000 rpm and placed in thermocycler for 3 min at 70° C. Plates were immediately cooled on ice and 4 μl of reaction master mix was added. Prior to PCR, plates were briefly centrifuged to collect sample in bottom of well. cDNA synthesis was carried out at 42° C. for 60 min, 95° C. for 10 min followed by a hold at 4° C. cDNA Samples were diluted 1:3 with molecular biology grade water and stored at −20° C. until further use.

For PCR, each sample was run in triplicate with two probe sets (MAPT: Taqman Expression assays Hs00902193_m1; RhoA: Taqman Expression assays; GAPDH Taqman Expression assays Hs01922876_u1). To each reaction 4 μl of previously diluted cDNA and 6 μL of master mix was added and plates were centrifuged. Samples were incubated at 95° C. for 20 sec follow by 40 cycles at 95° C. for 1 sec and 60° C. for 20 sec.

### Results

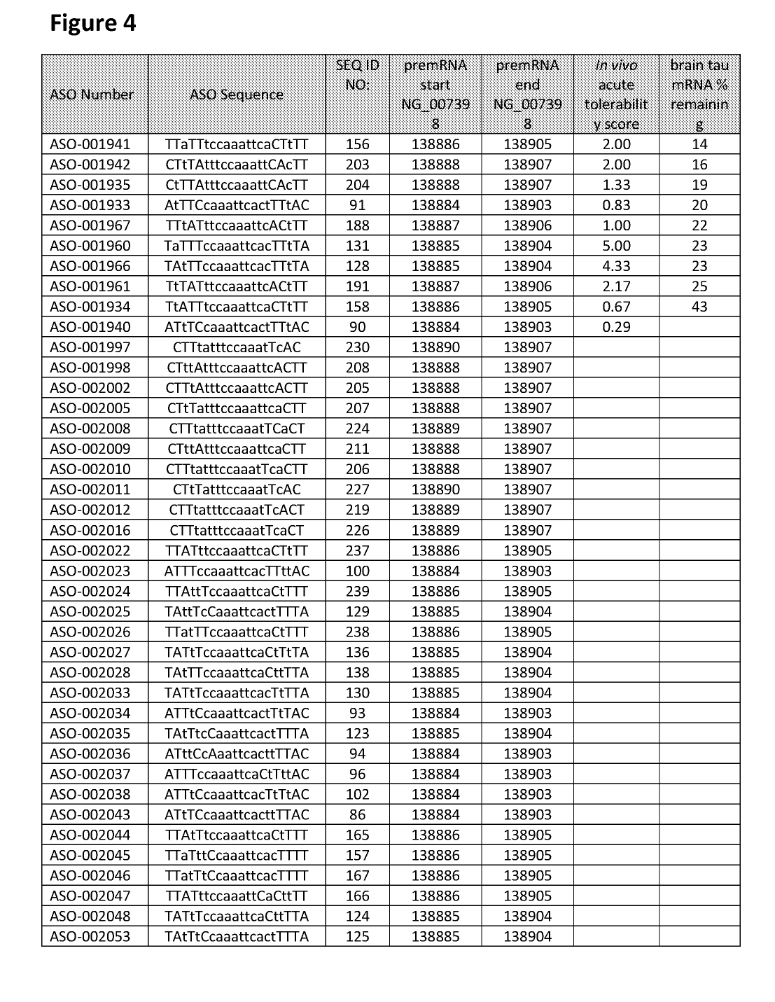

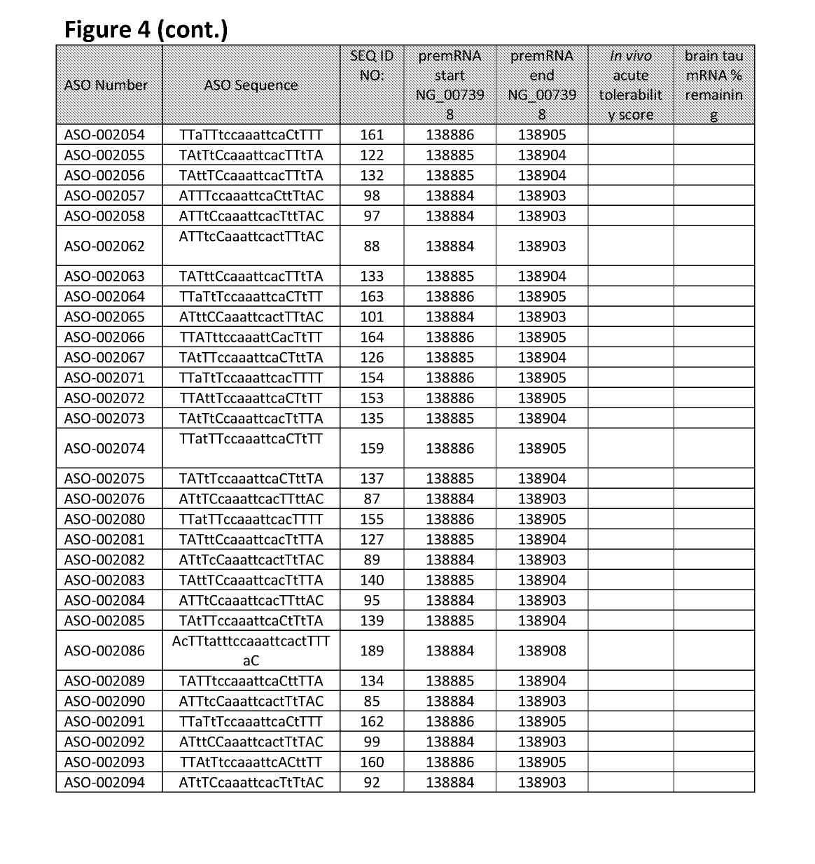

In vivo cumulative tolerability threshold following an ICV injection of 100 μg of an antisense oligonucleotide was set at 4. The correlation analysis shows that the oligomers having in vivo tolerability lower than 4 tend to have a sequence score equal to or higher than 0.2. Potent LNA oligonucleotides targeting MAPT at or partially overlapping nucleotides in the 3′UTR were identified and found to be well tolerated in primary neurons in vitro and following i.c.v. administration in vivo (See FIG. 4 below).

The In vivo acute tolerability score and brain tau mRNA % control data shown in FIG. 4 show that oligomers that hybridize to target MAPT mRNA sequences contained between 134,947-138,924 further identify validated sequence target site oligomers that are both well tolerated and potently reduce Tau mRNA In vivo.

### Example 6: Oligomer Prioritization

The assays described herein can be used in combination to selected oligomers for further testing. Properties of selected oligomers can be described as shown in Table 1. Based on these criteria, certain oligomers were selected for additional dose-response testing in vitro and in vivo.

TABLE 1
Summary of criteria used to prioritize
oligomers for additional testing.

| Assay | Prioritization Criteria |
|---|---|
| Tau protein reduction | >70% reduction in Tau protein (5 μM oligomer) |
| Calcium oscillations | <25% reduction in calcium oscillations |
| Sequence score | Sequence score ≧0.20 |

In other embodiment, oligomers can be selected based on the following characteristics: (1) Tau protein reduction >30% reduction in Tau protein (5 μM oligomer); (2) calcium oscillations <25% reduction in calcium oscillations; and (3) sequence score equal to or higher than 0.2.

### Example 7: In Vivo Data

Oligomers were injected into animals to determine their effect on Tau expression and on the behavioral properties of the animal.

### Research Animals and Administration Routes

The animals used in this Example are the same mice and rats described in Example 5 and were handled in the same manner as described in Example 5. Animals were injected as described in Example 5.

RT-PCR assays were performed as described in Example 5.

### Running Wheel Assay

The Home Cage Running Wheel assay measures spontaneous activity in a voluntary free-spinning running wheel (Columbus Instruments). Each wheel has a magnetic sensor that connects to a computer interface and records wheel revolutions at user-specified intervals. In this study, mice were placed individually into cages with a running wheel and wheel rotations were monitored continuously in 15 min increments. To allow for habituation and establish baseline activity levels, control and test mice were tested over 7 days, after which they were transferred into clean cages and dosed with either saline or 100 ug of an oligomer by ICV injection. Two weeks post treatment mice were returned to the running wheel cages to evaluate treatment effects over 7 days.

Brain Tau mRNA Analysis

### Brain Homogenization

Mouse brain tissue was homogenized as described in Example 5. CSF Collection:

All animal protocols were approved by the Wallingford BMS Animal Care and Use Committee. CSF was collected from the cisterna magna of mice following exsanguination as described by Barten, et al., J Alz. Res. 24: 127-141 (2011). In brief, CSF was collected with a P20 pipettor after puncturing the dura with a 30 gauge needle under a dissecting microscope. Body temperature was monitored and maintained at normal levels using heating pads and lamps. CSF was collected from rats after exposure of the cisterna magna and withdrawal using a 1 ml insulin syringe. CSF was placed on ice, centrifuged briefly to remove any red blood cells, transferred to another tube while measuring the volume, and frozen on dry ice. CSF Tau protein reduction measured by Tau Protein Enzyme-Linked ImmunoSorbant Assay (ELISA described below) was observed after 4 weeks following a single bolus i.c.v. injection of an oligomer (data not shown).

### Tau Protein Enzyme-Linked ImmunoSorbant Assay (ELISA):

For brain tissue, BT2 (antibody to Tau amino acid 194-198, Thermo Scientific) was used to coat 96 well black ELISA plates (Costar) at a concentration of 2.5 μg/ml for 1 hour at 37° C. After washing in TBST, the plates were blocked with 3% bovine serum albumin in TBS. Recombinant human Tau441 (rPeptide; Bogart, Ga.) or a 1:5000 dilution of the brain homogenates were diluted in 1% BSA+0.05% Tween-20 in TBS. Alkaline phosphatase conjugated Tau-5 (antibody to Tau amino acid 210-230, Covance, Emeryville, Calif.) was added to the samples at a 1:2000 dilution for co-incubation overnight at 4° C. with shaking. After washing in TBST, the signal was amplified with the Tropix CDP Star detection reagent from Applied Biosystems. The chemiluminescent signal was read on an Envision (Perkin Elmer). For CSF samples, this ELISA was done in a 384 well format to minimize the volume of CSF needed. 10 μl of a 1:2 dilution of CSF was added to each well.

In vivo reduction of human tau mRNA level was measured in mice (FIG. 4). Brain Tau mRNA and Tau protein reduction was measured over time following a single i.c.v. bolus of 100 μg oligomer administration into wild type C57 mice (N=12). Tau mRNA expression (normalized to GAPDH) was measured at 2, 4, 8 and 12 weeks post injection. Tau protein (% of saline) level was measured at 2, 4, 8 and 12 weeks post injection. This oligomer produced a durable reduction in Tau mRNA and protein with Tau protein remaining reduced following 12 weeks post single bolus i.c.v. injection. Other oligomers defined within this invention exhibit more profound reductions in Tau mRNA and protein with durable tissue oligomer exposure as measured by ELISA (further described below).

In-situ Hybridization (ISH) detection of Tau mRNA and the oligomers was performed on 20 um fresh frozen brain sections mounted. Slides were thawed, fixed in 4% paraformaldehyde for 10 minutes at room temperature, washed in phosphate buffered saline (PBS) and acetylated with 0.25% acetic anhydride/0.1M triethanolamine for 10 minutes at room temperature (RT). Following PBS washes, each slide was pre-hybridized in 0.7 ml pre-warmed hybridization buffer (HB), 50% formamide/5× saline sodium citrate (SSC), 100 μg/ml yeast tRNA, 1×Denhardt's, for 30 minutes at 67° C. 5′ FAM-labeled sense probe (complementary all LNA probe) was heated to 90° C. for 4 minutes, cooled on ice then diluted in HB. Slides were hybridized in 0.45 ml for 30 minutes at 67° C. with a hybrislip (Electron Microscopy Sciences, Hatfield, Pa.). They were subsequently dipped in 0.1×SSC then washed three times in 0.1×SSC at 67° C. Slides were then treated in 3% hydrogen peroxide for 10 minutes, washed in PBS, and blocked for 15 minutes at RT in 0.1M Tris-HCl, pH 7.5, 0.15M NaCl, 0.5% blocking agent (FP1020, Perkin Elmer Waltham, Mass.). This was followed by incubation in rabbit anti-fluorescein-horse radish peroxidase for 30 minutes at RT. Following TBST washes (Tris buffered saline with 0.05% Tween 20), tyramide signal amplification was performed (TSA Plus, Perkin Elmer). Slides were washed in TBST, nuclei stained using DAPI, and coverslip mounted using Prolong Gold (Invitrogen, Carlsbad, Calif.). For chromogenic detection, slides (post-TSA washes) were incubated for a second time with anti-fluorescein-HRP for 30 minutes at RT, washed in TBST and developed using DAB substrate (Quanto, Thermo Scientific, Freemont, Calif.). Tau mRNA and the selected oligomer ISH indicate uniform distribution of tau mRNA reduction and oligomer across the mouse brain following a single i.c.v. bolus injection of 100 μg oligomer (data not shown).

ResultsOligonucleotides show potent knockdown of Tau protein in primary hTau neurons with good tolerability in vitro and in vivo when administered directly into the cerebral spinal fluid (CSF) via intra-cerebroventricular or intrathecal dosing (see, e.g., FIG. 4). They also display robust, durable tau reduction in the brain following intra-cerebroventricular administration of 100 ug in C57 b16 mice (FIG. 4). Inhibition of calcium oscillations in primary neurons was not observed in primary neurons treated with these oligomers. This inhibition of calcium oscillations in primary neurons was a strong indication of acute in vivo tolerability issues related to network dysfunction when injected into CSF directly.

The oligomers sustained Tau reduction following a 100 μg intra-cerebroventricular (ICV) bolus injection. 100 μg/5 μl was injected into wt C57 mice, 3 Month study in wt mice; N=12. Robust and sustained Tau RNA and protein reduction was achieved; 3×33 ug intra-cerebroventricular bolus injections produced similar results (data not shown).

Dose dependent au RNA reduction was also observed following intrathecal (IT) injection of selected oligomers into lumbar ported rats (data not shown). A single bolus IT injection of 300 μg of the selected gapmers was injected into lumbar catheterized rats (as described above). Robust and sustained reduction of brain Tau mRNA was observed at both 3 days and 4 weeks following the single bolus administration using the proposed clinical route of administration of these representative oligomers. IT administration is the preferred clinical route for the treatment of tau dependent disorders.

### Tau Protein Reduction

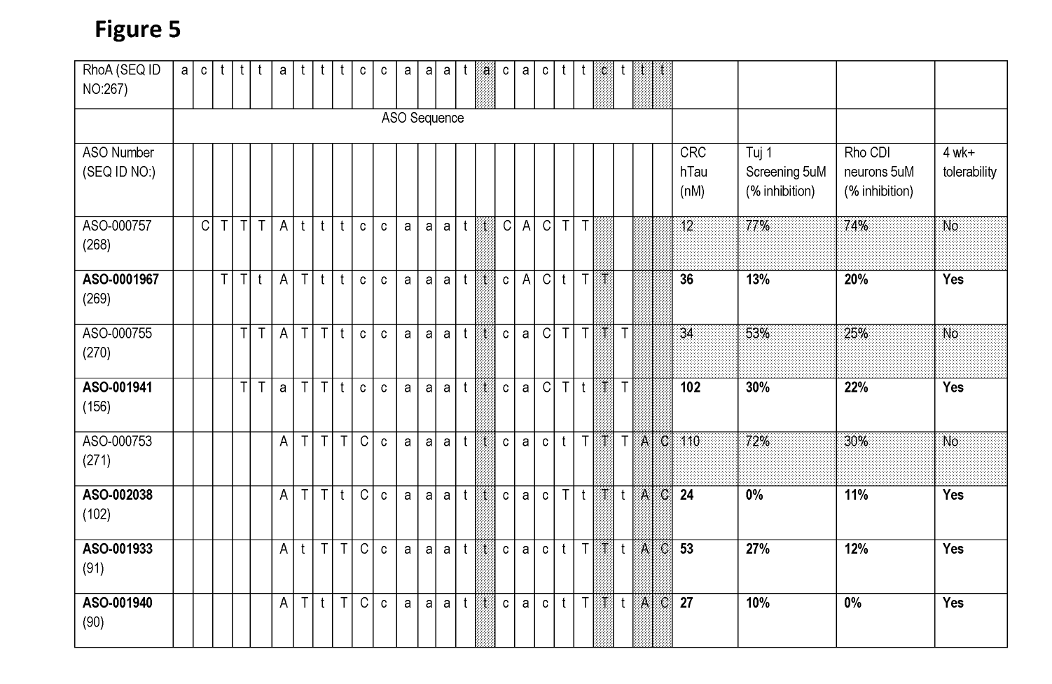

Tau ASOs in the 3′UTR were administered at 100 μg intra-cerebroventricular (ICV) to hTau or wild type B16 mice in order to understand the hysteresis of Tau protein reduction with respect to mRNA reduction. During these studies, many of the Tau ASOs were not tolerated beyond 4 weeks following a single 100 μg ICV bolus dose. Some of the most potent Tau ASOs in this region also reduced expression of an unintended target Ras homolog gene family, member A (“RhoA”). RhoA is a small GTPase protein of Rho family. While the effects of RhoA activity are not all well known, it is primarily associated with cytoskeleton regulation, mostly actin stress fibers formation and actomyosin contractility. In humans, it is encoded by the gene RHOA. The RHOA gene contains the sequence of actttatttccaaatacacttcttt (SEQ ID NO: 267). FIG. 5 shows that the RHOA gene fragment has one to four basepair mismatches with selected oligomers (e.g., ASO-000757, ASO-000755, or ASO-000753).

Certain traditional gapmer sequences were further modified in the gap design and the wing design. In particular, the traditional gapmer design was converted to an alternating gapmer design (e.g., ASO-001967, ASO-001941, ASO-001933, and ASO-1940). FIG. 5 shows that the traditional gapmers are not tolerated beyond 4 weeks following a single 100 μg ICV bolus dose while the alternating gapmers exhibit tolerability beyond 4 weeks.

FIG. 5 also shows that tubulin (Tuj 1) was highly correlated with long term tolerability for the ASOs shown. Rho A reduction greater than 25% also correlated with lack of long term tolerability (greater than 4 weeks following a single ICV bolus injection of 100 μg of each ASO shown in FIG. 5).

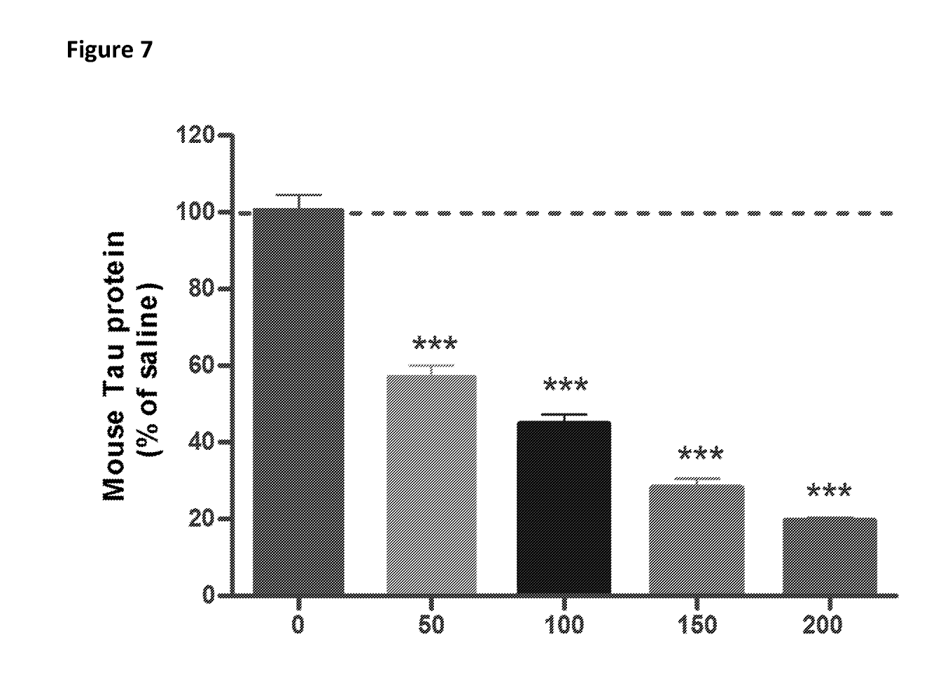

ASO-001933 (100 μg-200 μg) was administered as a single bolus intracerebroventricularly (ICV) in mice, as described above, and produced greater than 50% reduction of brain Tau protein that was sustained for 4-12 weeks in hTau mice. At these dose levels, there were no clinical signs of toxicity and no gross or histologic findings observed over the 20-week period following a single ICV dose in mouse. ASO-000013 was also administered and gave results similar to ASO-001933. A single ICV bolus injection of 100 μg produced no adverse changes in cognition as assessed by novel object recognition or contextual fear conditioning, motor function as assessed by catwalk, rotorod and running wheel (data not shown). In a Tau knock out mouse carrying the entire human tau gene (hTau), the EC₅₀ for reduction of human Tau brain mRNA and protein was ˜2.72 μg/g (414 nM). As FIG. 7 shows, ASO-001933 (Tau ASO) produces durable, dose responsive brain hTau protein reduction after a single intracerebroventricular (ICV) injection in hTau mouse brain. Saline or 50, 100, 150 and 200 μg of Tau ASO was injected ICV in hTau mice (n=10 per group). The frontal cortical region was dissected eight weeks post dose to determine total Tau protein levels by ELISA (BT2/HT7). Two-way ANOVA and Bonferroni post hoc analysis were used ***p<0.001. Error bars represent SEM.

### Example 8: Construction of Oligomers Targeting 5′ UTR and/or Exon 2

A number of oligomers were designed to target the 5′ UTR and/or exon 2 of MAPT pre-mRNA. For example, the oligomers were constructed to target nucleotides 72,802-73,072 of SEQ ID NO: 1. The exemplary sequences of the oligomers are described in FIGS. 6A and 6B. In some embodiments, the oligomers were designed to be gapmers or mixmers. FIGS. 6A and 6B show non-limiting examples of the oligomer design for selected sequences. The same methods can be applied to any other sequences disclosed herein. The gapmers were constructed to contain locked nucleic acids —LNAs (upper case letters). For example, a gapmer can have Beta-deoxy LNA at the 5′ end and the 3′ end and have a phosphorothioate backbone. But the LNAs can also be substituted with any other nucleotide analogs and the backbone can be other types of backbones (e.g., a phosphodiester linkage, a phosphotriester linkage, a methylphosphonate linkage, a phosphoramidate linkage, or combinations thereof). A reference to a SEQ ID number includes a particular sequence, but does not include an oligomer design.

The oligomers were synthesized using methods well known in the art. Exemplary methods of preparing such oligomers are described in Barciszewski et al., Chapter 10—“Locked Nucleic Acid Aptamers” in Nucleic Acid and Peptide Aptamers: Methods and Protocols, vol. 535, Gunter Mayer (ed.) (2009), the entire contents of which is hereby expressly incorporated by reference herein.

### Example 9: Tau mRNA and Protein Reduction in Cynomolgus Monkeys

Progressive supranuclear palsy (PSP) is a neurodegenerative syndrome that is clinically characterized by progressive postural instability, supranuclear gaze palsy, parkinsonism and cognitive impairment. PSP is defined neuropathologically by the accumulation of tau-positive neurofibrillary tangles in brain regions extending from the cerebral cortex, basal ganglia to the cerebellum and brainstem. The most severely affected brain regions include the brainstem substantia nigra, pontine nuclei and the cerebellar dentate nucleus. Tauopathy in these regions is believed to underpin several clinical features of PSP such as postural instability, dysarthria and gaze palsy. Suppression of Tau mRNA transcripts and, consequently, protein in the brain regions, may have therapeutic significance for treatment of PSP patients.

Subjects were male cynomolgus monkeys weighing 3.5-10.0 kg at the start of the study. Each was implanted with an intrathecal CSF catheter entering at the L3 or L4 vertebrae extending to approximately the L1 vertebra. The proximal end of the catheter was connected to a subcutaneous access port. CSF was collected through the port by gravity flow to a maximum of 0.5 ml CSF per sample. The CSF was centrifuged and the supernatent was kept at −90° C. until analyzed. Blood plasma obtained from an available vein was kept at −90° C. until analyzed.

Cynomolgus monkeys were administered with ASO-1933, which was dissolved in saline, at 0.33 ml/min in a 1.0 ml volume followed by a 0.5 ml sterile water flush. Total infusion time was 4.5 min.

Cynomolgus monkeys were administered the appropriate volume of a commercially available euthanasia solution while anesthetized with ketamine and isoflurane. Necropsy tissues were obtained immediately thereafter and the brain was transferred to wet ice for dissection. Areas of interest were dissected using 6 mm slices in an ASI Cyno Brain Matrix as well as free handed techniques. Samples were placed fresh in RNAlater, or frozen on dry ice for later analysis. Some slices were frozen intact for immunohistochemical analysis. Slices were placed in a weigh boat and floated on isopentane cooled with dry ice. Once frozen, slices were stored at −90° C. until analysis.

For brain block sectioning, the frozen brain blocks were cut on a cryostat coronal sections, and sections were thaw-mounted onto super frost slides, dried, re-frozen on dry ice, and stored at −80° C. until use. Brain sections collected from the cynomolgus monkey dosed with vehicle, ASO-1933 at 16 mg (1×16) or ASO-1933 at 16 mg twice (2×16, with 2 weeks apart) were used for the in situ hybridization (ISH) study.

In order to measure Tau mRNA expression using [³⁵S] labeled antisense ISH, a Tau DNA template and [³⁵S] labeled antisense probes were synthesized. A Tau DNA template (425 bp, 687-1111, accession number: XM_005584540.1) was amplified from a cynomolgus monkey cDNA library (Zyagen KD-201) by PCR using forward primer 5′-CAA GCT CGC ATG GTC AGT AA-3′ (SEQ ID NO: 339) and reverse primer 5′-AAT TAA CCC TCA CTA AAG GGA GA TTC TCA GTG GAG CCG ATC TT-3′ (SEQ ID NO: 340). Products of desired size were observed by gel electrophoresis. The Tau DNA template was transcripted with T3 RNA polymerase (Invitrogen AM1316) using [³⁵S]UTP (Perkin Elmer NEG-739) to produce a [³⁵S] labeled antisense ISH probe.

To measure Tau mRNA ISH using [³⁵S] labeled antisense probe, slides were thawed, fixed in 4% paraformaldehyde for 15 min at 4° C. followed by rinsing. Slides were then treated in acetic anhydride/triethanolamine followed by rinsing. Slides were pre-hybridized in pre-hybridization solution at 50° C. for 3 hours and hybridized with 1.5×10⁴ cpm/ul [³⁵S]riboprobe (0.75 ml/slide) in hybridization solution. After hybridization, slides were washed at room temperature. Slides were then treated with Rnase A at 37° C., washed twice, followed by a high stringency wash. The sections were dehydrated in 90% alcohol containing 0.3 M NH₄Ac, dried, and exposed against phosphor screen (Perkin Elmer PPN 7001487). After exposure, autoradiographic images on the screen were captured and analyzed using Cyclone storage phosphor system and OptiQuant Acquisition and Analysis software (PerkinElmer, Waltham, Mass.).

QUANTIGENE® ViewRNA tissue ISH was used to detect Tau mRNA expression at the subnucleus and cellular levels. An antisense probe (type-1) targeting Tau mRNA (2344-3300, accession number: XM 005584529) was synthesized by Affymetrix. Slides were fixed in 4% formaldehyde in phosphate buffered saline (PBS). After passing through alcohol gradients for 10 minutes each, slides were dried, followed by protease QF digestion. Subsequently, sections were washed and hybridized with the target probe. Slides were then washed in wash buffer and stored in storage buffer overnight. Slides were then processed through a series of sequential PreAmp and Amp hybridization steps. The sections were incubated with Label Probe AP followed by incubation with Fast Red Substrate, rinsed in PBS, and counterstained using either Gill's Hematoxylin or DAPI. Slides were coverslipped using DAKO ultramount mounting medium and stored. Labeled Tau mRNA was visualized using either a Leica brightfield microscope or a Leica confocal fluorescence microscope (excitation: 630 nm; emission: 760).

To measure Tau protein expression, Tau12 (BioLegend, San Diego, Calif., epitope to amino acids 6-18 on tau 441 sequence) and BT2 (Thermo Scientific, Rockville, Ill., epitope to amino acids 194-198) were used to coat Costar 3925 ELISA plates at 2.5 and 1 μg/ml, respectively. Plates were incubated for 1 h at 37° C. before washing with TBS with 0.05% Tween-20 (TBST). Non-specific binding was blocked by the addition of 3% bovine serum albumin (BSA) in TBS with 0.1% Tween-20 for 4 h at room temperature with shaking. Plates were washed with TBST before the addition of samples or standard curve generated with recombinant h-tau441 protein, both of which were prepared in TBST plus 1% BSA. Plates containing standard curve and samples were incubated overnight at 4° C. with shaking. The following detection antibodies were conjugated with alkaline phosphatase (AP) using the Lightning Link Conjugation Kit (Novus Biologicals, Littleton, Colo.): BT2 and HT7 (Thermo Scientific, epitope of 159-163). AP-conjugated detection antibodies were diluted in TBST plus 1% BSA and co-incubated with samples and standard curve for 1 h at room temperature with shaking. After washing with TBST, Tropix CDP-Star Ready-to-Use with Sapphire-II AP substrate (Applied Biosystems, Bedford, Mass.) was added for 30 min. Chemiluminescent signal was determined using a Perkin Elmer EnVision microplate reader (Waltham, Mass.).

The N-terminal tau sandwich ELISA (Tau12-BT2) consists of the anti-tau antibody Tau12 as capture and detection with an alkaline phosphatase (AP) conjugate of the anti-tau antibody BT2. The mid-domain tau sandwich ELISA (BT2-HT7) consists of the anti-tau antibody BT2 as the capture antibody and detection with an alkaline phosphatase (AP) conjugate of the anti-tau antibody HT7. High binding black well ELISA plates (Costar, Corning, Tewksbury, Mass.) were coated with anti-Tau BT2 monoclonal antibody (Thermo, Waltham, Mass.) at 2.5 μg/ml or Tau12 anti-tau monoclonal antibody (Covance) at 5 μg/ml in tris buffered saline (50 μL/well). The plates were washed with tris buffered saline containing 0.05% tween-20 (TBS-T) followed by blocking at room temperature with shaking in 3% BSA/TBS (BSA from Roche, Indianapolis, Ind.). The plates were rewashed as listed above followed by sample addition in triplicate (50 μL/well). Cynomolgus monkey CSF samples were diluted 1:30 (BT2/HT7) or 1:25 (Tau12/BT2) in 1% BSA/TBS-T. A Tau 441 (R-peptide, Bogart, Ga.) standard curve was made. The samples were incubated on the ELISA plate overnight at 4° C. with shaking. AP conjugated HT7 or BT2 was diluted to 0.25 μg/ml (HT7) or 0.1 μg/ml (BT2) in 1% BSA/TBS-T was added to the plates (50 μL/well) for co-incubation with standards and samples for 1 hour at room temperature with shaking. The plates were re washed followed by the addition of chemiluminescent substrate (Tropix CDP Star, Applied Biosystems, Grand Island, N.Y.) (100 μL/well) and incubation at room temperature with shaking for 30 minutes. The plates were read on a Perkin Elmer TopCount. Unknown sample values were read off the Tau-441 standard curve using GraphPad Prism software.

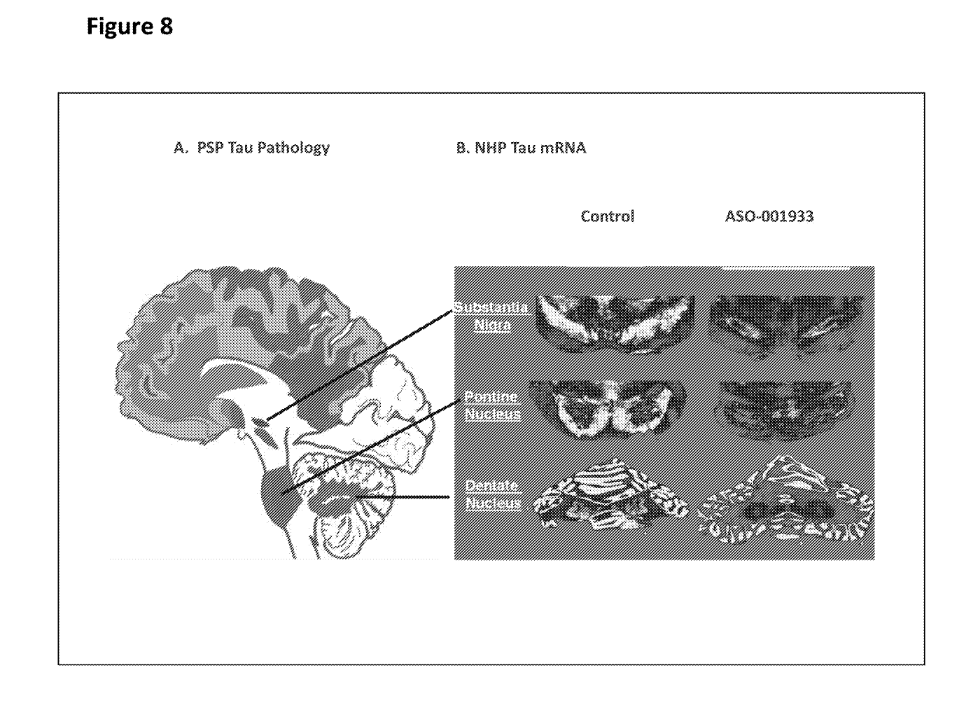

These studies demonstrate that intrathecally-applied Tau ASO distributes to the substantia nigra, pontine nuclei and dentate nucleus and suppresses Tau mRNA expression in these brain regions in Cynomolgus monkeys following intrathecal administration of ASO-001933 following two doses (2 week apart) of 16 mg (2×16). FIG. 7A show in situ hybridization (ISH) autoradiographic images of tau mRNA expression (lighter shades) in the substantia nigra, pontine nuclei and dentate nucleus in the monkeys dosed with vehicle or ASO-001933 2×16 mg (1 week apart). As FIG. 8B shows, ASO-001933 produced profound suppression of Tau mRNA expression in all three regions in both monkeys. The Tau mRNA knockdown effect produced by ASO-001933 was further demonstrated using the QUANTIGENE® ViewRNA ISH assay (data not shown). FIG. 8B shows that in the vehicle-treated monkey, a high intensity Tau mRNA labeling was present, primarily, in neuronal cell bodies in the substantia nigra, pontine nuclei and dentate nucleus. In cynomolgus monkeys, two single 16 mg intrathecal doses of ASO-001933, one week apart were administered to assess anatomic distribution of Tau mRNA reduction in anatomic brain regions where pathologic Tau accumulates in PSP (FIG. 8A).

In monkeys, a single intrathecal (IT) dose of 4 mg of ASO-001933 produced Tau mRNA reductions between 58% to 80% in cortical brain regions and 63% in cerebellum within 2 weeks post dose (data not shown). These areas of the brain are believed to be important for treatment of Tau-dependent dysfunction in PSP (neurodegenerative tauopathies) and Dravet syndrome (epilepsy and autism spectrum disorders), leading indications for Tau antisense molecules like ASO-001933.

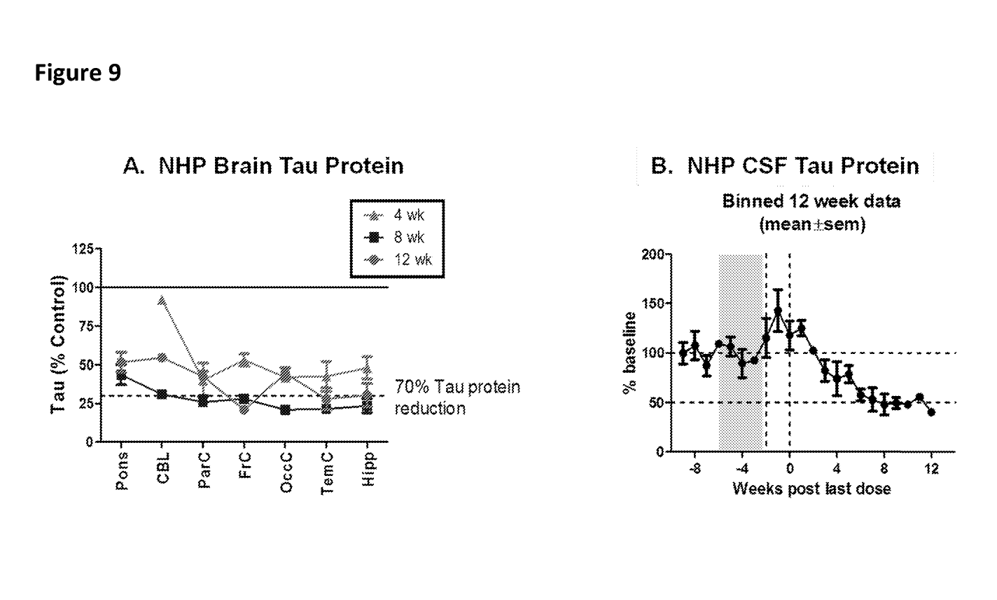

Consistent with Tau mRNA, FIGS. 9A and 9B show that the ASO-001933 administration as IT bolus injection (2 doses of 8 mg given 2 weeks apart) in monkeys is capable of reducing about 70% of Tau protein in the brain (FIG. 9A) about 60% in the CSF (FIG. 9B). The Tau protein expression was observed 12 weeks following the ASO administration. Similarly, the ASO-001933 administration as a single ICV intra-cerebroventricular injection (100 m) in mice is capable of reducing about 50% of Tau protein in the brain (data not shown) and about 34% of Tau protein in the CSF (data not shown). These data suggest that reduction of CSF Tau protein may be a clinically accessible biomarker of target engagement.

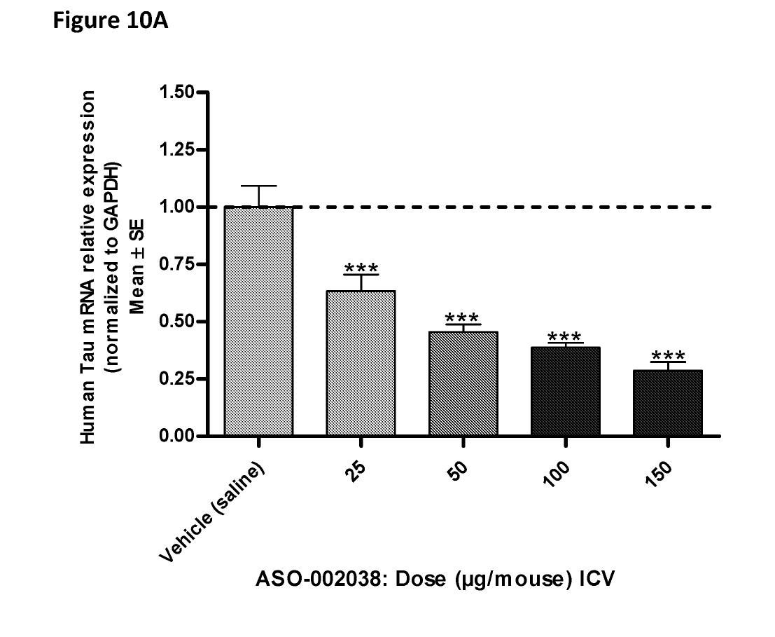

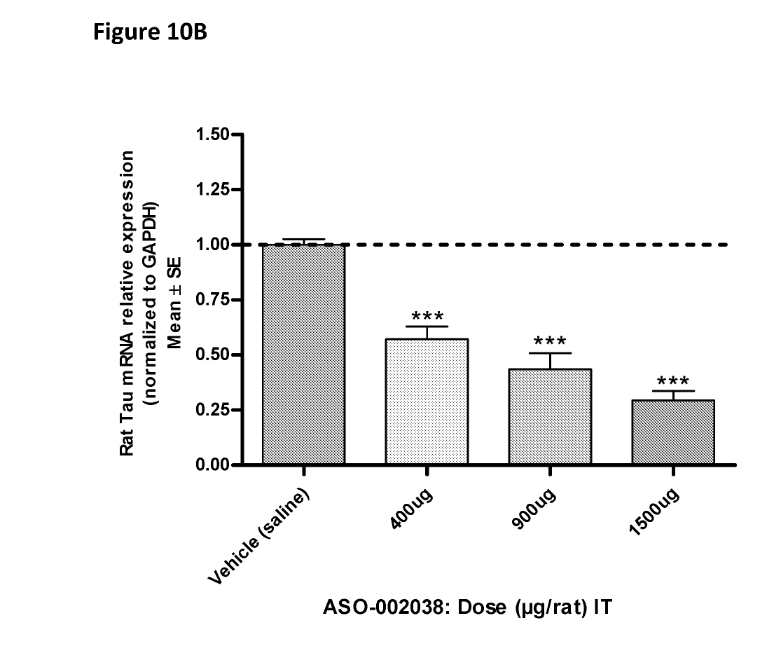

ASO-002038 was administered as a single bolus intracerebroventricularly (FIG. 10A: ICV at 25-150 μg) or intrathecally (FIG. 10B: IT at 400-900 μg) in mice or in rats, as described in Example 5. ASO-002038 produced dose dependent hTau mRNA reduction in the brain with a calculated EC₅₀ value ˜598 nM in mice. At these dose levels, there were no clinical signs of toxicity and no gross or histologic findings observed following a single ICV dose in mouse. Many ASOs including ASO-000013, ASO-001933, ASO-001967, ASO-001940, ASO-001941, and others produced similar hTau dose dependent reduction, EC₅₀ for reduction of human Tau brain mRNA, and were well tolerated in mice (data not shown).

### Example 10: Quantigene Analysis of Tau, Rho and Tubulin mRNA Expression

To measure tau, rhoA and tubulin mRNA reduction, primary neuronal cultures were established from the forebrain of E18 transgenic mice expressing the human tau transgene on a mouse tau knockout background. (Andorfer et al. J Neurochem 86:582-590 (2003)). Cultures were prepared as described in Example 2. Alternatively, iNeurons from Cellular Dynamics Inc., were used per manufacturer specifications.

Lysis: Cells were plated on poly-D-lysine coated 96 well plates at 50,000 cells per well and maintained in Neurobasal media containing B27, glutamax and Penicillin-Streptomycin. ASOs were diluted in water and added to cells at DIVO1 to a final concentration of 5 μM. For IC₅₀ determinations, neurons were treated with a top concentration of 5 uM and a concentration response dilution of 1:3 was used to define the IC₅₀. Following ASO treatment, neurons were incubated at 37° C. for 5 days to achieve steady state reduction of mRNA. Media was removed and cells were washed 1× in DPBS and lysed as follows. Measurement of lysate messenger RNA was performed using the Quantigene 2.0 Reagent System (Affymetrix), which quantitates RNA using a branched DNA-signal amplification method reliant on the specifically designed RNA capture probe set. The working cell lysis buffer solution was made by adding 50 μl proteinase K to 5 ml of pre-warmed Lysis mix and diluted to 1:4 final dilution with dH₂0. The working lysis buffer was added to the plate (150 μl/well), triturated to mix, sealed and incubated. Following lysis the wells were titrated to mix and stored at −80° C. or assayed immediately.

Assay: Lysates were diluted in lysis mix dependent on the specific capture probe used (tau, RhoA or tubulin). 80 μl/well total were then added to the capture plate (96 well polystyrene plate coated with capture probes). Working probe sets reagents were generated by combining nuclease-free water 12.1 lysis mixture 6.6 blocking reagent 1 specific 2.0 probe set 0.3 μl (human MAPT catalogue #15486, human RHOA catalogue #SA-11696, or human beta 3 tubulin catalogue #SA-15628) per manufacturer instructions (QuantiGene 2.0 Affymetrix). Then 20 μl working probe set reagents were added to 80 μl lysate dilution (or 80 μl lysis mix for background samples) on the capture plate. Plates were centrifuged and then incubated for 16-20 hours at 55° C. to hybridize (target RNA capture). Signal amplification and detection of target RNA was begun by washing plates with buffer 3 times to remove unbound material. 2.0 Pre-Amplifier hybridization reagent (100 μl/well) was added, incubated at 55° C. for 1 hour then aspirated and wash buffer was added and aspirated 3 times. The 2.0 Amplifier hybridization reagent was then added as described (100 μl/well), incubated for 1 hour at 55° C. and the wash was repeated as described previously. The 2.0 Label Probe hybridization reagent was added next (100 μl/well), incubated for 1 hour at 50° C. and the wash was repeated as described previously. Lastly, the plates were centrifuged to remove any excess wash buffer and 2.0 Substrate was added (100 μl/well). Plates were incubated for 5 minutes at room temperature and plates were imaged on a PerkinElmer Envision multilabel reader in luminometer mode within 15 minutes.

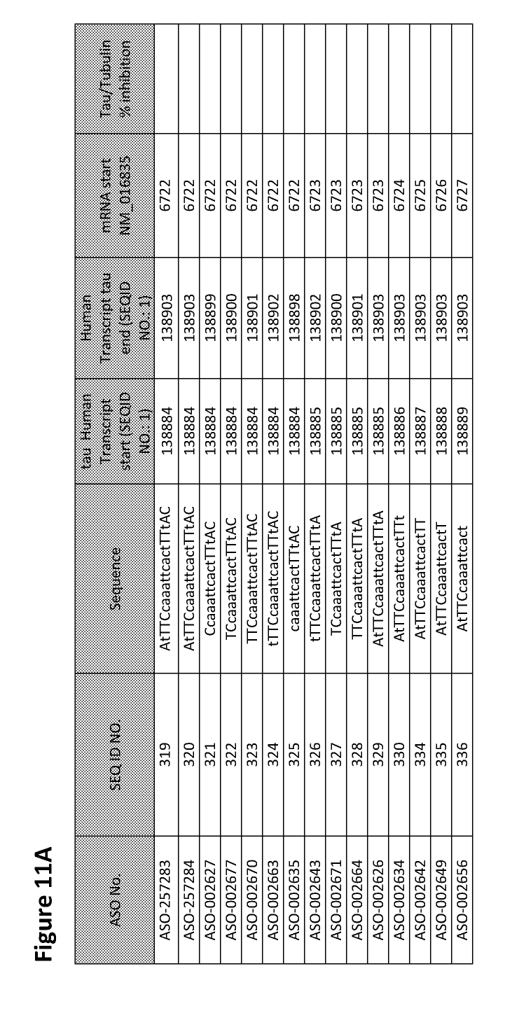

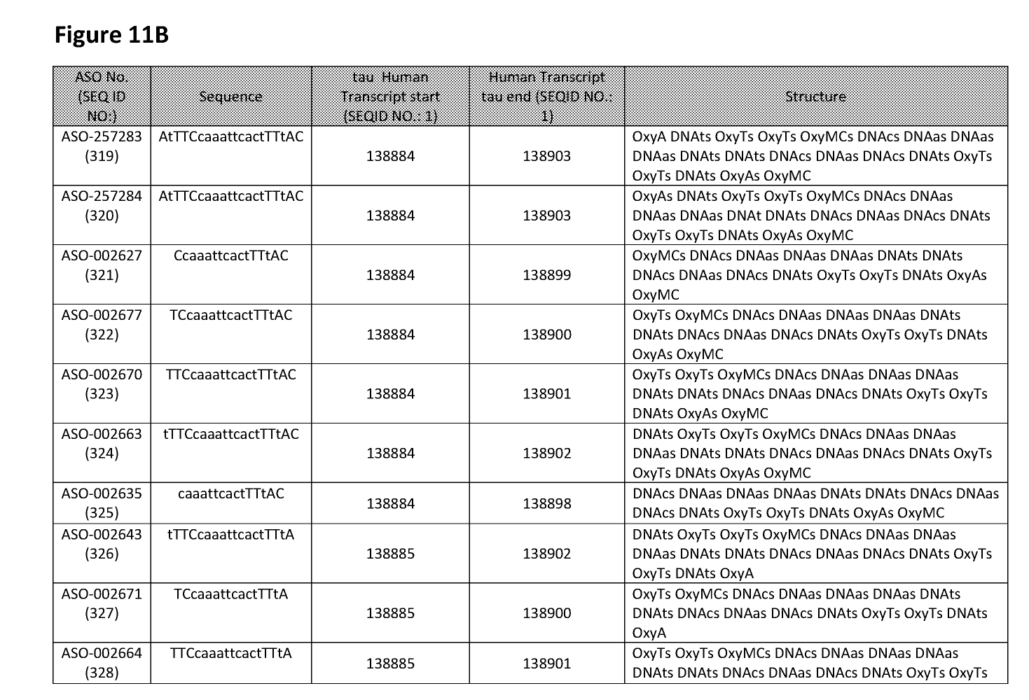

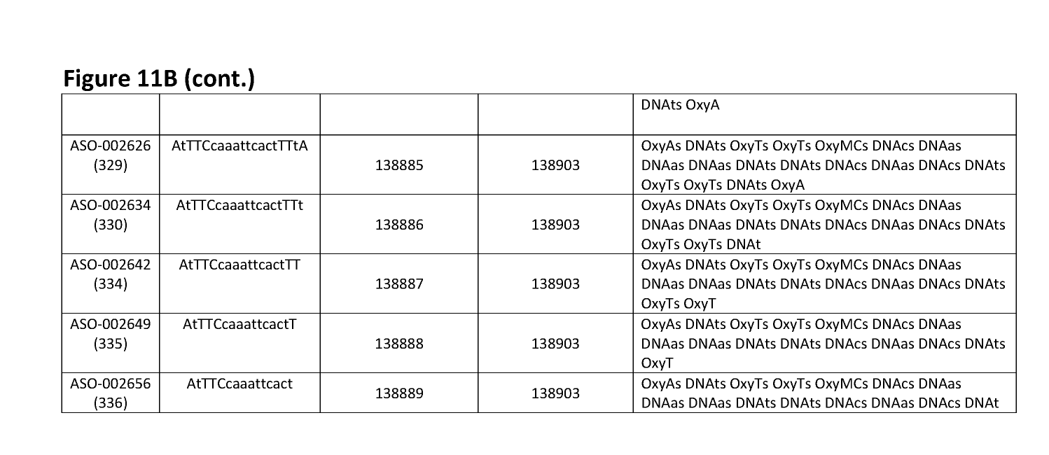

Data determination: For the gene of interest, the average assay background signal was subtracted from the average signal of each technical replicate. The background-subtracted, average signals were divided by the background subtracted average signal for the housekeeping tubulin RNA. The percent inhibition for the treated sample was calculated relative to control treated sample lysate. Results of Quantigene assays for cells treated with ASOs are shown in FIGS. 11A and 11B.

1. An oligomer of at least 10 contiguous nucleotides in length, comprising region A, region B, and region C (A-B-C),
wherein region B comprises at least 5 consecutive nucleoside units and is flanked at 5′ by region A of 1-8 contiguous nucleoside units and at 3′ by region C of 1-8 contiguous nucleoside units, wherein region B, when formed in a duplex with a complementary RNA, is capable of recruiting RNaseH, and
wherein region A and region C are selected from the group consisting of:
(i) region A comprises a 5′ LNA nucleoside unit and a 3′ LNA nucleoside unit, and at least one DNA nucleoside unit between the 5′ LNA nucleoside unit and the 3′ LNA nucleoside unit, and, region C comprises at least two 3′ LNA nucleosides; or
(ii) region A comprises at least one 5′ LNA nucleoside and region C comprises a 5′ LNA nucleoside unit, at least two terminal 3′ LNA nucleoside units, and at least one DNA nucleoside unit between the 5′ LNA nucleoside unit and the 3′ LNA nucleoside units, and
(iii) region A comprises a 5′ LNA nucleoside unit and a 3′ LNA nucleoside unit, and at least one DNA nucleoside unit between the 5′ LNA nucleoside unit and the 3′ LNA nucleoside unit; and region C comprises a 5′ LNA nucleoside unit, at least two terminal 3′ LNA nucleoside units, and at least one DNA nucleoside unit between the 5′ LNA nucleoside unit and the 3′ LNA nucleoside units.

2. The oligomer according to claim 1, wherein region A or region C comprises 1, 2, or 3 DNA nucleoside units.

3. The oligomer according to claim 1 or 2, wherein region B comprises at least five consecutive DNA nucleoside units.

4. The oligomer according to any one of claims 1-3, wherein region A comprises two 5′ terminal LNA nucleoside units.

5. The oligomer according to any one of claims 1-4, wherein region A has formula 5′ [LNA]₁₋₃ [DNA]₁₋₃ [LNA]₁₋₃, or 5′ [LNA]₁₋₂ [DNA]₁₋₂ [LNA]₁₋₂ [DNA]₁₋₂ [LNA]₁₋₂.

6. The oligomer according to any one of claims 1-5 wherein region C has formula [LNA]₁₋₃ [DNA]₁₋₃ [LNA]₂₋₃ 3′, or [LNA]₁₋₂ [DNA]₁₋₂ [LNA]₁₋₂ [DNA]₁₋₂ [LNA]₂₋₃ 3′.

7. The oligomer according to any one of claims 1-6, wherein region A has a sequence of LNA and DNA nucleosides, 5′-3′ selected from the group consisting of L, LL, LDL, LLL, LLDL, LDLL, LDDL, LLLL, LLLLL, LLLDL, LLDLL, LDLLL, LLDDL, LDDLL, LLDLD, LDLLD, LDDDL, LLLLLL, LLLLDL, LLLDLL, LLDLLL, LDLLLL, LLLDDL, LLDLDL, LLDDLL, LDDLLL, LDLLDL, LDLDLL, LDDDLL, LLDDDL, and LDLDLD, wherein L represents a LNA nucleoside, and D represents a DNA nucleoside.

8. The oligomer according to any one of claims 1-7, wherein region C has a sequence of LNA and DNA nucleosides, 5′-3′ selected from the group consisting of LL, LLL, LLLL, LDLL, LLLLL, LLDLL, LDLLL, LDDLL, LDDLLL, LLDDLL, LDLDLL, LDDDLL, LDLDDLL, LDDLDLL, LDDDLLL, and LLDLDLL.

9. The oligomer according to any one of claims 1-6, wherein the contiguous nucleotides comprise a sequence of nucleosides, 5′-3′, selected from the group consisting of LLDDDLLDDDDDDDDLL, LDLLDLDDDDDDDDDLL, LLLDDDDDDDDDDLDLL, LLLDDDDDDDDDLDDLL, LLLDDDDDDDDLDDDLL, LLLDDDDDDDDLDLDLL, LLLDLDDDDDDDDDLLL, LLLDLDDDDDDDDLDLL, LLLLDDDDDDDDDLDLL, LLLLDDDDDDDDLDDLL, LLLDDDLDDDDDDDDLL, LLLDDLDDDDDDDDDLL, LLLDDLLDDDDDDDDLL, LLLDDLLDDDDDDDLLL, LLLLLDDDDDDDLDDLL, LDLLLDDDDDDDDDDLL, LDLLLDDDDDDDLDDLL, LDLLLLDDDDDDDDDLL, LLDLLLDDDDDDDDDLL, LLLDLDDDDDDDDDDLL, LLLDLDDDDDDDLDDLL, LLLDLLDDDDDDDDDLL, LLLLDDDDDDDLDDDLL, LLLLLDDDDDDDDDLDLL, LLLLDDDDDDDDDDLDLL, LLLDDDDDDDDDDDLDLL, LLDLDDDDDDDDDDLDLL, LDLLLDDDDDDDDDLDLL, LLLDDDDDDDDDDLDDLL, LLLDDDDDDDDDLDDDLL, LLLDDDDDDDDLDLDDLL, LLLLDDDDDDDDDLDDLL, LLLLDDDDDDDDDLDLLL, LLLLDDDDDDDDLDDDLL, LLLLDDDDDDDDLDDLLL, LLLLDDDDDDDDLDLDLL, LLLLDDDDDDDLDDLDLL, LLLLDDDDDDDLDLDDLL, LLDLLDDDDDDDDDDDLL, LLDLLLDDDDDDDDLDLL, LLLDLDDDDDDDDDDDLL, LLLDLDDDDDDDDDLDLL, LLLDLDDDDDDDDLDDLL, LLLDLDDDDDDDLDLDLL, LLLLDDDDDDDDDLLDLL, LLLLLDDDDDDDDDLDLLL, LLLLLDDDDDDDDDLDDLL, LLLLDDDDDDDDDDLLDLL, LLLLDDDDDDDDDDLDLLL, LLLLDDDDDDDDDDLDDLL, LLLDDDDDDDDDDDLLDLL, LLLDDDDDDDDDDDLDLLL, LLLLLDDDDDDDDDLLDLL, LLLDDDDDDDDDDDLDDLL, LLDLLDDDDDDDDDLDDLL, LLLDLDDDDDDDDDDLDLL, LLLDLDDDDDDDDDLDDLL, LLLLDDDDDDDDDLDLDLL, LLLLDDDDDDDDLLDLDLL, LDLLLDDDDDDDDDDLLDLL, LLDLLDDDDDDDDDDLLDLL, LLDLDDDDDDDDDDDDLLLL, LLDDLDDDDDDDDDDDLLLL, LLLDLDDDDDDDDDDDLLLL, LLDLDDDDDDDDDDDDDLLL, LLDLLDDDDDDDDDDDLLLL, LLDDLDDDDDDDDDDDDLLL, LLLDDDDDDDDDDDLDDLLL, LLLDLDDDDDDDDDDDDLLL, LLDLLDDDDDDDDDDDDLLL, LLLLDDDDDDDDDDDLLDLL, LLLLDDDDDDDDDDLLDDLL, LLLDDLDDDDDDDDDLDLLL, LLDDLDLDDDDDDDDDLLLL, LLDDLLDDDDDDDDDLDLLL, LLLDLDDDDDDDDDLDLDLL, LLDLLDDDDDDDDDLDDLLL, LLLDLDDDDDDDDDDLDLLL, LLDLDDLDDDDDDDDDLLLL, LLLLDDDDDDDDDLDLDDLL, LLLDLDDDDDDDDDLDDLLL, LLDLDLDDDDDDDDDDLLLL, LLDLLDDDDDDDDDDLDLLL, LLDLDLDDDDDDDDDLLDLL, LLDDLLDDDDDDDDDLLDLL, LLLLDDDDDDDDDLDDLDLL, LLLDDLDDDDDDDDDLLDLL, LLDLLDDDDDDDDDLLDDLL, LLDLDLDDDDDDDDDLDLLL, LLLDLDDDDDDDDDLLDDLL, LLDDLLDDDDDDDDDDLLLL, LLDLLDDDDDDDDDLDLDLL, LLLLDDDDDDDDDDLDDLLL, LLLDDLDDDDDDDDDDLLLL, LLLDLDDDDDDDDDDLLDLL, LLLLDDDDDDDDDDLDLDLL, LLLLDDDDDDDDDDDLDLLL, and LLDDLLDDDDDDDDDDLDLL; wherein L represents a beta-D-oxy LNA nucleoside, and D represents a DNA nucleoside.

10. The oligomer according to any one of claims 1-6, wherein the contiguous nucleotides comprise an alternating sequence of LNA and DNA nucleoside units, 5′-3′, selected from the group consisting of: 2-3-2-8-2, 1-1-2-1-1-9-2, 3-10-1-1-2, 3-9-1-2-2, 3-8-1-3-2, 3-8-1-1-1-1-2, 3-1-1-9-3, 3-1-1-8-1-1-2, 4-9-1-1-2, 4-8-1-2-2, 3-3-1- 8-2, 3-2-1-9-2, 3-2-2-8-2, 3-2-2-7-3, 5-7-1-2-2, 1-1-3-10-2, 1-1-3-7-1-2-2, 1-1-4-9-2, 2-1-3-9-2, 3-1-1-10-2, 3-1-1-7-1-2-2, 3-1-2-9-2, 4-7-1-3-2, 5-9-1-1-2, 4-10-1-1-2, 3-11-1-1-2, 2-1-1-10-1-1-2, 1-1-3-9-1-1-2, 3-10-1-2-2, 3-9-1-3-2, 3-8-1-1-1-2-2, 4-9-1-2-2, 4-9-1-1- 3, 4-8-1-3-2, 4-8-1-2-3, 4-8-1-1-1-1-2, 4-7-1-2-1-1-2, 4-7-1-1-1-2-2, 2-1-2-11-2, 2-1-3-8- 1-1-2, 3-1-1-11-2, 3-1-1-9-1-1-2, 3-1-1-8-1-2-2, 3-1-1-7-1-1-1-1-2, 4-9-2-1-2, 4-7-1-3-3, 5-9-1-1-3, 5-9-1-2-2, 4-10-2-1-2, 4-10-1-1-3, 4-10-1-2-2, 3-11-2-1-2, 3-11-1-1-3, 5-9-2-1-2, 3-11-1-2-2, 2-1-2-9-1-2-2, 3-1-1-10-1-1-2, 3-1-1-9-1-2-2, 4-9-1-1-1-1-2, 4-8-2-1-1-1-2, 1-1-3-10-2-1-2, 2-1-2-10-2-1-2, 2-1-1-12-4, 2-2-1-11-4, 3-1-1-11-4, 2-1-1-13-3, 2-1-2-11-4, 2-2-1-12-3, 3-11-1-2-3, 3-1-1-12-3, 2-1-2-12-3, 4-11-2-1-2, 4-10-2-2-2, 3-2-1-9-1-1-3, 2-2-1-1-1-9-4, 2-2-2-9-1-1-3, 3-1-1-9-1-1-1-1-2, 2-1-2-9-1-2-3, 3-1-1-10-1-1-3, 2-1-1-2-1-9-4, 4-9-1-1-1-2-2, 3-1-1-9-1-2-3, 2-1-1-1-1-10-4, 2-1-2-10-1-1-3, 2-1-1-1-1-9-2-1-2, 2-2-2-9-2-1-2, 4-9-1-2-1-1-2, 3-2-1-9-2-1-2, 2-1-2-9-2-2-2, 2-1-1-1-1-9-1-1-3, 3-1-1-9-2-2-2, 2-2-2-10-4, 2-1-2-9-1-1-1-1-2, 4-10-1-2-3, 3-2-1-10-4, 3-1-1-10-2-1-2, 4-10-1-1-1-1-2, 4-11-1-1-3, and 2-2-2-10-1-1-2; wherein the first numeral represents an number of LNA units, the next a number of DNA units, and alternating LNA and DNA regions thereafter.

11. The oligomer according to any one of claims 1-10, wherein the LNA nucleoside units present in regions A and C are beta-D LNA units, such as beta-D-oxy LNA nucleoside units or (S)cEt nucleoside LNA units.

12. The oligomer according to any one of claims 1-11, wherein the oligomer comprises at least one phosphorothioate internucleoside linkage.

13. A conjugate comprising the oligomer according to any one 1-12 covalently attached to at least one non-nucleotide or non-polynucleotide moiety.

14. The conjugate according to claim 13, wherein the at least one non-nucleotide or non-polynucleotide moiety is selected from the group consisting of proteins, peptides, glycoproteins, antibodies, antibody binding domains, fatty acids, sterols, sugar residues, a GalNAc conjugate such as a trivalent GalNAc cluster, polymers, PEG, and two or more combinations thereof.

15. A pharmaceutical composition comprising the oligomer of any one of claims 1-12, or the conjugate of claim 13 or 14, and a pharmaceutically acceptable excipient, carrier, or diluent.

16. Use of the oligomer of any one of claims 1-12, or the conjugate of claim 13 or 14 for the manufacture of a medicament in treating a disease or condition.

17. An in vivo or in vitro method for modulating the expression of an RNA in a cell which expresses said RNA, said method comprising contacting an effective amount of the oligomer of any one of claims 1-12, the conjugate of claim 13 or 14 or the pharmaceutical composition of claim 15 with said cell.

18. A method of producing an oligomer having a reduced non-specific binding comprising synthesizing the oligomer of any one of claims 1 to 12.


SEQUENCE LISTING (340 sequences)

SEQ ID NO: 1 - DNA, 140924 bp, Homo sapiens
gggattacaggcgtgagccaccacacccagcccagaatgtttattagaatgcacaattaataccagaggcagtggggaaggaaggactgagcagaggaggaagttgagttgtgattcaacccaacaactgcctggctggcatggggagctctggagttaaatagggccatcagactttcccagtgtggggccaacatgactgggtctttatacccccacctctgtcagtcactcaacgtggtctccctgcaacaaggtgactcttgcagccgagacaatccctgaagggacagaggctgaagcctgtctgccaacagcactcccagtggctggaacaagtccttccctataggggaatctgggcggcacacctccatctccatgtccatcacatacgatatcacagacatttaaatattttgataactgtacataagagtttcctttataatcttatagatcttattttatgcatttgaaaatattcttctgagacagggcttttatcatattgccatagggtgccacgatataaaaaaggttaaatactctctgattcagaagtatccaatgatgacttctctctcatgcatttaattgaaaatctggtttttctccttctctgctagttctctacctctctccccacctcccacatcatagcctattcacatatgtctgaatctcatgatagacaagttcaggttcttttcccaggttctttttaccacatccccccacccccacataaaaagtatatatggcacagcctaggttccacccaaatcctttctcctcttcttcctgggcccacaactctcctacatacattggtataccttgcgcttagggatggccatgtgactaagttctaacagtggaacatgatcagatgccacttccagcctctaagacagccagtgtgtttcctccataagctccttctcttcctcccaactggagactctaaatgatgaccctgcctcaagcaagcaaacaacaagtccctcaggggtggtgtaggctgcaaatggaaggagcttgagtcccaaaccttccacggagaaggctggctaccaacctggatcactcacccaagactgctcgaagagttggtttgaaccattgtgttttggggtctatttattacaacagtttagcttgctttgtgaatagatttagtggcagagcctccaaattctatagatacattgatctcagtcctaaccgcatctggaacaccattaaataaaggaattgcaaacccagagaaggtaatgaatttgtctaaggtcatacaagatggctaggatcaggacccaactctccagttttctttcttctctgctattctgccttctgtgatcctacataagtgggcatgattgtataacatatgcggccatgagatttctctttcagcaagagaaagggacaggaagaaagagagggaatgcattttcttggcctgaattagtgtgagccattagttacctacattgactaaattatctggaatgaacattcaactctacatcacatatagttaaaatgacagatctgcttaagattgtttctagcatacgttatttcaatttaggcaaatgtgaccattcagtgtgaggggaccatactgtcattaggtccctgtcagttctcaattatactgttatcttagagggggaaaaatgtgaaatttgaatgtagacgagtgttgatttgactgctacagtttattttacgtatagaaataaaataatgtgtagcaaaagcattattacaaagatgataatgaaataactagtatttataatagtataatagtatagtatttataatagtatgatagtttaatgactatttgtcagatgttgtgtaagaaactttatacacacacacacacacacctcatttaattcctgtatcaatcaggatacaggacgctgtggtaacaactcctcaaatctcggtggcttgcacaacaaatgcttatttcttttttttttttgacaccaagtcttgctctgtaacaggctggagtgcaatggtgcaatctcggctcactgcagcctctgcctcctgggttcaagcgattctcctgcctcagtctctcgagtagctgggaacacaggcacgcgccaccacatctggctaatttttgtgattttagtagagatgggatttcaccatgttgctcaggctggccttgaactcctgacctcaagcgatccacccacctcagcctcccaaagtgctgggattacaggcatgagccactgcgcccagccccaaatgtttatttcttgctcatgtgacatgtacttcctcgagtttttccttcctgagatctaagctgaaggaacagctctctggagccacgccattctggtggcggaaaggaagagtaaaagtggtagaaccttgcaatgctcttgaagcgcctatttggaatgtctacatcatgtaaatggtaatggacaagtatgtataatccccacaccaaaaaaaggggacactattggggacaataaccacatttcaatgctgcaagacggatattgactgcacccccttcccactttcagaaagaagaagagtaattttgctgaactccttctagagactggaaatgtcccttccagttggggtgattagggaaggctttggtaaaatttgagctagagtttgaaggttaggtagactactggtgggtgaagaaagaacaaggacctttgtaggcaaaggaaaacctcagaattacagaggtggaaaaagagttctagtcaagccacttcagctggctacagagtaggtgggaaagaaaatgggaggacaagggctcagatgatggggggttggggcattggggggacacttgaaagctaaactaaggggttgaacttaatttaggaggcagttagaagcttttacatatttttgagcaagagagtgacataattaaaatgatctgggccaggtgtggtggctcacacctgtaatcccagcactttgggaggctgaggagcttgggtcacctgaggtcaggagatcgagaccagcctggccaacatggtgaaatcccgtcctactaaaaatacaaaaattagccgggagtggtggcatatgcctgtaatcccagtagctgggaggctgagacaggaaaatcgcttgaacccgggaaacaggttgcagtgagccgagatcgtgccactgcactccagcctgggcaacagagcgagactccatctcaaaaaaacaaaacaaacacacacaaaaaaccaaaaataaataaataaaatgatcacttctgaatactgatctaactaggggttgcagggtgggctgatatagggagaaactggagagcaaggagatcactaaggtccctacatgtccagaaccaagatagaggtcttgaactaggatggtggcagttagaacaacaacaacaaaaagtcaattccaggctgagtgcagtggctcatgcttgtaatcccaacgctttgggaggctgaggtgggagttagaaagcagcctgggcaacactgcaagacctcctctctaaaaaaaaaaaaaaaaaaaagttagccaggtgtggtggtgcccacctgtagtcccagcaactcagaaggctgaggtgggaagattgcttgagccccaggagttcaagcttgccgtgagctacgattgtgccactgcactccagcctgagcaagaccttgtctccaaaaaaaggtcaattccactgacttttctaaggtgtacaccatcaaggggcagctccatctccaggccattggctcatgagacattctgtagtcagaaggctagggcagattgctttgagcaagcccccatggtggttctcactcctacttctttgggtatatgcccctctgtttaaaaataaagttaatatgcatttaaaaaaaaaaaggagaaaaaggtcagttccagaaactgtgtgaataaagcattttacttgctttttctattaatctataacatatgttgattttttaaaaagaatataagagctatgcaaattggagcttcaagacaacttcccatctccctaggaggagatggctgccctaaacccccctacatagaaatcatcccactgcttgggcttaaacttgatgttggggaaatgaaaaatccaagctaaggccgaagcctggggcctgggcgaccagcagaatgaggaccactggtcagtttcaggctgaggtgcgtcttccaggggacaatctctagctggcccttaaacattcagacttcaagctctatttacagcataaaggtgtttcaaaagacgtgatacaaataactgcaaatgctctgcgatgtgttaagcactgtttgaaattcgtctaatttaagatttttttttctgacgtaacggttagattcacgtttctttttttttaagtacagttctactgtattgtaactgagttagcttgctttaagccgatttgttaaggaaaggattcaccttggtcagtaacaaaaaaggtgggaaaaaagcaaggagaaaggaagcagcctgggggaaagagaccttagccaggggggcggtttcgggactacgaagggtcggggcggacggactcgagggccggccacgtggaaggccgctcaggacttctgtaggagaggacaccgccccaggctgactgaaagtaaagggcagcggacccagcggcggagccactggccttgccccgaccccgcatggcccgaaggaggacacccacccccacaacgacacaaagactccaactacaggaggtggagaaagcgcgtgcgccacggaacgcgcgtgcgcgctgcggtcagcgccgcggcctgaggcgtagcgggagggggaccgcgaaagggcagcgccgagaggaacgagccgggagacgccggacggccgagcggcagggcgctcgcgcgcgcccactagtggccggaggagaaggctcccgcggaggccgcgctgcccgccccctcccctggggaggctcgcgttcccgctgctcgcgcctgcgccgcccgccggcctcaggaacgcgccctcttcgccggcgcgcgccctcgcagtcaccgccacccaccagctccggcaccaacagcagcgccgctgccaccgcccaccttctgccgccgccaccacagccaccttctcctcctccgctgtcctctcccgtcctcgcctctgtcgactatcaggtaagcgccgcggctccgaaatctgcctcgccgtccgcctctgtgcacccctgcgccgccgcccctcgccctccctctccgcagactggggcttcgtgcgccgggcatcggtcggggccaccgcagggcccctccctgcctcccctgctcgggggctggggccagggcggcctggaaagggacctgagcaagggatgcacgcacgcgtgagtgcgcgcgtgtgtgtgtgctggagggtcttcaccaccagattcgcgcagaccccaggtggaggctgtgccggcagggtggggcgcggcggcggtgacttgggggagggggctgcccttcactctcgactgcagccttttgccgcaatgggcgtgtgtgtgtgtgtgtgtgtgtgtgtgtgtgtgtgtgtgtgtgtggaggggtccgataacgacccccgaaaccgaatctgaaatccgctgtccctgccgctgttcgccatcagctctaagaaagacgtggatcgggttctagaaaagatgactccctgcacgcccctccctgcacctcccgagcagtgattccgacagggccttcactgcccctgattttaggcgggggccggccccctccccttttcctccttcagaaacccgtaggggacatttgggggctgggagaaatcgaggagatggggaggggtccacgcgctgtcactttagttgcccttccccctgcgcacgcctggcacagagacgcgagcagcgccgtgcctgagaacagtgcgcggatcccactgtgcacgctcgcaaaggcagggttcacctggcctggcgatgtggacggactcggcggccgctggtccccgttcgcgggcacgcacagccgcagccacgcacggatgggcgcggggctgcaggtgcatctcggggcggatttctttctcagcgctcggagcgcagggcgcccggcgtgtgcgctccctgccggaggcgcggggctggcgcgcagggctcgcccctcactgcggcagtgggtgtggaccctggtgggcgaggaagggggaggataggctgtgcctcctcccactcccgcccccagcccccctttttttccccctcggaacgcgaggtgccatcttttttcggcgtgtcacgtctttacggtgccatgccaaaccgggtggccgggcttcataggacagggcggggcctggcattaaagggagggggacaatcagcgctgaaatcttggcgttttgctgctgcgggcgtgagcactgggggcgttcgcccagcaccttcttcgggggctctttgctttgtctgtagaggttacgtgatctgcgctcccagccctggtttctggcttttattctgagggtgttcagtcaacctcccccctacgcccatgcgcctctctttcctttttcgctcctcatttccgagcccattgttggatctcgaggcttgctgggttcgatgaactcgagtcaaccccccgacccccggcacgcatggaacgggcgtgaccgcgcgcagcctcgtctcggagtctgccggcgccgggaagcttctgaagggatgggattcgagtctccgtgcgcgctgcgggcggcggcagagggatctcgcccctccctacaccccaagtgtcctgagggccacgccacaccaggttgcccagcgagggacgctggctacccatccggggatgggtggggagccctggcggggcctctccggctttacgccctgttgcttcgcctggccggagaatgtgaggaaggggcataaggttactggtgcttcggccacacccatctttctgagcccactggactgggcgcagaggggggattgccatggaaaccacaggtgtccggagaggggatcttggggctggcctcaccccttccctgcggagattggggaccctggggtagggggagccgcgcccagtcggcctcctggaggacacgggaggaagccccgaacccccgcgcctgaggctgtttctgattggcccctggaggccgcagacacgcagataggcggccctgggtgtatttttattaatattatgtccgtactgattaatattatttatcttaaataaatttcacccgtgtccaagttcaccgcgcccccaaaaccgagtctggggcggcagggggaactcctggccaacgaatccatgcctcgccctcctgtgatgaacctggtacgcacggttttctggttaattctatcgctgaaaactggtgcggggggcgcacttctgagacggaagagcatctaggagctgaatcctccacgcgggtcgcccaggttgatctgaatttctggggaatggcttggctgcccgcccgggaccaggccgaccctccttgacggtggcgtagagggctggagcctgggtactgcgaggctcctcgcatggctgggcccgccgcgaggggttgcagagcggctcagggatcgattcaagcatcgtctctcctccctcgcccccagacagagctgggcgcggggttccccttccagatggagcgagggtctcggggtggccccggaaaaggggagcccgcggccacggctacgtattgccatctcgcgagcagagatgtcacctcctgcctttggaggaaagggagcccggtggggatgagcgcatttagcccaatgctgggaacaaagcgcactccgcgcttctgcgatttcgctccattttgaaatgtgttggcgctttggtggggccgctgcggtgggcaaggccgggggcgctgttaatggaggaacctcagggggacggtccttcgtaggaaactctatcctggctctgcgcgcgctttaaggaaatggcttccctccaggacctcgagggatgcagcttttgcgcggatgacggtggggtgctgaaccagccggtgcgcctctggaaatgtctgggcacggatcctggggccatcgacgactcctccccattcccagcaggcgggagctcttacattccgagcgagtgacccctctcaccctctggcgctcacacacctgtaactccaaacctccgtctcagaatggtccaggctggaagggatgatgggggctccgacagcgactgcctagctcacccctctgcgtgctcaggctccaggctcagcaggaccaatttgagttctatctgatccccctcggccccttaactgacccatcctacaggagacagggaaatgtctttcctaccgcggttgattctggggtgtcattttgtgttttgtgatggctgcttatatttactgtataagcattgtatttactgtataagcattgtattataattactgtataagctgcttatatttactgtataagcatctccaaatcctccctctacgtaaacaaattaatggataaacagataagtgtatcccctgcccccacccctgctacgcaggtccggagtgactcttgaagctcatacattccttggccaagtttgcttctctaacagatgtttatatagcaataacctggcttggctcttgggttcacctttggacgatttggggaaggggcttgttggctttgctgggttttggatgagtgacagtccatgactgttcctgctggaagggcgtgacttttaagtggtttctaatatcaggcattgctcctccgacaggaacaaaagaaatggatactgcccataaattgttagaaaacttagaatcgctttgattgaggaaaggttagatttattccggttggaaaaagtggcctttctattaaacgtgccctttgaccctcatgcccttggaggtcggtgccagcctggagatgggataagattgtggttttccttctgcctttttaacatctgttgttacagtccatttgttgaaaatttaaagaaactgttttattccactttccctcagcatttatgtgtgtggtttcagtagctctgtggctatatgtacgaacacgtgttatttttccaattggacatgtgataattttccaactggaccttgccttctattgatgtatttatttagcatcttccttactccctccttgaaaaagaatcactcaaaaacaaataaaaacagccgtaggggcctaatacagtgctagacatacaagaggtattcggtccataccaaatggattttatccatgaaggataaatggggaaatacagtgggaagcaggtgggaaactgcgtttgactctgctctttcctccaccaccactttcctcatcaccgtgttcagagacccccaaagccccctcacactcccagaaacacccccctggccactcctaacttgccatgcccaggagttaggtgcttccactagtgacatggagctggcgtttggggggcacctcagcaggtgacgggaagagaagaccccagcctcaccagctgggctgcagcagggagaggagtcctcatgttccagcagggactctcagctgttttcctgtaaaaccatggttctcaactgggggccactgagatgtctagagagatgtttttgttttcacaactcggggagggtgctactgacatcttgtgggtagaggccaggaatgctgttaaacatcctacaaggaaggcacaggacagtctcctacatcaaaatatgacccagtcccaatgtcaccactgctggggttgacactggcactgctatcttaattacattcattgagtgtcttttaggaggccctattctaagtgcttgctaagattatctcatttaatcctcacaacacttccgctatgtagcaggtgctgttattatctccgtgatggggaaactgaagcacagagagggttagtaacttgctaaaggtcacagagccagtgggtggtggagctggttgcctgacactagttccctcccctctcagccacatgtgggtttacttggccattgtggactagtctgggaacccagatatgatctataacattgacccagtagaatattgattccaaaaccactgtctcacaaatgaatttttacaagagtctgtaatcggagcatgacccagaataaggttagggagatgtggagttaaagctctcaatttcttatctggccccgacacagagagcaaggcatttcactctacattggtgctctgtttataaaacaaagagcaaatatctcttcctaaggtccttaaacctcttcccccaatccagggtttctggactgctctgccatatgacggggcagctggtttgattgacccagggaaggctggaaatcaagactgggggatcaagacgtagattcagtgtggccaaggtcaagtctctgaggtttagggacatcagatccccagcttaggttctgtacctcggcaaggtgaaagcgttggcgcccactgatgaggcctgctctgagattgtgggtgtgggttgagttgggtgggcataggcaagtcctcttgtaagaatcttttggcaaagatgggcctgggaggcttttctcacttcctggggcccaggctttgcaataagtattccattatactgtggtaccttggggctacctgagaatcctctgtctcgcccctgttgccttgccaaagagtttgctgtccaagaattcctttcctgtctccaggtgccatgctcctgccacctctgccaggttccctgcctgcccagatggctcccaactgagtgtgaggaggaatttgagacaggttttgagctttctgggttctccagttaggaaactttctgtaagcatgcagatagaatgggcttcagcaaaatacaaactcgaacaacttccatgtatagtcccttaattttctttgcttttttcatatttcatcaggctccatgctgagcccaatcagggacccgatagaaatccaaacaccatgtcagcgagtccccaagaaatgcattttgtgccaaggctattcaaggaaggtttgggagcagctcaagggcagacactgttaccctcccccaggtccccagtgcagggcagtgttctgcatgtggaggcagtttggcctaatggttaaggaggtaggctctgatcgggcctcctgggcacaaatcccagctccctgctcactgtgagacctaagccatattgtttagctgcttggagagttttttgtcatccacaacttggagtatgatggtacctgtctcacgggttgccatggggttcacacaagctaacccggtactcactagggccaagcacatagtaactgctcagtaaatggcatcatcggcggtgtcctgtggatgagtgcttgtgattggctgaatgaccagaggggtctaaagatcctggtgatggaatcagttgtacagataaattgttacactgagtagggatcaagataggaaaagtcggcaactacccagctcccctgcaccaaactgggcagaagtggatcctctgaaaattgcacacacccatgtttaaatgtacacacagaactcttgccacaggcaagcggagatttgtcatctgctgtccctgcctcatcttcttcctgaaatccactccatgccaggaataaactgcatgctctccaccagcccaaactgacctgccttcccgccagccatcccgggcagggtgacctggcttagtacatcgggttcagagatctttccagtttactcgttgaataaaaagtgagggctgatcgagaaagtaatggcagtcagggaaggcgaaggaggtaaagaagagattttacaaatgaagtaattcaacagagtgctgacattggtaaactggcaaacagatttcagggtggttggttgagagtagagtagaaaaggattaaataaagcaaacttgtggtgtactgaatcttaggaattccatgtatccaataagtatagtcatttatgaattaataaattcggcctaagaagccttcttatcgcttaaatcaagactaagtaacaatatatcagttttaaaaagtcattatatcagaaaatcatttaaatgatacacatagatttccaagattttactttaaccgaaactatataaatgtgaatttgttcacccatcttttgacacagggctcaggtcttctcttggtgtctggatcagccagttgaaatttcttgtctgttttgcctatgccacattaataatgcactgtctgggtcctccgatttcagtttggattttgggtttacattgtggagtcatctgaatgcagaatccttcagggattttacttttttttttttttttcatggtctttaccatcccatttgatagtaaatattactcacctttatgaagtctttccaaaacattcaactaaattttcttaaaatcattgaatgatttgaagagcttattcctcagcacttttactccatcagcttgcaccttattttttaatctttttttgagacggagtctcgctctatcgcccaggcttaagtgcaatggcgcgatcttggctcactgcgacctccacctcctgggttcaagcaattccgcctcagcctccgccgtagccgggactacaggtacacaccataatgctcggctgatttttgtatttttgtagggatggggtatcgccatgttggccaggctggtcccgaacttctgacccaagtgatccacccacctcggcctcccaaagtgctgggattacaggtgtgagccaccgcgcccggccagcttgcaccttatttaggatatgtgattattatagcaagtctggtgtacatacaagattttgaatgggcacagatgacctttagtaagtgcttggctgtgataagaggcagtcctgactgcagatcaggctgtgtggaccccagccttgcatgtttacagaccttcatgtcttattcttacagggtatcagaagaacacctactggggaaacttataaattagtaaaaggtgggcattctccccgcccatcttctgtctgtctgccaggactagcacagcactttgaagtcattcacatagaatcccaacttaagagggtaaaatcctcctcaacagactgaaaataagtttaaattccctttgctatattaactcccctgaggaaagagtcttagatcaatgtccaacactaaaaacagttttaaatcagcaagtgagaattaaatctgaagcaattgataataatgtttcattcattcctctcctttggccccgtccaccctactgctaaatccaggcatcaaagagaagagggacataattatctctagtcccagctgctggttttccttccagcctatggcccagttttctgttttactgagaaggctggtgatgttatcttgggatctaagtctgcagtttcaccacaaaaagtccagggatgcactttcatgcttgtgtcctcctccctgggatagcaaggatattagaagacccctggctctgtaattgcttgtcatgtgctctacagacgccacagaatgccaagaacgaagtgctgggaaggacaaattcatggaaccgtgggacggtgctcctcccccagcgtaaaggacagctcctcctcctgaattggagccagcgttctaaatcatgtgtcaacagagttgtcctggatcggatccagttctgccattgatttgcaggtcatttcagtggtacctgtttccagttgttcttaattgaacagtggcaccaaactattgtcttgcctcatccccctcccatggcctgtcccccaaaaagagacttcttgggtaattaatcagggcaacatcaggcagtctgggcgcggtggctcacgcctgtaatcccagcactttgggaggccgaggcgggcagatcatgaggttaggagattgagaccatcctggctttgtgaaaccccgtctctactaaaaatacaaaaaattagccgggcgtggtggcgggcgcctgtagtcccagctactcgagaggctgaggcaggggaatggcgtgaacccgggaggtggaggttgcagtgagccgagatcgcaccactgcactctagcctgggcgacagagctagacttcttctcaaaaaaaaaaaaaaaaaggaatctctttggttttatatatttttttttatatatataatatatattaaaatataatatatatatttatataatataatatataaatatattatatattatatatttttatatattatatattatatatattatatattatatatttatatatttatatattatatatatttatatattatatatttatatatattatatatttatatataatatatattatatattatatattatatattatatattatatatttatatatattatatattatatatattatatattatatatttatatattatatatttatatatattatatattatatattatatatttatatattatatatttatatattatatatatttatatatattatatattatatattatatatgtatatattatatatgttatatattatatatatttatatatataatatattgtatatattatatatctaatatattatatatattatatatattatatattataatatatattatatattatatatatttttatatatataatatgtataatatataatatatataaaaacatatataatatatattatatattatatatatattatatatattatatatattaaatatattttatatatattatatatattatatatattaaatatattttatatatattatatatatatacacatatatatatataaatgaggccaggctcggtggctcacacttgtaatcccagcactgtgggaggatcacttgaagccaggagtctgagactagcctgggcaacaaaacaagatcctgtctctacaaaaggaaactgtaaaaattagctgggcatgatggcatgtgtctgtagccctagctacttgggaggccgaagcaggaggatcgcttgagcccaggagttcaaggctacagtgagctatgattgtcccatagcactccagcctgggtaacacagcaaggccctgtctctaaactttttttttttaattctatttatatttacatgtatttaaatgtgaatattcactacctatttgttgcatgcctgcattttttatactgggcttgccaaaaacccgaacagctttctactttgacaatgtatcagaatttaaatcagcaatatgttaataagccaagcaaaggttatatatgcaaataaaactgttgtctataacctcctgttacactggggcacagcaaaagtcatggtgtagtcgcatgtgaacctgtccctttcatagctgctcattgccaggaaacatcaggaatagccatttggaagagtcatcagccctcccaccatccgttttctgtcttgtcttttccctatgagcaggggaaattccacgctggccccaatccccagtgcagcggctcagcctctgcctctgctgctggtccccatgaggccagcttagaaacggaggattttgcagaacatccctaaatccgcttgaataatgaagtgatcattcataaactcacctgaaccttattaaaacctatttaatatttttcctggataatcctatagggataacttgcctcctgggcttctctccaccgggttcagttcttcctttagtggtgaagttcctcccttcttagcatctcaactgtgcctgagaaaaggccagtggcggctgcactctgttccctgtggagtgttaataaagactgaataaattgaaataaatccctttcaatgtcattaagtgctataaataatcatgaaccaatgttcgatggctgatgagaaatgcaagaaaaaatttttaatcagtaggattcataagttgacaatctgggccaagttaaaaaaaataaaaataaaaagacttttaaaaagatcttatcgtttgttaccagtaagactgaattccagaagcaagctactccctcatttgtgggcccctgttatcactggctgcttagggttgccaagccctgaattcatttgtcaactaagagatttttggccaagattaagatttcccatgcctccatatttccatctgagaaatggagattatactgtcttccccctcagaatggatgataatgtggtctctcttctgttcgcatagtcatagaactgaaataaaacaacttaagagaattcctttgagcttctcagaagtgctgcagggctgggggatgcctcccaggagccgcagtcaggtgctgatctgaagtctttggtgggctgactttagcctgacctgaaatagtatagctgctgccacctggctcccttagcgtcagtcagacggtgcagctggttcctaggggtgagggctgagccagcagggtccgtgcccaggagggatgcatgggtggccacagcccagcctgcactgatcttgtctgtccccttctttggaaggaaggagccccaaaccagggtgcaagacagtgggtgggggtgccttgagcatgacctcaagtgatttccagcccctgccagtgctgacttctctggggaagggctgggacttccttctgggctcaagtcacgacccttggatggaatttcctgggagcttttctgttttttctggagttttcagttttttcctaaccagacagggacttggtacagaatctcatattctaattatgcctaggagcagcctctccccaccactcacagtgtttagcatgtgacaggaatcgattaaggcatgagtgattaaattaaagccaggcattgacttggatggtgtaatattctgacatctgtttggtgtcaaaggcacggggcaggcgcgttaattgaactgcttgcacctggcatttgaattgagccagagcggggctaaagtcagtttgccttcaccctgtaaatggagggtttctccggagcgtggatggtgggaggtatttcagggtgtatgcataacccccaccctgacaatggcccatctcttctccagcgtggccaggtttgagtgccagtcctgggtgtccagtggccccatagccttgcgttttagtaaaatgctgcccccattaccacctggtctgtgcacttcggtcactggaatttgccatcttccagtcccgaatgtggcaagccatggagccttaagctcttctccctccacatcctggaacagacccgccagtttcttccaggcattgcctcagtttgcccctctgtttccagtcacactctcaccagcgataaaatgattttagaccttatcatctcaccctcggatccttatggaaacaataatgagttgttccctgtttcaattccaaaattcatatccaatccgttttgcatgccattgccaaattcctcccagagcaaccccgtcacctgccctggccctctccaagtgtggtcctgccatgggcatcgcctgctaagccaagctggcctcgagctgcctgcccgggtccccacaccttggctcacctccctgcccagtcccgcctcctgccagcctgccctgtggctccttcatagatgccgtgctctttctgccccttgctcacccatggcagccttgcccctctctccctgccccaccccctatttaaattgacctgaccttcctcagtgtccatcttccccgaagctttccccagccttggcactcaaggtccagaggctacgcgtttcctctcacctgtggcagcgccgtgctccccagtgcctcacagtttccttcttgcccccgcttcctgtgtaggactcatctgcccacaggttgcacgtcctgtgagggcaaggactgtgtcttatgtgactttccttctccagtcacagagctgggcacatagatagctcaaaaccctctttattaacacagttggatgttgagaaatcaaacaggccaatgtcaaatgagctctccttatttaaatcaagtcagttctccacctcctagcactcagttccagtactctatatacatggaaataataaaaaacacatttcctttgaaacattctataatcgttcctttgccctacttcagaccaacttaacgcactccccattggtccaaatgagttttgctatacgaagatgctgataataatagcagcagtggattattctgctaaaaccattgcctcgttaatcctcagtcccgaggtggggattattatcctcattttgcagagaagcaaactgagactcagagatttcacagctggggagggagccagctcatccctctgtccaggcccaagctctctcccgcttgccttcctgcctctgcaacctcagagcatcccccatctggttctactgcctgtgctagtcgtgcaggagccaaaagacacgtctttagtgctaaggactggagaagccatgccctccagcctctgtgaatgggtcatatgtaacatgagcctggagaaattatttgaaaccaaaggcaagcctctaaaccaggctgctgcttcatggcgccggtgacggcagaaccaaatttagtgctgtgggcaggtccacacttatcaaatagagaagctcatttttcttccggctcacatcaagcatgaaaaatgttcacacataccccccacacacacatgctttccggaggggtccatgtggctagaggctggaagatgtggatgagaggagcctggcaggtaagcccagggaagatgacattcagcttcccagacagcatctacagggagaaatttaattaaaagtggggcggtttccctgagcaaggcagacaaagtcagccctctactgttaagaaaaagggtcacagtgagaggggaggtgaggagactgagtctgtattttctagtctgttgggctacactacctgatcccccttcctcaaaaatccactttactttccccatgtctacaccaatgtggttcacactctgggaccaggaaaagggggagtgatggggaacagagaagggaggagctcacacagctgaggctggggttatgcatatcgaattacttagaatttgcaacctcacagggtactttcatggcgttgaaatacacttcccacagccaccctccctctaactaaaagcaagagtcatttctcagttctggtcttgcctcccacgttctcctccacatttaagaaaatccaccagctacaaagtgaagataccatatgtgatatcccaccctagtttctgttttatcagggtttggagcaggtggagcaggcagagggatcatttcagcctataaattgtattaagggtgagtactgagtcattcttcaagaaaagttttagaagcatccaaaactgaagggtggagccacctggagacagtatcatcagtcctggccccgagcatggcctgcataggcccccatggatcccagcgggagctgcagagtgcgggcaccttggcacacagccctgagtgcaaaattaggagctgggcagagggcatctctctgtcgccattgggcagcccagggcacactggtcatagccttagaccacgaacaccctgtgcccgggggacagatgcaaccagtgtgccctgggctgcccaatggcaacagagagatcgacacctggaccccatgtcacggggactccactactaaggctcctaagactgccaccttccagtgggataagccctgcctcctactgggcccacaatgtgcagagaacacttgggactacctggctttctggatacacaaatattgatccaatctggactaattagaaggtcagtcccaataacaaatcgaagtcagctgggcgtgatggctcactcctataatcccagcactttgggaggctgaggtgggcagatcatttgaagccagaagttcaagaccagcctgggcaacatagcaaaaccctgtctctactaaaaatacaaataattaggctgggtgtggtggctcatgcctgtaatcccaacagtttgggaggctgaggcaggtggtcacctgaggtcaggagtttgagaccagcctggccaacagggtgaaaccccgtgtctactaaaaacataaaaattagccaagcatgatggcatgtgcctataatcctggctactagggaggctgagacaggagagaatcgcttgaatccaggaggtggttgcagtgagctgagatggtgccactgcactccagcctggttgacagagcaagactctgtctcaaaaaaaaaaaaaaaaaaaaaaaaagccatgcctggtggagcactacgtgtaatctcagctatttgggaggctgaggcacgagaatcacttgaacctgggaggcagtggttgcagtgagctgagatcgcgccactgcactccagcctgggcgacagagtgagtgagactccatttcaaaaaaataataaatctgagtcactttaatattgttatttggatgtcaacctctaggtgtttgagacaggagagtgatatgggggcactggaaacacacaggcacggggtgtcctcacacttgggtagcccacacgatgtgatttcagggtgctgggaggtccccccactccccaaattactaacaagtggatagtactttacagtttatatgatctcatttgattcttaacatgagcctgtgagtgaaaaattccttcccctcttctacagattaggacgttgagattcagggaggttcagagggattcagggaagtcaagtggcacctggagtcccgtggctaatttgaggccggtaggggattcgaacccaggatttgtgcttcttatgcctgggcttctgctccctggggcatggtcttccccctagctttcccattcactgctttagcctaggggtcctaccctttattaaactgccagtgcctcactgcttttctcccccaaagacaaaaaaaaagtgtttttgcttttgttttgtttttcatgggcagagacctggaatttcagcttgagaatttgtgccatatgataaataaatcaacagatggctttttccttaaaaaaaaaaaaaaaaaaaactaagatgtatttgcagtgaggcataatttgtaccaaaaagtgctcaccacactgtagtcatgggggcaggaggcagccgcgggtgaagggagaaatcttggagtccaggcagcccccttctgggctgaactggggagctgggggtgctgccagccctgccaggttctcctaggaggcggcagctcatatggctgtgggaggaggcagagggagcctcatatgcacccacatttccagggatctagaagacagaaggaggaaaaccaccatcatgttaaagcagacagttaggtaacacatcctgtaatacaagttattttttccacatctaaaggctaaaaatagttgttagaatttaaagataattggtaaatgagtttctatccttctagtttcacatcaaatggaatcatgctgccttcacatcactagtgcccgttatttgtgtttaatttccacaatgttgtctaattccactctttgggcttccccagggatccagcctccctcactcgcccatcgcagggagatgctttattcatctttgtgtcttctgtgccgggcatagcgcatggcacagaataagcactcagtaattgattcacgagtgaataaatggatgagtgggtgagttcaatattgactacaaaaaccctaaggccacactggtgagtggctgcgcctgtagtcccagctgctggggaatctgaggcaggaggatctcttgagcccaggagtttgaaactagcctgggcgatatagcgagaacctgtctcaaatgacaaaaacagggccaggtgcagtggctcacgcctggaatcccagcactttaggaggccaagatgggaggatcacttgaggccaggagtccgagaccagcctgggcaacatagggagaccctgtctctacaaaaaattttttaaaaattagctgggcatggcggtgtgcgcttgtagtcccagctactcaggaggctgaggcaggaggatcacttgagcccaggaaattgaggctgcagcgagccatgatggcaccactgcactgcagcctgggcgtcagaacgagacctgctctcaaaaaaacaaacaaacaacaaaaaaaaaggctttcttaaagagacttgagaacagaaaggggaacagatacataacttatatatttatttgttcatctttccaccttcctggagggtggaggggaacaggtctgtatttggagttttgaatgctaaaagtgggaatacatgtactgtttgccatgatctgttcaaaagttaagccaaatgccttagattctcctgaaaactggaatgccactgtaaactataagccccacttcaaagataaaagatcttgatgaacagggctgggtctgtggactgggcctctccccaccacacaaggaagggtggtgccagttgaaggaaaatcacttaaatccttgctgtctcctaataaggtgtggtcccaggtagggctgtcagaattagcaaattaaaacacagggcatctgtgaaaattagaatttcagataacaacaaataattggcataggctgcataatgtccctcaaagatatcaggtcctaatctccagaacctgtaaatgtgatcttatttggaaaaggggtctttgtagatgtggttaaattaaggattttgagatggggggattatcctgtattatctaggtaggtcctaaatgcagtcacactcatccttgtaagaggaaggaagagagagatggaaaacacagaagagaagacaatgtggtgatggaggcagagattggagtgaggtggccacaagccaaggactgctggcagctaccagcagccagaaaagtccaggaaccaattctctcttggagctccagagggagtgtggccctgctgacaccttagcttcaacctagtgatcctgattttggactttggccttcagaagtgtgagggaatgaatatctgttgttttaagccaccaagtttatggtcatttcctacagcagccacaggaatcaaaaacagtaagtatgtcccatgcaatgtttgtgacacacaccaaaaatattacttgttgttcacctgaaattcaaatttaactgggtctcctgtattttatttggccaacctagttcccaggcccaaagaaagaggcttttgaaatttgcaagaaagctggttggagctgtcagaaagtggactttgtaaacacagtaccaccgaaccaatttgaactgtactacctctagacaaaagagagggcagtcagacagttgttcgtgatttcttctttcaacagtcatttgagcacttactacaaaacagaagctatgtgtaagggtggaggcgttagctgttaatcaggacctccaggctaagtttctgtattagtccgttttcacgctgctgataaagacatacccgagactggggaatttacaaaagaaagaggtttaattggacttacagttccaagtggctggggaagcctcacaatcatggcagaaggcaaggaggagcaagccacatcttacatggatggcagcagacagacagggagagagagcttgtgcaggggaactcctctttttaaaaccatcagatctcgttagacttattcactatcaagagaacagcacagaaaagacctgcccccatgattcagttacttcccaccagatccctcccacaacatgtgggaattcaagatgagatttgttaccatatcagttaccaacccttccagataaatcacgtgaaatatcgccattaacagagtgagctcaggtggttcttcagtgcatttctgatacctgaaccttccctgggaatttcacagaccatcaggctctccaccctttgatagcaggatagcagggcccaggttctgcaggaggagatgttaccacaggcctgaaagggagggaggggcagatgctacaggaagatgctggctctggattcgctggaggagctttcaagggaagtagatacacactgtctccatcatttcatgtccatcacactctaaaatgctttggacaagaagcaaatgttaaagacaaatgtggcccattttcctgtacaaagagggctgctcccatgccaggctattggcactggtgggcatgaggcttctctgctgccctggccggggggttctctcactcaccattggctctctgacacctggagagaccaccacccttgggctttcatgatgctcacagaatccacactgttggagctttaaggagcctggatcaactggaacaggcagggagtactaggacagcccagcattgccccaaaatatccaggcctgataaaagagaaaaacaggtagctcacaggaaaaggataaaaaaaggaggagggatttaacatgaaaaggtgcttgatctccctcataataaaaagactgctgattccatccaggcaagtgacagaaaaaaaaaatttaatttaaaaagactgctgataaaaccacagcgagacactgctgctcagggatctgagggtgtgggcagccaggctgccacgcatcatgggtcggagaggaagaccacacccctggagcagagggcggctgatctgtcagatgccctttgacagcacctcagcttccaagaattaaccctttctatgtgagcagaggcatccatggggggacacactggtgaatcatctgttatgtagaagtctggaaaacatcaggatggaactggtgaaataagtgtggcctctgacggaatggagcggtccgtctgcactgctgcgggtgcccctcagatcctgtgggtcagtgagaaaagcagtgaggaacaaggcaggtactgtgtactgtcctctgcgtgcaaggaaggccagcgcatgcaacagagtccacacagacatagcctaactctggaaggaagaatgagaatgcagtttcagtggtggcctctggtggggagaaactgggtgaagggagatgtcatttccatttctctactattaattttgtattaccatgcttaaatgttactttttacctttttttttttttttgagacagggtctctctctgttgcccaggcaggagtgcagtggtacaatcatggttcactgcagcctgaacctcccaggctcaagcaatcctcccacctcagcctcctgagtagctgggactataggcacgcataccaccgtgcccagctattttttttaatcaagatggagtttttctatgttgcccaggctggtctcaagctcctggactcaagcaatcctcctgcctcagcctcccaaagggctgagattaaaacgtgagtcaccctgcccagccaattgctttttaaaaaagattaaatgcatgtatacgctcaggcatcagcacacttggaaaggatgaaaatatccggaagaagggttcttttaaaaggctcctcaagtgatgctggcaggcatgacgaatgtccctggtcacaaaagctctgatctggcctaaccctgtcatgttagagactggagtgcgtgtgtgtgcgcgcaaagtgtggggggatgggggtgagtgtgtgtggtgtgtaagcatgagtgtgtatgtgtgtggtgtgggggtgtgtgctgtgtgagcgtgtgtgagtctgtgtgtgtagtgtgtgtgtgaagtatgtggtgtgtatgtgtgacgtgaggtgtgtgtggtgtgtgagttgtgtatggtgtgtgcatgagcatgtgtgtgggcatgtgatgtgtgtgtggtgtgtaagcatgtgtgagtgtgtatgtttgagcatgtgtggtgtgttgtgatatgtgtgtggtgtgtgagcatgtgtgtgtgatgtgtctgtgtgtggtgtgtgtgagcatgtgtgttgtgtgtgtggtgcatgtgtgtggcgtgtgagcgtgtgtgtgcattgtgtctgtgagcatgtgtgagtgtgtgtgtgttcagcatatataaggcatgtaactgaacacagcactttagagggctctcctggagtcagagggggtgggtaggaggagaagggaggtgggctagtgtgctgaagtatctactccttgtcatagtctgtgacaacccagactagcccatgagccaccctgttccctgcatttccaatgagacctcggtggacatgttccctgaggtgaggctgactgatgtcatttgacgatcttgatgccaaatccttttatatcaaaaacaaccagaacactctcttttctcttagtgctttcacccagatgaccacatttcatcctcccagccactctgggccaggtggcactgctggtttgaaagggaggtctcccctggagtaacttccgtgggcggattcacaccctgcccacagtcctgtcccagtcagcccaccatggtggtctccggttcctccagaattcccgcttttcagctcatccccacattcccggagggactgagagcgcagccccagggccctgctctttgggggccgtctctacacccagagaagcagcaaggcattcctaggtttctctttcagatgcagaacttcagtgttcagagatgttcccactggtcctgagagggctcagttcagctttaatgactgcgctgttgcgtgtgctctgcagagggcgggtggcccagcgtggctgactgcagttttcctgacgtggagcccgagcctgccccgctgtttattaattaaggatcactctgcttgcagaaccctgaactccccagaactgtgaggtgggagaaccccgagaggccacctggccccacttcccacctgctgcccaaaccccctctctgccttcctgacagtcaccccaactcccagtgatccccatcaaccatctgacaaggggactgagagggaagagaaaggaggggcccaaagaggaaggtaaaactgtcgggaacagcccccaaatgtgtgacagccttcagtggagttgcccactttcccttttctcctccctgcaggacctcccttctccccagtcctccccaacttctgaggttacattgagaaaagtctgcagagaggtgccagcatcacaaggtgttaaggaccacgagtttggcattttaacagatgccagagccacttgagaaatgtggtaactaagcccagagaggtacagttaacctccccagagtcacacagcaggttcatggcaaagctggactagcacaggtgtccttcccctgcagatccccttctgtgccccacatcacctccctccagtgtctgggccacctggagatgggccctcagactcacccggccagaggtgccatctcatgggagaggtctggccaggaagcatcgatatttgagatcccaagaaatgaagacttggcctgtcagatgacagacttcggtcatgggaacacgtgatctgttttacacatgcgtcccctcagcagcagctttccagaacattcccactttcttctgtagtgagaagaactctttccctgcagcctcctgcccaactcctccttcagtgtctttgcttcagtgtctttgataaaccattctgctttgcagagtgcgagctctgccttgcagggttcgcatctgcctgtgctgagtaaccaacgctaaggtcgagtggtcggtcacctctcataagagctagggttgtctcatgctgatgactaggacttgccctcaaggagaaaaataaatcaaaacaaaagcaaaaacagcaaacatgcatctcttaaagaaggctctgagtccaggtaaatttccttccactgaagcagccaggctgaattcgaattatctttgcccctgcttaaaaactaatgcaaattttcctagagaatatccactaattcctggagggggcatgggcattcctgatgcccatgagaggaccatttgctcttccctcagtatgctaaataacagaagcgacatttgttgctggaaagtatcagtgaagttaataaggtttttcttgcccagggtgagggaacagttcccaatgacaaatgctgtatgggaaggggctgtagaactgccagcccctttggtccatccgtaaagtgaactctgtggatcctggaggattccagcgtctttttttttttttctttttttttaagacagagccttgctgtcacccaggctggagtgcagtggcacgatctcagttcactgcaacctccgcctcccgggttcaagcgattctcatgtctcggcctcccgagcagcaagactacaggtgcgcaccaccatgcccgactaatttttgtattattagtagagacgggggtttcactctgttggccaggctggtctcaaactcctgacctcaggtgatccacccgcctcagcctcccaaagtgctgggattacaggcatgagccaccatgcccagccagcatctttcatttttctgtctgctttggccctttcctctctcactgtcttccttttccatttccaaagtcagtccatctcactattagcacaaaaactgctagagcgcttgtcattggtcatctctccctgcacctggctggtctgttcttggccactgaagcgtttcccccagctgttgctttaatcattttattgttattatgccttacttaagaaatggatatgagatgcatttacctgtctcttcctgccactctgcagagccagtaagatgtggtggaaagggcccaggctttggaggagggctggctggggttggatcttggctgccccctactagctgtgtgaccttgggtaagtagctggacctctctgagcctggttcggaatcatagcacctctctttcagggctgctgtaaggaatagcagtggtgtgtataaagcagagcgcacagccagcaactggcccctagccacactgctgagcacctactgtgataagctgccattgtggtgtgtgaagcaaaggggaaacatgcctgctgtagtgagcttcctgtagggcaggttgtagaaccagaggtgggttccaaggttacaaagggactcttagtgtattagtctgttctcacattactataaagacctacctgagactggatcatttataaagaaaagaggtttaattggctcacattggctgggtgcggtggctcacgcctgtaatcccagcattttgggaggccaaggccggcggatcacttgaggtcaggaatttgagaccagcctggccaacatggtgaaaccctgtctcttctaaaataaaatacaaaaattagctggccatggtggtgtgcgcctggaatcccagctactcaggaggctgaggtggaagaattgcttgagcccgggaggtggaggttgcagtgagccaagatcgccccactgcactctagcctgggcagcagactgagactctgtctcaataaaaaaaaaaaaaaagaaaagaaaaagaattgcaagaaataaattattgtttatgagctatatggtctgtggtaccttgttgtgggactgggagtcttggcgtctccctgaccctgcctgttgctgcagcaccgctcagccctgcctgctccctacctgcctcccctcggcctctcctgcctccaccgggcccctggtgcctcctctagagacagtcctcctgggaccgattgtgttctcacttacacgaggcatccaggactacagataaccagaggaaggggcgccccccccgcctgccctcctccctggcatcctcacgctgcagaggtcagagcctcatcccagccccttacctgcccctactctgtggagaaccgtggtcagttcgccaggccggatccacgaacggccttgtggaagatggtgagctcacacccagagctggctccgatgaccctgtctcctttacatgtttctaccttcccctccctaccttcccccactgctgggcgcagagtggaggcagatgaggtttaaagctcagaagggcttaaacgggttggggcgcagtggctcatgcctgtaatcccggcactttgggaggccaaggcagaggatcacttgagcccaggagttcgagaccaacctgagcaacatagtgagaccgcgtctctacaaaaaataaaataaataaaattagctttgcagggtggcatgcacctgcagtccctgctactcagaaggctgaggtgggaggatcgcttgtgcccaggagtttgaggctgcagtgagctatgctggcaccacagcactccagcctgagtaacagaatgagatcctgtctcaaaacaaacaaacaaacaaacaaaagaaggcttaaagggggctccaggtgggcttggcagcacaaagctatgaagttctatcttagacacaagttctgttactgggcctttgcaggctggcctgggtacctggctgccatagacagggaaccttccagatgagctgcaggcgtggagcacaggagccagggtgctcttcctgggctctgtccacaggcagaacgtacacagtctttgtacacgtccggcggctctggtgcctatttttgtttgtgtttttcttttgtttggggggatggatttggtttcccccgagccctctgtcctcctgtcacctggctggtgctcggcaatgttgaccagctgcctggctggagttggcagtggctaaggctgtgacagctaacatgttcctgagtcctctcatttcttcaccataatgccctgttgagtttgcagatactgtctctgtttttatctcccggggaaactgaggctcagagtggctaggccaccttcccatggtccctcagctcatgagggccacacagggcattgcggtggccttctcctcagccttgaccctccggccccagcattgctgcctcaaggggtctcctctgctgagccgtgcaccttctgcctggcagctccaactctgtggctgtgttcagtggctcagcactgccccttgaccctccctggccttctgcggatgccagactggagcactctgacaaggtctggggtggttgtatgggtcctgtgacctctatacacctcccagtgcctgggaatcctgcagatacaccctccttagccgtccctaaccatagaggacatttctgaggtccccgagagagtggggcacccctgcaggatccaactgctgggcccaggaaggatagcagcagcatgaggggttccattagccacaaactcacggcatggaaccttcacccacctcgcccctcatctgctgtttagcacctggcacgccgtgtatacttactgattattacattttaatggcaaattatagtggcaaacgtatgcatctttgcacaattgttgtacagcatgatgaacaagtcattaatagtaaagaataaatgtgaaagtgagaaaaatctgactgccaaagtttttactccttccttccctccccagacttttaaatgaaagtttagggataatcccttagttgtcctgctagtaggacttgcaattaaaagaattgggccaagaacacttctacgcttctccttttaggtttgggtgtaaattcggggtatttctcactgatgaaagcctggtgcagggcagaccgtgggaagctttcatttccggaatggaccatcaacatcccttggagaagaattctcttctccagacccagacctggtgtcctggcacccattgggcaagtgggtcctagaagacaaacctggtcagagcctggaggctgcttagcattccccacgcacattagcagctcggagagctcaggaagccgcagcccctccttgcctcaccagcctggatcaggacagcatcccctggaagacacacagggcctggcctctgattacccagcctggagggaaagctcaatcgagcatcatgtcacccggtgcccccatgcagggtggcactggtgagacccccaagccaatgataccacctcacaggagtgcaggcccattgtggccagatcatcttgacttttcaagataaatcagaaatcgtatttccatgagatatccctatttgcaagtgatggtgactaaattagaagtttttgaatattgtaacatgttcgtaggctgtttgtctggtttaaactctatctggaggaattcaagctagacttcaggaataacttcttgaggcaaggattttgagaccttagggaaagaaggacgtcttgggggtattctgactgttgtcctcctggaagggaagaacagagaactagaagactgcccttagcgaagttcaaagcacctaagcccgggaccctcagcaagtgttcttgagtcacagattctccctgaggcgcctctttctggctccatagaatggctgattctgtaactcggtgagtttgctttttttttttcctccatcacccaggctggagtgcagtgaagctggagtgccgtggagcgatcactgcaacctctgtctcccaggttcaagcaattctccttcctcagcctcccaagtagctgggattacaagcatgcagcaccacacctggctaatttttgtgtttttaatagagacggcccgaagtgctaggattacaggcatgagccaccgcggccagccataactctgtgactcttgttacaaaggccttatattttgctctttgagggtggttttggtttgatgcctgttggttgccatcttttaactagggatgttttatcaaaatgcccagccaaagtgtccaaacaaattataccttaaagtttgaaaatgtctggcacttctaattcaatgcctgttgtgccaggcactgggctgctgaggaactgagtcccgtccctgcaggctagctagagaacacacacacacacacacacacacacacacacagagtggtcttacaagtcagttttatattctacctatatgcaataaaggtattattatgttgaggtgccttgatataaaaatttttcttaaaggagaggatgcctaaaacaggcattacctgaaacctcctctctccagcattggttgtcttctgtcatgactcagggttttcactgagaatgggatggaaatgtggtctaaagatagggccaatgttgggactggatcccctctgggaagtcagaccaggctagggcaggtccttgaagccatcaggaaaagcctctggagccagaaacaaaacaaaaaaaaaatggtgttaactaaactcagtctcaaatcctgaataggactcaagtcaagcaaaataattaaaggagttagcaaagggcaagtcagagagaccgagcaacaccaatgtcttccgggagccctgtggcgagtgacagagcctggactctggagtagaactcatcttgtgtcttcttctgccactcgttagctgggtgaccttgagccaagccccttaacctcttggaccctatgttcttatctctaagtaggggctggtaatatcttcccctttgaggaatgccctctaaggggtgttgtgaagattcggtaaggtggcaggggtaggactcctggccagaaacaggcacataataaatgctaagtctctccttctctccacctgctggatgctgtagatactaaggatttcgatgtgaatgagacaaaacccctgccttccaggagcctttgagaatcagagaactagacccatttccagaacaaggggatgcagggtctggataaagttttggggatcaatagagcagagggctcccagaggatcccatagggttgactcctaactcaagggcatgagacaacccccaggaagggcaccctggaaggggtccggctgtccctgatttacttgtgggcactgggggaatgcccggagccatccagccctcagggctctgtgtgattctgggttcctcccataaaagataatcagattctttcacgttaatgtctttctccacctcattgcacatcatgcagctattcattgactcagcaagtatcagctttgcatgcgaccttggcctacccactttagcttttagtaatagctcccttcttgaataatacaaccagtggggaaacagaacctaactcttacctctgggaggcttatttgctttgagaacatatgtcctgcagttttgttcatatggcagtgaagtttcgtgcacacactctagagccaggcagcctgggttcaaagcgcagctctgccaggtcctaactgcatgaatttgggcaagtcgctcaacctctccatgcctgagtttcctcatctgtaagattggagcaatggtaatacctgctttttagggttgagaagagaattaaatgaattaagatgggtaaagtgcttagagtggagctttgcaagtagtaagtgctatgtaagtgttcgatttaaaatgaaagacccttaaatacattctttgttcatttcacaagcccttcatttcacaaccttacatttcacaaccaagctctgtctcccctggaatccagccataactctgctcacaagtgtgagacaggccccagcagagctgcacgaagaggagagaaggcagccccccagactcccaaccccctgtccaagatggcaaaaccagaacacagcctctgtaccaccccagcaggtattcagaatctgcaatctccaaagcccacttcaattgtaaatgtagagccacgtgcgctttaagtcacctgtcactctggaggctcttttgctcagttcctcaccattagcagggatgacagggagtgcaggagtgcggtcgactcccagatattggagagcgctgggctagctgcccattctcccggcctccactcctctttgctgtccagccatcacttgctctttgaaggcaaacaaaacagaaaacagtgccaaaagtatgggaagaaagccagcttctcccctggggtgcctgtgatgccatgcccaccctccctgaccacgcagcccctgtggaccctcagggccccaagcccccatttccatcacatgcgtacacccatgtgtgtccatagccgcccatctcagtcaataaggctgctcctgcccacttggaatagtggtgacaaccaggagtggcttatgggaactatcccaatggcctgacagcatgtccgctgcaaaccgctgaggtaggacactgccctcatgtctagctgatcagcaagaggcgcagttgctttcttaggtaacattgctgctgtgtcctggccattgctggggggtggcacttaatctacaccagatttttccctcctgtatcttccaagctgcttggatcttggtgctgaattaggttggactttgtcttgtggggaagggaggactatagaccctcaacgtaagcaatggtcagactattctaagaaaactcgccgaattaaagcatgaggtaaatttagttctgacttctgtccaccccactgccactgtccccttttatcccatgatcccttgcttttcttttcctcctctctccctatctcttgtgtttgacgcatgataggaattcagaaatatatgtttgtggatttgtttattcacgtagcaaaccatttcttgagtgcctaccatgggccaggtagaatgggcggccccgggctgcagtggtttcttcagcccctctccagggtttacactgtgcaagacggtttgtgatgggtcctcccatcgaggaccacactcttctttctctgtgccccttggtcctcagtctctgaccccacttcaaaggcagcattcactcagggaagctcccatacaatgctagtcagagtaaaagtttggacaaattgccaggaagcagcttgtcagtatgcataaacagcctttaaaatattactactctttgacccagaatttcacttctaggaatctgtcctaaggaagtagtcacatgcaaaagatttatgtaccaagatgttcatcaaagtgttgttttataacaggaagtctcagaagctggataaatatccaacctctggaaatggttagatagaatagtatgtagccattagaaaattatgtctatggggtttaaaatgtcatgggaaaacacttctgacataaaagagcatgagaactgtatatttagcataatcttaactatgttttagaatgcacaggaaaaaaatgtacaaacatattcatagtgatgtctctggtggtaggattatgatcagtaagtacttctgtctcttcatattttcctgtatttgataatacatgcatatgttgtttttaaaataagaaaaattttaagtttaaaattggagctgaaaagtgtttttaggtcaggcgaggtggctcacacctgtaatagcaccactttgggaggctgaggcagtcagatcacttgagcccaggagttcgagaccagcctggccaacatggtgaaaccccatctctactaaaaataaaaaaattagccatgtgtggtggcacacatctgtaatcccagctacttgggaggctgaggcatgagaattgcttgaacccaggaggtggaggttgcagtgagccaagatcgtgccactgcactctagtctgggcaacagagtaagactctatgtcaaagaaaaaaaaaaaagaaaagcctttttaaacagtagcagacataactatataatccttactaagctgtcggtcaaatttttatttatatatttattttattcatttattatttttagacagggtctcactctgttgcccaggctggagtacagtggcgtgatcatggctctcttcaaacttgacctcccgggctcaagtgatcctcccatcttagcctcccaagtagatgggaccacaggtgcataccaccacacctggctaattttttttattttttatttttagagatggtgtttactatgttgcccaggctagtctcaaactcctgggctcaagctatcctcccacctcggcctcccgaagtgctggggttaccagcatgagccactgtacccagccctcaaatttttaaaaatctataagagacattattggacaattagagaaattcacatatggacttataatagtatcagagtgtgtggtgtgatggttctggagggaatggactttttctttggagacaggcttttctatgcccacccttttatcttgctaacttatcatcatccaggttccagcagaaacattacttcccccaggaaatttcttaagggtgcagtatcatgatgtctgcagcaaattctcaaatagctcaggaaaaaagtacgtgtgtggtatgagtgtgtgtatgtatgtgtgtatatatatacacatatatacacatatatatacatatatgtgtatatatatacatatatgtgtatatatatacacacacatacacatatatatacacacacacatacatacatgtatttttatataattatatatgcagagagtgcaaatgttgccaagttaaagattggtgagtctaggtgaagggaatatggtatttattgtattatttgtgcaacttttcttaagtttgaaaattttcaaaacaaaaaattggaggaagaaggcatgccagtctaccccaagccctccattggaatgctgaaaatctaaacaatgtgatttggcaatttcatttcttttctgttgtgggccagtagtccttagatgttggggaagggggtagtcgctgaggtgtggttgacttaggatggaagaagcagaagtcaagactcccagggtcaaagtggtttgctctgctgacccaagtgtgggaggcccagagtcagcgtttcaggtgtgctaattcagcatggttctattcacggccaaagtccaccctgggcacctctctggcagcaatcttgggtgactctactaaggccaggcctccatgaccctatgtctggatcccatatctccacctctcccactgtctcaggaacggtgcttagctttttcttttccctctcctgtcttctttgccagcatgtagaaagtttaaataattcccctctttacaacaaaacaaaacatacccccttcagtcaaccaccctagctctcttctccttttcccagccagatttttttaaaagcatcctaggccaggcgcggtgactcacgcctgtaattccagcactttgggaggccaaggtgggtggatcacaaggtcaggagatcgagaccatcctggctaacatggtgaaaccccatctctactaaaaatacaaaaaagtagccgggagtggtggcaggtgcctgtagtcccagctactcgggaggctgaggcaggagaatggcgtgaacctggtaggcggaggttgcagtgagccgagatggcgccactgcactccagcctgggtgacagagtgagactccgtctcaggaaaaaaaaaaaaaaaaaaaaaaaaagcatcctcagcactttggcaactccatctcctcccaacatgtccctgttactggaatccagccaggactcagccccgatctttctactctaaccagttgtctcagttaacaaggacaggtttatgctgcagtgacaaacaagatcccaaattcttgtggcttcacacatctggcaccacctcatcttccagccttaggagtcatcttttagttccttgaaaactctttacagttttctgttggggccttgtcatatactattcccctggaatgttctttcctatcccctccctttcaccttgctaacttgtgcccatccttcaggtctcagcagaaacatcacttccttggggaagttttctccaacacccacactacacaggtgtcccatctacactcctatgactttgtggtacttgtctcacttcattttccactgccttccccacaaggcacctgcacaagggcaaggaccgtaccactgtacctatgtcactcattgctgtggtcacctgcactctggctgcctaccttaactacacattagaatcacctgaggagcttttaaagccacaatgcaagactccaccctaggccaattggatccaaatccctggggtagggccagacatcagtggagttatatatacatatatatattttgtttgtttgtttgtttgttttttgagacagagttttgctctgtcacccaggctggagtgcagtggcgcgatcttggctcactgcaagctccgcctctcgggttcacaccattctcctgcctcagcctcctgagtggctggaactacaagtgctcgccaccacgcccagctaatttttttgtgtttttagtagagatggggtttcaccgtgttagccaggatggtctcgatctcctgacctcatgatctgcctgcctcatcagcctcccagagtgctgggattacaggcatgagccactgcacccggccatcagtggatatatttttaaagcactgcagagaattctgttgcatcagcttgagaaccactgatctgccttgtgcttcacatttaaaacttttttttaatgaataaataaaccccaaaaaattaatctccctaagcctccctagaagataggatggtaaggatattttcctaggtaaaaatatgttaatttcatatttcatgaaatttcatgtttcatttcaatcaagctctgtcatacaccttacatggggcaagcccagtgcctgggcagggtgtaattatactcattacacaggcaaggaaaagtcacattaggtgatggagcacaaataggcagttaatggtttcagggctagttaggatatgtttgtctttcaattgcaagtaatagaagcccaaagaaattggttatttatataatataattgattggttcccaaatttgaaaaattcaggaatagacccagcttaggtacagctggatccagtcactcaaacaatgtcacaaagaaccctttgacaggaatgtatcctgtgttgactctactttgctctgagtagtctttccccaggtgatgataaaaatggtcatcatcgccaggcttgtgtcctgtttagtaggaatatacaagaagagctcagtaaatgctggccccaccactaagcaaaaacaaaacttttgttgttgttattgttgttttaaataacagcttagacctttcttctttccttgttattctctttcatctgtaatccagttttctacttctgaagtatagaatgttctgatgatttattcttcattacccacaacttgcacatgtttatttaaaaatgccaggattgcctggccgttgtgtgctgttaacctttgtttgctgttagtggatccctgaagttcaggctcccaggggagcagataatgggtatccagttcctgcaatatccaccctctggcaagccaagttccttcctgggtaaggttttgcctacctgcattcctagggaagtttctgggcctgaccaccaagccagctctgagaaggggtgcataagccccaccatgctttggctctgtccctatagaatattttatgttgttactgaaaactaaaggaagatgggtgcggtggctcatgcctgtaatcccagcactttgggaggccaagacagattgatcactcgatgccaggagttcaagaccagcctggccaacatggtgaaaccttgtctctacaaaaacaaaacaaaacaaaaattagccgggtatggtggcatgcacctgtggtaccagctactcaagaggctgaggcacaagaatctcttgaacctgggaggtagaggttgcagtgagccgagatcgcactactgcattccagcctgggtgacagagcaagattctgtctccaaaaaaaaaaaaaaaaagaaaaggaaagctaaaggagagagactaaaatgatatcaggttcctggagaacaaacagacatgattttgcttcatggcaggacagccggaagaagtgggattatatcctcacattacaaataagaaaactgagactcagaatggttaagtcacttgtcccaggccacacagccagtaaattacagaaacagaatttgaacccaaatcttccagctccaaagcttgtgttcttttcactacctcctgcttaattttttaatttctaagattagacccttcatctatccatgacacctgcctgtcatcccctgaaaaaaggtgaacgccgttcagaaatttttctagcctgagctcactcccagttcacttatttttgctttgtcatggctgcccagtccccacttgtagaccaggaataggtcatggctgcggggactacacgctgtcgctgctgcaagggccggcctctgtttccggggctgagtgggggccagacctgccaggagcaccatcttctgtgggtcctgcctggatgtcacatcccggccccaagaagtcactgcaaaccttcgtattattgagcttcacatcctagaatttgctgtcactgtggctgctgcatgaagttgtcctgagagaaacgggcattgtcattaacagggaaattgatggtctgggggaaaagtcatcctcattctcttgcagatctatgggtgattgagactggctgatgttgaaggggtttctcagccatcgtgtgccatgttatggaacagtggtgtagccagccatttgacacccagcgctgacctttgtttaacaacctcacctatatatgacaaaatgattgtcagaaataatcgtgtaatgaaatgactgtaataatggccagaaaagaaacgcagatagtaaaatgtttctcttgttgaactctgtacatataattgcaccaggatttttttcaaataaaaagtaaatattatactacaaaaaagggaaaaagcacaagcatttattaaatagctttctatatctttctgagttttgatcctttgattgcagactgatgtaatattttatgtaaatcattgcttggttactaagtgaactttaagaaaagtgagacgtctgcagaagttgcccataatttagcagctactgtattgtaccattgatgtacggctttattttcttgattaattatttaaacaatataattcacaattttaaaataataaatttccacttaaaatggtatttaaactcagcaaaatatatcatctatgagtaaaatttgtatttaccaagcaaaaatattacagtttgtggttcacatgctgtctcactgttttaaattttaaatacaaaaactccaagtaggctgggtgtggtggctcacacctgtaatcccagtactttgggaggctgaggcaggcatatcgcttgagttcaggagttcaagatttgcctgggcaacatagtgagatcctgtctctactgaaaacaattagctgggtgtggtggcacatgcctgcggtcccagctactcaggaggctgagataggaggatcacttgaaccctgggggacagaggttgcagtgaggcaagattgcaccactgcactccagcctgggtgacagattgagaccctgtctcaaaaaaagaaaaaaaaaaaagaaacacaaaaactccaggtggtcgcacagaatgacaggactgaagtaacttagctccaatttctgtcttcataatcactgtcctaccattgtctgtgcttagaatctacttgcttaatgcaggaacatgtgttctcacagagatggaaaatgcaaatggcgccagaagcaagctggaaattctgaaccattaagaatttactctctgccaggcacggtggctcacgcctgtaatcccaggactttgggaggctgaggcaggcagatcatctgaggtcaggagttcaagaccagcctggccaacatggtgaaacttcatctctacaaaaatacaaaaattagccaggcatgatggtgggtgcctgtaatcccagctactcgggaggctgaggcaggagaatcgcttgcacctgagaggtggaggttgcagtgagccgagatctatctgcaccattgcacttcagcctgggagacagagtaagactccatctcaaaaaaaaaaaaaaaaaaaaagaacttactctcaaaataaatacgtgtggctgactccacatatggtagggccaactgtataactagaagttctccaaataacttctgtggagaaaaaaaagtttattaaaggttaacttttttaaagtgctaactagaaccttactaacactgagatcgcaccaattgtttataacttagacagggccgggtgcagtggctcatgcctataatcccaacactttgggaggccgaggcaggtggatcacttgatgtcaggagttcgagaccagcctaaccaacatgatgaaaccccatctctactaaaaatacaaaaattagccaggcacggtggtacacgcctgtaatcccagctactggggagggtgaggcaggagaatctcttgaacccaggaggcggagattgcagtgggccaagatcgcaccattgcactctagccccagcaacaagagtgaaactctgtttcaaacaaacaaacaaaaaaaaaaacctcttggaccaggaaaatattttttaagggaggagtattttatcactggcattgtttaggattgcaggcacatgatgctaatgaaaagcagactaactattagttggttttattactgtttttgaactctctctctcccttttttttttttttgagacagagtctctctctctgtcacccaggctggaatgcagtgactgcagtctcagctcactacatcctctgcctcctcagttcaagtgattctcgtgcctcagcctcccgagtagctgggattacagggcaccacaccaggctaagtttttgtatttttagtagaggcagggtttcaccatgttgcccaggctggtctcaaactcctggcctcaagcgatctgcccatcttgacctcccaaagtgttgggattacaggcgtgagccaccgtgcctagccctgtttttgaactctctagagacagtccagccccttattacttgtcctgaggcagctgctcccttcacctggccccccgcattgtgttccggacccttgtcctggtggtgctaaagaatatctctgtcgatcctttggggactggggaaactgaggcccagtgccacgcgatgccatttgttcagggaagattaggtcatctgctaggtccccagtcacttgaccttcttcccagacaggaagaagctgctctgggtctctcagtgctccacgtgtctttgcacattgaaatgttttctgatttttttttttttttttttgctgttacatttacttttaaaaaataacaagcaataaaatgttacatttgagaaggttgaaatgagaattgatttgagttaaattctagcagatttttcttagaagaatgatatcatcatctccagctacctgcaattgatctactctgaattaagaaagagacttccatttgttgtttatattttgcactcttgatgtgtttctttaaattatggtcatgggccaggtgtaggagctcacacctgtaatcccagcaccttgggactctgaggagggaggatcactggaggccaggagttcaagacctcgtctgtacagtaaattttaaaaattagccaggcatggtagcattcacctgtagtcttagctacttgggaggctgagatgggaggattgcttgagccagaactttgaggctacagtgagttattttcacgccactgccctctagcctggctgacagagcaagacctgcctcaaaaaaataagtaaaaaataaattaaatttcaatcattagcagtcattaggatatttaaatacagtatgttgaatcaaagttacgcatgtgtgtatttttttttccagagagttgtttatcatgtgggttttaatttaactttaaaaaaatgttggctggacagttgcccaaatggtatcatcagccatttggttgagaacgtatgtcctgcgggctcctctgtcactggagttttgctagctgacagccactggctagttagagactgcagtcagcacagatgcaggcgtggacttgcgcacgtaaccatgtcaatgcaaagccatcacttcttaaaaattctgaaccctgctgtctgagatggtggtgcagcggatagaactctgctctaagaggcagtagctaattccatgtcttctttgcccttgactagctgagtgactttgcacatggggcttgcctctctgttgccttgtctgcaaagtggaatcatcttttccttgctagacagaaggtggaccctggacctatggcctttttgagtttcccccccgcttcttagaaggacctctgatcctactgagtttaatacccacgggttaataattgggaaaagcaaaggaagcgcttctgtttaggtaattatatgcatgtttttgtctttttctggctggaaagatatccaagccactgggaaggtccgtggctacccagggtagccctctctggggagggctgctatatccaagagcccctcatgagaatttgaaaatcgaccatggtagggcctgctgacttttgacagctaatggtgtgctgagaattgtccctccaaagatgcctttccattccctcgggagagtctgggcagcccctactgggggctgggatgctggctcttccctcagcctccaccccaactgctctcttccctcctcccctccccagccccctaatttctctcacaaggctttgttctgcagcaacctttcctaatgcagtcctggcctcttcgcagcttcattacataaccttccgtggactcctggtccaaggatcaccccagaaagccagtcagaggtaggcacgcagctggggtccatttacttaccttccccaccccctcggaactcagaggtggtgcaggaatttggactccaagaattaacagctccaccaccatcaccagagccaaaactcaggatgcatgtgcttcatctgctgcttatttccagctgagagccagtggtgccatggttccttagggagccggtcccctgatgccggctcctggccccaaatctctctgatccgggctcttccagaatgtcttgtctccaccatcgcctttgaccaatggtgtccctttgcctggtaatgtcccctttgcctgatgatggccctgtcactcctctctttagcacagaggaggctgtttcatcccttcaagcctgccctcccttcaagtcttagctcaagttcaccttctccgcagagccttctccaatcttcttgactacgtctcctctcagctccagcaacctctgtctctggcactgattccttacttagctaagagaatcacagacacttggggctcaggacaatctgctttctctcttcttacccatggccttggactgtgtgtacctctttgtctccactcccaaacccaacccccagagggcagagagcatgttgtctgtccctttgctcagcatgaagccatgcgtgtggtagatcggcagagttccataacttgtgttgaccgaggggtcactttgctctgaaattacccctgtgtccttcagtatttgcacagatagcttcctggccagaccgaatatatccaagggcatggcccacctctgctcctgtttccaggtccctggtgggggttagttcatgccttcctcataatctgcccactggcctggtcctcaaggtcttcccaactgctcagccagagttgagaaaatgggtcgctccatcctgtttgtgtcgttctctccttcctggcccactctcctgcccacaggtatccaggggctgcctgtagcattagaggacatacatgcacatgcgtgggcatgggacactcacgtagcctccaagcacagcatcaataatgcattctgtgctttatagcatggaaagctgctctaaactttattacacagtggacatgtctgaagcagctcccaaatccacccctgagtgtgttggaattggcaagcctatcacttgggagtctagtttttttgttcgttaataatagatgcttcctgtggccccagcttggcaattttgatttaaagtgatcttaactgaagagactaatggacgggtctgaatttgtgccttttaagcacaaagtattgctcttaattaactggattctatcctttgagcaggcagaggccttcccccaagggcgtcattaacgatccacatctggacatcttccaaagccttcttctgtttcaggccaaccgcaggtgtgttcctgaacacccaggaggctatgagagccacatatgcctcccaaatacacacagtgtgcatgcccagggacatagagcagtgtgcaaagtcccattccatctctctccacctgggagaggatggctcttctgtctgattcatggctcaaagtggtaaaggagctccccactccccgtcccacgcctactcagagtctgcaaatatgtatgcgatatgagagctcgtcagttagctgtcttcagtgtggcgcacatttgaggagtctgactcccctccagcacaggccaatgtgcactgctctcctatctttgtacccccactgttgcactgtgcagaggttggagccatagaagtaccagagctgtgaaaggagaggccccctctcacctctgccctggtctccatccccactttctctaggaagctagtaggtgctgacaggggagagaagggaggggaggggtccagaaacagtggctcatgcctgcaatcctagcactttgggaggctgaggcaggaggatcatttgaggtcaggagtttgagaccagcctgggcaatgtagcaagaccctatctctacaaaaagaaaaaatgtaattagctgggtgtggtggtgggcacctgtagtcctagctacttgggaggatgaggtgggaggattgcttgagcccaagagtttgaggttacagtaagctgtgattgcaccactgcactccagcctgggcaacagagctgagaccctatctcaaaaaaagaaaaaaaaaaagaaaggagagagagagaaagaaaagaaaagaaaaaaaaaaaagaagggaagggaaagcccagaagagtgtggggagaggaggcggccgtcattctggggccctcagtgtgcacaaccagataacacatgctctgtgggcttttgtaccattttgcttgagcataaagaaaggaaggctgcccctaaatagaaagcactctggaggcaaacaaatctgactccaatcctggccctgccactttcccagctgaggacttagacaagcaccctagcctcttggacattctcagagccatctgctgcaagtgggtgctgccatacccaccttactgggcaggcttgggggaccaagggtggtaaatggctcagtctttcatgatgcggccacacagcaggtgcgccatccaggtccatttctttccttcctttcccccaaatcaagttgtcattaaagtactagtccacattaatgaaatcaactgtattaattttctatttgctgctataataaatcatcagaaatttagtggcttaaaccaacacaaatgtattaccttacagttctggaggccagaagccctccataggtgtcactgggctgaaatcaaggttttggcaaggttgcggtcctttctggagggtccaggggagaatccattttcttcctttttccagcttctaaaggtttcatgcattccttggctcatgatcttctatagctatagtcagaaaaattttccatcaatcatcttcaaagccagcaatggcaggatgagtcctcacatcaccttgctctgacaccagttctctgcctccctcttccacatgtcaggaccctcatgattactttgggctcactctgataatctgggatgatctctctattttagagtcagctgactgggaaccttaattccatctacaaccccaattcctctttgccatgtacagtgacatattcacaggttctggggattaggacgagcctgtctctgaaaggctactttacatgaaaattcatttttttaattaagatttttttttcctcttgagacaaggtctcactctatggttcaggctggagtgcagtggtatgatcacagctcactgcagcctcgacgtctctgggctcaggtgatcctcccacctcagcttccctagtagctggaactacaggggtgagcccccatgcccagctaatttttttttttttttttttttgagacagagtctcactcagtcacccaggctggtgtgcagtggtgcaatctcagctcacagcaacctccgcctcctgggttcaagtgattcttgtgcctcagcctcccaaggagctgggactacaggtgtgcaccaccacgcccgactaatttttgtatttttagtaaagatggggtttcaccatgttggccaggctggtctcaaactcctgatctcaagtgatccaccaacctcagcctctcaaagtgctgggattacaggtgtaagccaacatgcccggccccagctaatttttaaatattttttttgtagagatggggttttaccattttgtctaggctggtcttgaactcctgggctcaagcaaacctcccaccttggtctcccaaagtgctgggattacagcatgagccactgcactcggccttaagagaagatttaataattaatactttacaacaagatctggaagaggtgggatgagtaactaaatgaggatacaagtaacccgggtcatatttgctaatacccttggtcacattgaacttgatatcttatcagattttcctaatcagctcctttagcagcagtgttgcagcatcttatctcattttgttttttgtttttttgcctagcacatgcctgtaaatcactggattgaggtgtttagatgtttgttgtcctttggatgcttcttataaatccatatttcatggctccctggaaagtgctatgcaaatgataagctgcaaggatggaaaggaaattgcagtgctcctgaattgtaaatgggcttttacgaggaggtttctaattactcgctctttctcttgaactgaggagttgaagtgtaggtggcagatccataacagataatcatgtgtgtgatgtgacttcagcctgagcgtcgaggaccaagtcacagagcaggaacagccactctccagtgtccttggggctacgtctgaggagaacctgggatttcatatatgacctgcactggctggggggctctcttgacgtaacgtgttccctctgagcatgttacagattctgacattcttatgttccttctgtggagagacatgtacttagtgacctaactcactttagcatatttttgctcatcgtttgtgtagcttaaaggaatcagataattaccccctccccactactttcggaagcacaaatgcaatgccctagaattgtactggggactcaaaaagaaaagagagtagtaaaatctattaaaggggacaaagacagcctatatactacaagctttctatttttatggcagagaatgccattttctaagtaaacagagaactgcatttgacctgcaatatcaaatgcatggatttgatgctttggaaagcaactgttttctgcgttaatctgggtgtcttccgtgaaatgtcctcctgcctttggcttaaacactagctttgtctacagccattccatcctgaacctgcccaatcttgtctgaatcctggtttcaccactgacaagctgtgtgtccttgggcaagttacttcacctgtctgtgcttcagagtcctcatctgtgagttggggaatctggacagaatctaccccatagggcgtagtgaggatgtgttgaattatcccaagtggctacacagagtaagcactcaaatgatgtcatcgttgtcatgattgctgttaccagagcctagagttcattctgatactcgagtctgtggcccatccagcccaggtaaggaatagttggaggagttgggcatgttcagcttgaagaggagacgacaggggatatgggatagttgaatctgtgaagggccccctgggatgaagaactggcatgttctgtgtggctccagggcactgagcaggacccatttgccaaagtctcagggacacagtttctagctatagacagaaaaattttctgtcactcagaggatgaaaatagaatgagcccccttaagaggtaatgagctccctgtcattggaaggattccagaagagctaggtaaccactttaggtgctatcaaggggcttttttctttaaagtcctttccaaaagcttctgagattgcataaacaataggaagccatcttggtgctttaacacaaactctccccagtgatgagggttgagccaaagccagattggcaagcagagaggagacttgtgtacaaggagttcctcgagtcaattgctttttccttgttctagccagccagagggctcctgttggaaaacaggagaccggagaggctgaggcctgaccaaaccagcttctgcaggccagctgggaggccacaactcctacctacgggaaaactgaagggcatctctatttttagattagcaaaagaaaataaatttaagtttgagtctcctttgcaacttttaaaagacatctttattgagatgatcattcacattctataaaattcccccactttgagttacaattcagtggttttagtcttccttgatgattttgatggtcttttcttaaggctcttggaagacccagaagcctctcagacacaggtgggtgtggagggcgtagcacagaggcagacttctcatttcctgggtctcccctttaatgactctcagagacccctccttccccctgcccctggcttctaccccaggggtgtagagttttgccattttccaagcagaacttcatttcctcttctgtgtctacactctttgtgcttctttcttgccagctttttctcctttgcccgcccttccttccttccttccctccctccctccttccctccttccctctttccctccttccccccttccacccttccccccttccccccttccctccttccttccttccctccttccttccttccttcctgccttccttccttcctgccttccttccttcctgccttccttccttccttccttccttccttccttccttcctggtatgtgactaatttctgtttcaggacataaatgttgtccaggctgttctttggtctttctgttggataatggacatttggcattgagagaggctgctttttctgaaatcatgttcttggggcccagaacctaggtgtgtgcttctgactttgttttcttcctgatccaaattctgatatgtccatttaaattgatctagacccacagggcactgtgggacagatcctcagtggaacatgactctgtaacgagagcattttgttttgtcaaaatgagaacatattattgcctttcatctgattgtaaacataatacatgtttataaaacagtataatgagacaaaaatgtagacactaataagggaaaatctccctaattgtatttctcttcacagagaaagcccctgttgggcatatatactctagtttgtttatttgtttgactacacatatatgtattcttttcttatgtataaaaattctgaacatgcacatttctgcaactactgttttcacttgatgatgcatggacctctctagagtgtacgtttcttcttccttacaaagcagttggcttcgcccagggtacaccaggacacggttttggctctgtccccagggtgtcacgggaccaggggatgatctcacagggtctgccatctgccctgcctggccggaggctgcatcgagagggccaaggggcaccacgtgtcgtgggtactgtcaaacaagagccttcagagccttccacagtctttcttttgcttcccagcattgcttccccgctggtggactctgaatctagaactagctccaggcgcctctccaaattcagacgggagctggggcactattataatgcaaatctaggcaaagccctcccaataccaggatccagaatggggtggggccctttgccctgaaaagctgtttagtttgaaaatacaaacaggagacagaaaagtttggctaaattaatggataaagttttaacgatggtaaccatagtagggttcatcgacagccagcgatggttctgaacacttgacatgtattaactcacctaatccccacattttacagacaatgcaaaggaggctctgggaggttgagtgacttgccccaaagtcgcacagctcctaagtgaaggattcggagtggactccaggcagcctggtctgactccctgcactgcgctgtgcttatctctggccccaatgccgccatgcagaagtgtctgggggcactttgtctctgtcagacagaattcggagatgtgtatgcttgccctggtatggcacttctctttttttgagacagaatctcactctgtcaccctggctggagtgcagtggcatgatctcagctcactgcaacctccgcctcccaggttcaagcaattcttgtgcctcagcctcccaagtagctgggattatagatgtgcaccatcgtgcctagctaaatttttgtacttttagtaaagatgttgttttgctgtgttggccaagctgatctcgaacttttggcctcaagtgatctgcctacctcagcctcccaaagtgctgggattacaggcatgagccaccatgcctggcagtgtggcacttcttacgtgtgttcagcggacactgtttatcttctgtccctccaagacggtgctgagctcaggtcgttcattactggcagacaactgctgatttccaacagaattgccatcctcttctcccctgcgactttcagagtgtgacctcagactcaaaaattagaagtgaaaacatcttaaaaactatcaccttttcttcctaatcctcctctcccctccctgtcttccttgttgtccccatctaatgaactatcatggcaaaaagagcccatttctggtcattttctgtggcctttcaaactcccacctaccccactgctcctgggtgcattacccgaaagctgagacttcagtgcagaaagtgccaggccctctgtccccccagatcgccttccttgtcttccctgtgcttgcctgtcacattgtgtgggttccagcgctggaaggaatgaggaacagattctctggttctccttttgaagtttaccttcgctccaccacttctgagaccttcccggaagttgccccttgtttctctcctctccagggctgccccagagctgcctctcacctcttcctgctgtcaccccaccaccatcagggcagaagttgggacaaagcctctcctactggctcctgcttttctcccttaggtccagcctcctcttctccatcttcaggagtctccttctccactcacacgtcatgacttcagcacctcgcatcagtccagaatatgactgcttgttcaagtgccacctttctcatgcatttttttctagtgacaatcacagccaccctgtggggcaggagtgtcatcatccccatgtttcaaatgaagaattgcagttcagagagggcaagtgactggcccagcctcaacagctagccagtggaccccaccagggcttctgactccagtccgggttccctttccacccaaatccatggagggagctgagccgagaacaggtgtccttcaggaagacgtgaagccaaagcctccacctccaaactcaggggcccagggagtccaggcacccatccactcacaaggctggatatggtgcattccaggagaggggttgggggcgagtggcctctctgtgtacccgtggggatagatgcgcaagtggcatcgccacatcgtgagtcctggcttcatgggtgagctccaggtccaacgagaagccaagcagggggcccttcaagctcagctttgggcccgggtcggggtacagggtagagcgggcctccccagcccctgccatgaggccaaggcagtgcatcgttcgcagcgtacattcagaaaccaaagcctaggagctggttatcattccggtttacagctgatggaagagcaggtgcttccgagaacccacagtgctctttggccagtgacccaagggtgcctctgagaggcctcgcagcacccggaggtgctgctgaggcaacgccctgactgtaagaaggaccattcatcctcagagagtggccgtgatgctgctgcgacagtcccaccatccctcccgactctcactcccaacagacttcccactgtaaagctgaactctccagcaaatcacctctcgccagactctctcctcactctctctgggtccactagaggttcctcagcctctctttgccttggttttcccagctgtaaaatggagcaaagagggcctatgtacccacaaaggtgtggttggagcgactcctcctacattagggcctcgagtggggcttcatgattggttggtggaggtctccaaacccacccagtgccaccgaaggctgagactgcagatgcaatgccacaggtgtccttcctcagcctgggcagctgaacatcatgtgtaaaacggggataataagataataacagccccttgcacctatgtggctgtgaggattaaacaagataaatgtgtaacagtgcctggctatagaaatatttactcttgttattaagggaagaatatgtgtggctaaaaagggatcgaagatgtaaaagccaatccctccccctctagcatatttaagggtaatgttgagttggtttgtggaccatttgctgcctgttagagctggaaggtagggaccccctctcaacagcgatgctacaaattatacccattggaggtcaaccaaaagacaaagcttattggctggacatggtggctcacacctgtaatcctagcactttgggaggccaaggcaggcggatcacttgagatcaggagttcgagaccagcctggccaacatggtgaaaccccatccctactaaaaatacaaaaattagctgggcgtggtggtgcacacctgtaatcccagctactcaggaggctgaggcaggagaatcactagaacccaggaggtgaaggttgcagtgagccgagatcgcaccactgtactcaaaccgaggcaacagagggagacgcaatctcaaaaaaaagaaaaaaagacaaagcttgttaataccagcatattgttaagggaataaagtaggctgcagaacaactggtgtaatatggtgccatgtagggaaaattacatgtgtgcataggagaggggtctgcaaggttgtgccctaagatgttagagtggttcctttgcttttctcttttataattttgtatttgacttttaaataaggaccataaatcacttttataaaatacattctctccagcccctactactcctttaaagaataagagtggtttgcccaagaaagacagttttttttgctctggttttcttgattctgacatcagaggaaactccttctcatccacttggggctctgggttcaggggattcatttcaggcagattaaagtggtgaccaggggcattcgtggacacagggagggacaggagcaccatcagtttgtctcacacaaccactgtcatcctcactgaaggctgttgcctgatcaaaaacagtattgggccaggcacggtggctcacacctgtaataccaccactttgggaggctgaggtgagtggatcacttgaggtcaggagttcgagatcaacctggccaacatggtgaaaccttgtctctactaaaagttcaaaaattagccaggcgtggtgggtgcctgtagtcccagctacttgggaggctgaggcaggagaattgcttgaacccgagaggtagaggttgcagtgagccgagatggcaccaccacactccagcctgggcgaccgagggggactctgtcttaaaaaaaaaaaaaaaaaaaaaaaatatatatatatatatatgtcaaaaatggggtagtttttagatctatagtagttctaaaaacaaaggccatccaagcatgacagatttacaagcactattggctattccagtagttacaatggaggagagaagcttttagttaaaacaaacaaacaacacaacaaacccagaaaccttaggtcaaaaccaaaattgtcctctcagacacaatctgggaattttctcatgacagtgggcattagccaactgacatcagcagcaaccatccgtgtgcacacagtggcaccacctcctcccaaaaagcagccttcatctatgccctcatacaatcgttgattattctctttggattgaggcccggaattatttaagtttcttcttgccagcatgagtctttcctttctgtatgctccttatcttctctctttaatttggcagttctgcttgaaatctgggtctttcattagtagtagttcaatttggttccagaacattctgtggtgtgatgcaatgtgaccagagctcacacttcagagctcttcaagggccagtcttactgagcacctcccagtggctgcctgtgtgctgggcgccacttgtggtgggcaggagagaggaggggacacaaaaggagacacagctccttcttagaagctcaaagttggggaccagctgccacagaagagtatgtttagcatctgagacaccaagatccagcgtcacaagggtgtttattaagcctcctcatctctttctttttctttttttttttttttttcctcaggcagtcttactctgtcacccaggctggagtgcagtggcatgatctcggctcactgcatgcaaccaccacctcccgggtttaagcaattctcctgcctcagcctccccagtagctgggattacaggtgcccaccaccacacccagctaatttttgtgtttttagtagagacagggtttcaccatgttggtcaggctggtctcgaactcctgacctcagatgattcacccacctcggcctcccagtgtgctgggattacaggtgtgagccaccgcgcctggccttgctgttgattcatctatagtatgtttgacttgatgacctccagttaccttagacagaggttctcatctaagctccaactttccatttcctttgtcctcgtctttccccttaacccctccacatttctctcaaaatcaccccacttctaaaaaatactgtttatttttcttttaaatttcaaattatctatactcattgaaataaatcaaaatagcatggaataagcgaaaaaaatggatcccacccttccccactcccattccctagggctaaccatagttaaccatttaatgactaggtttttttgttgttgttattttttatttatttattttgagacagagtcttactctgtcacccaggctggagtgcagtggtgtgatctcggctcactgcaacctctgcctcccaggttcaagcattctcctgcctctgcctcctgagtagctgggattacaggtgcctgccaccacacctggctaatttttgtacttttggtagagacagggtttctcaatgttagccaggctggtctcgaactcctggcctcaagtgatctgcccaccttggccttccaaaatactgggattaaggtatgagccaccgcacccagccctcctgggctcttttcctttagttgcactcgctccccgctcctggagtagagggatttccgagagactgtgggctccagccttcacctaggcccaggactaggatgcctgccctaacatttatctttataccttaaagcaaaacagctggaccataagcattcaagaacaaactgtgaataaggagaaagttctcccaggaaacaagagctttagttatgttgggccagcccttatattccttagctgttaccagtcactgcttgatttaatctcggctatcacttggcctgacaggtctgctgctggtgccaggatgtctgggttttgaagcctggctccattacatacttcctgtgtgaccttgggcaacttactcaacctgtctgttcctcagtttccccagctgtattatgtcagcataatagtttgttgtgtgaattaaatgaggtaataactggaaatgcttcaaacatggttcctatcatgagaaatcctgctttccgcctaaatgtgctggaaaattcctggtggtgcagaacaggagaccagagcaaaggaaagacagggtgcagaagccaaaaattaccttggagaacaaagcgcatgttaaggttatttttggattctaggtttatctctgcttggtcttcagttacctacaagagatccatttaggggatttttgtttgtttttaacgatagctttattgagatataattcatatgccataaaagtcactcttttaaaatgtttccggtatattcacaaggctgtgcagccttccctgtccttgattccagtctgagtttttaactgaagggataaggaggaccacgctttccccagaccagaaccgcgggccagggggcgattccgctgagtcaccgcgggcgcctggtgcgcggcggcggagcccgggaccttccttggctgccccctagcgagggccgcagcgcagcctgagacacccgccggggccgctccacggccgtcggatttagactggaagctcggtccaggtccccagcttgatgcgcccgcggtgtaggagaccagcccgactcgagcttcccctgagcccctggactcttgactccagcagggcctgggtaatgaacgtcagctcccctttcccaaaggggttgctctgttgggaaggcacccgtttgatacagtagcatagagatgggttttagcatcaaaatatcagaattcaagccttgctctctgcttactagctgtgtgaccctaaaaaggtttctgaacgtctctgagcttcagtttcctcatcattccttctcacggggtggttgtgagcattacagagatcctctctgtgaagcccctgtgagtggctcatcctgagggctgaaataaacatgttattaataatccaaaactggcaagggatgttgactggtccccctcccttgcccaaggagctttctagaacctgagttatcattaccaaactgtactgccttgagtaagaaagttagaaggaatgggaaggatggtggcaggtggaggaaggcggattggtcatcacctccttgcagcaagaaacagccccagatcgtgggaaacctacagacctgctagacagactaggagcaaaagctggggctttaagaatccccagggaggttctcctgagagagtagccagttggattttgtaagcagagatttgtttggggaggaggtgacaacgtagggagcagaggggcaaagctgtcgggaatcctgccttgagggcagggatgtgtgttggggggagttgggtcactggggctcggtggccttgggcaagtttctacctctcaggtcctttacccacctagggtcgccatcctgcccacctcacaggttacagtgagcctggatgcactgtcatgggcaggtgcccaggaaaatggcagacatgttccaaacagcacgcagcattccccagtgatgcccagggtcaccttggaggtgggcgagatgcctggggtttctcgtccaccccacaacacctcaggggacagccaaagctgtcccttcaggtaagctgcacagaagatgtgaactctgctgcaaagactctattctttgggagcaaaagggacccagggtctcacctgcacatccctgtccctgagggcctaggggttcttggaggccccagccttggcaaaatgaggaagaaggtgaaggttgtctgggcccctgccaggctccttcctcggccacgcactccccttcctgcacacacacccttctccctccaccccatctccattgttgtcagaaaagtcacaataaaaaggtccatattgtctagttcccatacttttaatttttaaaattttatttatttatttatttatgtattttttgagacagagtcttaacccaggctggagttcagtggcatgatctaggctcactgcaacctctccctcctgggttcaagtgattctcatgcctcagcctcccgagtagctgagattacagatatgtgccactatgcccagctaatttttgtatttttagtagagacggggtttcaccatgttggccaggctggtctcgaactcctggcctcaagtgatctgcctgcctgagcctccggaagtgctgggatttcaggtgtgagccaccgcactcggctccacacttttcacttattaaaagactgtggtgtccatcaatggatgaatgaataaaccaatgtggactatccctcccattacccaaggaatgaagcacggagccgtgccaagatctggattcacagtgaaagaagccagtcaccaaaagccacgtgctgtgtgacttcccttatacgaaatatccagaagagatacatccatggtgacagaaagtagatgagcagctggggactggcgaaggggagaagggggagcagctgtctatgaggtccagcctttcttctgggtttggtgagaatgttttggaactagatagaggtgatagttgtacaacattgtgaatgtactaaatgccactgaatcattcattttaaatcgttctttacgttgcatgaattttaagtcaatcaaaaacagttgtttgaaaagagaaaagcctatgggtagcggcagcagtgattggatttatgattcgattccatggctcatccctcccctgcctcaccccctcgccctccgacgtcttcttcttttactctgaactgttatctttgttctcatctctctctctctctctcaaccctgcagacacttttccctttctttgtctgcccccaccctccagatttccgtgtctccagtgtctccctacgaggcatgaattgagactgggagggtgtgattctgaagaaggcaccaacagtgactcagctagccccttcccccaccccgccccccgggcctcaatttagctaaaaaaccacagggacggactcaggaggcaatacctttccaagggtccctaaaaaatgtcccattttagtgtccaggtttcactcaactttagtgcctcccctaaaatgtgttccttacctcccaccccactgcatctaagtcactgcctgagaaaacaggattgaggaaaggagaaaggaagagagagagagaggaggagagagagagagagggaggaaggctgatggatttagaaaagaagaaaacaagtggtctgaggaaaacagccttggtgtgtttattttcctgtctgtgtatcgcttctcggccttttggctaagatcaagtgtattttcctgtctgtgtgtctcgcttagattacagggatctgtgggtgatgacacgtctggtccaggctgcgtagtcacctcaagggcatgcttattgatgtgtttttcaattcactatctttgcatgggagtcccaggccaagaggcacagctgcgccatttgtctgttggtttagatatcctttatccagttcttccagagaaatcatcctgcccttctggaggaggtgggcagcaggggtcagagatgggagggaaaggaaggagccaggtccttggctaggatgccagggtcccctgcctctcacctggcctgggctggaggcctcctgctgtcctgtcactgatcactaccccgccccagcctcctgagttagaagacacaggctaaagtagagtatttcttcattgaaaaacccatacaaaataaaggttcataaaaaataaaaatttagactgggtgctgtggctcacacctgtgatcccagcactttgggaggccaaggcaggtggatcgcttgagccctggggttcatgaccagcctgggcaacatagtgaaaccccatctctacaaaaaatacaaaaaattagccaggcatggtggtgcatacctgtggtcccagcttctcagcctatggacccacatagaatacaatgtcagcataagaagggagccctggggtcaccaaatggtttgggcggcaaagaacctgaaggttgagagaagtggcttggttacccagctgttggatgtgagacctggccactgcttcttccataccctagacctgcaccctgacatctcaagtaaaaagttgggggatgttttatggtccaggatgaaggaagggcagtgaggggcagcggagcatcactttgcatttctgtctgcctcttactggctgtgtgacctggggcaggtaacttcccagactcctgggaatcataacacctatgatgatgatgatgatgatgatgatgatgatgatgacacctacctcaaggattgccctgaagggtcacagagatgcctgcaaggcacctgcatggagcaagcgccccttctctggcaggtgctgggtgagcactacctgctgccaggccctggggctatggcactgcgtgaccctgcaagtcctacctggcgaagctgtcgttcttgtgctcagtcagtgttggttgtaagactgagaagagtcacttcattttgctctccagggacatctttctgggtcctattttctgcctatgtcaagtagcgcctcaaggatgctcctgaaaatgggcttgtctttcttaacatggcaggtaggtcccaaagcattagcatggggcagctgacctagcccagccaatgcagtgcagtgactcttgcaaccgagtctaatcagaaggtccatgaacctacgagcatttcctgtcccaggatcagggtggaggctgagcctccctgcttagagattcttcccatgcattccacttttttccccaaaagaaaatattgacccttgagaggcacacagtttatttattttgcatagtaaatagtagcctgtattttaaggatgagttgatttctgcatcagcccctgtaggtcatcagccttctattggtgcatctgactctctctagccctgcagggatggtggagggggaggggaaggagggatctttattggaaaccaggacagtgagactcattgccctgtcatctgctctgtggtgctgaatgaggcagcccaacagagaaataccctgagcgagcatccccagcctccaaaacagtggcgcattgccctgagtcctgggaatgacctttgattctcctgctcctgacttggaacccatggaaacctctagaagcagctgaggaaaacccaacatgaaaagcagaactccacactgagaatataggaggtgatcggaacatacaatgattcttgctaagaccgattcacagtttttcttttttttcgatcgaagaaatactggagaagcctaaagaaggagtctaaaaactctggcacgtgggccaaaactgtccttgagctaagaatgattttcacatttttaagtggttgaaaaatgaaataaaataagatgatgttttgtgacacatgaaagctatgggaaattcaaattctaatatctataaatagtgttttatcagaacacagtcatgctcatttatttatgctcgatggctgctttcccgctacaattacgttgagcagttacaacagagaccacgtggcccacaaagccttacaatatttactatctggccctttccagaaaaaaatgtgccgactcttgaccttaacctcagcaatttgggaggccgaggcaggcggatcgcttgagctctggagttcatgaccagcctgggcaacatagtaagactccatctctacaaaaaatacaaaacattagccaggcatggtggtgcacacctgtggtcctagccactcgggagactgaggtgggaggatcgcctgagcccaggaagtcgaggctgcagtgagctgtgatggcaccactgcacctcagcctgggcgacagagcaagaccttgtctccaaataaataaataatgcaaagtaaaataaataaaaccatataaaaaggaatcaatttaaaattataatgaaagctggccgggcatggtggctcacgcctgtaatcccagcactttgggaggctgaggtgggtggatcacgaggccaggagatcgagaccatcttggctaacacggtgaaaccccgtctctactaaaaatacaaaaaaaaaattagccgggcacagtggcgggcgcctgtagtcccagctactcgggaggctgaggcaggagaatgtcttgaacccgggaggtggagcttgcagtgagccgagatcgtgccacttgcagtccagcctgggcgaaagagcgagactccgtctcaaaaacaaaaacaaaaacaaaaacaaaaaaaaattataatgaaagccaaggggcatagtagaacaaattttctagagctcattaagtcaaatgagtcaccagttagtaaaacgcagtcacggggaagagagggcaggattctttgaagcagcggctctcctaaaaacaacccacccttgtccagctgccttccctcctgagggtgttccctttgactgtgtgacccccatcccctatttcccaaccgtccaagcccacctctagcataatacgagcttttaatccctctccctgaccccaacccgattttgaagcccagtctagtattttctcaaatacacttcttggctccattccttcctttccatcacctctgccttttcactgcatgcttggaccactgcagtcagctccctatgaacagttgctctctacccatccaatcggccccgcctgctgctgccaaattcaccgagggcacctctgtggtgctgcctgtggacaaagtccaagccagccacctcacccacctacaggtgagtggggagcagccagcgtgtccagtggtttaccccatcgccacagacttggtgatgtgtcgatgtgcagagaaggggtgttggcagccacaacacaagcaaccccgccccatgtgagatctaagatgggcgtgctgggagccacctctgagaatccaacagaaggcagaggggagaacggctcacacggcacaaacactccttccttttttttttttctttttcctttttgaaaggagtctcactctattgcccaggcaggagtgcagtggtgcaatctcagctcactgcaacctccgcctcctaggttcaagcgattctccagcctcagcttcccaagtagctgggattacaggtacactccaccatgcccggctaatttttgtgtttttagtagagacggggtttccctatgttggccaggctggtcttgagctcctgacctcaggtgatctgcctgccttggcctcccaaagtgctgggattacaggtgtgagccatggggcctagcctccttccatttaaatgtatgcctaatttgcccattgagaacggctgagacgcattttaagtggccagggtctacttagagttagtgctcatgaccaggcccaggtcaagcctggctggccagatggtgcctttgacctgctctgtctctgtgcaaaggaatgagctgaaggatgggggtgcagtgtgtgggcagtgggctggggctggcaggactcagtgactaagggaagagaactttcctcactaccagcctgtcttttcagggcaccgcggggggctttgggacttggtgatgaacacagcacagagagctgtccagcatgcgggtccctggcttctcacacttcccaggctccttcagaggctctctccaaagggagctgctctctctagaacccatgaatttggaatataggcaaccactgcattggggaccactgacctcaaacatagagaccagagcaaatggggctcatcacgtgaaactcatctggaactctagcaggttcttttatatatatatatatatatatatattttttattattatactttaagttctagggtacatgtgcacaacatgcaggtttgttacatatgtatacatgtgccatgttggtgtgctgcacccattaattcatcatttacattaggtatatctcctaatgctatccctccccactccccccaccccacaacaggccccagtgtgtgatgttccccttcctgtgtccaagtgttctcattgttcaattcccacctacgagtgagaacatgctgtgtttggtttttttgtccttgcgatagtttgctgagaatgatggtttccagcttcatccatgtccctacaaaggacatgaactcatcattttttatggctgcatagtattccatggtgtatatgtgccacattttcttaatccagtctatcattgttggacatttgggttggttccaagtctttgctattgtgaatagtgccgcaataaacatacgtgtgcatgtgtctttataacagcatgatttatattcctttggttatatacccagtaatgagatggctgggtcaaatggtatttctagttctagatccctgaggaatcgccacactgtcttccacaatggttgaactagtttacagtcctaccaacagtgtaaaagtgttcctatttctccacatcctctccagcagctgttgtttcctgactttttaatgatcgccattctaactggtgtgagatgttatctcatggtggttttgatttgcatttctctgatggccagtgatgatgagcattttttcacatgtctgttggcgaactctagcagcttcttttcacaagttcatggagagaggtttcccactgagggaatcacatctgtctgatcaaaagaggcttgggaaatggctctcctgttcattccctgaaaacctctgatggaaccactgccactgtggcagccccagcactggcaccccagccatgattggtgccccagccacatctctgctgtgagccccagagccctggttaattaatcatccacgtgttgatggggagaggcccattcacaaaagcgacataaagcccagggagacgtggccgtggcaagaagggtgtgggactacattccgcccccaactgagagattcagaaaccagaaaaaaatggaaaaacatactgtgctcttgggtgggaaaactaaatatcatgaagggagcaatttttatagttttggcctataatacaattccagccgaaatcccagtggaactttgagaatttgcaggaaaaaaaaaaatgtctaaagtacatctggaagacaaacttacaagaaggtcaaataattttgaaaaagaaaatgatatctaagcccacctagagaataagacttgagatccaaagctaaatcaggaggctctagcaaaattgacagataagcaggacagagtgcatggtgcattcacctggggaagagggcagattggtctacaaataggcctgggtccactgactttagctgttatatttggggagaaacttttcaacctcactccatcttaaacctaaaaatattccagatgaattaataaatataaaaaattagaccactaaaaatgtagaagaaaatggatgatctttctataccatagagcaatggaataaatcacaaaggaaaacagatttgactatataaaacttaaaccctgcccatcaaaaaccatcagaaaccaaaataaaaggcaaccaactggagaagatagttgccacaaatatgatcaagggttaatgttattcataaattaagagcccacacaagtcattagaataagcactgagacctgaacagacaagcaaaaagaatgagagtgggtcggcgcggcggctcatgcctgtaatcccagcactttggaaggctgaagcaggcggatcacttgatcccaggagttccaacaccagcctgagcaacatggtgaaaccctgcctctacaaaagtcataaatattagccgggtgtgatggcacacgcctgtagtcccagctactcaggaggctgaggtgggtggatcacttgagcccgggaggtagagtctgcagtgagccaagatcacaccgctgcactccagctggagcaacagagtgagaccctgacttaaaagaaaaaaaaaaaaaaagaggagaaaaatgctgatctcactagtaattaaaacatcaggccaggcgcagtggctcacacctttaatcccagcactctgggaggctgaggcaggcagatcacttgagatcaggagttctagaccagcttggccaacatggtgaaatcccgtctctacaaaaaatacaaaaattcgccaagcgtggtggcacatgcctgtgatcccagctactcgggaggctgagacaggagaattgcttgaacacgggaggcagaggttgcagtaagctgagatcgtaccattccagtccagcctgggctacagagcgagactctgtcccagaaaaaattaaaacatcacatatttaaacaactctaggatatcatttaaaaaaacattaatagactgttttttagagcacttttaggttcacagtgaaactgagtggaaggtacagagacttcccgtatgttccctgccctccacgtacagcctcccccactgccaacgtcctgcaccagagtggtacacttgttacaaccaatgaatcctcattaacatatcattatcacccaagttcatagtttacattagtaaaacatcatctttcatctataagcacaaaaattttttggcatttatttaggtgtatgattaactcagtgttgacaagactcacacttcatacccacttgcactgcatctgagaagcaattggtgtctacagccgctacaccctcaacaagcccgatcttgtttgaaaagcaattggtgatgcttctcaaaattctatggacaaagtcagccgggcatggtggctcatgcctgtaatccctaaactttgggaggccgaggcaggcagatcacctgaggtctggtgaaaccctgtctctactaaaaatgcaaaaattacccaggcatggtggctggggcctgtaatcccagctactcgggaggctgaggcaggagaatcgcttgaagcaaggaggcggaggtttcagtgagccaagattgcaccactgcactccagcctgggtgacaagagtgaaactccatctaaaaaaaaaaaattatggacaaagtttttcaaaaagatatttaatgcaactttatttgtaatattggaacatctgaggccatttcagtgctaactattaggggatggttaggaaaatatggtacatatgtggaaaggaacatttggtagttagtgcccctgatgtttacaaaggcttttagtgaccaacaaatgctcatgctataatcttatgtgaaaaaagcaagtagcataattgcaactatatttttaatgcatagaataaaaggctagaaggaaatatcacagatccttgacatacattcccaaacctttgtaaatccgcggattcatgaaaacagacacatttgcacaagtgcctgatcttttctgttatacattcattagaagtcaagccctggtgccacaaagtatctgccttttcaaatgtgatcagaatgttctcttttgcttcaaggccatttttcacgaagcagtggcatttttgcctcttcatcagagtcaccgtgtgccctggaggactgagaacagcagagccgttttaggatgggacagggcagccaggaggattgggctcactccctactgagtgcctcactcccgtacagcccccatagaggaagaggggttcaaatttattcctcagccagatggcatgtgccgcctgtcctggaatttcacatcacttatgatggaccaaaattccaaaagctgaatccatgattgtcaaagtctggtatggcaggatgtcaacagtaatcgtttctgggcagagggatgattttctcttcccatcttgctttgtataaatacattttctataataaggttgtattacttttctcatcaagaaatagcaaagtactgttttactcaaaatatgaatagagccaggcatggtggcagcttatgcctgtaatcccaacactttgagaggcggatatgggaggatcactttagcccaggagtttgagaccagcctgggcaacatagtgagacccccgtccccactcccccaaagaaaacccacaaagcatttatcctggattattcacaggggccaaaaaaaaaaaaaaattcaggcctcctatagccatgagctacgaatatgaaaatatgcaaatgtgtaagaaaagccagcacatccgatttttacttttactttcacacctctgtccaccatgttccaagagaagaaacttggtcattgaaaggaatagatcaaatccaaagaacaaaaccactgtgctcattaaacttcttagtgttcacaaagctttagctgcaggttgaatggggcaacccgaattggctggctcacctgggctgcagggagcagagatcgcgacactgcactccagcctgggcaacaaagcgagactctatctcaaaaaaaaaaaagttcataaattcaaagttatgaattatttttaaaataataataatttacaataaagatgaggacaaagtgtgagtaaatggtggtttctatccagctctgttgagctgaagtggcatctccctgctggggcttttggggaagaagggtgtgtgttgctcttcagatcccaagcctcatgcccctactgggccctgtggggtgcttctcagcccaccaggagagccaccgttggaacacacacgtgggggacctggtgggtgccggtgtggtgaatgggggccacagcctgactccaggaagccagcaaactcggagctggaggagtcaggacacccccgatgagtcaagagttggttttgctgccagttgacatctgattgaaccatctcttcacttctccgtgcctcactttccttaccagacaggctctgctgatgctgtccctctcctgttcagtcgtgccctcaccgttaaagagaaagagcaaactgctgggcagcagcattgatttttttaatgaagtggaaagagagctgggaataacaagtcgggcccacctcacctgcctcacctggtgggtttatttgttttgtttttttttttttgttttgagacagagtttcaccctgtcacccaggctggagtgcagtggtgtaatctcagctcactgcaacctccacctgccaggttcaattgattctcctgcctcagcctccccagtagctgggattacaggcacctgccacatgcctggctaattattgtatttttagtagagatggggttttaccatgttggccaggctggtctcgatctcctgacctcaggtgatccacccacctcggcctcccaaagtgctgagatcacaggcgtgagccaccatgcctggccgtcacctggtggtgttgaatatgaactgctgcggtgttggtaaattaagcaagcagatagatgtaaataacgcttgggcaggaatatggagcacgggatgaggatgggcggccaactgttagagagggtagcagggaggctgagatctgcctgccatgaactgggaggagaggctcctctctctcttcacccccactctgccccccaacactcctcagaacttatcctctcctcttctttccccaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaaggttagtggacagccatgcacagcaggcccagatcactgcaagccaaggggtggcgggaacagtttgcatccagaattgcaaagaaattttaaatacattattgtcttagactgtcagtaaagtaaagcctcattaatttgagtgggccaagataactcaagcagtgagataatggccagacacggtggctcacgcctgtaatcccagcactttggaaggcccaggcaggaggatcccttgaggccaggaatttgagaccggcctgggcaacatagcaagaccccgtctctaaaataatttaaaaattagccaggtgttgtggtgcatgtctatagtcctagctactcaggatgctgaggcagaaggatcacttgagcccaggagttcaaggttgcagtaagctgtgattataaaactgcactccagcctgagcaacagagcaagaccctgtcaaaaaaaaaagaaaagaaaaaagaaagaaagaaatttaccttgagttacccacatgagtgaatgtagggacagagattttagggccttaacaatctctcaaatacagggtactttttgaggcattagccacacctgttagcttataaatcagtggtattgattagcatgtaaaatatgtgactttaaacattgctttttatctcttacttagatcaggcctgagtggcctctctttagcaagagttggttagccctgggattcttactgtagccacattaataaacaacatcgacttctaaacattctataataccatcttttggccaaattgacttcgcctcttcctctctctttccaaatgaaatgtgtttcatttcactgtcagaccacatggttggggaccccacagagcacacagccctccctctgccttcccatgctggcccttcacccactgctggagtgccaggttggtccaagggttggaccaagttgtctgaggttgtctcaaggttggtcgaggctgtctccgcgctgggttgtgctacaaggagcccttctttccatgggtgtggctggcagtgagtgctcacagcaacagcccacagtgcagcccgagggcaggatggactcagtccctgcctccatacccatttctaaggaggcaaaatggcaaacactctacttttctcttttaatgctaaaaataagaaaacaccttgcagcccagggtatgggtagtgcatggaagccgtggagttgtgaggtgggaagtgacctctgctggatatgtctattcaggaagattgctggagtgggtggggtctctgggaggtcccctgagtgtgggaagctgggaccaccagctttctcgcacagggagtggccatcccagcttggagaggttccaggactggttgggaggcacgtttcagatttctatctgttgaatcagcgaagatattggattatgaggaatttgggaattaggaaagtgggtgcaggtgggttgggggtaggtgaaggaagacatgggcgtattgggggagcaggggctgctcagaggtgttccagaagctctgggtgaggaggtgagagggaccggggaatgcagctcggcccagcctccctgcctgaggtcagccatcacgtggtgatggcaagatggaaatgtgctttctgactgctccagccagtgctgccagattcagctccccagggagggcacctgagaggctccaagccaggagatctgttttctcctttgttttgtttttttttgttttgttttgttttattatactttaagttctagggtacatgtgcacaacgtgcaggtttgttacatatgtatacatgtgccatgttggtgtgctgcacccatcaacttgtcatttacattaggtatatctcctaatgctatccctcccccctccccccaccccctgttttctcctttgaatccttcttagaggccgggtgcggtggctcacgcctgtaatcccagcactttgggaggctgcggcaggaggattgcttgagcccaggagttccagaccagcctgggcaacatagtgagacctcgtctctacagataataattttaaaaattatccgggcatagtggcatgcacctatagtcccagctactcaagaggcagaggcaggaggatcacttgagcccaggaggcggaggttgccgtgagccaagatcccaccactgcactccagcctgggcgacagagacccccatgtcaaataataataataataaataaatccttctcagtcccttcctcactgtgtccccctccactgaatttttccacctcctctcccacttcccccactcccgctttccctctccttctctccccactccatctttttctttctctgctgtttctcgtccctccctcctctccatcccacaacactgcctaccctgtccctgccccaccctggtgctcaggatgtgtgaagtgaggggtggtagcccccaagacctcaaccccgaaggttagcctgttgaaaccactttctcccagctgcccccctggcagttggtgctgctgggggaaactgggattgggggccagattttgcctcttttcctgacaaagagagatgaagagttctctcaccaggtgcctgggactggggtgtgggtgtcccagcctatcccagcgcatctgttctgcatcatgattaatagtgctgctttcagccgggcgcggtggctcacacctgtaatcccagcactttgggaggctaaggtgggcagatcacaaggtcaggagttcgagaccagcctggccaacatggtgaaacctcgtctctactaaaaatacaaaaattaaccaggtgtggtggtgggtgcctgtagtcccagctacttgggaggctgaggcaggagaatcacttgaatctgggaagcagaggttgcagtgagccaagatcgtgccactgcactccagcctgggtgacagagcgagactccgtcctaaaaaaaaaggagttttgctctgtcgcccaggctggagtgtagtggcgccatctcggctcaccgcaacctgcgcctcccgggtgcaagcgattctcctgcctcagcctcccaagtagctaggattacaggcgcctaccaccacgcccggccagttcttgtatttttagaagagacggggtttcaccctgttggccaggctcgtctgggactcctgacctcaggtaatccgcccacctcagcctcccaaagtgctgggattgcaggcatgagccaccgtgcccagtcaactccttctcaaaaaaaaaaaaatagtgctgctttctctttcaagtgtcctgatttgggtgatagtaaatgccactctacttataagggatctacctcagaatgctaattgggacatttttgtagcactctactgttggcagcaggtgatgctcacaacagcccgtgagggtggatgacgtccgcttcacagatgacaaaggagcctcatgctcagaccgtgggctgccagagcaggtccatggctgcagccccacatggaccatatttcccccttgtcactctttccaccaagctcccttggaacttcagttattaagctctcttgggtggaatccaagttagaatcacaacatgtgcctcatatggattgtgccagtgaaaaatgacattctatttagaggcagggcagcctggcttagagtcagtttaaaatatgtattatgctgcaacaaatgtaccatgatcctgtaagatgttcacaacaagggaactggatgtggggtatactgtctgtactaacttcacaagttttctgtaaatctaaaactgttccaaaataacaagttcgtttaaaattaactccaggagaccaggtacggtagctaatgcctataatcccagcacttcggaaggctgaggcaggtggattgcttgagcccaggagtttgagacaagcctgggcaacatggtgaaatcctgtctctaaaaaaaatcacaaaaattagccaggtgtggtggcgcattcctgtagtcccagctacttgcggggctgaggtgggagaatcatctgagcccaggagtttgaggctgcagtgagctgtgattgtaccactgcactccaacctgggcaacagagcaagaccctgtctcaaaaaacaaaaatgaaataaagtccaggaaagaagtaggttttaccactcttattttctgaagagaaaactaaatttaatgtgtaaagtgaggacaagttcaccaagttagtgtttgagttgcctaaaatatgtttgctaaaactattcaaagctttcacataaaacatgatcagaagttctatgccaaaacatatgtgtgtgtatatatatatgcactatatatactgtatataaaaatgcaaaatctaaattgccaaccttttagaaattgctctgaaaggaaagcatttcaagataatttgcttacccaaagaatatactttccaagaaagcaagtaatacttaaggtgttcataatcctcatcaaattaattcttgctactgaaagcttacaaggagctgttttgatgtcgggtgtgacaggtttgacttggcagaaggtgtcactttactaacaacattttaaataagtgacagaagacaagaaactacacgttaaatgccagaacaaagagtgtctaagtggatgctaagagttgaaatatggctggatacctgcccaagagagctgaaaagtagatgaaagttggttacctataaactagtgcaccctaatgaattaaaaggtgttgatgagttaacttgttatgccttccagataagacatgcaaatggggcttcttcctccttcactacttccaagggatttaacaaggagaccaatgcaaatgataaggactgtagggctcaagctggggacagattggggaaagggggaccatcatgcccatatagatgtccctgtgccctggcagtcaaggctgctgaaaaataacaaaacccagaagtctgcgtgatgctgcctctccatttgtccaaagccttcttgcggcagtttgcaggcttttgcaaaagctccaggaccaaggagctatgttcatgctggaagcttgttcaggattagctgttctttgtgggatgggtgcagccagggccaggtgtccagggacagtgttttaacaaagggcatgaggtgtctgatctcacagtggaactccacttgcctttttttcatcttctcattctgcttcatgcacagaaccagccccatcctgaaactgactctaaattactcccgccccaggtggagtgcctttctcggagttcaacagagccttcctgtcgcccaagggacaactccactgaatgcccaagccacacccaaaacctaacaagtaaaaaccaaattctgtgctcccccatcctgggccattcctggtttctctactgctgttggtgataccaccatcagcttgtccatcatgaccctggccagttcctcccacaaccctccacagcacccagggacctcacctccattccatccgacacagatctcctcaccacaaaccttggttttgcaacagcagccatgagacctttacaccctccgcccttcatcctgtcccccactgaggccccagagccattccttaaagcagcgcgccacaaactataacccacaagccaattctggtacccagcctgttttgcacagccagtgaactgacaatgatcttttcatacagccagaaaaacaaaacaaaacaaaaaacaacaaaaaaaaaccccaccattctgagcatgtgacttccatgttcaagatgtctcatgttcagaaaggcccctggaaaaggaggaaggggagctgggcacaaagggagaccctctcagctgagctcctcccatccagacattttcctggacttcctatccaatgacttcccttagcttcttatcagccacccctgtctgcccaggaggctggaagatgtggccttttaactgggcacagctctgtcctctatcatatcagggctctgttcccaaggagggtagagagaatggacaccaggtggaccctcagcagtctgtgccacagagggagtgtttgcaatttccagactaaaagtccccatgtgcttgacggggtatgtgactacaacgtgatgcttgacttttcctcatatgaccagagccactttgtccatctggtacaatgtcagctatctgctaggggccctccaggattcccagtcaattccatatctgcatcaccaccattggcactaaataaaataaaatactcaagttcctgctggtgagcatgagcagtgctacactgggcccttcaaccaaggtgacatgataatgactgaaaataatcactgccacttattggggacgtctcatctgccaggcatggtacaaagtgctttaaataagcattcaacaatttcatgctgacagaagccctgtgagccagtggagctactactatgcccattatacaggggagaaaactgaggcagagagaggttaggtaattcgctcagcctcacacaaccaataggtggtggagccaggatttgggccccatctgcctgactctctagaggctctatcttccagtcttccagagttgagtctaagccatgaataggacaattagacagcagaggaaacccattcagccaccatgtgcatgaagagtaaggaatttctgtcatacagaggggagtgaattcactgagctgagagctgaggaaccattgatctgatggctgagacaccactgggaagactggagaggcttttctgggcatgcagtgccaggcacaggaggagctgagggaagatgactaagaggtactggcaaagaattcagaaattctgatggaagctttacatgttaccatcacatccatccatctatccacccatccatccacccatatcttcctccctccacccaatcatgcatacatccagtcatctatacaccacccacccacccatccatccatccatccatcccttcatccatcccatcatccatccaattatacatacatccaatcatatatctgtacataatccattcttccctcggttcatccatccatccattcatccatccatccacccatcccttccttcatccttcctatcatccatccaatcatatatctgtacataatccattcttccctcggttcatccatccatccattcatccatccatccacccatcccttccttcatccttcctatcatccatccaatcatacatatatccaatcatacatctgcacatcaccagctcatccatctatccatttatccatccatccttccttccatccatcattcatccatcatacatacatctaaccatacatctctacatcattcattcttccatcgattcatccaattatccatcattccttcctccatccatcccattatccatttgatcatacatatatcatctatacatcatccattcatccatccatccatccatccacccatatcttcatccaatcaatcatacatacatcgaatcatctacacatcacccatccatccatccatccattcatctatccacccatccatccatccatccatccattcatctatccacccatccatccatccatccatccatccatccatgtaaccatccagtcatatatccaattacacatccatccagttatacattcatacatgcatctaatcattcaattatacatacacacatccatataattctacatccaattatacctccatccaattacacattcatacacccacctaataaattattaattcatatatccatccatataattatacatcaattatacatccatctaatcattcagtaattcacccaccatccagtcatctatccaataatacattcatccaatcatccatccatccatccacccattcatccatccatccgtccgtccacccatcatggtatgagccatgatttaccacgatggtcccctgtggacagcccaggtggggcagaactgaagggaagcccagggctgcccccataaacatttgcctcctttacatggatgagaactagatccacatgtataaatcctcatgatttgaaggtgcttttaccaacattcactcatgggattctcccaggagctctaggaggaggcaggtagagttgaggtcatctcacgcattttacagatgaggaaacggaggccctgagaggcaggtccaaggccacctgaccagaaagaagtggaactgggacttgaacccagccatcttgccccttggtcccatgctctctagcctgtaactcctgcttcctggtggggcatctccaggaggaccctatcggctggccatgggcctgccctggagtcttttgctctgtgtggccatccttcctccctcaggagagtgtgtgctcccagagcacaggctgtatcttctgagcattttgtcccttcccagtacctagcactcagctctgtatacattgggctctcaagaattctcaaccttccagagtgtaaggccttgacctgctcagccctggatactgcatgatgcattgataagcccataaaataaccagggcagattgactcccagtggccaaagtgccacagggaagggacaattcagcccttctaggaggaggaggaggtagttttctcatttctattaaggcaacaaaagctgccttactaaggacattcttggtggagggcgtgactgtcaaccactgtgatcatttgggcctctcttgcccaggcttcccattctgaaaggacagttttattgtaggtacacatggctgccatttcaaatgtaactcacagcttgtccatcagtccttggaggtctttctatgaaaggagcttggtggcgtccaaacaccacccaatgtccacttagaagtaagcaccgtgtctgccctgagctgactccttttccaaggaaggggttggatcgctgagtgtttttccaggtgtctacttgttgttaattaatagcaatgacaaagcagaaggttcatgcgtagctcggctttctggtatttgctgcccgttgaccaatggaagataaacctttgcctcaggtggcaccactagctggttaagaggcactttgtcctttcacccaggagcaaacgcacatcacctgtgtcctcatctgatggccctggtgtggggcacagtcgtgttggcagggagggaggtggggttggtcccctttgtgggtttgttgcgaggccgtgttccagctgtttccacagggagcgattttcagctccacaggacactgctccccagttcctcctgagaacaaaagggggcgctggggagaggccaccgttctgagggctcactgtatgtgttccagaatctcccctgcagacccccactgaggacggatctgaggaaccgggctctgaaacctctgatgctaagagcactccaacagcggaaggtgggccccccttcagacgccccctccatgcctccagcctgtgcttagccgtgctttgagcctccctcctggctgcatctgctgctccccctggctgagagatgtgctcactccttcggtgctttgcaggacagcgtggtgggagctgagccttgcgtcgatgccttgcttgctggtgctgagtgtgggcaccttcatcccgtgtgtgctctggaggcagccacccttggacagtcccgcgcacagctccacaaagccccgctccatacgattgtcctcccacacccccttcaaaagccccctcctctctctttcttcaggggccagtaggtcccagagcagccatttggctgagggaaggggcaggtcagtggacatctgatcttggtttagtatccttcattttgggggctctgggtgtggcctgggcctctggactttggccacggtgtttgttccagcccttctcctaacctgtcctttccagacactcggcatctaggttattagcacctcgcatactttctgacatgctcctcagtcctgattttgaccatcttctcttgcttcccatctgtgtcagtcaagactgcatttggctgtaagaaacagaaaccccaactaactgtggcatttacatgaagaggtttacttttctcacataatcagatgtctagacttggccagcacctcaagggtcattgatgctctcctgtctttattttctgtcatctttagtggttggattgttgcctcatggttacaaagtggctgctgcacttccaggcatcacatctgcctttgaagcaggaacaagttgcaaagtaaagtggccaaaagggccctgaaactaaatgtgtccccttaggaaagcaggagttttcttgcaagtggcaatcttctgcttatgtctcattggccagagctgggtcttacggccaccccttgctgcgagcaaggctgggacattgagcattttgccgtccaacctctttagcagaataaaccaagggggaagaacgttaatagtggcttttgagtcactagttggcagtatctgcccctctatctttccatcctccccatggagtttcaaggttcctttctcagtacttcttcaggctctgcacgttcatttggatcttgtgtcttggggtgaaaaactggcccaagtgtctccccaagcatccacctttggattaatttggaaaatggctgtcaagtgcccgcctcttgcttggtataatgctacagctttagaggacgcagcaggcatgggccttgccgctgaggttcttagcctcatgagaatatccagatcagattctcttggctccttcttagagccagtgatgcaagacacttcctgctcatcttgtcgggacggttttacaagttgcctgccatcctgagaaagtctacaaaacgatgccagacctcatgccagcttcccaagccttgactctcagtgctccctcaacaggattctggaagaatctcccaaacaagtcgcaatcccctctggaccctgtgcaggcatgagactcaagagcattggctcccacccctggtggagggaacactgctggggctgggatcttgcctggttgctccgcctgcacccaagacaaccataattaaaatgtccttcattgaacttggaaagccttcaaagctgacaactccttatgtgtacccggaaaggcctgggagtgtgccagggcattgctcgggagggacgctgatttggaagcatttacctgatgagagactgacagcagctcctggtagccgagctttccctcctgcctctgctgtgaaggtggacccatccaacagtcaaatgcctgactctggacaggagcggacctatttattgccatgcaagggactctgcacttttgaattgtgggtcatgggcttggatttaggggttagagctgggagaagtcttggaagtcacctagagatgacactgccattttgcagatgaggaaaccgtccaatcaaaatggaccaaggacttgcccaaagcctcacagcaaaaccataggcccccgcactaaccccagagtccctgtgctgtcttaagaatcaaatagttgtaagcaatcatctggttttcagtatttcttcttttaaaatgcctggggccatgcccagcagtctgtttcactgcagcgtttacacagggctgccgggctttcctggtggatgagctgggcggttcatgagccagaaccactcagcagcatgtcagtgtgcttcctggggagctggtagcaggggctccgggccctacttcagggctgctttctggcatatggctgatcccctcctcactcctcctccctgcattgctcctgcgcaagaagcaaaggtgaggggctgggtatggctcgtcctggcccctctaaggtggatctcggtggtttctagatgtgacagcacccttagtggatgagggagctcccggcaagcaggctgccgcgcagccccacacggagatcccagaaggaaccacaggtgagggtaagccccagagacccccaggcagtcaaggccctgctgggtgccccagctgacctgtgacagaagtgagggagctttgcgtgtttatcctcctgtggggcaggaacatgggtggattctggctcctgggaatcttgggttgtgagtagctcgatgccttggtgctcagttacctccctggctgcctgccagcctctcagagcatttagggccttctggacttctagatgctcctcatcttgcctcagtcagcgcgtcagttccagagacttctctgcagggttttctggggcaggtggtggcagacccgtgccttcttgacacctgaggtcagtccaccctcctgctcagactgcccagcacagggtcacctcccaaggggtggaccccaagatcacctgagcgcacagagggtgcagatgactggaccacaccttttggtgatcttaatgaggtggtcccagaggagctcagacatgcaatctagcatccagttctgggactctgtctccttttcaaacgtattcatgtagaacaggcatgacgagaatgccttgtcaacatgggtgatggggaatcaatcagacagggcgccgggctcaaggctgcagtcacccaagagtggctcagcccaccaggccctaggaaacgcctgcacagcctggagctcctggagtcatttccttcatgtcttcttcactgcacttacgtaaagatgccagccattggtttggtgatttggagggtgcccagttgcccaacaagaaatgcagaagaggcctagccaggatttcaccagcagtggagagtagagaagatgtggccagaaaagagtttcctttccctcctaaagatggtactccctgcagctactggggaagcctgcagcattctctagggctctgtgtgttgagagcagccccaccctggccccttctgagtgcatttctgctttgtgacttgatccgtgaagtcccctgagatgggcagaggggatgtcctcgaagctggggcagagcctcatccttgaacgtgaaggacgtttgaagactgtggcatgatcacaggatgagatcacagggaacttgagtttctctcctcctctcccttcacagttatttcactgagggaaatccctcccctgcccagaatgaaaactctagccaactcttgacttttccatcactccaaagtagttgaaagtacattagtctccacagtggcaaaacagtgtgcaaaagctaaataattagaacagccagtcccatgtgacagtcaaagcttctaactccattcaaagttgcagccattcccctcgagggctggcagggaggggaggggtaagagaaacaggaaggttcttactgagttggtcctggtgtgagctgcgtcacactccctgcagaggtttcaaggagactctctctctctctgtctccatggggaccttatttgaattcttctactcttaccccagcctgccatctccagctatcctcccctgaagagcccttctgctgcgctggattctggtggccatgtcatctcctcggccccgtgggagtctgaagatctggctgcagcctcacctctgaggtcctgctagttgccacctcttaaacatgatctgaggctcccatgcactctgacctgtgcccacatggggcccacgggaaacacgctggcaagcaaactgtgggtgtgcagacggttctcagggctgcagcacctgtcctttgctctgcccccaaagcaaggccagcccatcttccatcctctagtgttccttggtggggccctgaccacagtccaccaggtccctaaccagaggggacacacaccaggtgtcctcaatgtattgccttgaaacagttgtgctgggactgtgatggggggtggccatgtagccacccccaccacccccaagccactctctccaaggaaatcctcctaaagatccctttacatcctccatgtggtggggaggttctagagttgggtgcatgtgtcttcagctactgacaatgcagaccttagttggcacctcgctctggcctatcctgtttgctgttcttggcgctccagtgaaactccccatgggccatccagttggggtgcagtgtggccacccccttgcaggttcctgccttgctggagagcacagggccctcctggctcttgtaaaacactccccatggtacagagaggccagcagtgatgtgaggcccaacctccctccatggtgttcccaagcagctccctttctggggtcaaggggtggcaaagacagtgcagcgtccaatttctgactcaagccgggcctggctatcgcagctctgcactgtgtgtgacagcaaggcaactcacccagtgccgtggcagtgaccgtgtccgaggaagcctcctcacaccctctgtctcaaggactctggcatttagctggacttgctgtagctctgagcctttctgccattgccatcaccttgtcagaaactcaggccgaatctgcactcagagttgtgcccaggcagttgagccaacacttgctcagcgatattgtcacatgacaaggcactgtcaccactgggcgtcgtgggtagcgcagtgtcggctggatggacccggagggtgtctgtgtcatgctagtgctagtgatgggagccccgtgagcccattgcccgccctcccatgccctcagcagctgcctggggacagccaatggcctgggtgtttctgaggctaccacatggcttccaggaaactcgagaacctttctctcccttgcctacactcttcacacaggcctgtgctggccagcggtggggatccggcattcctatcttaggtgcagaaagtgactgactcattgcaggcctgggagataagactgatggcccagccagcaagatgtatggatttctcagaggcagtggcctctgtcattgtcctcaggaaatgctggtgattctggtggcctgaggtcaatgcatgtcaacgtggccaacttgccttataaactttttttctggacaattgcgtgcactgtcctgtaacagtgtcctgttgtttatgatgcagaaataggtgtttttaaagcctattgattttggtactattaatgtggtcaggaactttctcagtctttcttgtttggggtgagctgtggcttcctaaacaggaacccaagacacccccaaaagctgctcaccagcactgccagcctccctcttaccaagtagcacccgttcaggacattctgcgaaaggcatttgcccagaagttgggaggaaggaaatgtaacattttggggcacctaccatatgccaggcaccaggctaaacgtgttcacacaaattctcttactaaccctcaccatccttctacaagacaaactagtatcttcatcttggggttcaagatgaggaaatggaggctcagagaggttgaatgaatgccggtgcctggatatgaaccccatctgcctgactccgcaacccaggcaaagtctttccttgaacttcccagcagccactgcttagacacagcctccacaaccatggctcagcagcaaattgcttctctgacctcactcagcctgtgtgtccttgttgagtgaggcattcaggaccctggtcccaaagtggagaaagtctttcctactaggtcatagctacacctgcatgtgggtgctgtgccttttgtttagtgaacttttatcaccagcatcctcagcaatgacatttgcagagaagccagagctgaggcaccttggtattcttgggatgtgactttcctgaatgtttaagggaaaatgcccgaaggtacagagagcttggtttctagtaaacaataactgtcttgcttttaccccccttcatttgctgacacatacaccagctgaagaagcaggcattggagacacccccagcctggaagacgaagctgctggtcacgtgacccaaggtcagtgaactggaattgcctgccatgacttgggggttggggggagggacatggggtgggctctgccctgaaaagatcatttggacctgagctctaattcacaagtccaggagattttagggagttggttcttatcaaaggttggctactcagatatagaaagagccctagtggtttttttctaataccatttctgggtaattcctaaggcatttagtgttctgaaagatgctagccttgtccagcctgggagttgagaatgaatgtctaacagaaactctaggccgggcgtggtggctcacgcctctaatcccagcactatgggagacccaggtgggcagatcacctgaggtcaggagtttgagaccagcctggccaacatgtgaaatcctgtctcactacaaataaaaaaattagccgggtgtggtggtaggtgcctataatcccagctactcaggaggctgaggcaggacaatcgctcgaacccaggaggtggacgttgcagtgagccgagatcgcatcattgcactccagcctgggcaacaaaagcaaaactccgtctcaaaaaaaaaaaagaaactcaaatatgtgtgacaggcgattctcactgcaggctgccctgtggctgatccaggagcaaggccttaaccatgtcatccccaagcgattgcttgtaaactttcttctgtgcagccttcaacccttattatgattttcttctcaggaaccaaactgctgtattcaagaaaggcagctttgtgtaatcatttatcataaatatcttaagaaaaatcctagagattcctaattttaggaaatgggagacctatggtactgatataatgtgggctgggcttgttttctgtcatttgctagataaatgaacttgagagcctactgtaaaatgtggaagcttctagattgcagaagggctggaaagacactgttcttttctcccgagtgatgggatctgtccagtatttagagctgcctctgaggccatctgattctaggagactctgcctcgttgaggatattttgaggcctaactacacattcctgcccccagagaggtcacagcctatagcaggctgatgtttctcatgtcacatggcacagaaaggcacattttcgttctcaggctaacaaagagcttcaaaaactattagaagggacagtggctataagagaagaacctcagtcaatgtgtgaaattaactaggaacctggctcctgtttcttttaggtcatgtttttcagcttaggtaaaactagaggctttgataaagcatgacctctagaaatcattgcttttcataaatggaagtgggtttgagttttttctactgattgttagtgcaggtgatgtctacatgcccccagaacatattccatgcaacaaaaaaagcccaggtcaccgtctttgctgggaacttgacttttgtgctcactgaattttaagctttctgacagcagcctggaatcatggagggataaagtacctattagtaagatggaaaaaggtgtttcaggttggagctgcagtctgttgagagtaagctatgggaaggcctgtatacgaggggtggacttttcttctgtaagtgtccagagaccaggcctcctgaagagggcatgggggcttaacttacctggactactgtgtttacaatactcatttatcttgaactcctcctaacccctgagaattgctacatttagtatttgctgagtacttcctagcatcctagggaatcaatagaacattctcccaaccaggctgggtgcggtggctcatgtctgtaatcccagcactttgggaggccaaggtaggcagatcccttgaggccaggagtgcaagactagcctggctgacatggtgaaaccccgtctttactaaaaatacaaaagttagccaggcatggtggtacacacctgtaatcccagctacatgggaggagtaggaggcaggagaattgcttgaacctgggaggtggaggttgctgtgagccgagatcatgccactgcactccagcctgggcgacagagtgagtgagactctgtttaaaaaaaaaaaaaaaaaagaacattctcctaacctggcttcttcctccaggggtgtaattaatcatgtcagtttcctcattgatacacacacacacacactacaatcctgtatccattacttttcaaggtacatttactatttacgtttggggtccttgtctcttttttaatagtgtttcttaaagtcttgtattatatcagagtacagtaacatcccagtcaagagcactctagtaagctctaggaggaaagcgacttccggaaggcagtggagacctgtcctgttggggcagcataggggcagcccctgcctctggtcagttctggcgctcaggctcagggttgcctctgggctgttcttcccagagactgacaaagggctcccataaggcacctgcagagcctgtgagaagctgaagtcaatgttttcctgacaccagttgatctgtgcaggatccattgatttaaccacctgctgtgtggcatgcactgtggtcgatgccaggaacaggaattggaggggcccatgagcatggccagtatcacaggctggaggtgctgctgcgctctgaccgggcctcttggggatgagcccatgtcaaccaccttgcctccgatggggtcgggcccacaggttacctttgtgtgtccatgaccacaccttcctccccgacctcatccaaatctctttcttttccaagcccctgaatccttcagggctgcaggttttgtttaaagcagagctggtgagttgcataggttgttgcgttgggactagatggggtgttcaaagagttgggagttaaaaaacataaagggtatttattaggagaaccaaggagtgtaattctcctgttcttaatatgcggccaggttaatgaatgtcacgtgaatgaaccagaaaaaaatgaagtgtgcccttgatcagctgggttggtgtgcagcaagctgtgtgaccaggggacagcagtggtcctgagggccgtcactgtctgccgtgcagagcccttcctcccacgggggcctacctcacctgtgccaagggcttgtctgtggtcagtgacctggatagatctgaatggggcttctttttcgaggagtcttatggcaggtctctcagtaaagactccattcttgatgatcacacattttggattttccaaatctgtcagagaatgggcttgaggcggggtttgtgggcactagtttcactggtttcatttaccaaaaaggggagcagaagtcaagtatggtggctcatccctgtaatcccagaggcaagagaattgcttgagcccaggagttcgagaccagcctgagcaacataaggagaccccgtctccacaaaaatgaaaaataacattttagtcagacgtggtggcatgcatctgtggtcccagctgcttgggagggtgagatgggagggttgtttgagccctggagttaaagttgcaatgagctgtgattgcaccactgcactctagcctgggtgacagaacgagaccctgtctcaaaaaaaaaaaaaaagaaagaaaaaaaggaaaaaaaaaactcatgcctgtaatcccagcactttggggaccggggtgggcagatcacgaggtcaggagatcaagactatcctagccaacatggtgaaaccccgtttctactaaaaatacaaaaattagccaggtgtggtggcacgtgcctgtaatcccagttactcgggaggctgaggcaggagaatcgcttgaaccagggagtcagaggttgcagtgagctgagatcgtgccactgtactccagcctgggcgacagagtgagactctgtctcaaaccaaaaaaaaggggtggggggcgggggcaggagaacagtgagaggtagggagaggaaaggggattctcgctacacccaaaccagataccatctagaggctagaatctttgggaggctcaaattccctagaaagcaggagaagcttctgtagccctcccgctttcccagtagattaagcccagggcggctccagatgtgtgacatgctctgtgcccaaccagagcccatcataggcagaggaataacacccacaccagaagggccctcggaggtcaccacgtccaagaaccctctttacagatgaggaaactgaggcccagagaggggagagccacctagcgagctggtggcggctagaccaggagagctgtcattccaagcaagcaaaggcaacgagacgagcccagagctgtgctcccatctctttgttagggggcctgggatgccctctcagtgtcattttgtccaggatgatgctccctctcttaagcgattaatgcgcccttgctaaccttttgctatcgctgcctcttcaaaccagaggagttgagagttccgggccggcagaggaaggcgcctgaaaggcccctggccaatgagattagcgcccacgtccagcctggaccctgcggagaggcctctggggtctctgggccgtgcctcggggagaaagagccagaagctcccgtcccgctgaccgcgagccttcctcagcaccgtcccgtttgcccagcgcctcctccaacaggaggccctcaggagccctccctggagtggggacaaaaaggcggggactgggccgagaagggtccggcctttccgaagcccgccaccactgcgtatctccacacagagcctgaaagtggtaaggtggtccaggaaggcttcctccgagagccaggccccccaggtctgagccaccagctcatgtccggcatgcctggggctcccctcctgcctgagggccccagagaggccacacgccaaccttcggggacaggacctgaggacacagagggcggccgccacgcccctgagctgctcaagcaccagcttctaggagacctgcaccaggaggggccgccgctgaagggggcagggggcaaagagaggccggggagcaaggaggaggtggatgaagaccgcgacgtcgatgagtcctccccccaagactcccctccctccaaggcctccccagcccaagatgggcggcctccccagacagccgccagagaagccaccagcatcccaggcttcccagcggagggtgccatccccctccctgtggatttcctctccaaagtttccacagagatcccagcctcagagcccgacgggcccagtgtagggcgggccaaagggcaggatgcccccctggagttcacgtttcacgtggaaatcacacccaacgtgcagaaggagcaggcgcactcggaggagcatttgggaagggctgcatttccaggggcccctggagaggggccagaggcccggggcccctctttgggagaggacacaaaagaggctgaccttccagagccctctgaaaagcagcctgctgctgctccgcgggggaagcccgtcagccgggtccctcaactcaaaggtctgtgtcttgagcttcttcgctccttccctggggacctcccaggcctcccaggctgcgggcactgccactgagcttccaggcctcccgactcctgctgcttctgacgttcctaggacgccactaaatcgacacctgggtgcagctgctccactccctcggcctcctcccgtgctcaggctgtggccgcacgcgcccctcacgcttgcccgccactctgcatgtcaccagcacccccgctccgtgctccccaccttgtttgactctctggccacttgatttgtccacaacggcccatcagcccacaggaggtttggtgggtgccttccaccgacaggatgacgggtgccctcatggtgtctagaactctccaaccctcccatgtaggcataagcagccccactttgcagatgaggaaacggaggctcagagaagtacagtaacttgccgaaggccaatgagtagtaagtgacagagccaggtttgggatccaggtaggttgtctctgaaagacacgcctgtcctgcatcccacaacgcctcccaggaggtgctggagtgtggacgcctaacacagagatgtgcagggcacacacagcaggtgacacacacagcatccagaggtggcccagagctcatgctgtgcctttggcccagtgccctgcccccacccactctgccttgtggcaggaagacaaggagcagacacaagatctccctggtccacatgccaccacctccctctgcagaggacaaggggatcctcatgctggcattggagggggttgagcagggcccaccttgagccctcaggagcacgaccacagcagccctgcagggagggattggtgggaggagagtcccaagtatcagggagaggagagttggtgtcccacaggagacctcagagccacaaggcgagcttgttcataaatttgggacccttagcatttcacagttatttgcagagcccagaaatggatgttactgaagctcacagttgcaagcatctgttaaatttttattagattttacttttagggaaaactttgaaatgctataaagaagcctgtgtttaaaagttaagacagaggctgggggcgatggctcacgcctgtaatctcagcactttgggaggccaaggcaggtggatcatttgaggttaggagttcgagaccagcctggccaacatggtgagaccctgtctctactaaaattacaaaaaattagctgggcgtggtggcgggcacctgtagtcccagctactggggaggctgaagcaggataagtgcttgaacccaggaggcggaggttacagtgagccaagatcacaccactgtaccctaagcctgggcgacagagtgagactctgtctcaaaaaataaaataaaataaagttaagagagaaaaaaatatatcctatatcctttgttaaattccaaaacagtaggggacaaataactgacttgacaggttactacaatatttcctgaaatgatgttttcttgaatactggcctactagaggttcataggtgtgtttggattaaaaaagagttccatggcccagtgactgggggaaaaaaataaaagactaaagtaagttaaacaggcttttctgctgcaggacttgtcagagcctttaatgtactaatggccattgtgaccctctgagaaggtcacagagtgggtttcccaaacttacttgattctacctgctaacatttcctggaggaagtttgggaaatgccgatttagcagattcttttgttgtgccgtggatggtgctggttgatgtgggcaaaacaaagaacacgtgagtcagatccgcctggggctcttactaaagtgcaggttcccaggtgccactttaggcttacagacccagttgtggggtaagcctgggagtcttttagcaggtgattctgccacatagtatagttggaaaacctctgggcatactcattgctggtccctctagaaatccaggtgacaatagccaatgagaagctccaagagacccagttgtccatggggtagagggaatgtgatattgaaaccaaagaagaaaatctatgatcagttttcagcagtgactgtcaagagaaggagaagggtgagttagcgctgatgctggctgacaggtcagcgggttggtttcaccaaggagtgtgatgaaggctgatgttgtctgtgggaatgtatgatggtaactggtttgtagctaatttggggaagcagtgagaattcgtgccctttgaagaccagtaagtggcaagaaacccaccaggcctggctcagggctgggctgggcttggctcgtctcagagcagctggggctggtggccaaagccaccattagtgaggggcaggccctgggggtacaaccagcaactaggggacaaagacaaccctgccagcctctcctattctggaggcgtgtgaccagaaatggagatgggttggtcagcataagatggccaggaaggtggaaatcaggactgctggcaatctagccacatgggcaggggagccgggtggttccaggcagtttccaaggccaagagggtgagcaggcacctcacagggaatcagggccaagcctggctgcagtgtggagacaatgcacccacccccatccttggatcttgcaggaggctgggtcctcactgagctaccaacatccatggccctgaggcttttaaaacacccatccatggagtggggctggtcccagtggggtgaggctgaccctggcagaaacagggcaggagcctgtgggttagggagactgcaccttccttagatagcctccatgccatcatgtccccgtgacagtttctgctgcgtcccctctgcatggtcccaccctcggccagcctgctgccccctcttgccaggttgcgctaatcagtgaccccagtgtgctgtgttgatactaacaatgcgaggcctagcagattcaagggaaaagagaaccaactgggtttccaccagacccaactaaacaaacatggacctatcccagagaaatccagcttcaccacagctggctttctgtgaacagtgaaaatggagtgtgacaagcattcttattttatattttatcagctcgcatggtcagtaaaagcaaagacgggactggaagcgatgacaaaaaagccaaggtaagctgacgatgccacggagctctgcagctggtcaagtttacagagaagctgtgctttatgtctgattcattctcatatataatgtggggagtatttgtcactaaagtacagctgtcatttaaagtgctttgtattttggggcaggcttttaaaaagtccagcatttattagttttgatacttaccccagggaagagcagttggcaggttcatgaagtcatgctcctaattccagctttcttagtgtactttcagtgagaccctgacagtaaatgaaggtgtgtttgaaaaccaaacccaggacagtaaatgaaggtgtgtttgaaaaccagccctaggacagtaaatgaagccatcttctcactgcataaactgcacccagatctttgcccatccttctcagtatttcacttcacccattgtttactgtctcaatgactggggaaatgtctggggaaatgctcccgtaattgcacagtggcgtttttcctggaaaatcccaccatggctctagataagacctatttttcttaaaggtatctaaaatttccagcataaattctgtctgaaacacctgaattttaatcagtactggagcccggagggcatctccagttgccacatagctctgagcattcagtggtgtgttgagggctgctcccggaagtgcctgcagagtcagggctccccagcctcatctagtgaggcagtggaagggcctgtggggatttggagagctggcctgggtctctgaagtgatagtgacagctgcttgtcaatcacggtgcacatttagtgccgggggcagggggcagggaataccagcctcatgcatgcatgcattcatttgttccttccttcattcattcattcagtacacatgggtacaacatccctgccctggagttgcccagagtctagggaggggaaagatctattaccctgggcctcggccagctggggagtgctgctggtggagaggggccgtgtgcagcgagggaaggaggagtcgtcaatacccccaccccagctttgctttcttgtcatcagccccagggccccagcctgtgtccctcctctcccattgctacttcatctcctgggtcctccttaccaagcctgaccacacagagggccttggccgcttccatggggaattggaaagcaataagatagcatcccctagaagcccagtgaagtctgggacaggacccttctctgagctctgacttgctcttggaaacacttcgaggcttagcctccccactttgtttcccaagagtgtgacctgttcccctccaaacacccccttctcctccagggccatgcccacccgtcaaaatcccccacgggcaggacgaactgtgggtgtcagtcaccatctatcctgcatcctggttccagggccccccccagccccgcctccatagggacaggcgtgcagacacccgtccctggctgcttcctcttgtggaatgggttcaaaagtaagcagtgttgtttacactgacaaactgaaaaaaaaagaaaaagagataacattggaggcttggcacagtggctcatgcctgtaatcccagcactttgggaggctaaggtgggaggatgtccccagcccaagagttctagaccagcctgggcaacatagcaagaccccatctcaaaaaaaaaatttaattggccaggcagaggtgggaggatcacttgaacccaaagggtggaggctgcagtgagccgtgatggcaccactgcactccagccagggcaacagagggagaccctgtctctaaaacaaacaaacaaacaaacaaacaaaagagttaacattggccagattaggattcaccagatagtgttaatattagtttgatttgagactttaatcagaaagcacatgtgtggtgggggtgggtgtaacctaagtcaggtagaatctttccaacttggggggggcacactcctgattgtagccatatgagtctgtcagtgtggtggaagagaccatgggttaatgggcaggtaaaaaagcaccttgcctggaattgagtagaaagtaaggcccttcagaccccgtgacacacttggggacattttcttgagtaacatcctaagattcatgtaccttgatgatctccatcaacttactcatgtgaagcacctttaaaccagtcgtctccaaattcaggggcacagtaacatccaacaggctggagaaagaacgtactagaacttccattcctttttcatgtcctcttctaaaagctttgtcagggccaggcgcggtggctcacgcctgtaatcccagcactttgggaggccgagacgggtggatcacgaggtcaggagatcgagaccatcctggctaacacagtgaaaccccatctctactaaaaatacaaaaaaacgagccgggcgtggtggtgggcgcctgtagtcccagctactcgggaggctgaggcaggagaatggcgtgaacccaggaggcagagcttgcagtgagccgagattgcaccactgcagtccagcctgggcgacagagcgagactccgtctcaaaaaagaaaaagaaaaagaaaaagaactgtgattggggaggacggtcactttcctgttcttactgatcagaagggatattaagggtacctgattcaaacagcctggagatcactgctttcaaccattacctgccttatttatttttagttactgtccttttttcagtttgtttccctcctccatgtgctgacttttattttgattttatttatgtttatgtttaagacatccacacgttcctctgctaaaaccttgaaaaataggccttgccttagccccaaacaccccactcctggtagctcagaccctctgatccaaccctccagccctgctgtgtgcccagagccaccttcctctcctaaacacgtctcttctgtcacttcccgaactggcagttctggagcaaaggagatgaaactcaaggtaaggaaaccacctttgaaaagaaccaggctgctctgctgtggtttgcaaatgtggggtttgtttatttgttttttagcctcaaagacctttcttcaaatgagttctggcatagaagcaccgtgtaaaatagttagaattctgggcaaaggggaaaagagagctgggggccatccctctcagcaccccacaggctctcatagcagcagctcctaagacacctggtgggaccttggtttcgaaatcgctactctaaggctgggcacggtggctcacacctgtaatcccagctctttaggaggccgaggagggtggatcacctgagatcaggagttcgagaccagcctggctaacatggcaaaaccctgtctctactaaaaatacaaaaattagccgggcgtggtgttatgcgtggtggtaatcgcagctactcgggaggctgaggcacaaggattgcttgaaccccagaggcagaggttgtagttagctccagcttgggcgacagagcaagaccctgtcgcaaaaattgtttaaaaaacaaacccaaaattgctactctcattgggttcctttgcccattcctgattttggcaagagaaatgcttccagattgccctgatctgggtaggacagcatcacgccatagcaacactgccccgtgagctcactgccccctcaactagcttgtggtccttggttaatgtcagtttcttttttgagtttgtgttatgtctaagggtcatctgctgggtaacggaacccagggactgccctagtccctagactgtgccatgcccgactctgccagctttgtcagtgatgctggtgctcgcctcctcgggtgctcgcctggtctgagcacacccaaggagttcttgaggccttagggttgtttgcgagagaatgaaagaacacgacctagctctctttagcatccttggtcaggttcaacactgcccccaggggcctctggtggagccaaccaccatcagccaaataaatccataattagagtcagaaaatggatgtctgcatatgtgtagtgcactaatgtcctgccgatgattgacatggagtggagagtgacctgatcattgctgtgagctctgctggccttggcacaactcatgctgataactaatgcacacagttcctctgggaggaaatgtcctcagggaacttggagtttgggtggggatgtgggtttgtgtgcccagcaagcccttgtggttgtagcagacactagtggcatctaggaggcaaagggtcaccccagtcttagccacgttttgagtcaaggtggcggagtggggctggtgttgactcttggtggcagtaacttttcccaatggtgaaaaacccctctatcatgtttcatttacagggggctgatggtaaaacgaagatcgccacaccgcggggagcagcccctccaggccagaagggccaggccaacgccaccaggattccagcaaaaaccccgcccgctccaaagacaccacccagctctggtaagaagaacgttctcttgaatcttagaggaagctgaagctctcagaggtacagccttcattttaggaggccttaggccactgagaatgaataacccctggcagctggtcagcagcttgcagtttactaagcactggagtcttcattgccttctcagtccttttgatttctgaggcaaatgttgaatccctacctttttttttttttttcttttgagacagagtttcgcttttgttatccaggccggagtgcagtggtgtgatctcagctcactgcatcctccacctcccaggttcaagcgattctcctacctcagcctccctagtagctgggattacaggcacctgccactatgcccggctaattttttgtatttttagtagagacagggtttcaccatgttggccaggctggtctcgaacgcctgacctcaggtgatccacctgcctcggcctcccaaagtgctgggattacaggcatgagccaccactcccagcctgaatcctcactttttatcaatgaagaaattgaggctgattctgcagcatgataaaaaaaaatacagaaaaaggaaaaaaaagaaagaaatcgagcctctgagagtttgcttgactgagtctaaccagctcattttaaacccgaggaaaatgcagtcacatgactactaagtggcagctctcggagcctctctggccccaagtccagggttccatagaggcagccccagcatggcatgttttcagtccccaaatgagactctggagacaaatgtctctggagacagagcagcagcctggataagtcacaatgggtgacgtcactcagggctcaacccctgggcagcttaacttgctagggacgttaggagtctgctgcaaaacctgagggtcttagctgagcagtcacaggctgggcccgttgccctgggctcctgtgagtaaaacccagtcaattttgagtacccagtaaggcatccattgagttattttgcagccaggagtgctattaagaacagtcgcggctgggcgtggtggctcatgcctgtaatcccagcactttgggaggccaaggtgggcggatcacctgaggtcaggagttcgagaccagcttggccaacatggcaaaaccccgtctctaataaaaatacaaaataattagctgggcgtggtggcgggcgcctgtaatcccagcttctcaggagggtgaggaaggagaatcacttgaacccaggaggcagaggttgcagtgagctgagatcgcaccattgcactccagcctggatgacaaaagtgagattccttctcaaaaaaaaaaaaaaaaaaacagtcgtcctctttggggattagggacagcctgcctgcctgcccgagcacttctctcttccattgccccagtgaagtattccaggcccctgggtttagactctgcaccatgtaggggtgtctgacctgcacttgctccttggtggcacgggcagcctatggcacttgctgcgggctgtgaccaaagcctggcctggatcttggatcttggtgactctgcttctccctggcctgagggagctgcccagagcctgcccaccacctgctgcgtgtctttgcggtggcatttctcgcacacatgccgtgcggtggcacccccaaggatggccattcactaaggcccattgtttttgtcttttcgcttcgtgttttctggcctggtgtttttctcatatacatgtgatccagggataattcccagaattttgacaggattttaagtagcgtttggatcctgctgtttttttttcacttaacatcgggccagttgactcacactctgttttttgttgttgtttttttgagacggagtctcactgtgtcacccaggctgaagtgcagtggcacaatcttggcatactgcaacctctgcttcccaaattcaagcagttttcctgcctcagcctcctgagtagctgggactacaggcacaggccaccacgccctgctaatttttgtatttttagtaaagacagggtttcaccattttggccagcctagtctcgaactcctgacctcaagtgatccgcccacctcggcctcccaaagtgctgggattacaggggactcacactttgtaacaacctgaaacaacgtgatgcatttccctttgggtcttacctgctcttcggtggctgcctgcaggtggagagaccctcccccttgggcccctcgaccttgtttcagaatggggcccctgctgggccagctgtgggtgcctgccacgtgaaggactcattaaggccctgtttaagcctgatgataataaggctttcgtggatttttctctttaagcgactaagcaagtccagagaagaccaccccctgcagggcccagatctgagagaggtactcgggagcctacttcgctgggagcagcctccctttgcgtgtgtggccattcactggcttgtgtttctagagccgggaggacccttttctgcaatgcagggttcacacagggttcgcagcctgaagatggagcagtccgaattctcttccctgtgcagtttgcgcagctgtgtttgtctgatgggctttctaatcctgtgtgctctccttgacttcagggacaatggcattacaggcatgagccaccatgcctggctgtctccctatgtttcagatgaagacataggcttaaggaggtcaggtgacttgcccacgaccactctgtaaataagaggcatgaaaagtatttggagccaccaccaccaagcccactggtcaccctgggtctctgaagtcagggaggcaggaggatgggaggtctgaggaggcagagaggctgagcctggaggccctggaggccgaggccccatctgttgtttccttatgtggaaaataagaggcttcatttgtcctattgccacagagcgtactacttcaggaacatccaagacatggaaatccgcagggcacggtggctcacgtctataatcccggcactttgggaggttgaggtgggagaatcgcttgaggccagaagttcaagaccagcctgagcaacatagtcagaccccgtctctataaaaaacattatttttaaaaaagacatggaagtcaaattctaaaaactggtgctggctgggtgcggtggctcatgcctataatcccagcactttgggaggccgaggcgggtggatcacctgaggtcaggagttcaagaccagcctggccaacatggtaaaacctctactaaagaaatctttactgaaaatacaaaaatccagtctctactaaaataagtctctactaaaaatacaaaaattagccaggcgtggtgctgcacacctgtaatatcagctactcgggaggctgaggcaggagactcgcttgatcccatgcagcggaggttgcagtgagccgagatcacgccattgcactccagcctgggcatcagaataagactccgtctcaaaaaaaaaaccacaaaaaaacaaaacaacaacaaaagaaaactagtgcttattcgtcactggccaagctgcccattggctacatgggtgcttcaaagagctgcccttctccaggtctggccagcaggtatgtgttacagcaaatgcctggggcagcggcaggggcattgctgcgggaagcttctggacttgcaggaaagctaagttctcagactgcaggggagctaagcacacctcggcacagggtgaggcctgcggttctcagacttcagtctttgtggagcttgagaaaaatgaggctttgcaggtcccacccctagagattctgctctatccactcttgaaggggatcgagaaatttgcattttgcaactcccactttcctccttgaaagctccggagattctgacgcagggttccgtgggccacactttggaaaatacagacccatgagatagaataccagactgttgaagtgtaacgggggcctgggaagtgcagtaacagaagcaagtttgagggtaaaggacacccagaggagggagggacagcatctgcatggagaggagaagagaccccccagcagcttccagggtgttggaagggtgcgctagtaactgctatgcatggcaggtggggaactgtacgtcagggcacagcagcatgaagcggtatggctcgtgtggacagctagggacaggcaggcgtggagcaggcatcctgttctgaaggccaaatcccacagaggagccagggtgctggcaggagccctgaactagccgaacagctgaacagctgaacattcaccctgtggggaaagggtcagaagcgtccaggcttgagggcacagctgggtctcgtcactgcatcacccttatttaggataaaggccctgaagaattgtattagaggttggcaaagcatatctaccacctcctggagccacgctggccgcagggattataattatttccattttcaaattaaggcctctgagctcagagaggggaagttacttgtctgaggccacacagcttgttggagcccatctcttgacccaaagactgtggagccgagttggccacctctctgggagcgggtattggatggtggttgatggttttccattgctttcctgggaaaggggtgtctctgtccctaagcaaaaaggcagggaggaagagatgcttccccagggcagccgtctgctgtagctgcgcttccaacctggcttccacctgcctaacccagtggtgagcctgggaatggacccacgggacaggcagcccccagggccttttctgaccccacccactcgagtcctggcttcactcccttccttccttcccaggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgagagtggctggctgcgcgtggaggtgtggggggctgcgcctggaggggtagggctgtgcctggaagggtagggctgcgcctggaggtgcgcggttgagcgtggagtcgtgggactgtgcatggaggtgtggggctccccgcacctgagcacccccgcataacaccccagtcccctctggaccctcttcaaggaagttcagttctttattgggctctccactacactgtgagtgccctcctcaggcgagagaacgttctggctcttctcttgccccttcagcccctgttaatcggacagagatggcagggctgtgtctccacggccggaggctctcatagtcagggcacccacagcggttccccacctgccttctgggcagaatacactgccacccataggtcagcatctccactcgtgggccatctgcttaggttgggttcctctggattctggggagattgggggttctgttttgatcagctgattcttctgggagcaagtgggtgctcgcgagctctccagcttcctaaaggtggagaagcacagacttcgggggcctggcctggatccctttccccattcctgtccctgtgcccctcgtctgggtgcgttagggctgacatacaaagcaccacagtgaaagaacagcagtatgcctcctcactagccaggtgtgggcgggtgggtttcttccaaggcctctctgtggccgtgggtagccacctctgtcctgcaccgctgcagtcttccctctgtgtgtgctcctggtagctctgcgcatgctcatcttcttataagaacaccatggcagctgggcgtagtggctcacgcctataatcccagcactttgggaggctgaggcaggcagatcacgaggtcaggagttcgagaccaacctgaccaacagggtgaaacctcgtctctactaaaaatacaaaaatacctgggcgtggtggtggtgcgcgcctataatcccagctactcaggaggctgaggcaggagaatcgcttgaacccaggaggcagaggttgcagtgagccgagatagtgccactgcactccagtttgagcaacagagcgagactctgtctcaaaacaaaataaaacaaaccaaaaaaacccaccatggcttagggcccagcctgatgacctcatttttcacttagtcacctctctaaaggccctgtctccaaatagagtcacattctaaggtacgggggtgttggggaggggggttagggcttcaacatgtgaatttgcggggaccacaattcagcccaggaccccgctcccgccacccagcactggggagctggggaagggtgaagaggaggctgggggtgagaaggaccacagctcactctgaggctgcagatgtgctgggccttctgggcactgggcctcggggagctagggggctttctggaaccctgggcctgcgtgtcagcttgcctcccccacgcaggcgctctccacaccattgaagttcttatcacttgggtctgagcctggggcatttggacggagggtggccaccagtgcacatgggcaccttgcctcaaaccctgccacctccccccacccaggatcccccctgcccccgaacaagcttgtgagtgcagtgtcacatcccatcgggatggaaatggacggtcgggttaaaagggacgcatgtgtagaccctgcctctgtgcatcaggcctcttttgagagtccctgcgtgccaggcggtgcacagaggtggagaagactcggctgtgccccagagcacctcctctcatcgaggaaaggacagacagtggctcccctgtggctgtggggacaagggcagagctccctggaacacaggagggagggaaggaagagaacatctcagaatctccctcctgatggcaaacgatccgggttaaattaaggtccggccttttcctgctcaggcatgtggagcttgtagtggaagaggctctctggaccctcatccaccacagtggcctggttagagaccttggggaaataactcacaggtgacccagggcctctgtcctgtaccgcagctgagggaaactgtcctgcgcttccactggggacaatgcgctccctcgtctccagactttccagtcctcattcggttctcgaaagtcgcctccagaagccccatcttgggaccaccgtgactttcattctccagggtgcctggccttggtgctgcccaagaccccagaggggccctcactggcctttcctgccttttctcccattgcccacccatgcacccccatcctgctccagcacccagactgccatccaggatctcctcaagtcacataacaagcagcacccacaaggtgctcccttccccctagcctgaatctgctgctccccgtctggggttccccgcccatgcacctctgggggcccctgggttctgccataccctgccctgtgtcccatggtggggaatgtccttctctccttatctcttcccttcccttaaatccaagttcagttgccatctcctccaggaagtcttcctggattcccctctctcttcttaaagcccctgtaaactctgaccacactgagcatgtgtctgctgctccctagtctgggccatgagtgagggtggaggccaagtctcatgcatttttgcagcccccacaagactgtgcaggtggccggccctcattgaatgcggggttaatttaactcagcctctgtgtgagtggatgattcaggttgccagagacagaaccctcagcttagcatgggaagtagcttccctgttgaccctgagttcatctgaggttggcttggaaggtgtgggcaccatttggcccagttcttacagctctgaagagagcagcaggaatggggctgagcagggaagacaactttccattgaaggcccctttcagggccagaactgtccctcccaccctgcagctgccctgcctctgcccatgaggggtgagagtcaggcgacctcatgccaagtgtagaaaggggcagacgggagccccaggttatgacgtcaccatgctgggtggaggcagcacgtccaaatctactaaagggttaaaggagaaagggtgacttgacttttcttgagatattttgggggacgaagtgtggaaaagtggcagaggacacagtcacagcctcccttaaatgccaggaaagcctagaaaaattgtctgaaactaaacctcagccataacaaagaccaacacatgaatctccaggaaaaaagaaaaagaaaaatgtcatacagggtccatgcacaagagcctttaaaatgacccgctgaagggtgtcaggcctcctcctcctggactggcctgaaggctccacgagcttttgctgagacctttgggtccctgtggcctcatgtagtacccagtatgcagtaagtgctcaataaatgtttggctacaaaagaggcaaagctggcggagtctgaagaatccctcaaccgtgccggaacagatgctaacaccaaagggaaaagagcaggagccaagtcacgtttgggaacctgcagaggctgaaaactgccgcagattgctgcaaatcattgggggaaaaacggaaaacgtctgttttcccctttgtgcttttctctgttttcttctttgtgcttttctctgttttcaggatttgctacagtgaacatagattgctttggggccccaaatggaattattttgaaaggaaaatgcagataatcaggtggccgcactggagcaccagctgggtaggggtagagattgcaggcaaggaggaggagctgggtggggtgccaggcaggaagagcccgtaggccccgccgatcttgtgggagtcgtgggtggcagtgttccctccagactgtaaaagggagcacctggcgggaagagggaattcttttaaacatcattccagtgcccgagcctcctggacctgttgtcatcttgaggtgggcctcccctgggtgactctagtgtgcagcctggctgagactcagtggccctgggttcttactgctgacacctaccctcaacctcaaccactgcggcctcctgtgcaccctgatccagtggctcattttccactttcagtcccagctctatccctatttgcagtttccaagtgcctggtcctcagtcagctcagacccagccaggccagcccctggttcccacatcccctttgccaagctcatccccgccctgtttggcctgcgggagtgggagtgtgtccagacacagagacaaaggaccagcttttaaaacattttgttggggccaggtgtggtggctcacacctaatcccaacacctggggaggccaaggcagaaggatcacttgagtccaggagttcaagaccagcctgggcaacatagggagaccctgtctctacaattttttttttaattagctgggcctgttggcactctcctgtagttccagctactctagaggctgaggtgggaggactgcttgagcctgggaggtcagggctgcaatgagccatgttcacaccactgaacgccagcctgggcgagaccctgtatcaaaaaagtaaagtaaaatgaatcctgtacgttatattaaggtgccccaaattgtacttagaaggatttcatagttttaaatacttttgttatttaaaaaattaaatgactgcagcatataaattaggttcttaatggaggggaaaaagagtacaagaaaagaaataagaatctagaaacaaagataagagcagaaataaaccagaaaacacaaccttgcactcctaacttaaaaaaaaaaatgaagaaaacacaaccagtaaaacaacatataacagcattaagagctggctcctggctgggcgcggtggcgcatgcctgtaatcccaacactttgggaggccgatgctggaggatcacttgagaccaggagttcaaggttgcagtgagctatgatcataccactacaccctagcctgggcaacacagtgagactgagactctattaaaaaaaaaatgctggttccttccttatttcattcctttattcattcattcagacaacatttatggggcacttctgagcaccaggctctgtgctaagagcttttgcccccagggtccaggccaggggacaggggcaggtgagcagagaaacagggccagtcacagcagcaggaggaatgtaggatggagagcttggccaggcaaggacatgcagggggagcagcctgcacaagtcagcaagccagagaagacaggcagacccttgtttgggacctgttcagtggcctttgaaaggacagcccccacccggagtgctgggtgcaggagctgaaggaggatagtggaacactgcaacgtggagctcttcagagcaaaagcaaaataaacaactggaggcagctggggcagcagagggtgtgtgttcagcactaaggggtgtgaagcttgagcgctaggagagttcacactggcagaagagaggttggggcagctgcaagcctctggacatcgcccgacaggacagagggtggtggacggtggccctgaagagaggctcagttcagctggcagtggccgtgggagtgctgaagcaggcaggctgtcggcatctgctggggacggttaagcaggggtgagggcccagcctcagcagcccttcttggggggtcgctgggaaacatagaggagaactgaagaagcagggagtcccagggtccatgcagggcgagagagaagttgctcatgtggggcccaggctgcaggatcaggagaactggggaccctgtgactgccagcggggagaagggggtgtgcaggatcatgcccagggaagggcccaggggcccaagcatgggggggcctggttggctctgagaagatggagctaaagtcactttctcggaggatgtccaggccaatagttgggatgtgaagacgtgaagcagcacagagcctggaagcccaggatggacagaaacctacctgagcagtggggctttgaaagccttggggcggggggtgcaatattcaagatggccacaagatggcaatagaatgctgtaactttcttggttctgggccgcagcctgggtggctgcttccttccctgtgtgtattgatttgtttctcttttttgagacagagtcttgctgggttgcccaggctggagtgcagtggtgcgatcatagctcactgcagccttgaagtcctgagctcaagagatccttccacctcagcctcctgagtagttgggaccacaggcttgcaccacagtgcccaactaatttcttatattttttgtagagatggggtttcactgtgtcgcccaggatggtcttgaactcctgggctcaagtgatcctcctgcctcagcctcgcaaattgctgggattacaggtgtgagccaccatgcccgaccttctctttttaagggcgtgtgtgtgtgtgtgtgtgtgtgggcgcactctcgtcttcaccttcccccagccttgctctgtctctacccagtcacctctgcccatctctccgatctgtttctctctccttttacccctctttcctccctcctcatacaccactgaccattatagagaactgagtattctaaaaatacattttatttatttattttgagacagagtctcactctgtcacccaggctggagtgcagtggtgcaatctcggctcactgcaacctccgcctcccaggttgaagcaactctcctgcctcagcctccctagtagctgggattacaagcacacaccaccatgcctagcaaatttttatatttttagtagaggaggagtgtcaccatgtttgccaagctggtctcaaactcctggcctcaggtgatctgcctaccttggtctcccaaagtgctgggattacaggtgtgagccaccacgcctgcccttaaaaatacattatatttaatagcaaagccccagttgtcactttaaaaagcatctatgtagaacatttatgtggaataaatacagtgaatttgtacgtggaatcgtttgcctctcctcaatcagggccagggatgcaggtgagcttgggctgagatgtcagaccccacagtaagtggggggcagagccaggctgggaccctcctctaggacagctctgtaactctgagaccctccaggcatcttttcctgtacctcagtgcttctgaaaaatctgtgtgaatcaaatcattttaaaggagcttgggttcatcactgtttaaaggacagtgtaaataattctgaaggtgactctaccctgttatttgatctcttctttggccagctgacttaacaggacatagacaggttttcctgtgtcagttcctaagctgatcaccttggacttgaagaggaggcttgtgtgggcatccagtgcccaccccgggttaaactcccagcagagtattgcactgggcttgctgagcctggtgaggcaaagcacagcacagcgagcaccaggcagtgctggagacaggccaagtctgggccagcctgggagccaactgtgaggcacggacggggctgtggggctgtggggctgcaggcttggggccagggagggagggctgggctctttggaacagccttgagagaactgaacccaaacaaaaccagatcaaggtctagtgagagcttagggctgctttgggtgctccaggaaattgattaaaccaagtggacacacacccccagccccacctcaccacagcctctccttcagggtcaaactctgaccacagacatttctcccctgactaggagttccctggatcaaaattgggagcttgcaacacatcgttctctcccttgatggtttttgtcagtgtctatccagagctgaagtgtaatatatatgttactgtagctgagaaattaaatttcaggattctgatttcataatgacaaccattcctcttttctctcccttctgtaaatctaagattctataaacggtgttgacttaatgtgacaattggcagtagttcaggtctgctttgtaaatacccttgtgtctattgtaaaatctcacaaaggcttgttgccttttttgtggggttagaacaagaaaaagccacatggaaaaaaaatttcttttttgtttttttgtttgcttgtttttttgagacagagtttcactctgtcgcccaggctggagtgcagtggtgcgatctccgcccactgcaagctccacctcccgggttcatgctattctcctgtctcagcctcccaagtagctgggactgcaggtgcccgccaccacacctggctaatttttttgtatttttagtagagacggggtttcaccgtgttagccaggatggtctcaatctcctgacctcgtcatctgcctgcctcggcctcccaaagtgctgagattacaggcgtgagccaccgtgcccggccagaaaaaaacatttctaagtatgtggcagatactgaattattgcttaatgtcctttgattcatttgtttaatttctttaatggattagtacagaaaacaaagttctcttccttgaaaaactggtaagttttctttgtcagataaggagagttaaataacccatgacatttccctttttgcctcggcttccaggaagctcaaagttaaatgtaatgatcactcttgtaattatcagtgttgatgcccttcccttcttctaatgttactctttacattttcctgctttattattgtgtgtgttttctaattctaagctgttcccactcctttctgaaagcaggcaaatcttctaagccttatccactgaaaagttatgaataaaaaatgatcgtcaagcctacaggtgctgaggctactccagaggctgaggccagaggaccacttgagcccaggaatttgagacctgggctgggcagcatagcaagactctatctccattaaaactatttttttttatttaaaaaataatccgcaaagaaggagtttatgtgggattccttaaaatcggagggtggcatgaattgattcaaagacttgtgcagagggcgacagtgactccttgagaagcagtgtgagaaagcctgtcccacctccttccgcagctccagcctgggctgaggcactgtcacagtgtctccttgctggcaggagagaatttcaacattcaccaaaaagtagtattgtttttattaggtttatgaggctgtagccttgaggacagcccaggacaactttgttgtcacatagatagcctgtggctacaaactctgagatctagattcttctgcggctgcttctgacctgagaaagttgcggaacctcagcgagcctcacatggcctccttgtccttaacgtggggacggtgggcaagaaaggtgatgtggcactagagatttatccatctctaaaggaggagtggattgtacattgaaacaccagagaaggaattacaaaggaagaatttgagtatctaaaaatgtaggtcaggcgctcctgtgttgattgcagggctattcacaatagccaagatttggaagcaacccaagtgtccatcaacagacaaatggataaagaaaatgtggtgcatatacacaatggaatactattcagccatgaaaaagaatgagaatctgtcatttgaaacaacatggatggaactggaggacattatgttaagtgaaataagccagacagaaggacagacttcacatgttctcacacatttgtgggagctaaaaattaaactcatggagatagagagtagaaggatggttaccagaggctgaggagggtggaggggagcagggagaaagtagggatggttaatgggtacaaaaacgtagttagcatgcatagatctagtattggatagcacagcagggtgacgacagccaacagtaatttatagtacatttaaaaacaactaaaagagtgtaactggactggctaacatggtgaaaccccgtctctactaaaaatacaaaaattagctgggcacggtggctcacgcctgtaatcccagcactttgggaggccgaggcgggccgatcacgaggtcaggagatcgagaccatcctagctaacatggtgaaaccccgtctctactacaaatacaaaaaaaagaaaaaattagccgggcatggtggtgggcgcctgtagtcccagctactcgggaggctgaggcaggagaatggcgtgaacccgggaggcggagcttgcagtgagccgagatcgcgccactgcactccagcctgggcgacaaggcaagattctatctcaaaaaaataaaaataaaataaaataaaataataaaataaaataaaataaaataaaataaaataaataaaataaaatgtataattggaatgtttataacacaagaaatgataaatgcttgaggtgatagataccccattcaccgtgatgtgattattgcacaatgtatgtctgtatctaaatatctcatgtaccccacaagtatatacacctactatgtacccatataaatttaaaattaaaaaattataaaacaaaaataaataagtaaattaaaatgtaggctggacaccgtggttcacgcctgtaatcccagtgctttgtgaggctgaggtgagagaatcacttgagcccaggagtttgagaccggcctgggtgacatagcgagaccccatcatcacaaagaatttttaaaaattagctgggcgtggtagcacataccggtagttccagctacttgggagaccgaggcaggaggattgcttgagcccaggagtttaaggctgcagtgagctacgatggcgccactgcattccagcctgggtgacagagtgagagcttgtctctattttaaaaataataaaaagaataaataaaaataaattaaaatgtaaatatgtgcatgttagaaaaaatacacccatcagcaaaaagggggtaaaggagcgatttcagtcataattggagagatgcagaataagccagcaatgcagtttcttttattttggtcaaaaaaaataagcaaaacaatgttgtaaacacccagtgctggcagcaatgtggtgaggctggctctctcaccagggctcacagggaaaactcatgcaacccttttagaaagccatgtggagagttgtaccgagaggttttagaatatttataactttgacccagaaattctattctaggactctgtgttatgaaaataacccatcatatggaaaaagctcctttcagaaagaggttcatgggaggctgtttgtattttttttttctttgcatcaaatccagctcctgcaggactgtttgtattattgaagtacaaagtggaatcaatacaaatgttggatagcaggggaacaatattcacaaaatggaatgggacatagtattaaacatagtgcttctgatgaccgtagaccatagacaatgcttaggatatgatatcacttcttttgttgttttttgtattttgagacgaagtctcattctgtcacccaggctggagttcagtggcgccatctcagctcactgcaacctccatctcccgggttcaagctattctccttcctcaacctcccgagtagctgggttgcgcaccaccatgcctggctaacttttgtatttttagtacagacggggtttcaccacgttggccaggctgctcttgaactcctgacgtcaggtgatccaccagccttgacctcccaaagtgctaggattacaggagccactgtacccagcctaggatatgatatcacttcttagagcaagatacaaaattgcatgtgcacaataattctaccaagtataggtatacaggggtagttatatataaatgagacttcaaggaaatacaacaaaatgcaatcgtgattgtgttagggtggtaagaaaacggtttttgctttgatgagctctgttttttaaaatcgttatattttctaataaaaatacatagtcttttgaaggaacataaaagattatgaagaaatgagttagatattgattcctattgaagattcagacaagtaaaattaaggggaaaaaaaacgggatgaaccagaagtcaggctggagttccaaccccagatccgacagcccaggctgatggggcctccagggcagtggtttccacccagcattctcaaaagagccactgaggtctcagtgccattttcaagatttcggaagcggcctgggcacggctggtccttcactgggatcaccacttggcaattatttacacctgagacgaatgaaaaccagagtgctgagattacaggcatggtggcttacgcttgtaatcggctttgggaagccgaggtgggctgattgcttgagcccaggagtttcaaactatcctggacaacatagcatgacctcgtctctacaaaaaatacaaaaaatttgccaggtgtggtggcatgtgcctgtggtcccagctacttgggaggctgaagtaggagaatcccctgagccctgggaagtcgaggctgcactgagccgtgatggtgtcactgcactccagcctgggtgacaaagtgagaccctatctcacaaagaaaaaaaacaaaacaaaaaacccaaagcacactgtttccactgtttccagagttcctgagaggaaaggtcaccgggtgaggaagacgttctcactgatctggcagagaaaatgtccagtttttccaactccctaaaccatggttttctatttcatagttcttaggcaaattggtaaaaatcatttctcatcaaaacgctgatattttcacacctccctggtgtctgcagaaagaaccttccagaaatgcagtcgtgggagacccatccaggccacccctgcttatggaagagctgagaaaaagccccacgggagcatttgctcagcttccgttacgcacctagtggcattgtgggtgggagagggctggtgggtggatggaaggagaaggcacagcccccccttgcagggacagagccctcgtacagaagggacaccccacatttgtcttccccacaaagcggcctgtgtcctgcctacggggtcagggcttctcaaacctggctgtgtgtcagaatcaccaggggaacttttcaaaactagagagactgaagccagactcctagattctaattctaggtcagggctaggggctgagattgtaaaaatccacaggtgattctgatgcccggcaggcttgagaacagccgcagggagttctctgggaatgtgccggtgggtctagccaggtgtgagtggagatgccggggaacttcctattactcactcgtcagtgtggccgaacacatttttcacttgacctcaggctggtgaacgctcccctctggggttcaggcctcacgatgccatccttttgtgaagtgaggacctgcaatcccagcttcgtaaagcccgctggaaatcactcacacttctgggatgccttcagagcagccctctatcccttcagctcccctgggatgtgactcgacctcccgtcactccccagactgcctctgccaagtccgaaagtggaggcatccttgcgagcaagtaggcgggtccagggtggcgcatgtcactcatcgaaagtggaggcgtccttgcgagcaagcaggcgggtccagggtggcgtgtcactcatccttttttctggctaccaaaggtgcagataattaataagaagctggatcttagcaacgtccagtccaagtgtggctcaaaggataatatcaaacacgtcccgggaggcggcagtgtgagtaccttcacacgtcccatgcgccgtgctgtggcttgaattattaggaagtggtgtgagtgcgtacacttgcgagacactgcatagaataaatccttcttgggctctcaggatctggctgcgacctctgggtgaatgtagcccggctccccacattcccccacacggtccactgttcccagaagccccttcctcatattctaggagggggtgtcccagcatttctgggtcccccagcctgcgcaggctgtgtggacagaatagggcagatgacggaccctctctccggaccctgcctgggaagctgagaatacccatcaaagtctccttccactcatgcccagccctgtccccaggagccccatagcccattggaagttgggctgaaggtggtggcacctgagactgggctgccgcctcctcccccgacacctgggcaggttgacgttgagtggctccactgtggacaggtgacccgtttgttctgatgagcggacaccaaggtcttactgtcctgctcagctgctgctcctacacgttcaaggcaggagccgattcctaagcctccagcttatgcttagcctgcgccaccctctggcagagactccagatgcaaagagccaaaccaaagtgcgacaggtccctctgcccagcgttgaggtgtggcagagaaatgctgcttttggcccttttagatttggctgcctcttgccaggagtggtggctcgtgcctgtaattccagcactttgggagactaaggcgggaggttcgcttgagcccaggagttcaagaccagcctgggcaacaatgagacccctgtgtctacaaaaagaattaaaattagccaggtgtggtggcacgcacctgtagtcccagctacttgggaggctgaggtgggaggattgcctgagtccgggaggcggaagttgcaaggagccatgatcgcgccactgcacttcaacctaggcaacagagtgagactttgtctcaaaaaacaatcatataataattttaaaataaatagatttggcttcctctaaatgtccccggggactccgtgcatcttctgtggagtgtctccgtgagattcgggactcagatcctcaagtgcaactgacccacccgataagctgaggcttcatcatcccctggccggtctatgtcgactgggcacccgaggctcctctcccaccagctctcttggtcagctgaaagcaaactgttaacaccctggggagctggacgtatgagacccttggggtgggaggcgttgatttttgagagcaatcacctggccctggctggcagtaccgggacactgctgtggctccggggtgggctgtctccagaaaatgcctggcctgaggcagccacccgcatccagcccagagggtttattcttgcaatgtgctgctgcttcctgccctgagcacctggatcccggcttctgccctgaggccccttgagtcccacaggtagcaagcgcttgccctgcggctgctgcatggggctaactaacgcttcctcaccagtgtctgctaagtgtctcctctgtctcccacgccctgctctcctgtccccccagtttgtctgctgtgaggggacagaagaggtgtgtgccgcccccacccctgcccgggcccttgttcctgggattgctgttttcagctgtttgagctttgatcctggttctctggcttcctcaaagtgagctcggccagaggaggaaggccatgtgctttctggttgaagtcaagtctggtgccctggtggaggctgtgctgctgaggcggagctggggagagagtgcacacgggctgcgtggccaacccctctgggtagctgatgcccaaagacgctgcagtgcccaggacatctgggacctccctggggcccgcccgtgtgtcccgcgctgtgttcatctgcgggctagcctgtgacccgcgctgtgctcgtctgcgggctagcctgtgtcccgcgctctgcttgtctgcggtctagcctgtgacctggcagagagccaccagatgtcccgggctgagcactgccctctgagcaccttcacaggaagcccttctcctggtgagaagagatgccagcccctggcatctgggggcactggatccctggcctgagccctagcctctccccagcctgggggccccttcccagcaggctggccctgctccttctctacctgggacccttctgcctcctggctggaccctggaagctctgcagggcctgctgtccccctccctgccctccaggtatcctgaccaccggccctggctcccactgccatccactcctctcctttctggccgttccctggtccctgtcccagcccccctccccctctcacgagttacctcacccaggccagagggaagagggaaggaggccctggtcataccagcacgtcctcccacctccctcggccctggtccaccccctcagtgctggcctcagagcacagctctctccaagccaggccgcgcgccatccatcctccctgtcccccaacgtccttgccacagatcatgtccgccctgacacacatgggtctcagccatctctgccccagttaactccccatccataaagagcacatgccagccgacaccaaaataattcgggatggttccagtttagacctaagtggaaggagaaaccaccacctgccctgcaccttgttttttggtgaccttgataaaccatcttcagccatgaagccagctgtctcccaggaagctccagggcggtgcttcctcgggagctgactgataggtgggaggtggctgcccccttgcaccctcaggtgaccccacacaaggccactgctggaggccctggggactccaggaatgtcaatcagtgacctgccccccaggccccacacagccatggctgcatagaggcctgcctccaagggacctgtctgtctgccactgtggagtccctacagcgtgccccccacaggggagctggttctttgactgagatcagctggcagctcagggtcatcattcccagagggagcggtgccctggaggccacaggcctcctcatgtgtgtctgcgtccgctcgagcttactgagacactaaatctgttggtttctgctgtgccacctacccaccctgttggtgttgctttgttcctattgctaaagacaggaatgtccaggacactgagtgtgcaggtgcctgctggttctcacgtccgagctgctgaactccgctgggtcctgcttactgatggtctttgctctagtgctttccagggtccgtggaagcttttcctggaataaagcccacgcatcgaccctcacagcgcctcccctctttgaggcccagcagataccccactcctgcctttccagcaagatttttcagatgctgtgcatactcatcatattgatcacttttttcttcatgcctgattgtgatctgtcaatttcatgtcaggaaagggagtgacatttttacacttaagcgtttgctgagcaaatgtctgggtcttgcacaatgacaatgggtccctgtttttcccagaggctcttttgttctgcagggattgaagacactccagtcccacagtccccagctcccctggggcagggttggcagaatttcgacaacacatttttccaccctgactaggatgtgctcctcatggcagctgggaaccactgtccaataagggcctgggcttacacagctgcttctcattgagttacacccttaataaaataatcccattttatcctttttgtctctctgtcttcctctctctctgcctttcctcttctctctcctcctctctcatctccaggtgcaaatagtctacaaaccagttgacctgagcaaggtgacctccaagtgtggctcattaggcaacatccatcataaaccaggtagccctgtggaaggtgagggttgggacgggagggtgcagggggtggaggagtcctggtgaggctggaactgctccagacttcagaaggggctggaaaggatattttaggtagacctacatcaaggaaagtgttgagtgtgaaacttgcgggagcccaggaggcgtggtggctccagctcgctcctgcccaggccatgctgcccaagacaaggtgaggcgggagtgaagtgaaataaggcaggcacagaaagaaagcacatattctcggccgggcgctgtggctcacgcctgtaattccagcactttgggaggccaaggtgggtggatcatgaggtcaggagattgagaccatcctggctaacacagtgaaaccccgtctctactaaaaatacaaaaaattagccgggcgtggtggtgggcgcctgtagtcccagctactccggaggctgaggcaggaaaatggcgtgaacccggaaggcggagcttgcagtgagcggagtgagcagagatcgcgccactgcactccagcctgggcgacagagcgagactccgtctcaaaaaaaaaaagcacatgttctcgcttctttgtgggatccaggagatagagaatagaaggatggttaccagaggctgggaagggtagtgaggggatggtggggggatggtcaatgggtacaaaaaaaatagaataagacctagtatttgatagtgcaacagggtgactatagtcaataataatttaattgtacatttaaaaataactaaaagatagccgggtgcagtggcttacgtctgtaatcccagtactttgggaggctgaggtgggcgtttgagaccagcctggccaacatggtgaaaccccatctctactaaaaatacaaaaattagccaggcatggtggcgggcgcctgtaatcccagctactcgggaggctgaggcaggagaatcacttgaacctgggaggcagaggttgcagtgagccgagatcttgccactgcactccagcctgggtgacagtgaaactccgtctcaaaaataaaaataaaaatacagctgggcacggtggctcacgcctgtaatcccagcactttgggaggccgaggcgagcggatcacaaggtcaggagatatagaccatcctggctaacacggtgaaacccggtctctactaaaaatacaaaaaattagccaggcgtggtggcaggtgcctatagtcccagctactcacaaggctgaggcaggagaatggcatgaacctgggaggcggagcttgcagtgagccgagattgtgccactgcactccagcctgggcgagagagtgagactccgtctcaaaacaaaaacaaaaacaaaaacaaaaacaaacacacaacaaaaacctaaaagaatataaatggattgtttgtaacacaaaggacaaatgtttgaggggatggataccccattttccatgatgtgattattatacattgtgtgtctgtatcaaaacatctcatgagccccataaatatatacacctaactatgtacccacaaaaattaaaaaaatatattttttaaggtgaagagggaggcgagatgctggccttaacccctaacccgttgttctccctgcaagctgtccacagggcctctcagactcgaggttcagctatatggatgcatgagcttggtccccagccaacatgggagacacttcaccatcggcagcagctacagcacaggaaccctgggtcactgccatgtcccctctgtgactttgtttaaacagaaaatgatgctctgggccggctgtggtggcccacacctataatcccagcaccttgggaggcgggggtgggcagattgcctgaggtcaggagttggagatcagcctggccgacatggcgaaaccccatgtctactaaaaatacaaaaactagccaggcatggtggcacatgcctgtaatcccagctacttgggaggctgaagcaggagaatcacttgaacccaggaggcagaggctgagtgagccaagatcgtgccaatgcactccagcttgggtgagggagtgagactccgtctcaaaaaaaaaaaaaaagaaagaaaaagaaaagaaagtgatcctactggaaccatgcttactcccctccccacctcacactgtgtagaaattagtgctgtcggccaggcgcggtggctcatgcctgtaatcgcagcactttgggaggccaaggcaggcggatcacgaggtcaggagatcaagaccatcctggctaacacagtgaaaccctgtctctactaaaaatacaaaaaattagccgggcatggtggcaggcacctgtagtcccaactacttgggaggctgaggcaggagaatggcatgaacctgggaggcggagcttgcagtgagccaagatcgcgccactgcataccagcctaggtgacagagtgagactcagcaaaaaaagaaagaaagaaagaaagaaatcagtgctgtctatacttctttctgcagtgatggaaatattctgtatctgtgctgtccagtatagtagccactagctacatgtggcacttgaaacatggctggtacagttgaggaagagtggctgccatatcggacgacacagctatagattctgtcaccccaccccgagagtccagagcggggacttctgccttaggccctattcagggctgatttttacttgaacccttactgtgggaagagaaggccatgagaagttcagtctagaatgtgactccttattttctggctcccttggacactttgtgggatttagtctccctgtggaaagtattccacaagtggtgccactaccccagctgtgagagcagctgggagctgcttttgtcatctttccctggaaagtcctgtgggctgtctcttcctcatgccttgtcccatgcttgggcatggtgtcaagcgtcaggagggagaaagggtccttatttatttatttagagagggacccttcttctgttcccaggctggagtgcagtggtgcgatctcggctcactgcaacctccgcctcctgggttcaagtgattctcctgcctcagcctcctgagtagctgagattacaggcacatgccaacatgcccggctaattttttttttttttttttttttttttttttttttttttttgagatggagttgtactctcattgcccaggctggaatgtaatggcacaatctcggctcactgcaacctccacctcctggattcaagcaattctcctgtctcagcttcccaagtagctgggattacaggtgcccgccaccatgctcaactaatttttgtattttttttttagtagagacgaggtttcaccatgttggtcagactggtctcaaactcctgacctcaggtgatccacctgcctcggcctcccaaagtgctaggattacaggcatgagccaccacgcccggcctgaaagggttcttatttagtgtgcattttgacattcaatttaattccaaggtcttgtggggtcatggtttacaggatgttgatatagaaaagacttcacttaatgggccgggcgcagtggctcatgcctgtaatcccagcactttgggaggccgaggcaggcagatcaggaggtcaggagattgagaccatcctggctaacacagtgaaaccccatctctactgaaaatacaaaaaattagctgggcgtggtggcaggcacctgtagtcccagccactcggttggctgaggcaggagaatggcatgaacccgggaggcggagcttgcagtgagcagagaccatgccactgcactccagcctgggcgacagagcaagactctgtctcaagaaaaaaaaaaaaaaacagactttacttactggaagccaaccaatgtatatttagagtaatttttcctgggctgagctgtcatttacttttgcagtatctcaagaagaagagtttacagtgtaaatatttgatgcacactttgattatatagatgaagcaaactattttcaagagctttgcaaggacttacttgtatccaaacaccattctaaaaggagtcttacctacttctaaaggctggtctctacttggaaccacttgcttggccctggttcaagtcctgctgcaaacctggaagtcctgtcattgtcttcttccctccagagcagtggcacccaatctaatttttgctgtgccccagcagcccctggcactttgccctgtagactgcagacctcatgtaatgtatgttaagtccacagaaccacagaagatgatggcaagatgctcttgtgtgtgttgtgttctaggaggtggccaggtggaagtaaaatctgagaagcttgacttcaaggacagagtccagtcgaagattgggtccctggacaatatcacccacgtccctggcggaggaaataaaaaggtaaagggggtagggtgggttggatgctgcccttgggtatatgggcattaatcaagttgagtggacaaaggctggtccagttcccagaggaggaaaacagaggcttctgtgttgactggctggatgtgggccctcagcagcatccagtgggtctccactgcctgtctcaatcacctggagctttagcacgtttcacacctgggccccaacctggagaggctgaccaatgggtctcaggggcagctcggttgctggagtttttgtttttatttatttttatgtatttaaggcagggtctctgtattagtccattctcacactgctaataaagacatacccaagactgggtaatttataaaggaaagaggtttaatggactcacagttccacatggctggggaggcctcaaaatcatggcggaaggcaaaggagaagcaaaggcatttcttacatggcgacaggcaagagagcgtgtgcaggggaactcccatttataaaaccatcagacctcatgagatttattcactatcatgagaacagcatgggaaagacccgcccccatgattcagttacctcccactgggtccctcccatgacacatggaattatgggagctacaattcaagatgagatttgggtggggacacagccaaaccatatcagtctccctctgtcatccaggctggagtgcactggcatgatctcggctcactgcagcctctacctccctgggtcaggtgatcttcccacctcagcctcccaggtagctggaactacaggtacctgccactatgcctggctaaatattttgtatttcctgtggagacgaggttttgccacgttgcccaggctggtcttgaactcctgaggtcaagcaatatgcccacctcggcctcccaaggtgctgggattacaggtgtgagccacagtgctcggcctaagtcactgcagtttttaaagctcccaggtgattcttcagtgcagtcaaaagtgagaactggctgggtgcggtggctcatgcctgtaatcccagcaccttgggaggcgaaggtgggcagatggcttgaggtcaggagttcaagaccagcctggccaacatggtaaaaccccatctctactaaaaatacaaaagttagctgggtgtggtggtgcgtgcctgtaatcccagctacttgggaggctgaggcatgagaattgcttgaacccaggggacagaggttgtagtgagccgagatcgtgccactgcactccagcctgggcaacagagtgagattccatctcacaaaaaaaaaaaaaagcgagaaccactgtcctaggccctgatgtttgcaggcaactaaaaaaggaagtggacatccccagtcagctgtggcgcaccaagaacaagtcatgggaacataacctaattttctaaatgggttactaggcacttagagcaaaacaatgatgccgaaatcctgatttcagcaaagcctctgcctgcctgtcttggaagtatccacatgaggctgctggggccttggtgtccccagcagtttctagtctctaggtcttgctgtgggtgtctgtgcagtgagggtgtgtgtggcgctgggtgagctctgtctaggcctggcacaggatgcggtctggtagctgctgcttctcttctgcagaagcgcagccaagcaccctctggggtttcaggcccacacccagcctgaagttctgggagtggctcactttccaaccttcagggtctcccagcagctgactggggagtggtggagggaaaagggattgtattagtccgttttcacgccgctgatgaagacatacccgatactgggcagtctaaaagatagaggtctgatggactcacagttccacgtgactggggaggcctgacaatcatggtggaaggtgaaaggcttgtctcacacggtggcagacaagagaaaagagcttgtgcaggggaactcccctttataaaaccatcagatctcgggagacttattcactatcatgagaacagcacgggaaagaccctcctctatgattcaattacctcccaccaggtccctcccacaacatgtaggaattgtgggaactacaattcaagatgacatttgggtggggacacagccaaaccatatcagggcgtcccagaaagggtatagggtctgagacccaagtcagcatgagaaagtatgcttctcatggtggcccagttgggtggaagtggcagccgggccgtctttccaccaggccactcaagtagcagctgagagacccctgccctggccagtccccgccctcccctcttgccactgcctctggttctgaacagatgggcaccctcatcttgtatttgtgattaatgtctaacaatgtagttttgtgagaagggtttgctgatacagccttgctgcagatgctgcgaactgtggcctggggcagaccttacctccagacacgccctgaggcaggggagggcactggcccgtagctggccgagagctctcgggttgcgcgacagggatacttttcagcggctgggtcgctatccaaagtgagaaaacgaggagggaccaggaggctgtccgcctcaagagatgtgggggccaggtccagttatctggggaagcagtaagcttctctgctgtttctaaccccaggcctcccctggtctaaggcagggcctcccagcctcggggcactttaaagatatctgggcctggccccatccccacagtctgactgagtgggtctggatagggcctgagcattggtgatttcctgggtgaaaggaggcccctcacagtctctggaagcttctctgtgttaggaaaagctctgggcttgactctgctttgaaagtcaagatccgcaaatcctctcagcctcagtttctccttcagcaagatgaaatggaaatgctgtacctacgtcccggggtggttgtgagacccaaaaaagacaatgttctggaaggttcctggtgcgttgcagtcctctaagaacctgagttagagccacgctgagtctcagcttcttggctccttctgtttcaaactcgtccatgtgatagctcaggaagggtaggcagggccctgccccctactcagaaaacaccatcctggtcctggggatccccgcagcattagtcccctgttttcccagtgtattgagaaaaattgctaacaagcagtggggcacaccaccagcctcctgggttcctttcagtttggggatttttggacattcccaggaatgtcttaaaaaacacttcaaaaaacattaacataaatatttttatcaaagcctgtattaaatggtctttcaagaaaatacagtaacaggtcaggcatggtggctcatgcctgtaaccccagcactttgggaggccaaggcaggcagatcacctgaaatcaggagttcaagaccaacctggccaacacagccaaatcccatctctacaaaaaatacaaaaattagctgggtgtggtggcacacacctgtagtcccagctacttgggaggccgaggcaggagaattgcttgatcccggaggcggaggttgcagtgagccgagatcgtgccactgcactccagcgtgggtgacaaggtgaatctttgtctcaaaaaaaaaaaaaaaaaaaagataaaatacagtatacagtaatagagaacaatccttttttcaaagtagtgaccccaaatgaacaaaatatgcatctagcttaaatgcgaacctggttttctctacgcccattcaagcccctgcaataggggcccttcaccccgcatccatggactcctaaaattatatggaaaatggctgtgtgtgagtgtggatggacatgtgcacacatatttttggctttaccagatgctcaaagagcctaggacccaaaaagggctgagaatgaccgtgtcggccacttcagggtcatcaggaattgctgtgcactgctcacttctccagtgaacactttctgcttctgtgtttcctggtatcctttgggactcctggctaggtcatgtgtttctctactttcaaaagggcttcagccaggcacgatggcatgagcctgtagtcccagttgctctggaggttaaggtgggaagattgcttgagcccaggaatttgaggccagcctgggcaagtagataggtagatgattgatagatagatagatagataaatagatggatagataagtcgctagacagtcatccatccacccatccacacataaaaaggcctttgtcatgtcatgttttgtggcccacctgccagtgttgcccacagttgctgcccctccaaactcatcagtcactggcaaacaggaggaatgtgtggctcatgtctgggcatcagtggctgtgggagacatccttgatcttctccagcttctccttccacattttcctttgcaatctggcaatatctattaaaataaaatgtgcatgccttttgacctaagagcttcacttctaggacccacttacacgtgtgtgacatgatgttcatacgggtttatttatctgaggttgttcatacacaccattgcctgtaatcactaaaggcgggagcagcctacacatccatccacagaggagtagatgccttttggtacatccgtggcgacggaatactaagcagcctgtgtatctatacactcacacgtgtttgtttatgtgtggaatatctctggagggtacacaagaaacttaaaatgatcactgtctctggggagggtacctgggtgcctgggaggcaggtcagggaaggagtgggcacaggtattaccaattggaagacaataaaaacaacagctcctggccaggcgcagtggctcacgcctgtaatggcagcactctgagaggctgaggcgggcagattgcttgcgtccaggagttcaagaccagcctgggcaacatagcaaaaccccgtttctattaaaaatacaaaaaattagccaggtgtggtggcatgcacctgtaatcccagctactcgggaggctgaggtgggagaatcacctgagcctgggaggtcaaggctgcagtgaggtgagattgtgccaccgcactctagcctgggcgatagagcaagaccctgtctcaaaaacaaacaaaaaacagtccctggcactctgggccaggcctggcagggcagttggcagggctggtctttctctggcacttcatctcaccctccctcccttcctcttcttgcagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcacccctgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtgggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcggcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaataaggtctccattcatggattccaaggacaagaaagtcatatagaatgtctattttttaagttctttcccacgcacccttagataatttagctcagaacaggaaatgatagtattaataaaagctggacatcaggattaacagctctctctggggccctgaaggtgagagttctcagacttgctcatttgcagttgcttctttgtgatgctggcaaaccatcctagtcccattcaaagggcaatacaaagccttgtggctgacctcacgatgcagcactcagtttgcaagaccggcaccagtgtatgcaaacctgagaaggttggggatgaggatatgggatctttcatccctggaaatttagtccagaggcctggggctggagcagaacaccaagccaatcagcttaatgaatggcttagattcctgctaggtttgcagagctgccttctttcctttggtaccttattatagattgaggagtatttctgctaaaccaagatagggataaccagatagcatcttcatagcaatgccacaaaggaaaacaaaaacaaaacagtaatccatcatattattccttagtaactatgccaaggtcatgatactgaatccttagattgtttcaaaatactacttttctttgctcttcctgatgtgtttgccaccgcaggcagatgtttaagtaaaacagattttaactgcagctacaaaagcagcaacaggccagcaaaagagaagtgctatctcagagagcatggctttcagagccacaagagacagcctcactggctgtttcagcttgactgccatgcaaagaagagagcagagggagaaccagccccacccacttattcatcttgtacaaaaaaaaagcacctaccagcctaggctacatagtgagacactatctccacaaaaaacccacgaaaactagctgggtatggtggcacatgcctacagtcccagctactggtaaggctgtggtgggaggatctcttgaggccaggaaggagatccaggctgcagtgagccaagattgcaccactgcactccagtctggacaatcgagcaagatcccatctcaaacaataaaaaaaaaaagcgtgtaacctcctcagaagaaagatgttataatctcaggcagcaggcaagaaccaatccaggctctaagcaaattatgtatctcactgaccccaccaaacctcagaaaaatttaacagtgagaagcaaaatctcctttaaagagcaacttagaacagatagaaaatatcatacagctgacttcactagagagaaagtgcatcaactgctttcactcaacaaaaagaaaaaagagatgatcaatgcagatcccctctcctcctggcagcccttaccctcagtgaaaagccaccaccattctctctctggtggccatcagatcaacctgcggcgttcccacaagacagaatggagattttccaaggtatagagcaagtcagagtaccccaaagaacggcggcagagagccagctccgaaactgccaacactaccatgcatacacagttcagtaagtcaagaaaggcctggtacacagcattctgtaacttttttttttatttttttcaatttttccttcttttttttttttaagcactagtctgtgctttgcgaacagaatcaagacattaacaaagatcagcttctctgaagaaaagcatttctatagaacaaagacagctacatgtttcgctgccattacacagctccaaagcaggaaaagaaaatatttacaaaatacaaggttttttttttccattttttgtttttgttttttttttcaatgctaaaagggttattcagaattttcaaccttataaatagaagaagcactttatgcatagggatatggtgcattattgtatttttttttaaagaaacaatgacaaaccctttaacttgcaaacagaaaaaaaaatcactaatgttgaaaattgtgaaaaaaccccaaccattaa

SEQ ID NO: 2 - DNA, 17 bp, Artificial Sequence
  ASO-002192
ccttaatttcaccctca

SEQ ID NO: 3 - DNA, 17 bp, Artificial Sequence
  ASO-002109
ccttaatttcaccctca

SEQ ID NO: 4 - DNA, 17 bp, Artificial Sequence
  ASO-002121
tcccttaatttcaccct

SEQ ID NO: 5 - DNA, 17 bp, Artificial Sequence
  ASO-002133
tcccttaatttcaccct

SEQ ID NO: 6 - DNA, 17 bp, Artificial Sequence
  ASO-002145
tcccttaatttcaccct

SEQ ID NO: 7 - DNA, 17 bp, Artificial Sequence
  ASO-002157
tcccttaatttcaccct

SEQ ID NO: 8 - DNA, 17 bp, Artificial Sequence
  ASO-002169
tcccttaatttcaccct

SEQ ID NO: 9 - DNA, 17 bp, Artificial Sequence
  ASO-002154
cccttaatttcaccctc

SEQ ID NO: 10 - DNA, 18 bp, Artificial Sequence
  ASO-002159
cccttaatttcaccctca

SEQ ID NO: 11 - DNA, 18 bp, Artificial Sequence
  ASO-002183
cccttaatttcaccctca

SEQ ID NO: 12 - DNA, 18 bp, Artificial Sequence
  ASO-002195
cccttaatttcaccctca

SEQ ID NO: 13 - DNA, 18 bp, Artificial Sequence
  ASO-002196
cccttaatttcaccctca

SEQ ID NO: 14 - DNA, 18 bp, Artificial Sequence
  ASO-002200
cccttaatttcaccctca

SEQ ID NO: 15 - DNA, 18 bp, Artificial Sequence
  ASO-002204
cccttaatttcaccctca

SEQ ID NO: 16 - DNA, 17 bp, Artificial Sequence
  ASO-002178
cccttaatttcaccctc

SEQ ID NO: 17 - DNA, 18 bp, Artificial Sequence
  ASO-002208
cccttaatttcaccctca

SEQ ID NO: 18 - DNA, 18 bp, Artificial Sequence
  ASO-002212
cccttaatttcaccctca

SEQ ID NO: 19 - DNA, 18 bp, Artificial Sequence
  ASO-002216
tcccttaatttcaccctc

SEQ ID NO: 20 - DNA, 18 bp, Artificial Sequence
  ASO-002220
tcccttaatttcaccctc

SEQ ID NO: 21 - DNA, 18 bp, Artificial Sequence
  ASO-002224
tcccttaatttcaccctc

SEQ ID NO: 22 - DNA, 18 bp, Artificial Sequence
  ASO-002197
tcccttaatttcaccctc

SEQ ID NO: 23 - DNA, 19 bp, Artificial Sequence
  ASO-002222
tcccttaatttcaccctca

SEQ ID NO: 24 - DNA, 19 bp, Artificial Sequence
  ASO-002226
tcccttaatttcaccctca

SEQ ID NO: 25 - DNA, 19 bp, Artificial Sequence
  ASO-002199
tcccttaatttcaccctca

SEQ ID NO: 26 - DNA, 19 bp, Artificial Sequence
  ASO-002203
tcccttaatttcaccctca

SEQ ID NO: 27 - DNA, 19 bp, Artificial Sequence
  ASO-002207
tcccttaatttcaccctca

SEQ ID NO: 28 - DNA, 19 bp, Artificial Sequence
  ASO-002211
tcccttaatttcaccctca

SEQ ID NO: 29 - DNA, 19 bp, Artificial Sequence
  ASO-002219
tcccttaatttcaccctca

SEQ ID NO: 30 - DNA, 17 bp, Artificial Sequence
  ASO-002119
cccttaatttcaccctc

SEQ ID NO: 31 - DNA, 17 bp, Artificial Sequence
  ASO-002131
cccttaatttcaccctc

SEQ ID NO: 32 - DNA, 17 bp, Artificial Sequence
  ASO-002382
ccttgatttcgccctca

SEQ ID NO: 33 - DNA, 17 bp, Artificial Sequence
  ASO-002299
ccttgatttcaccctct

SEQ ID NO: 34 - DNA, 17 bp, Artificial Sequence
  ASO-002311
ccttagtttcaccctcg

SEQ ID NO: 35 - DNA, 19 bp, Artificial Sequence
  ASO-002349
tcccttgatttcaccctca

SEQ ID NO: 36 - DNA, 19 bp, Artificial Sequence
  ASO-002361
tcccttaatttcaccctcg

SEQ ID NO: 37 - DNA, 19 bp, Artificial Sequence
  ASO-002373
acccttaatttcaccctca

SEQ ID NO: 38 - DNA, 19 bp, Artificial Sequence
  ASO-002385
tcccttgatttcgccctca

SEQ ID NO: 39 - DNA, 19 bp, Artificial Sequence
  ASO-002302
tcccttagtttcaccctcg

SEQ ID NO: 40 - DNA, 19 bp, Artificial Sequence
  ASO-002314
acccttgatttcaccctca

SEQ ID NO: 41 - DNA, 17 bp, Artificial Sequence
  ASO-002346
ccttgatttcaccctca

SEQ ID NO: 42 - DNA, 17 bp, Artificial Sequence
  ASO-002358
ccttagtttcaccctca

SEQ ID NO: 43 - DNA, 17 bp, Artificial Sequence
  ASO-002370
ccttaatttcaccctcg

SEQ ID NO: 44 - DNA, 17 bp, Artificial Sequence
  ASO-002155
cccttaatttcaccctc

SEQ ID NO: 45 - DNA, 17 bp, Artificial Sequence
  ASO-002179
cccttaatttcaccctc

SEQ ID NO: 46 - DNA, 17 bp, Artificial Sequence
  ASO-002191
ccttaatttcaccctca

SEQ ID NO: 47 - DNA, 17 bp, Artificial Sequence
  ASO-002120
ccttaatttcaccctca

SEQ ID NO: 48 - DNA, 17 bp, Artificial Sequence
  ASO-002132
ccttaatttcaccctca

SEQ ID NO: 49 - DNA, 17 bp, Artificial Sequence
  ASO-002144
ccttaatttcaccctca

SEQ ID NO: 50 - DNA, 17 bp, Artificial Sequence
  ASO-002156
ccttaatttcaccctca

SEQ ID NO: 51 - DNA, 17 bp, Artificial Sequence
  ASO-002168
ccttaatttcaccctca

SEQ ID NO: 52 - DNA, 18 bp, Artificial Sequence
  ASO-002201
tcccttaatttcaccctc

SEQ ID NO: 53 - DNA, 18 bp, Artificial Sequence
  ASO-002205
tcccttaatttcaccctc

SEQ ID NO: 54 - DNA, 18 bp, Artificial Sequence
  ASO-002209
tcccttaatttcaccctc

SEQ ID NO: 55 - DNA, 18 bp, Artificial Sequence
  ASO-002213
tcccttaatttcaccctc

SEQ ID NO: 56 - DNA, 17 bp, Artificial Sequence
  ASO-002190
cccttaatttcaccctc

SEQ ID NO: 57 - DNA, 18 bp, Artificial Sequence
  ASO-002217
tcccttaatttcaccctc

SEQ ID NO: 58 - DNA, 18 bp, Artificial Sequence
  ASO-002221
tcccttaatttcaccctc

SEQ ID NO: 59 - DNA, 18 bp, Artificial Sequence
  ASO-002225
tcccttaatttcaccctc

SEQ ID NO: 60 - DNA, 18 bp, Artificial Sequence
  ASO-002198
tcccttaatttcaccctc

SEQ ID NO: 61 - DNA, 18 bp, Artificial Sequence
  ASO-002202
tcccttaatttcaccctc

SEQ ID NO: 62 - DNA, 18 bp, Artificial Sequence
  ASO-002206
tcccttaatttcaccctc

SEQ ID NO: 63 - DNA, 19 bp, Artificial Sequence
  ASO-002210
tcccttaatttcaccctca

SEQ ID NO: 64 - DNA, 19 bp, Artificial Sequence
  ASO-002218
tcccttaatttcaccctca

SEQ ID NO: 65 - DNA, 18 bp, Artificial Sequence
  ASO-002372
tcccttgatttcaccctc

SEQ ID NO: 66 - DNA, 18 bp, Artificial Sequence
  ASO-002384
tcccttaatttcgccctc

SEQ ID NO: 67 - DNA, 18 bp, Artificial Sequence
  ASO-002301
tcccttagtttcaccctc

SEQ ID NO: 68 - DNA, 18 bp, Artificial Sequence
  ASO-002313
tcccttgatttcgccctc

SEQ ID NO: 69 - DNA, 18 bp, Artificial Sequence
  ASO-002325
tcccttagtttcgccctc

SEQ ID NO: 70 - DNA, 18 bp, Artificial Sequence
  ASO-002337
acccttgatttcaccctc

SEQ ID NO: 71 - DNA, 17 bp, Artificial Sequence
  ASO-002193
tcccttaatttcaccct

SEQ ID NO: 72 - DNA, 17 bp, Artificial Sequence
  ASO-002110
tcccttaatttcaccct

SEQ ID NO: 73 - DNA, 17 bp, Artificial Sequence
  ASO-002122
tcccttaatttcaccct

SEQ ID NO: 74 - DNA, 17 bp, Artificial Sequence
  ASO-002146
tcccttaatttcaccct

SEQ ID NO: 75 - DNA, 18 bp, Artificial Sequence
  ASO-002111
cccttaatttcaccctca

SEQ ID NO: 76 - DNA, 17 bp, Artificial Sequence
  ASO-002166
cccttaatttcaccctc

SEQ ID NO: 77 - DNA, 18 bp, Artificial Sequence
  ASO-002123
cccttaatttcaccctca

SEQ ID NO: 78 - DNA, 18 bp, Artificial Sequence
  ASO-002135
cccttaatttcaccctca

SEQ ID NO: 79 - DNA, 17 bp, Artificial Sequence
  ASO-002323
tcccttgatttcaccct

SEQ ID NO: 80 - DNA, 17 bp, Artificial Sequence
  ASO-002335
tcccttaatttcaccca

SEQ ID NO: 81 - DNA, 17 bp, Artificial Sequence
  ASO-002347
tcccttaatttcgccct

SEQ ID NO: 82 - DNA, 17 bp, Artificial Sequence
  ASO-002359
tcccttgatttcaccca

SEQ ID NO: 83 - DNA, 17 bp, Artificial Sequence
  ASO-002371
tcccttgatttcacccg

SEQ ID NO: 84 - DNA, 17 bp, Artificial Sequence
  ASO-002383
tcccttagtttcgccct

SEQ ID NO: 85 - DNA, 20 bp, Artificial Sequence
  ASO-002090
atttccaaattcacttttac

SEQ ID NO: 86 - DNA, 20 bp, Artificial Sequence
  ASO-002043
atttccaaattcacttttac

SEQ ID NO: 87 - DNA, 20 bp, Artificial Sequence
  ASO-002076
atttccaaattcacttttac

SEQ ID NO: 88 - DNA, 20 bp, Artificial Sequence
  ASO-002062
atttccaaattcacttttac

SEQ ID NO: 89 - DNA, 20 bp, Artificial Sequence
  ASO-002082
atttccaaattcacttttac

SEQ ID NO: 90 - DNA, 20 bp, Artificial Sequence
  ASO-001940
atttccaaattcacttttac

SEQ ID NO: 91 - DNA, 20 bp, Artificial Sequence
  ASO-001933
atttccaaattcacttttac

SEQ ID NO: 92 - DNA, 20 bp, Artificial Sequence
  ASO-002094
atttccaaattcacttttac

SEQ ID NO: 93 - DNA, 20 bp, Artificial Sequence
  ASO-002034
atttccaaattcacttttac

SEQ ID NO: 94 - DNA, 20 bp, Artificial Sequence
  ASO-002036
atttccaaattcacttttac

SEQ ID NO: 95 - DNA, 20 bp, Artificial Sequence
  ASO-002084
atttccaaattcacttttac

SEQ ID NO: 96 - DNA, 20 bp, Artificial Sequence
  ASO-002037
atttccaaattcacttttac

SEQ ID NO: 97 - DNA, 20 bp, Artificial Sequence
  ASO-002058
atttccaaattcacttttac

SEQ ID NO: 98 - DNA, 20 bp, Artificial Sequence
  ASO-002057
atttccaaattcacttttac

SEQ ID NO: 99 - DNA, 20 bp, Artificial Sequence
  ASO-002092
atttccaaattcacttttac

SEQ ID NO: 100 - DNA, 20 bp, Artificial Sequence
  ASO-002023
atttccaaattcacttttac

SEQ ID NO: 101 - DNA, 20 bp, Artificial Sequence
  ASO-002065
atttccaaattcacttttac

SEQ ID NO: 102 - DNA, 20 bp, Artificial Sequence
  ASO-002038
atttccaaattcacttttac

SEQ ID NO: 103 - DNA, 20 bp, Artificial Sequence
  ASO-002039
atttccaaattcacttttac

SEQ ID NO: 104 - DNA, 20 bp, Artificial Sequence
  ASO-002291
gtttccaaattcacttttac

SEQ ID NO: 105 - DNA, 20 bp, Artificial Sequence
  ASO-002303
atttccagattcacttttac

SEQ ID NO: 106 - DNA, 20 bp, Artificial Sequence
  ASO-002315
ttttccaaattcacttttac

SEQ ID NO: 107 - DNA, 20 bp, Artificial Sequence
  ASO-002327
gtttccagattcacttttac

SEQ ID NO: 108 - DNA, 20 bp, Artificial Sequence
  ASO-002339
atttccaagttcacttttgc

SEQ ID NO: 109 - DNA, 20 bp, Artificial Sequence
  ASO-002351
atttccagattcgcttttac

SEQ ID NO: 110 - DNA, 20 bp, Artificial Sequence
  ASO-002326
atttccaaattcacttttac

SEQ ID NO: 111 - DNA, 20 bp, Artificial Sequence
  ASO-002338
atttccaaattcacttttac

SEQ ID NO: 112 - DNA, 20 bp, Artificial Sequence
  ASO-002362
atttccaaattcacttttac

SEQ ID NO: 113 - DNA, 20 bp, Artificial Sequence
  ASO-002236
atttccaaattcacttttac

SEQ ID NO: 114 - DNA, 20 bp, Artificial Sequence
  ASO-002269
atttccaaattcacttttac

SEQ ID NO: 115 - DNA, 20 bp, Artificial Sequence
  ASO-002237
atttccaaattcacttttac

SEQ ID NO: 116 - DNA, 20 bp, Artificial Sequence
  ASO-002246
atttccaaattcacttttac

SEQ ID NO: 117 - DNA, 20 bp, Artificial Sequence
  ASO-002254
atttccaaattcacttttac

SEQ ID NO: 118 - DNA, 20 bp, Artificial Sequence
  ASO-002284
atttccaaattcacttttac

SEQ ID NO: 119 - DNA, 20 bp, Artificial Sequence
  ASO-002238
atttccaaattcacttttac

SEQ ID NO: 120 - DNA, 20 bp, Artificial Sequence
  ASO-002263
atttccaaattcacttttac

SEQ ID NO: 121 - DNA, 20 bp, Artificial Sequence
  ASO-002239
atttccaaattcacttttac

SEQ ID NO: 122 - DNA, 20 bp, Artificial Sequence
  ASO-002055
tatttccaaattcactttta

SEQ ID NO: 123 - DNA, 20 bp, Artificial Sequence
  ASO-002035
tatttccaaattcactttta

SEQ ID NO: 124 - DNA, 20 bp, Artificial Sequence
  ASO-002048
tatttccaaattcactttta

SEQ ID NO: 125 - DNA, 20 bp, Artificial Sequence
  ASO-002053
tatttccaaattcactttta

SEQ ID NO: 126 - DNA, 20 bp, Artificial Sequence
  ASO-002067
tatttccaaattcactttta

SEQ ID NO: 127 - DNA, 20 bp, Artificial Sequence
  ASO-002081
tatttccaaattcactttta

SEQ ID NO: 128 - DNA, 20 bp, Artificial Sequence
  ASO-001966
tatttccaaattcactttta

SEQ ID NO: 129 - DNA, 20 bp, Artificial Sequence
  ASO-002025
tatttccaaattcactttta

SEQ ID NO: 130 - DNA, 20 bp, Artificial Sequence
  ASO-002033
tatttccaaattcactttta

SEQ ID NO: 131 - DNA, 20 bp, Artificial Sequence
  ASO-001960
tatttccaaattcactttta

SEQ ID NO: 132 - DNA, 20 bp, Artificial Sequence
  ASO-002056
tatttccaaattcactttta

SEQ ID NO: 133 - DNA, 20 bp, Artificial Sequence
  ASO-002063
tatttccaaattcactttta

SEQ ID NO: 134 - DNA, 20 bp, Artificial Sequence
  ASO-002089
tatttccaaattcactttta

SEQ ID NO: 135 - DNA, 20 bp, Artificial Sequence
  ASO-002073
tatttccaaattcactttta

SEQ ID NO: 136 - DNA, 20 bp, Artificial Sequence
  ASO-002027
tatttccaaattcactttta

SEQ ID NO: 137 - DNA, 20 bp, Artificial Sequence
  ASO-002075
tatttccaaattcactttta

SEQ ID NO: 138 - DNA, 20 bp, Artificial Sequence
  ASO-002028
tatttccaaattcactttta

SEQ ID NO: 139 - DNA, 20 bp, Artificial Sequence
  ASO-002085
tatttccaaattcactttta

SEQ ID NO: 140 - DNA, 20 bp, Artificial Sequence
  ASO-002083
tatttccaaattcactttta

SEQ ID NO: 141 - DNA, 20 bp, Artificial Sequence
  ASO-002350
tatttccaaattcactttta

SEQ ID NO: 142 - DNA, 20 bp, Artificial Sequence
  ASO-002374
tatttccaaattcactttta

SEQ ID NO: 143 - DNA, 20 bp, Artificial Sequence
  ASO-002386
tatttccaaattcactttta

SEQ ID NO: 144 - DNA, 20 bp, Artificial Sequence
  ASO-002227
tatttccaaattcactttta

SEQ ID NO: 145 - DNA, 20 bp, Artificial Sequence
  ASO-002245
tatttccaaattcactttta

SEQ ID NO: 146 - DNA, 20 bp, Artificial Sequence
  ASO-002261
tatttccaaattcactttta

SEQ ID NO: 147 - DNA, 20 bp, Artificial Sequence
  ASO-002276
tatttccaaattcactttta

SEQ ID NO: 148 - DNA, 20 bp, Artificial Sequence
  ASO-002228
tatttccaaattcactttta

SEQ ID NO: 149 - DNA, 20 bp, Artificial Sequence
  ASO-002285
tatttccaaattcactttta

SEQ ID NO: 150 - DNA, 20 bp, Artificial Sequence
  ASO-002230
tatttccaaattcactttta

SEQ ID NO: 151 - DNA, 20 bp, Artificial Sequence
  ASO-002256
tatttccaaattcactttta

SEQ ID NO: 152 - DNA, 20 bp, Artificial Sequence
  ASO-002279
tatttccaaattcactttta

SEQ ID NO: 153 - DNA, 20 bp, Artificial Sequence
  ASO-002072
ttatttccaaattcactttt

SEQ ID NO: 154 - DNA, 20 bp, Artificial Sequence
  ASO-002071
ttatttccaaattcactttt

SEQ ID NO: 155 - DNA, 20 bp, Artificial Sequence
  ASO-002080
ttatttccaaattcactttt

SEQ ID NO: 156 - DNA, 20 bp, Artificial Sequence
  ASO-001941
ttatttccaaattcactttt

SEQ ID NO: 157 - DNA, 20 bp, Artificial Sequence
  ASO-002045
ttatttccaaattcactttt

SEQ ID NO: 158 - DNA, 20 bp, Artificial Sequence
  ASO-001934
ttatttccaaattcactttt

SEQ ID NO: 159 - DNA, 20 bp, Artificial Sequence
  ASO-002074
ttatttccaaattcactttt

SEQ ID NO: 160 - DNA, 20 bp, Artificial Sequence
  ASO-002093
ttatttccaaattcactttt

SEQ ID NO: 161 - DNA, 20 bp, Artificial Sequence
  ASO-002054
ttatttccaaattcactttt

SEQ ID NO: 162 - DNA, 20 bp, Artificial Sequence
  ASO-002091
ttatttccaaattcactttt

SEQ ID NO: 163 - DNA, 20 bp, Artificial Sequence
  ASO-002064
ttatttccaaattcactttt

SEQ ID NO: 164 - DNA, 20 bp, Artificial Sequence
  ASO-002066
ttatttccaaattcactttt

SEQ ID NO: 165 - DNA, 20 bp, Artificial Sequence
  ASO-002044
ttatttccaaattcactttt

SEQ ID NO: 166 - DNA, 20 bp, Artificial Sequence
  ASO-002047
ttatttccaaattcactttt

SEQ ID NO: 167 - DNA, 20 bp, Artificial Sequence
  ASO-002046
ttatttccaaattcactttt

SEQ ID NO: 168 - DNA, 20 bp, Artificial Sequence
  ASO-002317
atatttccaaattcactttt

SEQ ID NO: 169 - DNA, 20 bp, Artificial Sequence
  ASO-002329
ttatttccaaattcacttta

SEQ ID NO: 170 - DNA, 20 bp, Artificial Sequence
  ASO-002341
ttatttccaaattcactttg

SEQ ID NO: 171 - DNA, 20 bp, Artificial Sequence
  ASO-002353
atatttccagattcactttt

SEQ ID NO: 172 - DNA, 20 bp, Artificial Sequence
  ASO-002365
ttatttccaagttcactttc

SEQ ID NO: 173 - DNA, 20 bp, Artificial Sequence
  ASO-002377
ttatttccagattcgctttt

SEQ ID NO: 174 - DNA, 20 bp, Artificial Sequence
  ASO-002253
ttatttccaaattcactttt

SEQ ID NO: 175 - DNA, 20 bp, Artificial Sequence
  ASO-002283
ttatttccaaattcactttt

SEQ ID NO: 176 - DNA, 20 bp, Artificial Sequence
  ASO-002270
ttatttccaaattcactttt

SEQ ID NO: 177 - DNA, 20 bp, Artificial Sequence
  ASO-002277
ttatttccaaattcactttt

SEQ ID NO: 178 - DNA, 20 bp, Artificial Sequence
  ASO-002247
ttatttccaaattcactttt

SEQ ID NO: 179 - DNA, 20 bp, Artificial Sequence
  ASO-002271
ttatttccaaattcactttt

SEQ ID NO: 180 - DNA, 20 bp, Artificial Sequence
  ASO-002278
ttatttccaaattcactttt

SEQ ID NO: 181 - DNA, 20 bp, Artificial Sequence
  ASO-002248
ttatttccaaattcactttt

SEQ ID NO: 182 - DNA, 20 bp, Artificial Sequence
  ASO-002264
ttatttccaaattcactttt

SEQ ID NO: 183 - DNA, 20 bp, Artificial Sequence
  ASO-002272
ttatttccaaattcactttt

SEQ ID NO: 184 - DNA, 20 bp, Artificial Sequence
  ASO-002286
ttatttccaaattcactttt

SEQ ID NO: 185 - DNA, 20 bp, Artificial Sequence
  ASO-002231
ttatttccaaattcactttt

SEQ ID NO: 186 - DNA, 20 bp, Artificial Sequence
  ASO-002240
ttatttccaaattcactttt

SEQ ID NO: 187 - DNA, 25 bp, Artificial Sequence
  ASO-002020
actttatttccaaattcacttttac

SEQ ID NO: 188 - DNA, 20 bp, Artificial Sequence
  ASO-001967
tttatttccaaattcacttt

SEQ ID NO: 189 - DNA, 25 bp, Artificial Sequence
  ASO-002086
actttatttccaaattcacttttac

SEQ ID NO: 190 - DNA, 25 bp, Artificial Sequence
  ASO-002029
actttatttccaaattcacttttac

SEQ ID NO: 191 - DNA, 20 bp, Artificial Sequence
  ASO-001961
tttatttccaaattcacttt

SEQ ID NO: 192 - DNA, 25 bp, Artificial Sequence
  ASO-002095
actttatttccaaattcacttttac

SEQ ID NO: 193 - DNA, 25 bp, Artificial Sequence
  ASO-002021
actttatttccaaattcacttttac

SEQ ID NO: 194 - DNA, 25 bp, Artificial Sequence
  ASO-002068
actttatttccaaattcacttttac

SEQ ID NO: 195 - DNA, 20 bp, Artificial Sequence
  ASO-002294
attatttccaaattcacttt

SEQ ID NO: 196 - DNA, 20 bp, Artificial Sequence
  ASO-002306
tttatttccaagttcacttt

SEQ ID NO: 197 - DNA, 20 bp, Artificial Sequence
  ASO-002318
gttatttccaaattcacttt

SEQ ID NO: 198 - DNA, 20 bp, Artificial Sequence
  ASO-002330
attatttccagattcacttt

SEQ ID NO: 199 - DNA, 20 bp, Artificial Sequence
  ASO-002342
tttatttccaggttcacttt

SEQ ID NO: 200 - DNA, 20 bp, Artificial Sequence
  ASO-002354
cttatttccaagttcacttt

SEQ ID NO: 201 - DNA, 20 bp, Artificial Sequence
  ASO-002006
ctttatttccaaattcactt

SEQ ID NO: 202 - DNA, 20 bp, Artificial Sequence
  ASO-002017
ctttatttccaaattcactt

SEQ ID NO: 203 - DNA, 20 bp, Artificial Sequence
  ASO-001942
ctttatttccaaattcactt

SEQ ID NO: 204 - DNA, 20 bp, Artificial Sequence
  ASO-001935
ctttatttccaaattcactt

SEQ ID NO: 205 - DNA, 20 bp, Artificial Sequence
  ASO-002002
ctttatttccaaattcactt

SEQ ID NO: 206 - DNA, 20 bp, Artificial Sequence
  ASO-002010
ctttatttccaaattcactt

SEQ ID NO: 207 - DNA, 20 bp, Artificial Sequence
  ASO-002005
ctttatttccaaattcactt

SEQ ID NO: 208 - DNA, 20 bp, Artificial Sequence
  ASO-001998
ctttatttccaaattcactt

SEQ ID NO: 209 - DNA, 20 bp, Artificial Sequence
  ASO-001994
ctttatttccaaattcactt

SEQ ID NO: 210 - DNA, 20 bp, Artificial Sequence
  ASO-002013
ctttatttccaaattcactt

SEQ ID NO: 211 - DNA, 20 bp, Artificial Sequence
  ASO-002009
ctttatttccaaattcactt

SEQ ID NO: 212 - DNA, 20 bp, Artificial Sequence
  ASO-002363
ctttatttccagattcactt

SEQ ID NO: 213 - DNA, 20 bp, Artificial Sequence
  ASO-002375
ctttatttccaaattcactg

SEQ ID NO: 214 - DNA, 20 bp, Artificial Sequence
  ASO-002292
ctttatttccaaattcgctt

SEQ ID NO: 215 - DNA, 20 bp, Artificial Sequence
  ASO-002304
ctttatttccagattcacta

SEQ ID NO: 216 - DNA, 20 bp, Artificial Sequence
  ASO-002316
ctttatttccaggttcactt

SEQ ID NO: 217 - DNA, 20 bp, Artificial Sequence
  ASO-002328
ctttatttccgagttcactt

SEQ ID NO: 218 - DNA, 19 bp, Artificial Sequence
  ASO-002004
ctttatttccaaattcact

SEQ ID NO: 219 - DNA, 19 bp, Artificial Sequence
  ASO-002012
ctttatttccaaattcact

SEQ ID NO: 220 - DNA, 19 bp, Artificial Sequence
  ASO-001987
ctttatttccaaattcact

SEQ ID NO: 221 - DNA, 19 bp, Artificial Sequence
  ASO-001992
ctttatttccaaattcact

SEQ ID NO: 222 - DNA, 19 bp, Artificial Sequence
  ASO-002000
ctttatttccaaattcact

SEQ ID NO: 223 - DNA, 19 bp, Artificial Sequence
  ASO-001996
ctttatttccaaattcact

SEQ ID NO: 224 - DNA, 19 bp, Artificial Sequence
  ASO-002008
ctttatttccaaattcact

SEQ ID NO: 225 - DNA, 19 bp, Artificial Sequence
  ASO-002015
ctttatttccaaattcact

SEQ ID NO: 226 - DNA, 19 bp, Artificial Sequence
  ASO-002016
ctttatttccaaattcact

SEQ ID NO: 227 - DNA, 18 bp, Artificial Sequence
  ASO-002011
ctttatttccaaattcac

SEQ ID NO: 228 - DNA, 18 bp, Artificial Sequence
  ASO-001988
ctttatttccaaattcac

SEQ ID NO: 229 - DNA, 18 bp, Artificial Sequence
  ASO-001993
ctttatttccaaattcac

SEQ ID NO: 230 - DNA, 18 bp, Artificial Sequence
  ASO-001997
ctttatttccaaattcac

SEQ ID NO: 231 - DNA, 18 bp, Artificial Sequence
  ASO-002320
ctttatttccagattcac

SEQ ID NO: 232 - DNA, 18 bp, Artificial Sequence
  ASO-002332
ctttatttccgaattcac

SEQ ID NO: 233 - DNA, 18 bp, Artificial Sequence
  ASO-002344
ctttgtttccaaattcac

SEQ ID NO: 234 - DNA, 18 bp, Artificial Sequence
  ASO-002356
ctttgtttccagattcac

SEQ ID NO: 235 - DNA, 18 bp, Artificial Sequence
  ASO-002368
ctttatttccaggttcac

SEQ ID NO: 236 - DNA, 18 bp, Artificial Sequence
  ASO-002380
ctttgtttccaagttcac

SEQ ID NO: 237 - DNA, 20 bp, Artificial Sequence
  ASO-002022
ttatttccaaattcactttt

SEQ ID NO: 238 - DNA, 20 bp, Artificial Sequence
  ASO-002026
ttatttccaaattcactttt

SEQ ID NO: 239 - DNA, 20 bp, Artificial Sequence
  ASO-002024
ttatttccaaattcactttt

SEQ ID NO: 240 - DNA, 25 bp, Artificial Sequence
  ASO-002049
actttatttccaaattcacttttac

SEQ ID NO: 241 - DNA, 25 bp, Artificial Sequence
  ASO-002019
actttatttccaaattcacttttac

SEQ ID NO: 242 - DNA, 17 bp, Artificial Sequence
  ASO-002640
ccttaatttcaccctca

SEQ ID NO: 243 - DNA, 17 bp, Artificial Sequence
  ASO-002632
ccttaatttcaccctca

SEQ ID NO: 244 - DNA, 17 bp, Artificial Sequence
  ASO-002647
ccttaatttcaccctca

SEQ ID NO: 245 - DNA, 20 bp, Artificial Sequence
  ASO-002666
atttccaaattcacttttac

SEQ ID NO: 246 - DNA, 20 bp, Artificial Sequence
  ASO-002659
atttccaaattcacttttac

SEQ ID NO: 247 - DNA, 20 bp, Artificial Sequence
  ASO-002652
atttccaaattcacttttac

SEQ ID NO: 248 - DNA, 20 bp, Artificial Sequence
  ASO-002645
atttccaaattcacttttac

SEQ ID NO: 249 - DNA, 20 bp, Artificial Sequence
  ASO-002638
atttccaaattcacttttac

SEQ ID NO: 250 - DNA, 20 bp, Artificial Sequence
  ASO-003270
atttccaaattcacttttac

SEQ ID NO: 251 - DNA, 20 bp, Artificial Sequence
  ASO-003269
atttccaaattcacttttac

SEQ ID NO: 252 - DNA, 20 bp, Artificial Sequence
  ASO-003268
atttccaaattcacttttac

SEQ ID NO: 253 - DNA, 20 bp, Artificial Sequence
  ASO-002673
atttccaaattcacttttac

SEQ ID NO: 254 - DNA, 20 bp, Artificial Sequence
  ASO-002661
tttatttccaaattcacttt

SEQ ID NO: 255 - DNA, 20 bp, Artificial Sequence
  ASO-002654
tttatttccaaattcacttt

SEQ ID NO: 256 - DNA, 20 bp, Artificial Sequence
  ASO-002668
tttatttccaaattcacttt

SEQ ID NO: 257 - DNA, 17 bp, Artificial Sequence
  ASO-002658
ccttaatttcaccctca

SEQ ID NO: 258 - DNA, 20 bp, Artificial Sequence
  ASO-002629
atttccaaattcacttttac

SEQ ID NO: 259 - DNA, 20 bp, Artificial Sequence
  ASO-002621
atttccaaattcacttttac

SEQ ID NO: 260 - DNA, 20 bp, Artificial Sequence
  ASO-002665
tttatttccaaattcacttt

SEQ ID NO: 261 - DNA, 20 bp, Artificial Sequence
  ASO-002399
atttccaaattcacttttac

SEQ ID NO: 262 - DNA, 20 bp, Artificial Sequence
  ASO-002482
atttccaaattcacttttac

SEQ ID NO: 263 - DNA, 20 bp, Artificial Sequence
  ASO-002437
atttccaaattcacttttac

SEQ ID NO: 264 - DNA, 20 bp, Artificial Sequence
  ASO-002425
atttccaaattcacttttac

SEQ ID NO: 265 - DNA, 17 bp, Artificial Sequence
  ASO-002170
tcccttaatttcaccct

SEQ ID NO: 266 - DNA, 20 bp, Artificial Sequence
  ASO-001989
ctttatttccaaattcactt

SEQ ID NO: 267 - DNA, 25 bp, Artificial Sequence
  RhoA
actttatttccaaatacacttcttt

SEQ ID NO: 268 - DNA, 20 bp, Artificial Sequence
  ASO-000757
ctttatttccaaattcactt

SEQ ID NO: 269 - DNA, 20 bp, Artificial Sequence
  ASO-0001967
tttatttccaaattcacttt

SEQ ID NO: 270 - DNA, 20 bp, Artificial Sequence
  ASO-000755
ttatttccaaattcactttt

SEQ ID NO: 271 - DNA, 20 bp, Artificial Sequence
  ASO-000753
atttccaaattcacttttac

SEQ ID NO: 272 - DNA, 20 bp, Artificial Sequence
  ASO-001936
ccagcgtgatcttccatcac

SEQ ID NO: 273 - DNA, 20 bp, Artificial Sequence
  ASO-001937
tcccagcgtgatcttccatc

SEQ ID NO: 274 - DNA, 20 bp, Artificial Sequence
  ASO-001943
ccagcgtgatcttccatcac

SEQ ID NO: 275 - DNA, 20 bp, Artificial Sequence
  ASO-001944
tcccagcgtgatcttccatc

SEQ ID NO: 276 - DNA, 20 bp, Artificial Sequence
  ASO-001963
cccagcgtgatcttccatca

SEQ ID NO: 277 - DNA, 20 bp, Artificial Sequence
  ASO-001964
cgtcccagcgtgatcttcca

SEQ ID NO: 278 - DNA, 20 bp, Artificial Sequence
  ASO-001969
cccagcgtgatcttccatca

SEQ ID NO: 279 - DNA, 20 bp, Artificial Sequence
  ASO-001970
cgtcccagcgtgatcttcca

SEQ ID NO: 280 - DNA, 16 bp, Artificial Sequence
  ASO-002100
catcacttcgaactcc

SEQ ID NO: 281 - DNA, 16 bp, Artificial Sequence
  ASO-002101
ccatcacttcgaactc

SEQ ID NO: 282 - DNA, 17 bp, Artificial Sequence
  ASO-002102
catcacttcgaactcct

SEQ ID NO: 283 - DNA, 17 bp, Artificial Sequence
  ASO-002103
tccatcacttcgaactc

SEQ ID NO: 284 - DNA, 18 bp, Artificial Sequence
  ASO-002104
cttccatcacttcgaact

SEQ ID NO: 285 - DNA, 20 bp, Artificial Sequence
  ASO-002105
cttccatcacttcgaactcc

SEQ ID NO: 286 - DNA, 22 bp, Artificial Sequence
  ASO-002106
tcttccatcacttcgaactcct

SEQ ID NO: 287 - DNA, 16 bp, Artificial Sequence
  ASO-002112
catcacttcgaactcc

SEQ ID NO: 288 - DNA, 16 bp, Artificial Sequence
  ASO-002113
ccatcacttcgaactc

SEQ ID NO: 289 - DNA, 17 bp, Artificial Sequence
  ASO-002114
ccatcacttcgaactcc

SEQ ID NO: 290 - DNA, 17 bp, Artificial Sequence
  ASO-002115
tccatcacttcgaactc

SEQ ID NO: 291 - DNA, 18 bp, Artificial Sequence
  ASO-002116
tccatcacttcgaactcc

SEQ ID NO: 292 - DNA, 16 bp, Artificial Sequence
  ASO-002125
ccatcacttcgaactc

SEQ ID NO: 293 - DNA, 17 bp, Artificial Sequence
  ASO-002126
ccatcacttcgaactcc

SEQ ID NO: 294 - DNA, 18 bp, Artificial Sequence
  ASO-002128
tccatcacttcgaactcc

SEQ ID NO: 295 - DNA, 20 bp, Artificial Sequence
  ASO-002129
tcttccatcacttcgaactc

SEQ ID NO: 296 - DNA, 16 bp, Artificial Sequence
  ASO-002136
catcacttcgaactcc

SEQ ID NO: 297 - DNA, 16 bp, Artificial Sequence
  ASO-002137
ccatcacttcgaactc

SEQ ID NO: 298 - DNA, 17 bp, Artificial Sequence
  ASO-002138
ccatcacttcgaactcc

SEQ ID NO: 299 - DNA, 18 bp, Artificial Sequence
  ASO-002139
ccatcacttcgaactcct

SEQ ID NO: 300 - DNA, 20 bp, Artificial Sequence
  ASO-002141
tcttccatcacttcgaactc

SEQ ID NO: 301 - DNA, 16 bp, Artificial Sequence
  ASO-002149
ccatcacttcgaactc

SEQ ID NO: 302 - DNA, 17 bp, Artificial Sequence
  ASO-002150
ccatcacttcgaactcc

SEQ ID NO: 303 - DNA, 18 bp, Artificial Sequence
  ASO-002151
ccatcacttcgaactcct

SEQ ID NO: 304 - DNA, 20 bp, Artificial Sequence
  ASO-002153
ttccatcacttcgaactcct

SEQ ID NO: 305 - DNA, 16 bp, Artificial Sequence
  ASO-002160
catcacttcgaactcc

SEQ ID NO: 306 - DNA, 17 bp, Artificial Sequence
  ASO-002161
catcacttcgaactcct

SEQ ID NO: 307 - DNA, 18 bp, Artificial Sequence
  ASO-002164
ttccatcacttcgaactc

SEQ ID NO: 308 - DNA, 20 bp, Artificial Sequence
  ASO-002165
ttccatcacttcgaactcct

SEQ ID NO: 309 - DNA, 16 bp, Artificial Sequence
  ASO-002172
catcacttcgaactcc

SEQ ID NO: 310 - DNA, 17 bp, Artificial Sequence
  ASO-002173
catcacttcgaactcct

SEQ ID NO: 311 - DNA, 17 bp, Artificial Sequence
  ASO-002174
tccatcacttcgaactc

SEQ ID NO: 312 - DNA, 18 bp, Artificial Sequence
  ASO-002176
ttccatcacttcgaactc

SEQ ID NO: 313 - DNA, 20 bp, Artificial Sequence
  ASO-002177
ttccatcacttcgaactcct

SEQ ID NO: 314 - DNA, 16 bp, Artificial Sequence
  ASO-002184
ccatcacttcgaactc

SEQ ID NO: 315 - DNA, 17 bp, Artificial Sequence
  ASO-002185
catcacttcgaactcct

SEQ ID NO: 316 - DNA, 17 bp, Artificial Sequence
  ASO-002186
tccatcacttcgaactc

SEQ ID NO: 317 - DNA, 18 bp, Artificial Sequence
  ASO-002187
cttccatcacttcgaact

SEQ ID NO: 318 - DNA, 20 bp, Artificial Sequence
  ASO-001978
tcccagcgtgatcttccatc

SEQ ID NO: 319 - DNA, 20 bp, Artificial Sequence
  ASO-257283
atttccaaattcacttttac

SEQ ID NO: 320 - DNA, 20 bp, Artificial Sequence
  ASO-257284
atttccaaattcacttttac

SEQ ID NO: 321 - DNA, 16 bp, Artificial Sequence
  ASO-002627
ccaaattcacttttac

SEQ ID NO: 322 - DNA, 17 bp, Artificial Sequence
  ASO-002677
tccaaattcacttttac

SEQ ID NO: 323 - DNA, 18 bp, Artificial Sequence
  ASO-002670
ttccaaattcacttttac

SEQ ID NO: 324 - DNA, 19 bp, Artificial Sequence
  ASO-002663
tttccaaattcacttttac

SEQ ID NO: 325 - DNA, 15 bp, Artificial Sequence
  ASO-002635
caaattcacttttac

SEQ ID NO: 326 - DNA, 18 bp, Artificial Sequence
  ASO-002643
tttccaaattcactttta

SEQ ID NO: 327 - DNA, 16 bp, Artificial Sequence
  ASO-002671
tccaaattcactttta

SEQ ID NO: 328 - DNA, 17 bp, Artificial Sequence
  ASO-002664
ttccaaattcactttta

SEQ ID NO: 329 - DNA, 19 bp, Artificial Sequence
  ASO-002626
atttccaaattcactttta

SEQ ID NO: 330 - DNA, 18 bp, Artificial Sequence
  ASO-002634
atttccaaattcactttt

SEQ ID NO: 331-333 - skipped (3 entries)

SEQ ID NO: 334 - DNA, 17 bp, Artificial Sequence
  ASO-002642
atttccaaattcacttt

SEQ ID NO: 335 - DNA, 16 bp, Artificial Sequence
  ASO-002649
atttccaaattcactt

SEQ ID NO: 336 - DNA, 15 bp, Artificial Sequence
  ASO-002656
atttccaaattcact

SEQ ID NO: 337 - DNA, 20 bp, Artificial Sequence
  Synthesized nucleotide sequence
tatttccaaattcactttta

SEQ ID NO: 338 - DNA, 20 bp, Artificial Sequence
  ASO-002255
tatttccaaattcactttta

SEQ ID NO: 339 - DNA, 20 bp, Artificial Sequence
  Forward Primer
caagctcgcatggtcagtaa

SEQ ID NO: 340 - DNA, 43 bp, Artificial Sequence
  Reverse Primer
aattaaccctcactaaagggagattctcagtggagccgatctt